(19) United States
(12) Patent Application Publication
(10) Pub. No.: US 2020/0299306 A1
(43) Pub. Date: Sep. 24, 2020

(54) COMPOUNDS WITH A BENZO[A]CARBAZOLE STRUCTURE AND USE THEREOF

(71) Applicant: UNIVERSITA' DI PISA, Pisa (IT)

(72) Inventors: Armando ROSSELLO, Pisa (IT); Elisa NUTI, Pisa (IT); Elisabetta ORLANDINI, Pisa (IT); Susanna NENCETTI, Pisa (IT); Claudia MARTINI, Pisa (IT); Barbara COSTA, Pisa (IT); Chiara GIACOMELLI, Pisa (IT); Simona DANIELE, Pisa (IT)

(21) Appl. No.: 16/645,267

(22) PCT Filed: Sep. 4, 2018
(86) PCT No.: PCT/IB2018/056737

§ 371 Date: Mar. 6, 2020

(30) Foreign Application Priority Data
Sep. 7, 2017 (IT) 102017000100116

(51) Int. Cl. C07D 501/18; C07D 495/04; C07D 209/94; C07D 513/04; C07D 491/052; C07D 471/04; C07D 519/00

(52) U.S. Cl. CPC C07D 501/18; C07D 495/04; C07D 209/94; C07D 519/00; C07D 491/052; C07D 471/04; C07D 513/04

(57) ABSTRACT

Disclosed are compounds of general formula (I), and their use for the treatment and diagnosis of degenerative disorders characterized by high cell proliferation and/or tissue degeneration.

### TECHNICAL FIELD OF INVENTION

The invention relates to compounds with a benzo[a]carbazole structure which act as apoptosis modulators, and their use for therapeutic and diagnostic purposes.

### PRIOR ART

The immortality of cancer cells associated with loss of life/death control mechanisms such as apoptosis regulation is a common stage in tumours. The mitochondria play an important role in regulating apoptosis, and the key stages in said event are increased permeability of the outer membrane of the mitochondria, mediated by opening of the mitochondrial permeability transition pore (MPTP), and release of death-inducing soluble protein factors. Tumour-suppressor protein p53 promotes apoptosis at mitochondrial level through opening of the MPTP. P53 is a transcription factor that controls the cell response to stress by inducing arrest of the cell cycle or apoptosis. Another protein, called Murine Double Minute 2 (MDM2), inhibits the activity of p53 by binding to the transactivation domain of p53 and preventing its transcription activity. In response to stress, phosphorylation of p53 reduces its affinity for MDM2 and activates p53. It has been observed in various human tumours that overexpression of MDM2 inhibits the p53 pathway, leading to uncontrolled cell proliferation. Inhibition of the p53/MDM2 interaction therefore represents a potential therapeutic target for cancer treatment. More recently, the role of p53 and MDM2/MDMX has been correlated with the development of infectious disorders mediated by bacteria, viruses and protozoa, and in some cases also with the pro-carcinogenic role of such types of infections involving this type of oncogenic pathway [Gonzalez, E. et al., Nat. Commun. 2014, 5, 5201, doi: 10.1038/ncomms6201; Sato, T et al, Rev. Med. Virol. 2013; 23: 213-220; Kaushansky, A et al, Cell Rep. 2013, 28, 3, 3, 630-637]. The MDM2/p53 complex consists of three critical p53 residues: Phe19, Trp23 and Leu26, which are inserted in a deep hydrophobic pocket on the surface of MDM2 [Riedinger, C et al. JACS 2008, 130, 16038-16044].

The more recent literature on synthetic ligands which act as MDM2-p53 interaction antagonists refers to some heterocyclic structures that constitute a central scaffold able to interact with the previously described binding region on MDM2 that recognises p53. The structural role of the central heterocyclic core of MDM2-p53 interaction inhibitors which have been developed to date is basically designed to reinforce the interaction between the synthetic molecules designed and developed and said subpockets identified on MDM2, so as to obtain powerful p53-antagonist ligands on said site. The heterocyclic cores most extensively studied in this field include type A (4,5-dihydro-1H-imidazoles, nucleus present in nutlin a and the analogues thereof), type B (3,4-dihydro-1H-benzo[e][1,4]diazepin-2,5-diones, which are present, for example, in TPD222669), type C (indolyl-2-ones, as in MI43), and type D (chromenotriazolopyrimidines); while as regards the substituents introduced to decorate said nuclei for the appropriate interactions with the above-mentioned p53 subsites, various types of substituents are able to give both hydrophobic and hydrogen-bond interactions (Scheme 1).

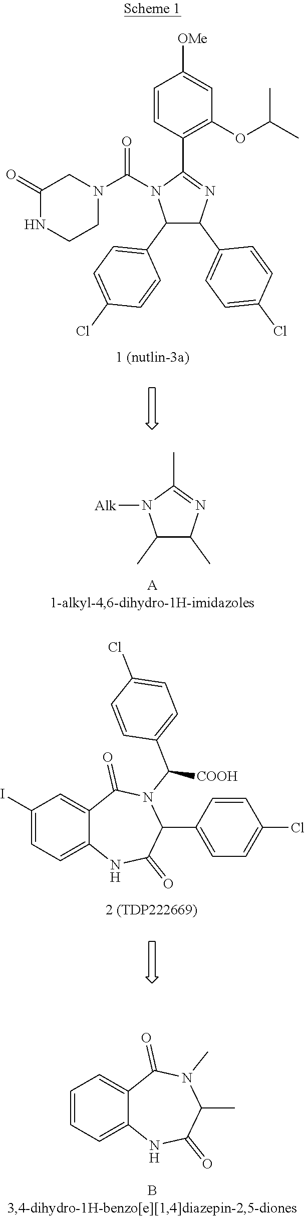

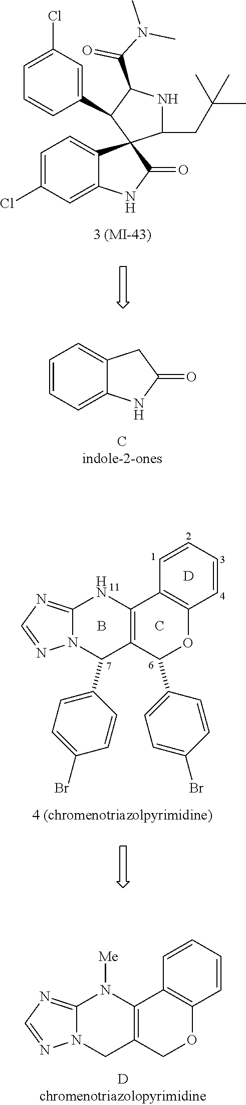

Said central cores exhibit some structural analogies with a heterocyclic nucleus of type E (carbazole, Scheme 2), and by analogy with nutlins A and spirooxindoles C could be suitable for decoration to interact with said MDM2 binding region [for a recent review see: Zhao Y. et al, J Med Chem J. Med. Chem. 2015, 58, 1038-1052]. In practice, some heterocyclic structures containing carbazole nuclei for antibacterial activity [A. Rossello et al, Il Farmaco, 51, 75 (1996)] had already exhibited some cytotoxic properties in cell tests on tumour lines.

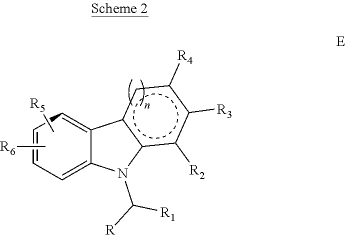

There is therefore still a need to identify new compounds usable for therapeutic and diagnostic purposes in the field of tumours.

### LIST OF FIGURES

FIG. 1 shows the stabilisation of p53 in the presence of RM37 or Nut-3.

FIG. 2 shows the effect of RM37 and Nut-3 on activation of the gene transactivation function of p53.

FIG. 3 shows the effects of RM37 on stabilisation of the intracellular levels of protein p5.

FIG. 4 shows the in vitro antitumoral effect of RM37 compared with Nut-3 (dead cell count).

FIG. 5 shows the in vitro antitumoral effect of RM37 compared with Nut-3 (live cell count).

FIG. 6 shows the effect of RM37 on the cell cycle.

FIG. 7 and FIG. 8 show the effect of RM37 on cell apoptosis.

### SUMMARY OF THE INVENTION

The present invention relates to compounds of general formula (I):

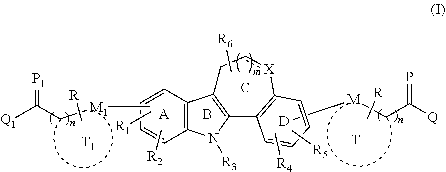

as defined in the detailed description below.

The present invention also relates to the use of the compounds with general formula (I) for the treatment and diagnosis of degenerative disorders characterised by high cell proliferation and/or tissue degeneration.

### DETAILED DESCRIPTION OF THE INVENTION

It has now surprisingly been found that compounds of general formula (I) as hereinafter described, which are functionalisable on their central carbazole core, can be used not only as therapeutic agents but also as diagnostic probes in endogenous or exogenous degenerative disorders (ie. those induced by infectious agents such as bacteria, viruses, protozoa or fungi) characterised by high cell proliferation and/or tissue degeneration.

The compounds of the invention have no endogenous or exogenous protease-inhibiting activities, such as the ability to inhibit activity on bacterial peptidases (e.g. bacterial transpeptidases and beta-lactamases), and have no modulating activity on steroid receptors. Their action takes place solely on the cell cycle, by modulating the functions of oncogenic proteins. Another aspect is modulation of the tumour-suppressor functions of genes, such as that of oncogenic protein p53 or its mutated forms, and of their modulators. The compounds of the invention modulate, for therapeutic purposes, the processes of arrest of the cell cycle and apoptosis in degenerative cells characterised by alteration of the gene array or by oncogenic effects due to sequestration of control proteins; in the latter case, for example, characterised by the MDM2-p53 or MDMX-p53 interaction. For example, the compounds can bind MDM2 or MDMX and modulate the activity of p53; this can lead to a control of the cell cycle with activating/inhibiting effects on proliferation and/or programmed cell life/death.

As regards the diagnostic aspect, the compounds of the invention comprise one or more labelled residues with one or more residues for imaging, by means of which they can specifically target cells characterised by high proliferation in degenerative tissues or infectious tissues generated by pathogens (bacteria, viruses, protozoa or fungi) and characteristic of all pathological forms associated with oncogenic imbalance. The disorders which can be treated or diagnosed with the compounds of the invention are cancers of various kinds, and other disorders such as infectious diseases caused by pathogens wherein physiological homeostatic tissue control has been lost, and control of proliferative activity and cell apoptosis is important.

The subject of the present invention is compounds of general formula (I):

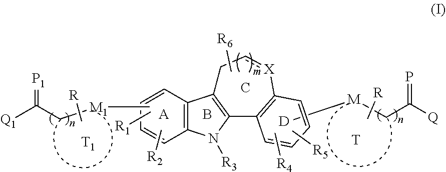

wherein

n is an integer between 0 and 12;

m is an integer between 0 and 2;

the phenyl rings A and D condensed in tetracyclic system A-D can be optionally substituted (ring A only, ring D only or both), in any of the substitutable positions, with the chain of formula II or II′:

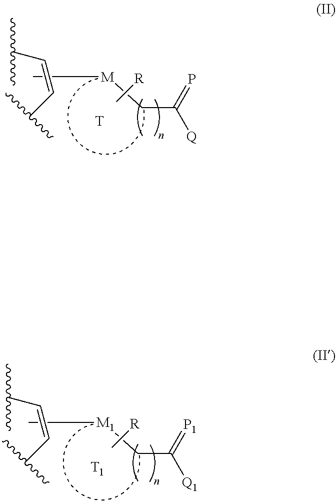

The chain of formula II can consist of a natural or non-natural amino acid, which may be the linear type of formula III (with T absent) or the cyclic type of formula IV (with T present); or of a peptide sequence, containing natural and/or non-natural amino acids type (III) and (IV), of formula (V); or of a peptide sequence of formula (VI) terminating with an amino acid of type (III) or (IV); or of a sequence of formula (VII) or (VIII):

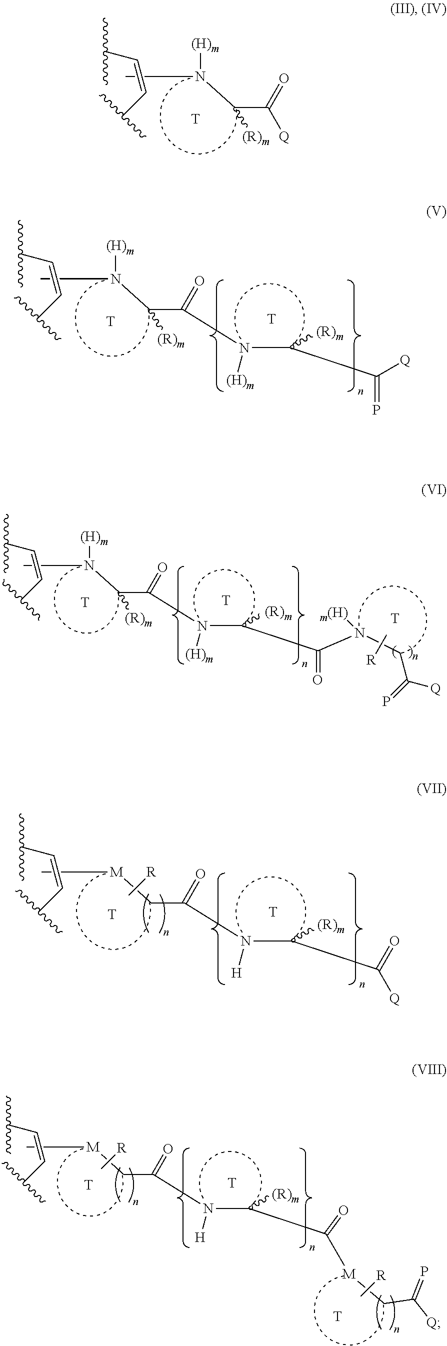

R is selected from the group consisting of hydrogen or an R₁ group or a G group wherein G can be a carbon atom, which may be halogenated or polyhalogenated with F or Cl or Br or I atoms, or also a carbon atom of a monocyclic or bicyclic aryl, or a carbon or nitrogen atom belonging to an aromatic or non-aromatic heterocyclic system selected from the group comprising pyrrole, pyrrolidine, 3-pyrroline, 2H-pyrrole, 2-pyrroline, indole, isoindole, 3H-indole, indolizine, indoline, furan, benzofuran, isobenzofuran, 2H-pyran, 4H-pyran, benzo[b]thiophene, thiophene, pyridine, piperidine, 4H-quinolizine, isoquinoline, quinolines, tetrahydroquinoline, 1,8-naphthyridine, acridine, oxazole, isoxazole, benzoxazole, benzothiazole, isothiazole, thiazole, imidazole, 2-imidazole, imidazolidine, tetrazole, 1,2,3-triazole, 1,2,4-triazole, 1,2,3-oxadiazole, benzoimidazole, purine, 1,4-dioxane, 1,3-dioxolane, 1,3-dithiane, 1,4-dithiane, 1,3,5-trithiane, morpholine, thiomorpholine, phenothiazine, pyrazole, 2-pyrazoline, pyrazolidine, quinazoline, cinnoline, pyrimidine, pyrazine, pteridine, phthalazine, 1,2,4-triazine, 1,3,5-triazine, pyridazine, piperazine, quinoxaline, phenazine and 1H-indazole;

R can also be a chain of saturated or unsaturated, straight or branched C₁-C₁₀ carbon atoms optionally substituted with a substituent selected from R₁, G, a hydroxyl, —O-alkyl, —ONO₂, —O-G, phenyl, substituted phenyl, heteroaryl, substituted heteroaryl, —NH-G, —NG₂, —NR₇R₈, ═N—O-G, —NH—O-G, —COOH, —(CH₂)ₚ—COOH, —(CHR₂)ₚ—COOH, —(CH₂)ₚ—CO—NHR′, —(CHR₂)ₚ—CO—NHR′, wherein p can be a number between 0 and 12 and wherein R₇ and R₈ are selected independently from hydrogen, all the meanings of R₁, and all the meanings of R₂;

or R is a chain of saturated or unsaturated, straight or branched C₁-C₆ carbon atoms optionally substituted with aryl, —CO-alkyl, —CO-aryl, —CO-heteroaryl; —CONH— alkyl, —CONH-alkyl-ONO₂; —CONH-acyl; —CONH-acyl-ONO₂, —CONH-aryl, —SO₂-alkyl, —SO₂NH₂, —SO₂NH-alkyl, —SO₂NH-aryl, wherein aryl can be phenyl, substituted phenyl, heteroaryl or substituted heteroaryl, and wherein R′ is selected from alkyl, alkyl-O—NO₂, aryl, acyl and acylaryl, wherein aryl can be phenyl, substituted phenyl, heteroaryl or substituted heteroaryl or as indicated in the meanings of G, when m=2 there are 2R groups and there can be two substituents which are the same or different, selected from the meanings of R; in some of said substitutions the two R groups can jointly constitute a cycle whose closing is the carbon bearing R, on the proviso that when m is 2, T is absent;

R₁ is selected from H, F, Cl, Br, I, -aryl, -heteroaryl, —R, —NO₂, —NH₂, —NHCH₃, —NH-alkyl, —NH-aryl, —NH-heteroaryl, —NH—R, —N(R)₂, —NRR₁, —N(CH₂)₂, —N(CH₂)₃, —N(CH₂)₄, —N(CH₂)₅, N(CH₂)₄O, —N(CH₂)₄S(O)ₘ, —N(CH₂)₄N—R, —N(CH₂)₄N—R₁, —NHCOCH₃, —NHCO-alkyl, —NH—CO-cycloalkyl, —NHCO-aryl, —NHCO-heteroaryl, —NHCO—R, —NHSO₂CH₃, —NHSO₂-alkyl, —NHSO₂-cycloalkyl, —NHSO₂-aryl, —NHSO₂-heteroaryl, —NHSO₂—R, —SCH₃, —SR, —SO₂CH₃, —SO₂-alkyl, —SO₂-cycloalkyl, —SO₂-aryl, —SO₂-heteroaryl, —SO₂—R, —SO₂NH₂, —SO₂NHCH₃, —SO₂NH-alkyl, —SO₂NH-cycloalkyl, —SO₂NH-aryl, —SO₂NH-heteroaryl, —SO₂NH—R, —SO₂NHCOCH₃, —SO₂NHCO-alkyl, —SO₂NHCO-aryl, —SO₂NHCO-heteroaryl, —SO₂NHCO-cycloalkyl, —C(CH)₄N (e.g. 2-pyridine, 3-pyridine, 4-pyridine), —C(CH)₃O (e.g. 2-furan, 3-furan), —C(CH)₃S (e.g. 2-thiophene, 3-thiophene), —CH(CH₂)O (e.g. oxirane), —CH(CH₂)S (e.g. thiirane), —CO₂H, —CO₂CH₃, —CO₂-alkyl, —CO₂-aryl, —CO₂-heteroaryl, —CO₂-cycloalkyl, —CO₂R, —CONHCH₃, —CONH-alkyl, —CONH-aryl, —CONH-heteroaryl, —CONH-cycloalkyl, —CONH—R, —CONRCH₃, —CONRR, —CONHSO₂CH₃, —CONHSO₂-alkyl, —O—CH₂-alkyl, —O—(CH₂)ₙ—R, —O-acyl, —O-aryl, —O-heteroaryl, —O-cycloalkyl, —O—R, —O—CH₂OCH₃, —O—CH₂OCH₂CH₃, —O—CH₂(OCH₂CH₂)ₙ—OCH₃, —O—(CH₂)ₙ—NH-alkyl, —O—(CH₂)ₙ—N-(alkyl)₂, —O—(CH₂)ₙ—NH— cycloalkyl with cycle from 4 to 6 carbon atoms, —O—(CH₂)ₙ—R with group R as defined above, —O—CH₂(NHCH₂CH₂)ₙ—OCH₃, —O—CH₂(CH₂)ₙCO(NHCH₂CH₂)ₙ—OCH₃, —O—CH₂(CH₂)ₙSO₂(NHCH₂CH₂)ₙ—OCH₃, —N(CH₂CH₂)₂N—(CH₂CH₂)ₙ—NH, CH₃N(CH₂CH₂)₂N—(CH₂CH₂)ₙ—, CH₃(CH₂)ₙCO—N(CH₂CH₂)₂N—(CH₂CH₂)ₙ, CH₃(CH₂)ₙSO₂—N(CH₂CH₂)₂N—(CH₂CH₂)ₙ—, O(CH₂CH₂)₂N—(CH₂CH₂)ₙ—, a monocyclic or bicyclic aryl, or an aromatic or non-aromatic heterocyclic system selected from the meanings of R;

M is selected from O, CH₂, CH—R₂, C═CH—R₂, C(R₂)ₘ, —CH—OH, —CH—O—R₂, C═CH—O—R₂, C═O, C═ONR₂, NH, N—R₂, N—OH, N—O—R₂, NR₂CO—, S, S═O, S(═O)₂, wherein R₂ is independently selected from the meanings of R, or R₁, R₂, R₃, R₄, R₅, R₆, R₇ and R₈;

M₁ is independently selected from the meanings of M, as defined above, or is absent;

R₂, R₃, R₄, R₅, R₆, R₇ and R₈ are equal or different and independently selected from the meanings of R, or R′, R₁;

P is selected from O, Q, CH₂, CH—R₂, C—(R₂)ₘ, CH—OH, CH—O—R₂, CH—O-acyl, NH, N-acyl, N—R₂, N—OH, N—O—R₂ and N—O-acyl, wherein R₂ is independently selected from the meanings of R, or R′, R₁, R₂, R₃, R₄, R₅, R₆, R₇ and R₈;

P₁ is independently selected from the meanings of P, as defined above, or is absent;

Q is selected from H, R₂, O—R₂, O-acyl, NH₂, NH—R₂, N—(R₂)ₘ, NH-acyl, NHCOO—CH₂Bn, NHCOO-t-Bu, NHCOO—R, NH—OH, NH—O—R₂, N(R₂)—O—R₂ and NH—O-acyl, wherein R₂ is independently selected from the meanings of R, or R′, R″, R₁, R₂, R₃, R₄, R₅, R₆, R₇ and R₈; or Q is a beta-lactam structure of formula (IX′), (IX″), (IX′″):

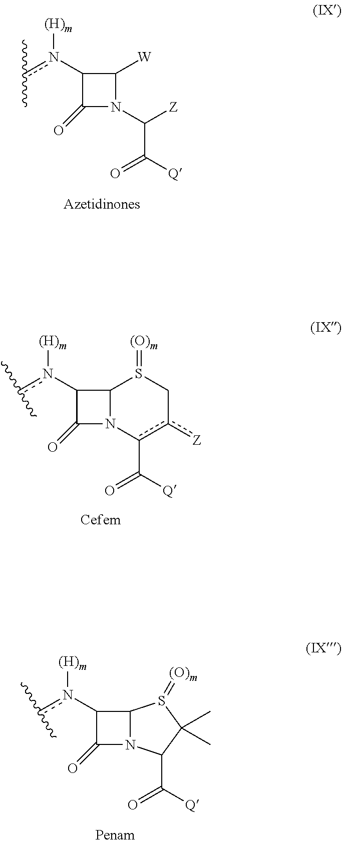

wherein Q′ is selected from H, R₂, O—R₂, O-acyl, NH₂, NH—R₂, N—(R₂)ₘ, NH-acyl, NHCOO—CH₂Bn, NHCOO-t-Bu, NHCOO—R, NH—OH, NH—O—R₂, N(R₂)—O—R₂ and NH—O-acyl, wherein R₂ is independently selected from the meanings of R, or R′, R″, R₁, R₂, R₃, R₄, R₅, R₆, R₇ and R₈;

Q₁ is independently selected from the meanings of Q, as defined above, or is absent;

T is a cyclic ring which is saturated or contains unsaturations and consists of 3 to 8 atoms bonded to one another and containing carbon atoms and/or N, O or S(O)ₘ, atoms, or substituted as in the meanings by R, R′, R″, R₁, R₂, R₃, R₄, R₅, R₆, R₇ and R₈ or by X, M, P, W o Z or as explained in the meanings of the cyclic systems of G for R and/or R₁;

T₁ is independently selected from the meanings of T, as defined above, or is absent;

X is selected from the —CH₂—, —CH(R″)—, ═C(R″)—, —O—, —S(═O)ₘ—, ═N—, —N(R″)—, —C(═O)—, —C(═P)—, —N(R″)CO, —CON(R″)— groups, wherein R″ is H or can represent one of the meanings defined for R, R′ and R₁, R₂, R₃, R₄, R₅, R₆, R₇ and R₈;

W is selected from H, or the meanings of T or of Z or of R, or also of R′, R″, R₁, R₂, R₃, R₄, R₅, R₆, R₇ and R₈;

Z is selected from H, CH₃, CH₂OCOCH₃ or, taken together with the carbon atom to which it is bonded, Z is selected from ═CH₂, ═CH(R₂), C(═O), C(═P); or Z is selected from the meanings of R, R′, R″, R₁, R₂, R₃, R₄, R₅, R₆, R₇ and R₈, or the meanings of M, P.

According to one embodiment, the compounds of general formula (I) are characterised by a substructure of formula (X), or by a substructure deriving from the substructure of formula (X), having formula (XI) or (XII):

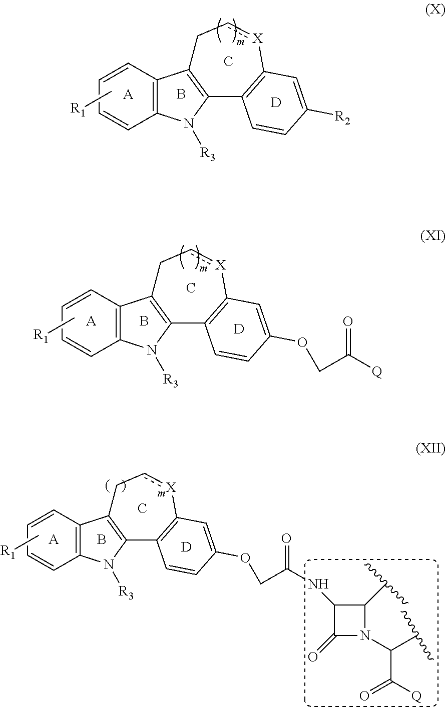

wherein

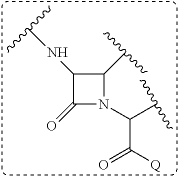

is a substituent of formula IX′, IX″ or IX

wherein m, X, R₁, R₂ and R₃ are as defined above.

According to a further embodiment, the compounds of formula (XII) can have formula (XIII), (XIV) or (XV):

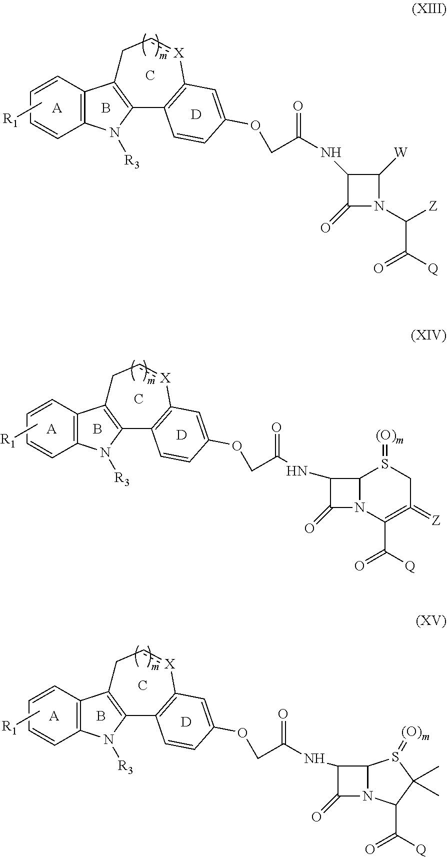

wherein m, X, R₁ and R₃ are as defined above.

According to a preferred aspect of the invention, the compounds of formula (X) can have a formula (Xa-Xt), as reported below:

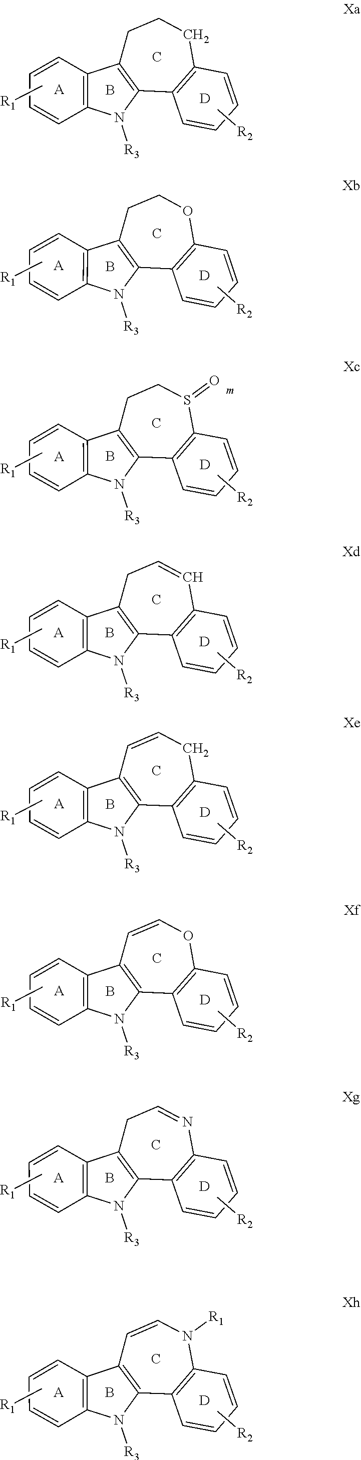

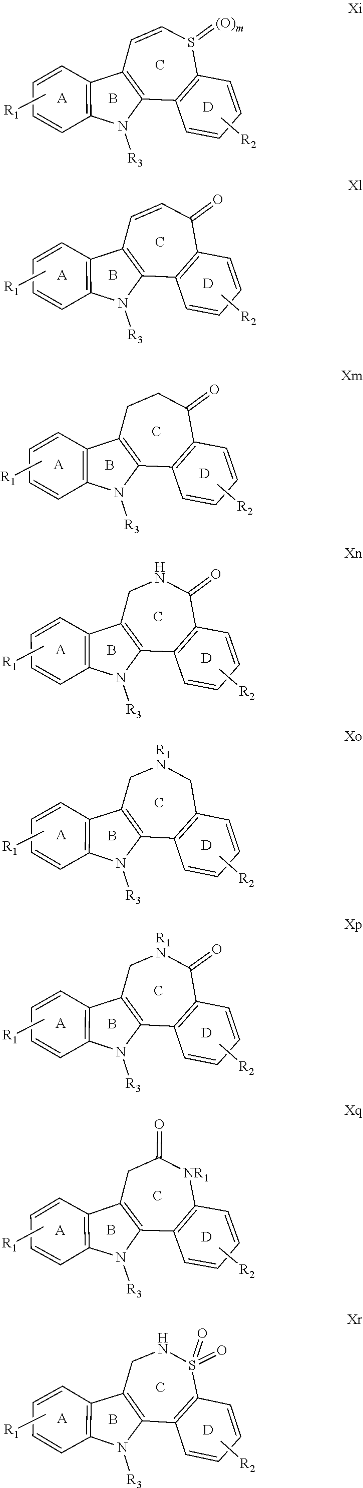

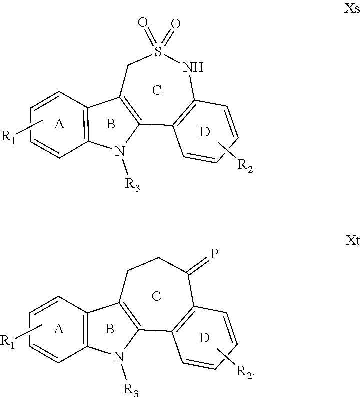

or (X′a-X′m), as reported below:

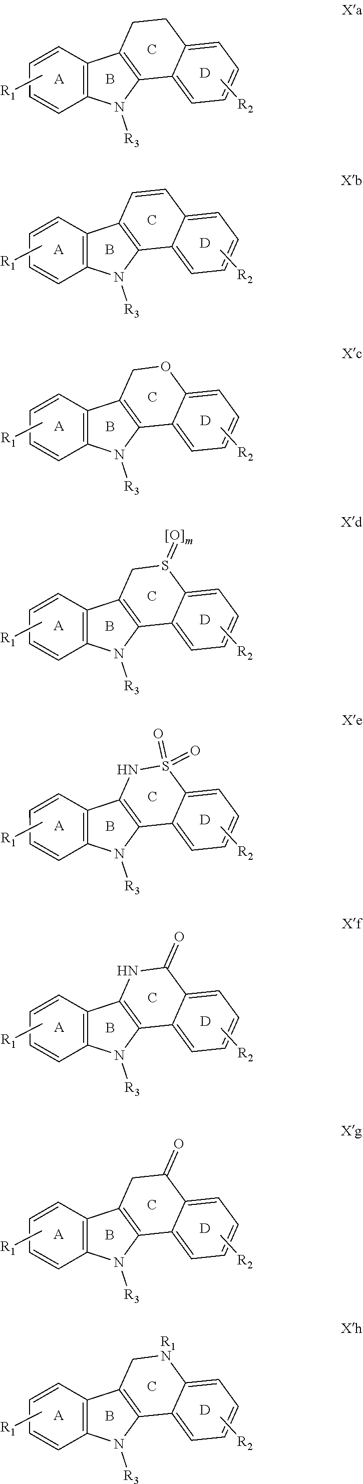

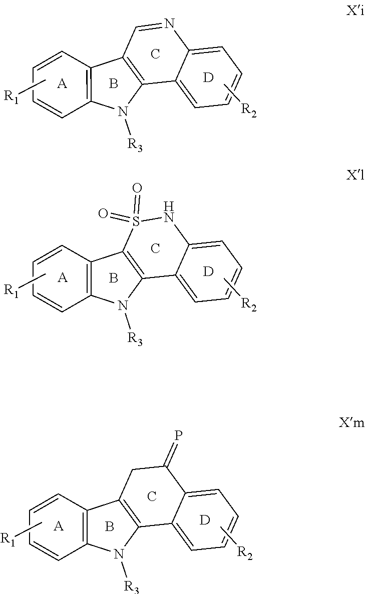

wherein m, P, R₁, R₂ and R₃ are as defined above.

According to a further preferred aspect of the invention, the compounds of formula (XI) can have formula (XIa-XIt) or (XI′a-XI′m), as reported below:

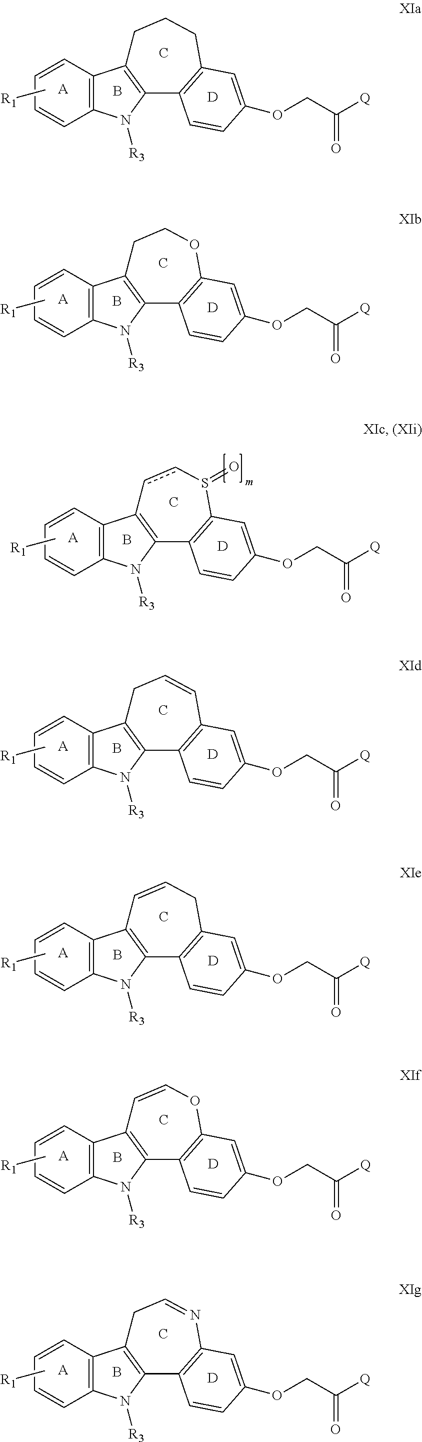

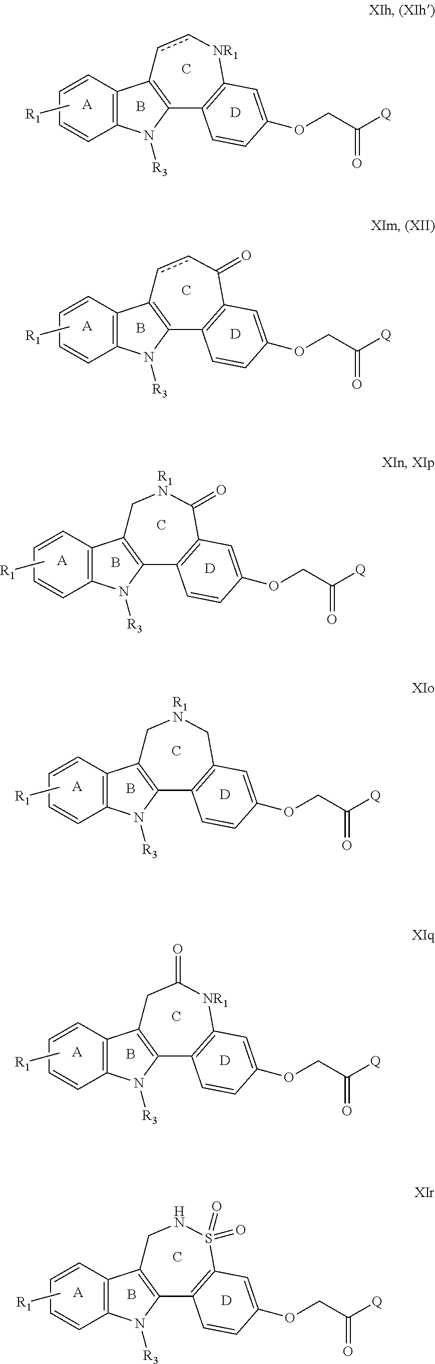

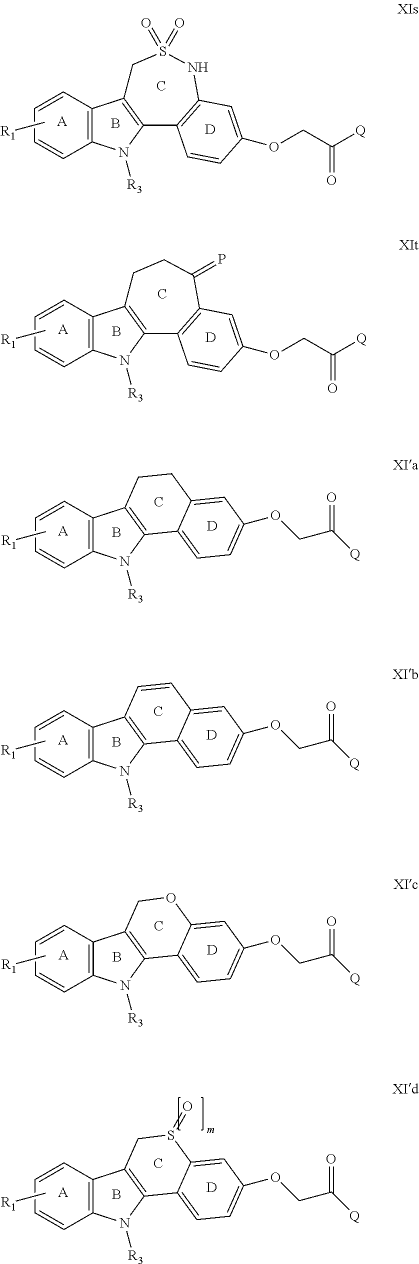

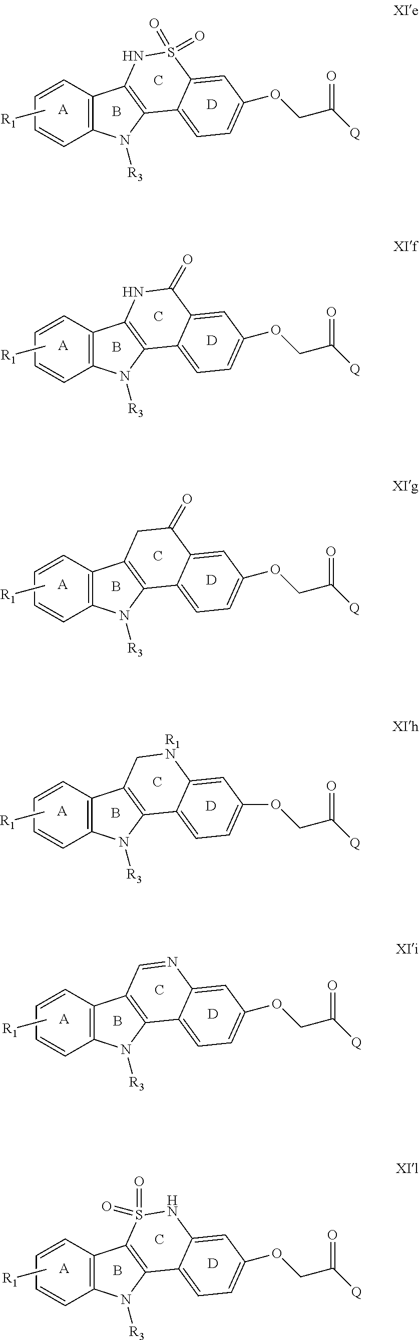

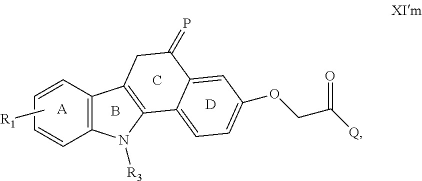

wherein m, P, R₁ and R₃ are as defined above.

If Q is an azetidinone nucleus not substituted with the T ring as in formula (XIII), there can be structures of formula (XIIIa-XIIIt), (XIII′a-XIII′m):

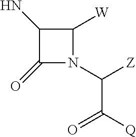

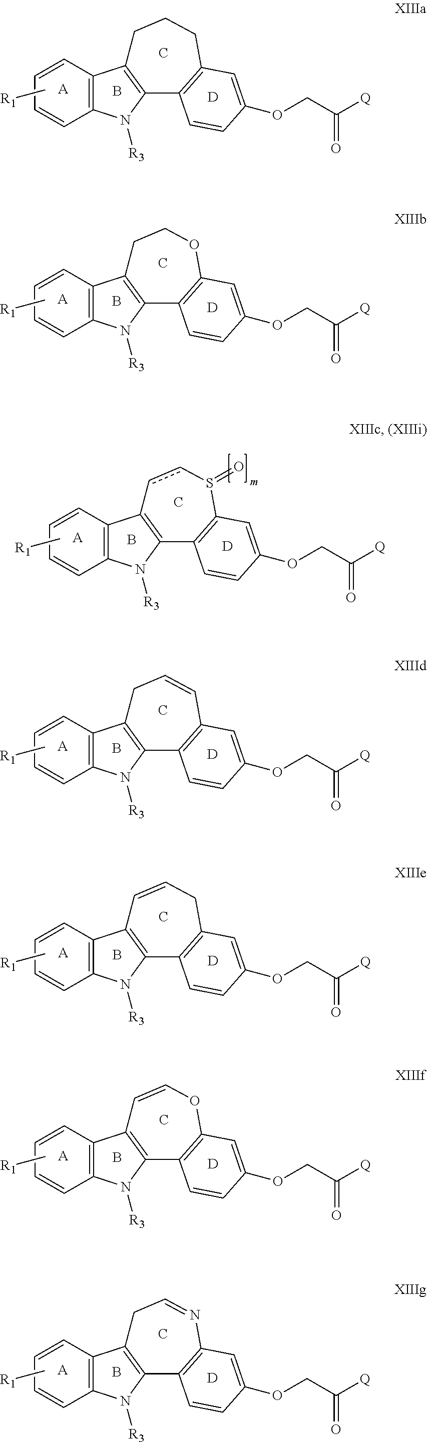

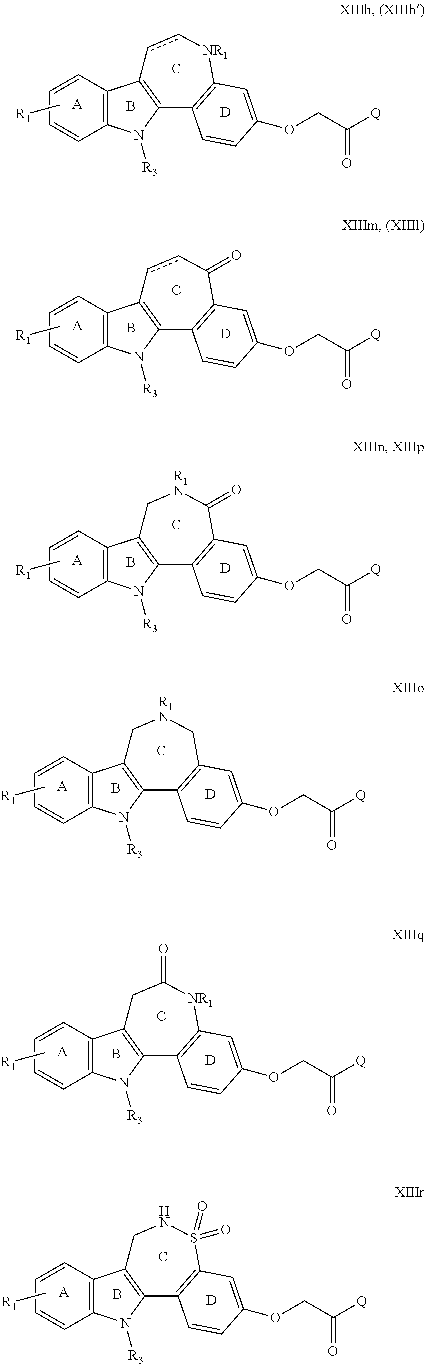

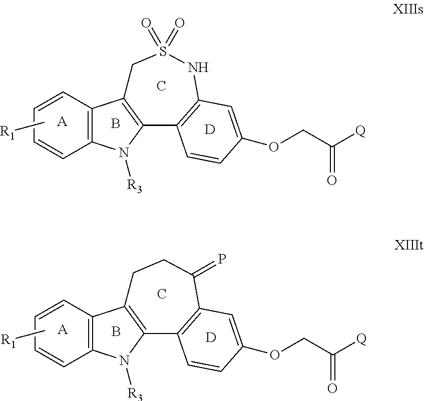

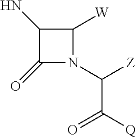

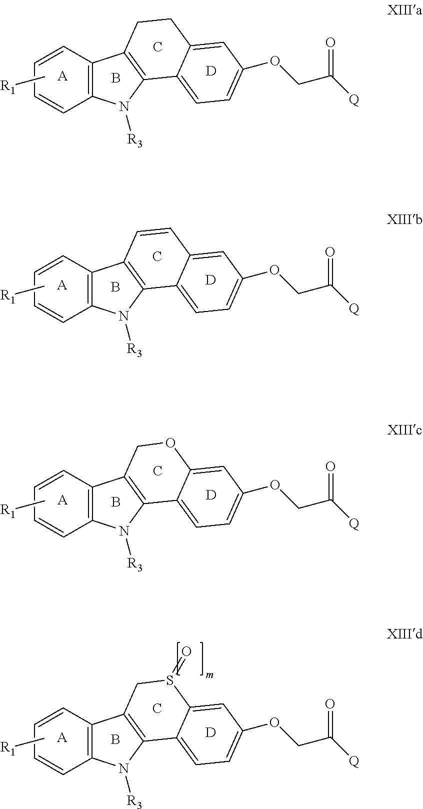

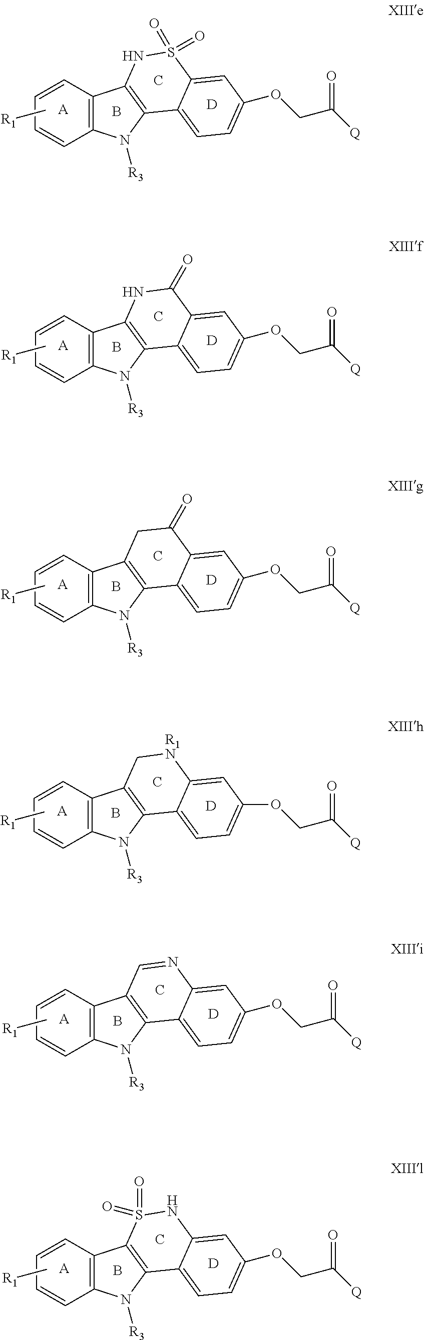

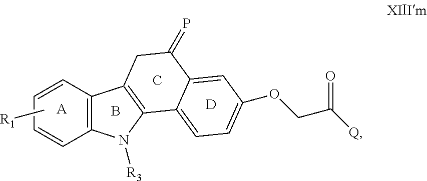

wherein m, Q, R₁ and R₃ are as defined above.

If Q is a bicyclic system containing the beta-lactam nucleus 7-aminocefem substituted as in formula (XIV), there can be structures of formula (XIVa-XIVt), (XIV′a, XIV′m):

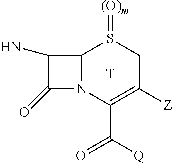

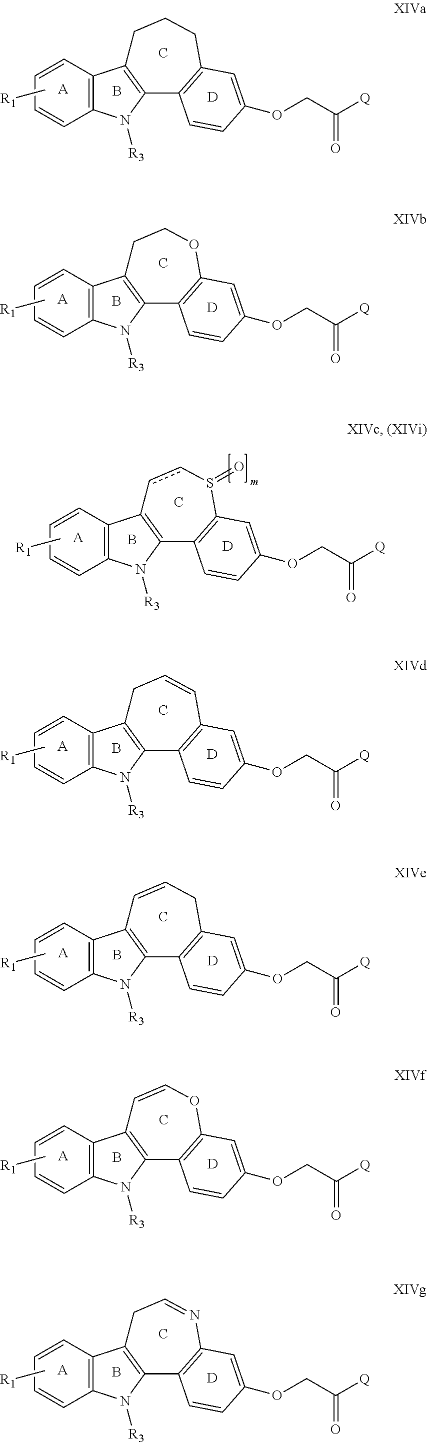

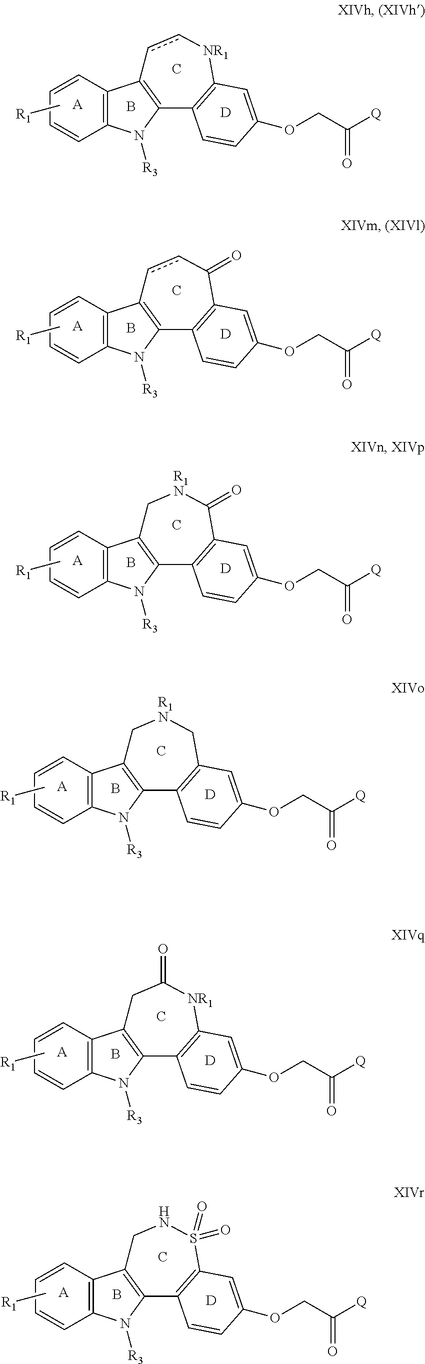

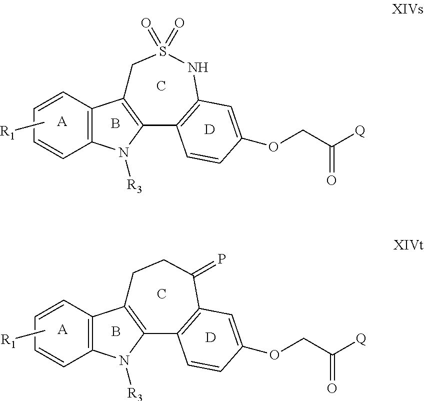

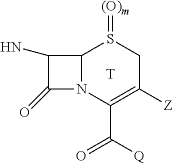

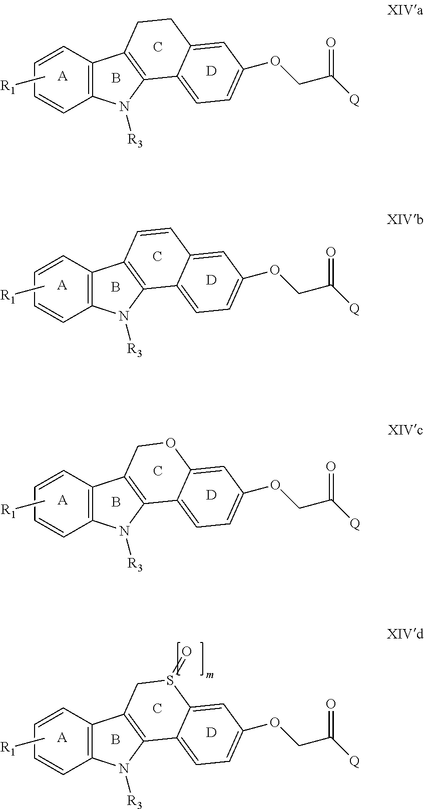

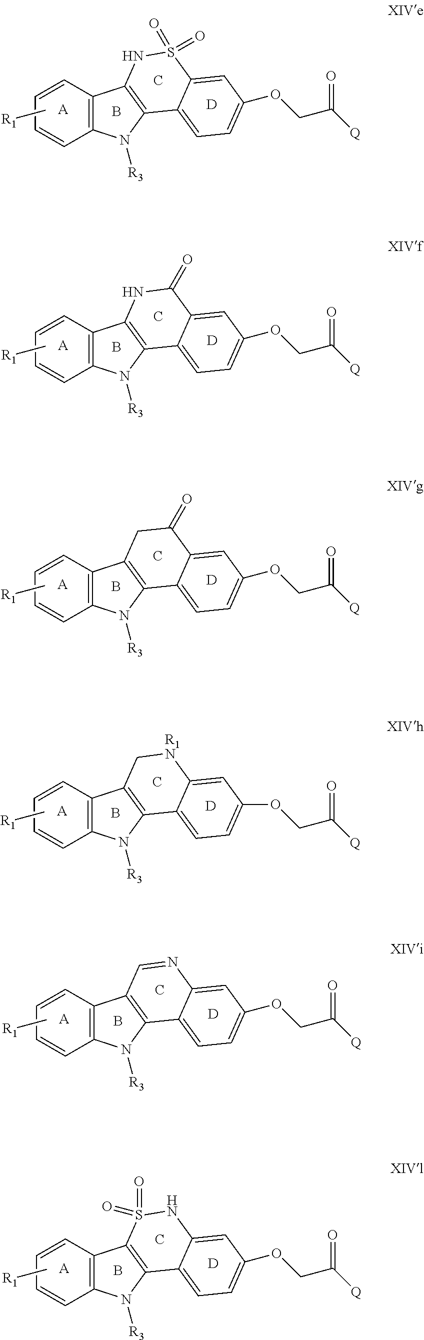

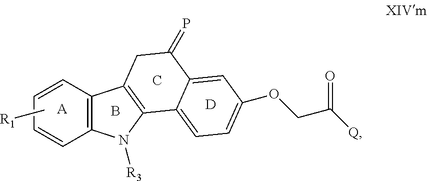

wherein m, P, Q, R₁ and R₃ are as defined above.

If Q is a bicyclic system containing the beta-lactam nucleus 6-aminopenam substituted as in formula (XV), there can be structures of formula XVa, XVb, XVc, XVh, XVm, XVn, XVo, XVp, XVq, XVr, XVs, XVt, XV′a, XV′b, XV′c, XV′d, XV′e, XV′f, XV′g, XV′h, XV′i, XV′l, XV′m.

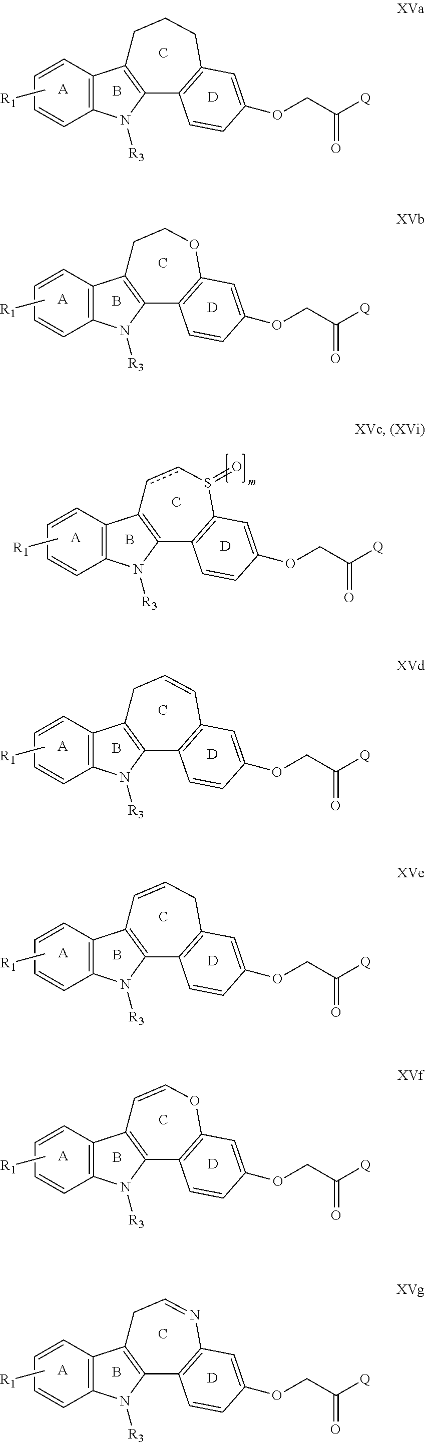

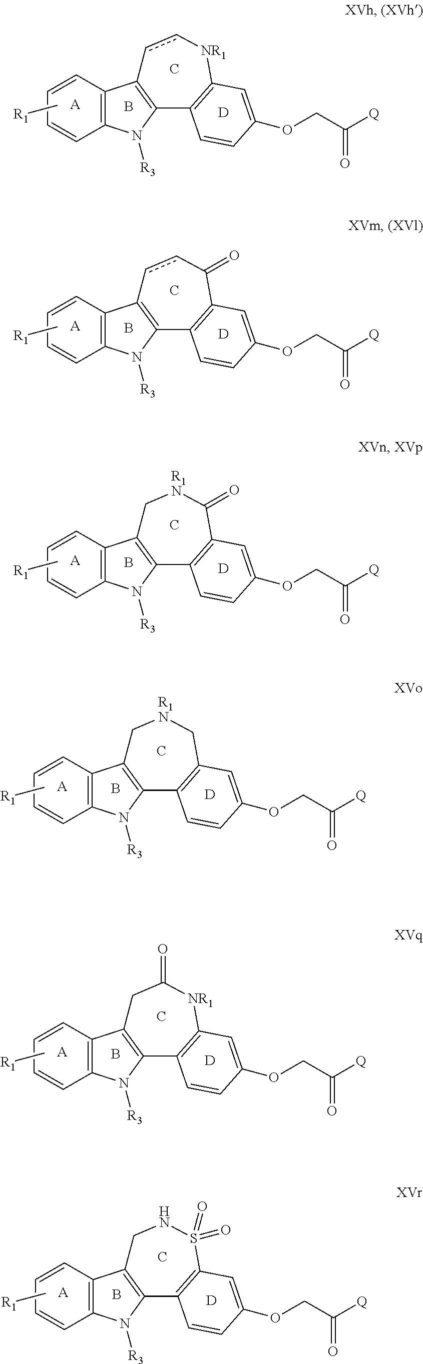

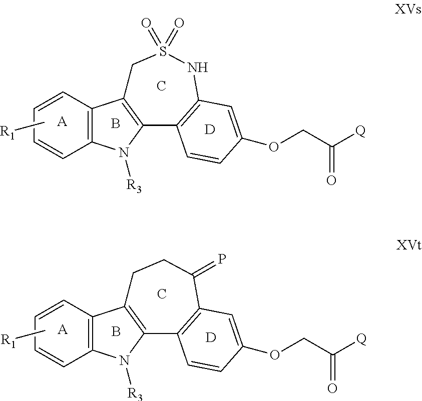

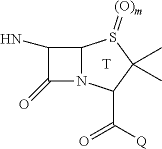

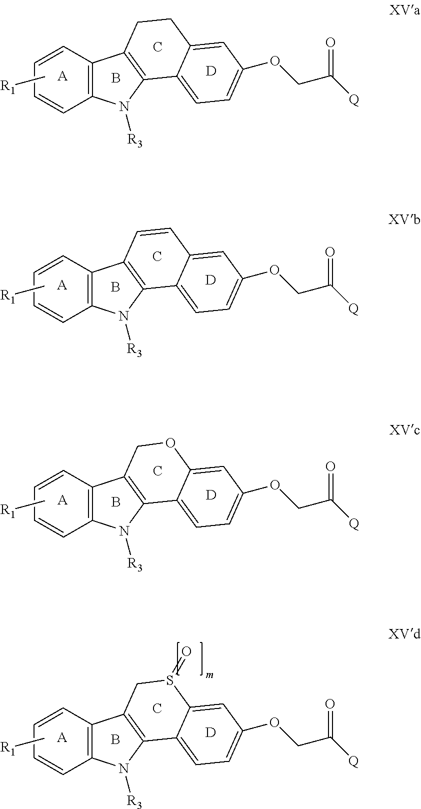

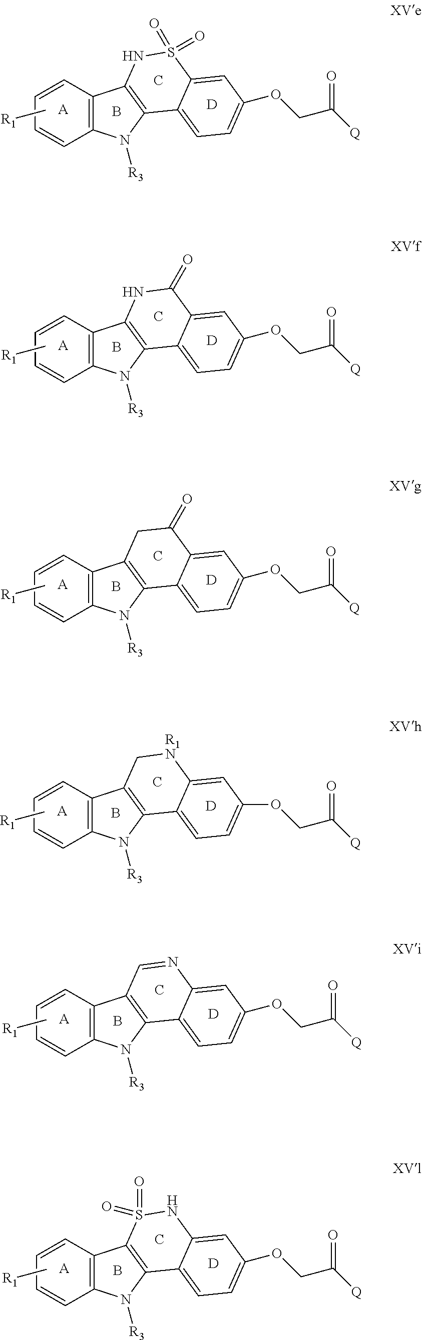

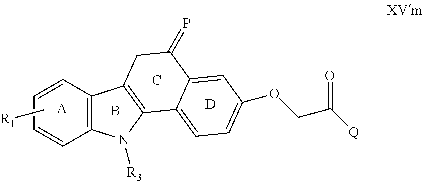

wherein m, P, Q, R₁ and R₃ are as defined above.

According to a particularly preferred aspect of the invention, the compounds of general formula (I) are those listed in Table 1 below:

TABLE 1

| # substructure | # Code | Structure |
|---|---|---|
| XIc XIl | RA1 | 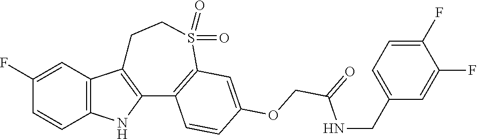 [SMILES: O=C(COc1ccc2c(c1)S(=O)(=O)CCc1c-2[nH]c2ccc(F)cc12)NCc1ccc(F)c(F)c1] |
| XIa | RA2 | 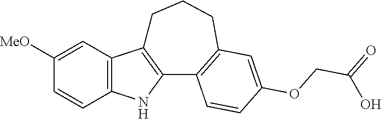 [SMILES: COc1ccc2[nH]c3c(c2c1)CCCc1cc(OCC(=O)O)ccc1-3] |
| XIr | RA3 | 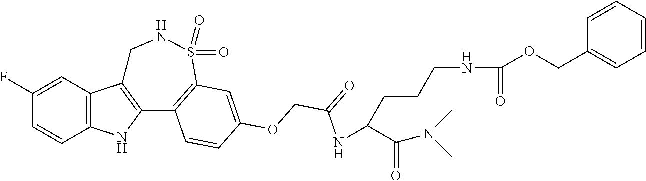 [SMILES: CN(C)C(=O)C(CCCNC(=O)OCc1ccccc1)NC(=O)COc1ccc2c(c1)S(=O)(=O)NCc1c-2[nH]c2ccc(F)cc12] |
| Xa | GA17M8 | 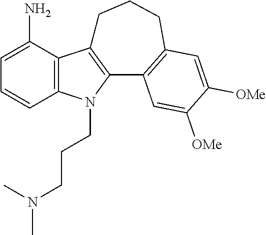 [SMILES: COc1cc2c(cc1OC)-c1c(c3c(N)cccc3n1CCCN(C)C)CCC2] |
| Xa | GA17M10 | 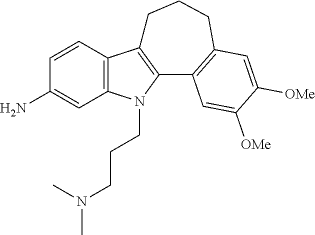 [SMILES: COc1cc2c(cc1OC)-c1c(c3ccc(N)cc3n1CCCN(C)C)CCC2] |
| XIb | GA11S | 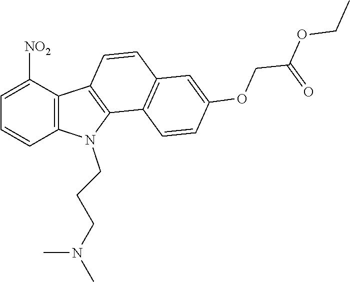 [SMILES: CCOC(=O)COc1ccc2c(ccc3c4c([N+](=O)[O-])cccc4n(CCCN(C)C)c23)c1] |
| XIb | RM66 | 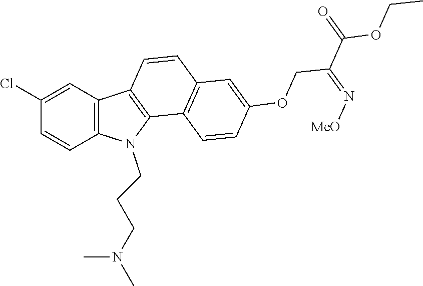 [SMILES: CCOC(=O)/C(COc1ccc2c(ccc3c4cc(Cl)ccc4n(CCCN(C)C)c23)c1)=N/OC] |
| XIb | RM58 | 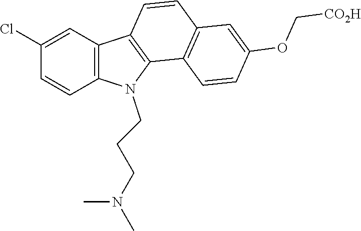 [SMILES: CN(C)CCCn1c2ccc(Cl)cc2c2ccc3cc(OCC(=O)O)ccc3c21] |
| XIb | RM70 | 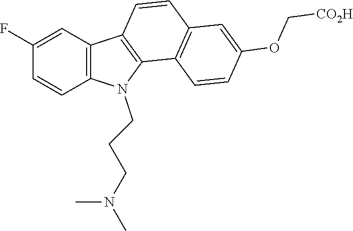 [SMILES: CN(C)CCCn1c2ccc(F)cc2c2ccc3cc(OCC(=O)O)ccc3c21] |
| XIb | RM85 | 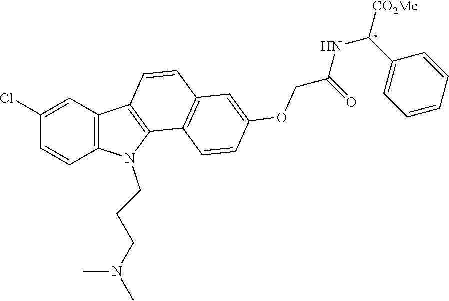 |
| XI’d | RA6 | 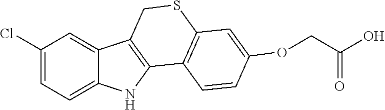 [SMILES: O=C(O)COc1ccc2c(c1)SCc1c-2[nH]c2ccc(Cl)cc12] |
| XI’d | RA7 | 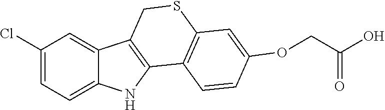 [SMILES: O=C(O)COc1ccc2c(c1)SCc1c-2[nH]c2ccc(Cl)cc12] |
| XI’d | RA9 | 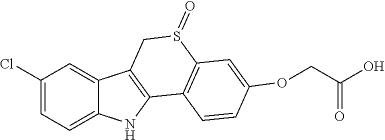 [SMILES: O=C(O)COc1ccc2c(c1)S(=O)Cc1c-2[nH]c2ccc(Cl)cc12] |
| XI’d | RA11 | 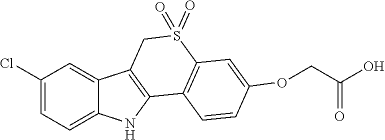 [SMILES: O=C(O)COc1ccc2c(c1)S(=O)(=O)Cc1c-2[nH]c2ccc(Cl)cc12] |
| XI’d | RA12 | 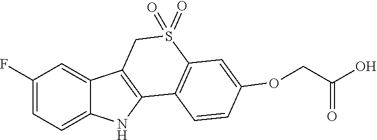 [SMILES: O=C(O)COc1ccc2c(c1)S(=O)(=O)Cc1c-2[nH]c2ccc(F)cc12] |
| XI’d | RA13 | 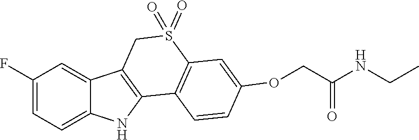 [SMILES: CCNC(=O)COc1ccc2c(c1)S(=O)(=O)Cc1c-2[nH]c2ccc(F)cc12] |
| XI’g | RA14 | 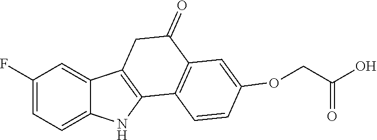 [SMILES: O=C(O)COc1ccc2c(c1)C(=O)Cc1c-2[nH]c2ccc(F)cc12] |
| X’c | RA15 | 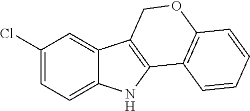 [SMILES: Clc1ccc2[nH]c3c(c2c1)COc1ccccc1-3] |
| X’c | RA16 | 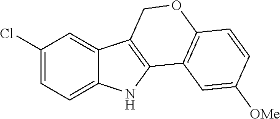 [SMILES: COc1ccc2c(c1)-c1[nH]c3ccc(Cl)cc3c1CO2] |
| XI’c | RA17 | 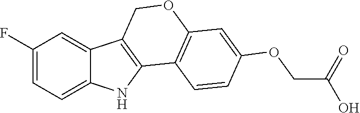 [SMILES: O=C(O)COc1ccc2c(c1)OCc1c-2[nH]c2ccc(F)cc12] |
| XI’c | RA18 | 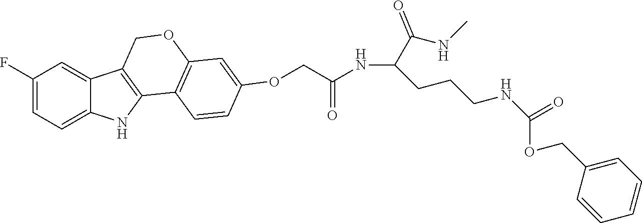 [SMILES: CNC(=O)C(CCCNC(=O)OCc1ccccc1)NC(=O)COc1ccc2c(c1)OCc1c-2[nH]c2ccc(F)cc12] |
| XI’c | RA19 | 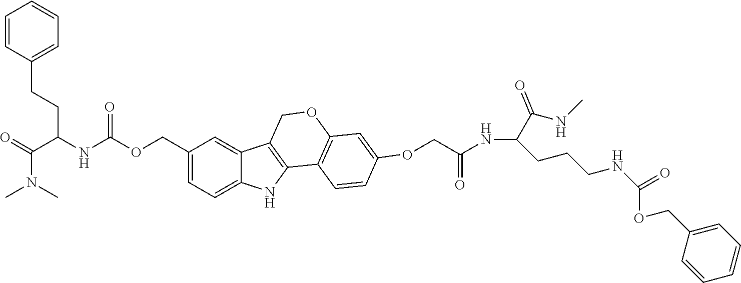 [SMILES: CNC(=O)C(CCCNC(=O)OCc1ccccc1)NC(=O)COc1ccc2c(c1)OCc1c-2[nH]c2ccc(COC(=O)NC(CCc3ccccc3)C(=O)N(C)C)cc12] |
| XI’h | RA20 | 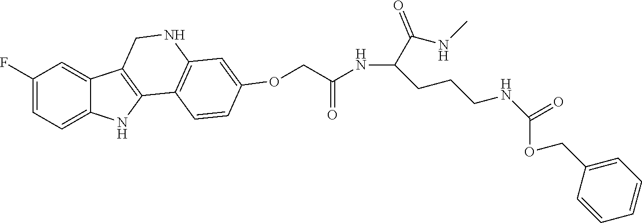 [SMILES: CNC(=O)C(CCCNC(=O)OCc1ccccc1)NC(=O)COc1ccc2c(c1)NCc1c-2[nH]c2ccc(F)cc12] |
| X’e | RA21 | 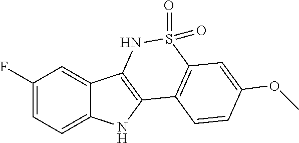 [SMILES: COc1ccc2c(c1)S(=O)(=O)Nc1c-2[nH]c2ccc(F)cc12] |
| XI’e | RA22 | 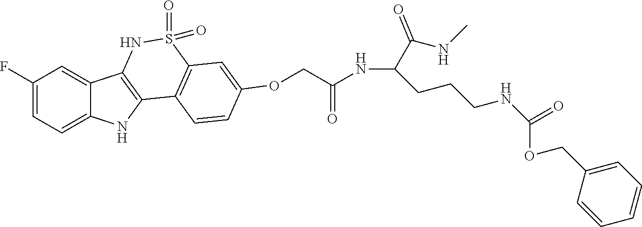 [SMILES: CNC(=O)C(CCCNC(=O)OCc1ccccc1)NC(=O)COc1ccc2c(c1)S(=O)(=O)Nc1c-2[nH]c2ccc(F)cc12] |
| XIV’a | GA09a | 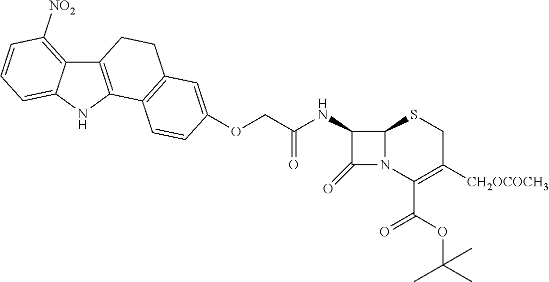 |
| XIV’a’ | GA09a’ | 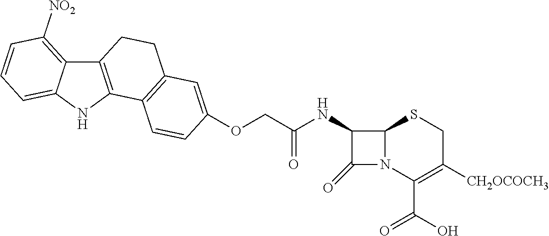 |
| XIV’a | GA09b | 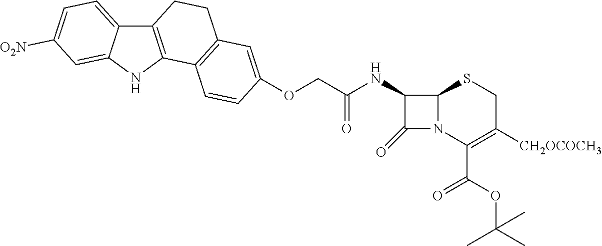 |
| XIV’a’ | GA09b’ | 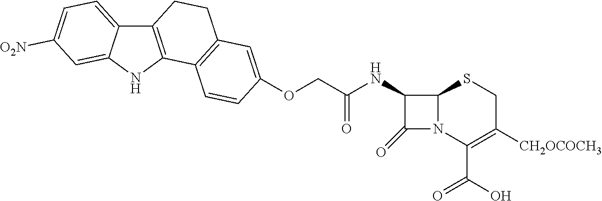 |
| XIV’a | RM36 | 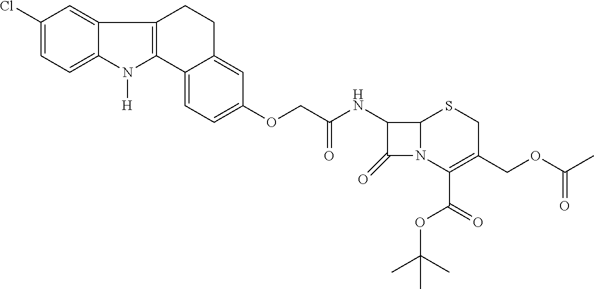 [SMILES: CC(=O)OCC1=C(C(=O)OC(C)(C)C)N2C(=O)C(NC(=O)COc3ccc4c(c3)CCc3c-4[nH]c4ccc(Cl)cc34)C2SC1] |
| XIV’a | RM37 | 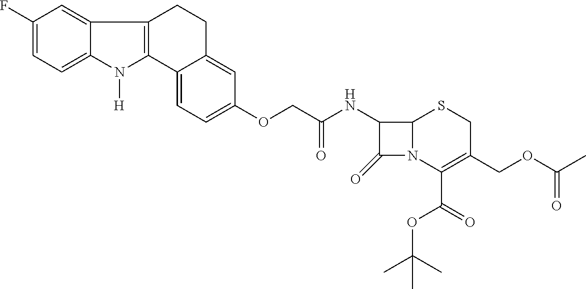 [SMILES: CC(=O)OCC1=C(C(=O)OC(C)(C)C)N2C(=O)C(NC(=O)COc3ccc4c(c3)CCc3c-4[nH]c4ccc(F)cc34)C2SC1] |
| XIV’a’ | RM53 | 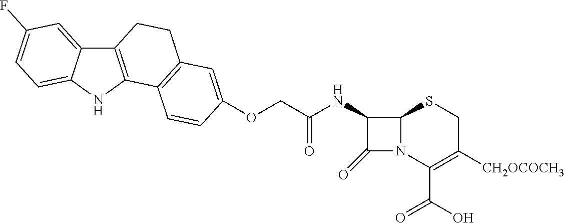 |
| XIV’a | RM47 | 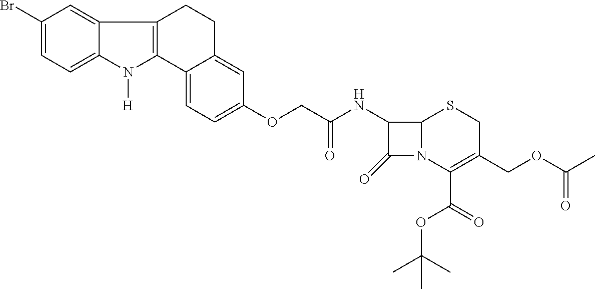 [SMILES: CC(=O)OCC1=C(C(=O)OC(C)(C)C)N2C(=O)C(NC(=O)COc3ccc4c(c3)CCc3c-4[nH]c4ccc(Br)cc34)C2SC1] |
| XIV’d | RA23 | 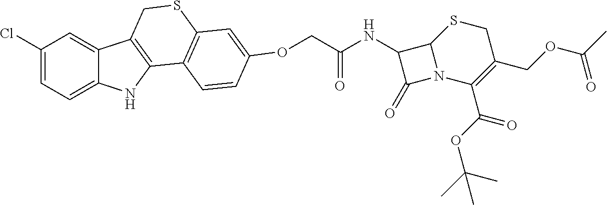 [SMILES: CC(=O)OCC1=C(C(=O)OC(C)(C)C)N2C(=O)C(NC(=O)COc3ccc4c(c3)SCc3c-4[nH]c4ccc(Cl)cc34)C2SC1] |
| XIV’d | RA24 | 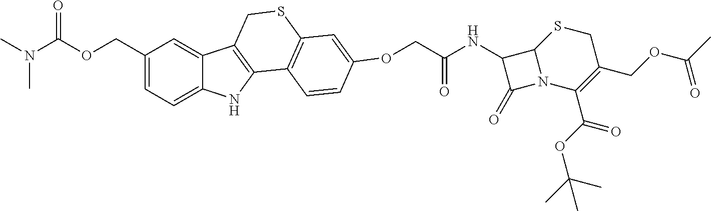 [SMILES: CC(=O)OCC1=C(C(=O)OC(C)(C)C)N2C(=O)C(NC(=O)COc3ccc4c(c3)SCc3c-4[nH]c4ccc(COC(=O)N(C)C)cc34)C2SC1] |
| XIV’d | RA25 | 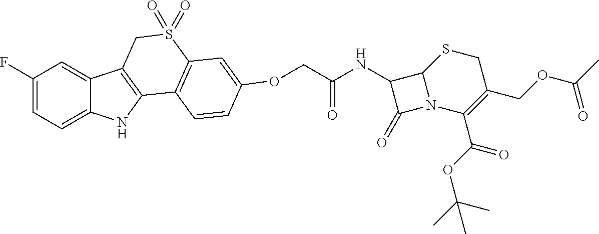 [SMILES: CC(=O)OCC1=C(C(=O)OC(C)(C)C)N2C(=O)C(NC(=O)COc3ccc4c(c3)S(=O)(=O)Cc3c-4[nH]c4ccc(F)cc34)C2SC1] |
| XV’e | RA26 | 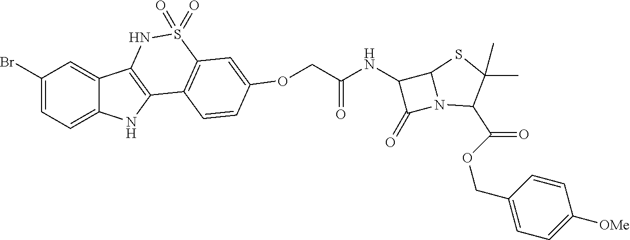 [SMILES: COc1ccc(COC(=O)C2N3C(=O)C(NC(=O)COc4ccc5c(c4)S(=O)(=O)Nc4c-5[nH]c5ccc(Br)cc45)C3SC2(C)C)cc1] |
| XV’d | RA27 | 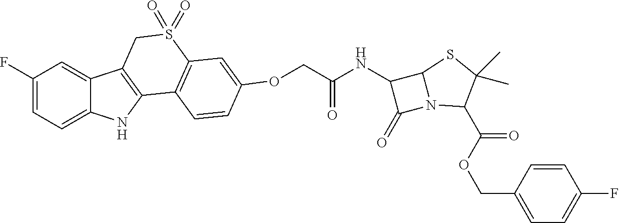 [SMILES: CC1(C)SC2C(NC(=O)COc3ccc4c(c3)S(=O)(=O)Cc3c-4[nH]c4ccc(F)cc34)C(=O)N2C1C(=O)OCc1ccc(F)cc1] |
| XV’d | RA28 | 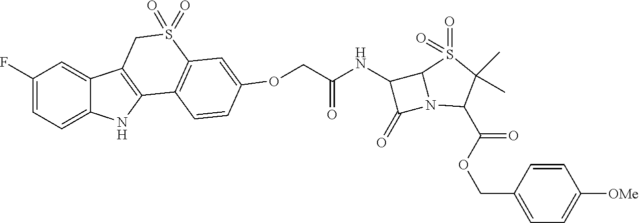 [SMILES: COc1ccc(COC(=O)C2N3C(=O)C(NC(=O)COc4ccc5c(c4)S(=O)(=O)Cc4c-5[nH]c5ccc(F)cc45)C3S(=O)(=O)C2(C)C)cc1] |
| XV’g’ | RA29 | 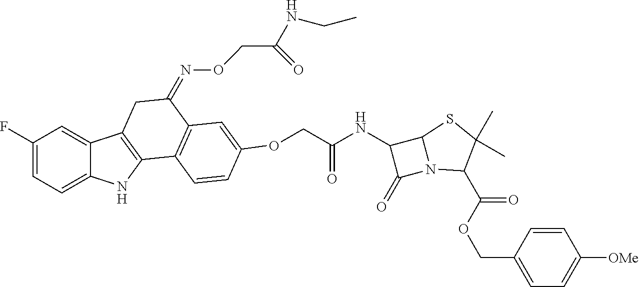 [SMILES: CCNC(=O)CO/N=C1/Cc2c([nH]c3ccc(F)cc23)-c2ccc(OCC(=O)NC3C(=O)N4C3SC(C)(C)C4C(=O)OCc3ccc(OC)cc3)cc21] |
| XIIId | RA30 | 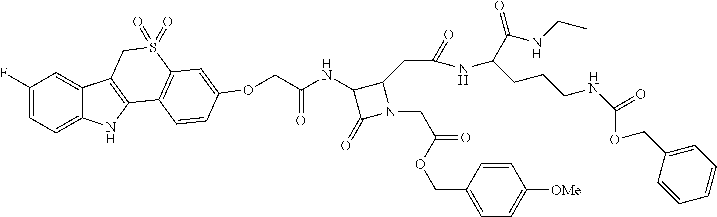 [SMILES: CCNC(=O)C(CCCNC(=O)OCc1ccccc1)NC(=O)CC1C(NC(=O)COc2ccc3c(c2)S(=O)(=O)Cc2c-3[nH]c3ccc(F)cc23)C(=O)N1CC(=O)OCc1ccc(OC)cc1] |
| XIV’c | RA31 | 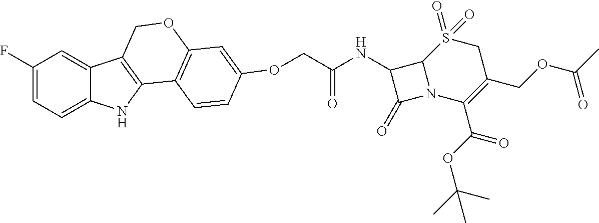 [SMILES: CC(=O)OCC1=C(C(=O)OC(C)(C)C)N2C(=O)C(NC(=O)COc3ccc4c(c3)OCc3c-4[nH]c4ccc(F)cc34)C2S(=O)(=O)C1] |
| XIV’c | RA32 | 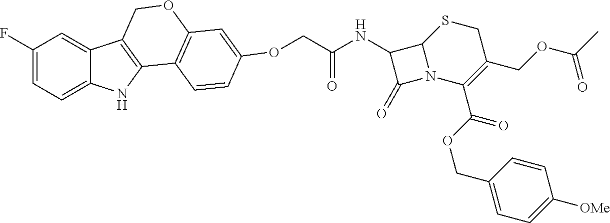 [SMILES: COc1ccc(COC(=O)C2=C(COC(C)=O)CSC3C(NC(=O)COc4ccc5c(c4)OCc4c-5[nH]c5ccc(F)cc45)C(=O)N23)cc1] |
| XIV’c | RA33 | 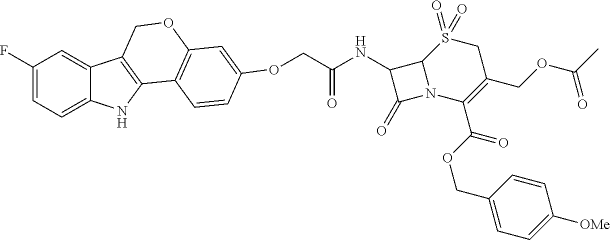 [SMILES: COc1ccc(COC(=O)C2=C(COC(C)=O)CS(=O)(=O)C3C(NC(=O)COc4ccc5c(c4)OCc4c-5[nH]c5ccc(F)cc45)C(=O)N23)cc1] |
| XIII’c | RA34 | 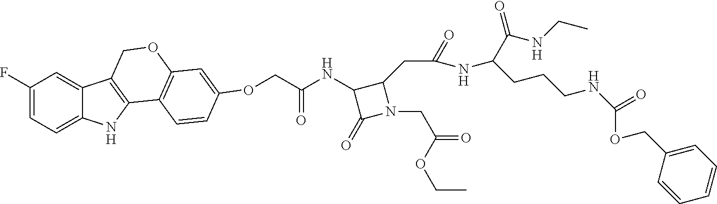 [SMILES: CCNC(=O)C(CCCNC(=O)OCc1ccccc1)NC(=O)CC1C(NC(=O)COc2ccc3c(c2)OCc2c-3[nH]c3ccc(F)cc23)C(=O)N1CC(=O)OCC] |
| XV’c | RA35 | 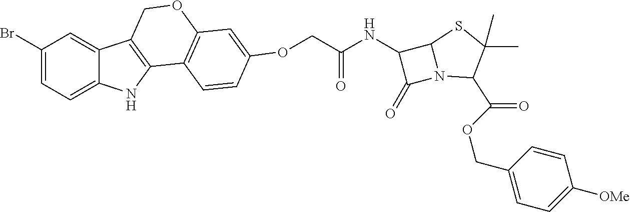 [SMILES: COc1ccc(COC(=O)C2N3C(=O)C(NC(=O)COc4ccc5c(c4)OCc4c-5[nH]c5ccc(Br)cc45)C3SC2(C)C)cc1] |
| XIV’e | RA36 | 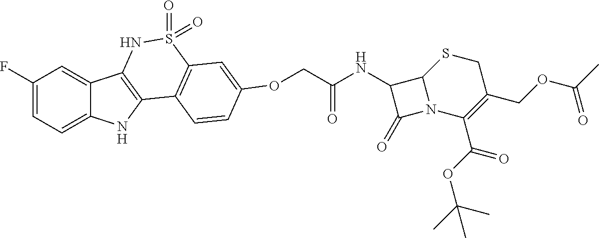 [SMILES: CC(=O)OCC1=C(C(=O)OC(C)(C)C)N2C(=O)C(NC(=O)COc3ccc4c(c3)S(=O)(=O)Nc3c-4[nH]c4ccc(F)cc34)C2SC1] |
| XIV’g’ | RA37 | 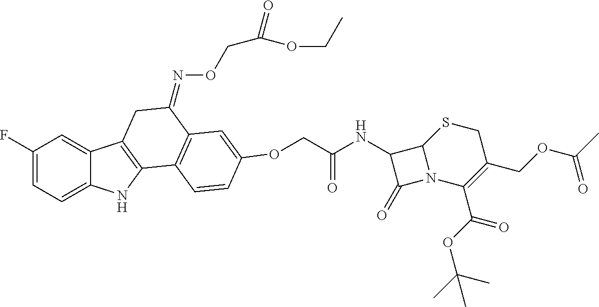 [SMILES: CCOC(=O)CO/N=C1/Cc2c([nH]c3ccc(F)cc23)-c2ccc(OCC(=O)NC3C(=O)N4C(C(=O)OC(C)(C)C)=C(COC(C)=O)CSC34)cc21] |
| XIV’g’ | RA38 | 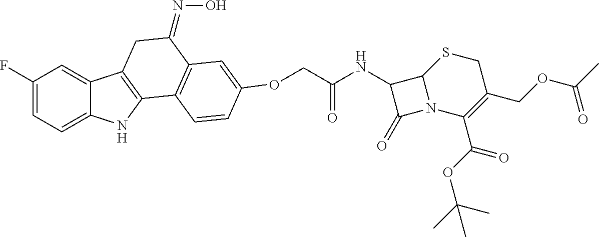 [SMILES: CC(=O)OCC1=C(C(=O)OC(C)(C)C)N2C(=O)C(NC(=O)COc3ccc4c(c3)/C(=N\O)Cc3c-4[nH]c4ccc(F)cc34)C2SC1] |
| XIV’g | RA39 | 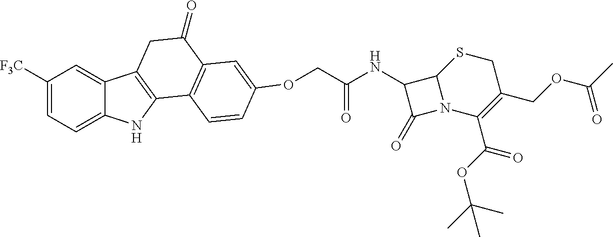 [SMILES: CC(=O)OCC1=C(C(=O)OC(C)(C)C)N2C(=O)C(NC(=O)COc3ccc4c(c3)C(=O)Cc3c-4[nH]c4ccc(C(F)(F)F)cc34)C2SC1] |
| XIVr | RA40 | 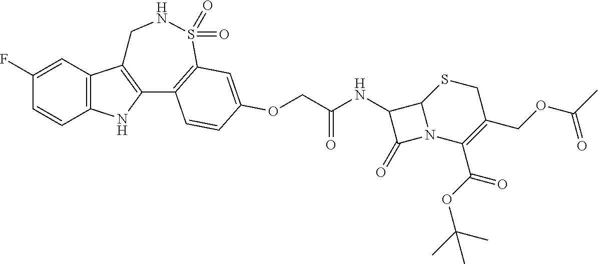 [SMILES: CC(=O)OCC1=C(C(=O)OC(C)(C)C)N2C(=O)C(NC(=O)COc3ccc4c(c3)S(=O)(=O)NCc3c-4[nH]c4ccc(F)cc34)C2SC1] |
| XIVc | RA41 | 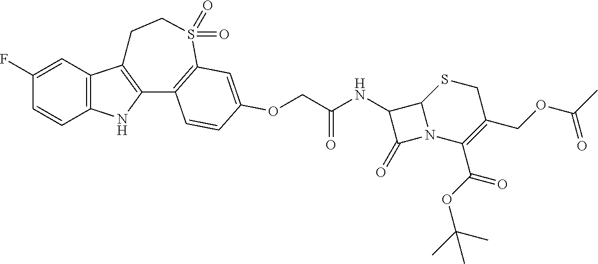 [SMILES: CC(=O)OCC1=C(C(=O)OC(C)(C)C)N2C(=O)C(NC(=O)COc3ccc4c(c3)S(=O)(=O)CCc3c-4[nH]c4ccc(F)cc34)C2SC1] |
| XVr | RA42 | 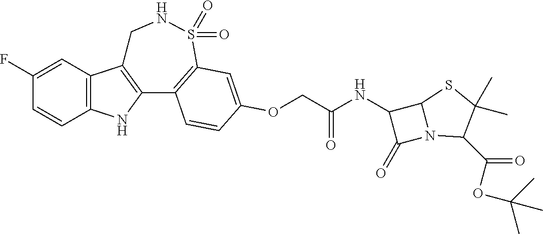 [SMILES: CC(C)(C)OC(=O)C1N2C(=O)C(NC(=O)COc3ccc4c(c3)S(=O)(=O)NCc3c-4[nH]c4ccc(F)cc34)C2SC1(C)C] |
| XIVc | RA43 | 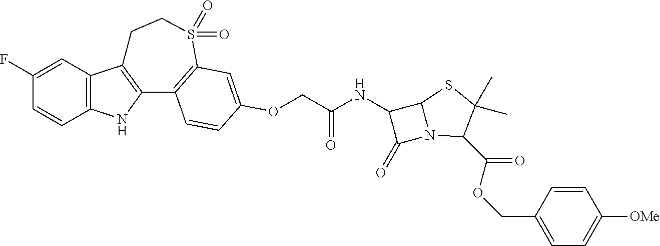 [SMILES: COc1ccc(COC(=O)C2N3C(=O)C(NC(=O)COc4ccc5c(c4)S(=O)(=O)CCc4c-5[nH]c5ccc(F)cc45)C3SC2(C)C)cc1] |
| XIIIr | RA44 | 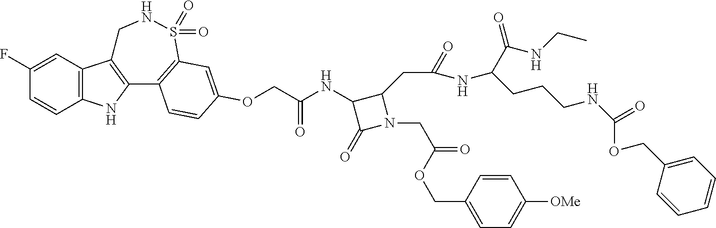 [SMILES: CCNC(=O)C(CCCNC(=O)OCc1ccccc1)NC(=O)CC1C(NC(=O)COc2ccc3c(c2)S(=O)(=O)NCc2c-3[nH]c3ccc(F)cc23)C(=O)N1CC(=O)OCc1ccc(OC)cc1] |

The condensed heterocycles A-D, substituted as in general formula (I), can be synthesised, depending on the type of heterocyclic system A-D or the need for substitution on the A-D system, with the various groupings R, R₁, R₂, R₃, R₄, R₅, R₆, R₇, R₈, M, M₁, P, P₁, T, T₁, Q, X, W and Z, by means of:

1—condensation of ring A with the carbonyl system of the preformed heterocycle C-D with or without isolation of an intermediate hydrazone system (iia-iiq or ii′a-ii′i) and subsequent and/or simultaneous formation of the indole junction cycle B which gives rise to the desired heterocycle A-D. In this type of condensation, both aromatic systems are already substituted and/or substitutable with the various necessary groupings: R, R₁, R₂, R₃, R₄, R₅, R₆, R₇, R₈, M, M₁, P, P₁, T, T₁, Q, X, W and Z, General Scheme (Ia).

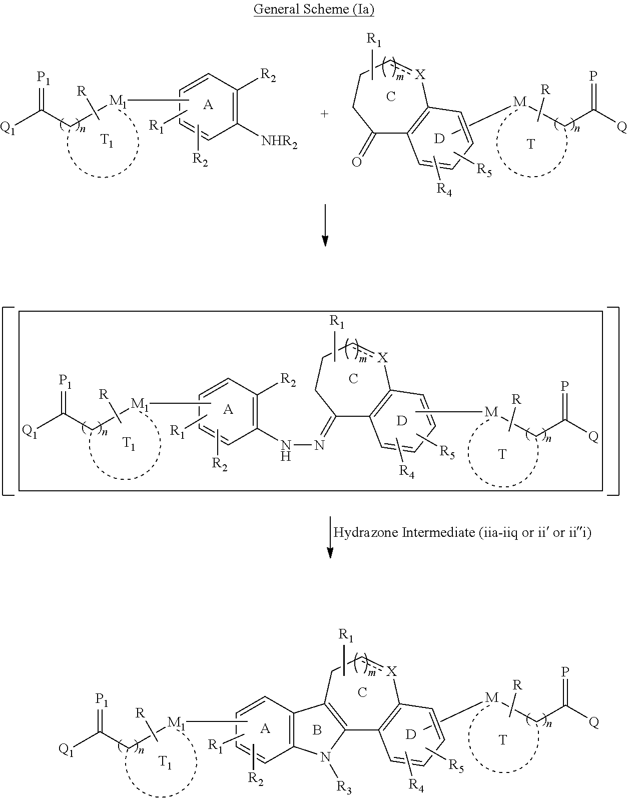

2—Condensation of a preformed indole system A-B already substituted with ring D to form junction ring C. In this type of condensation, as in 1-, both aromatic systems are already substituted and/or substitutable with the various necessary groupings: R, R₁, R₂, R₃, R₄, R₅, R₆, R₇, R₈, M, M₁, P, P₁, T, T₁, Q, X, W and Z, General Scheme (Ib).

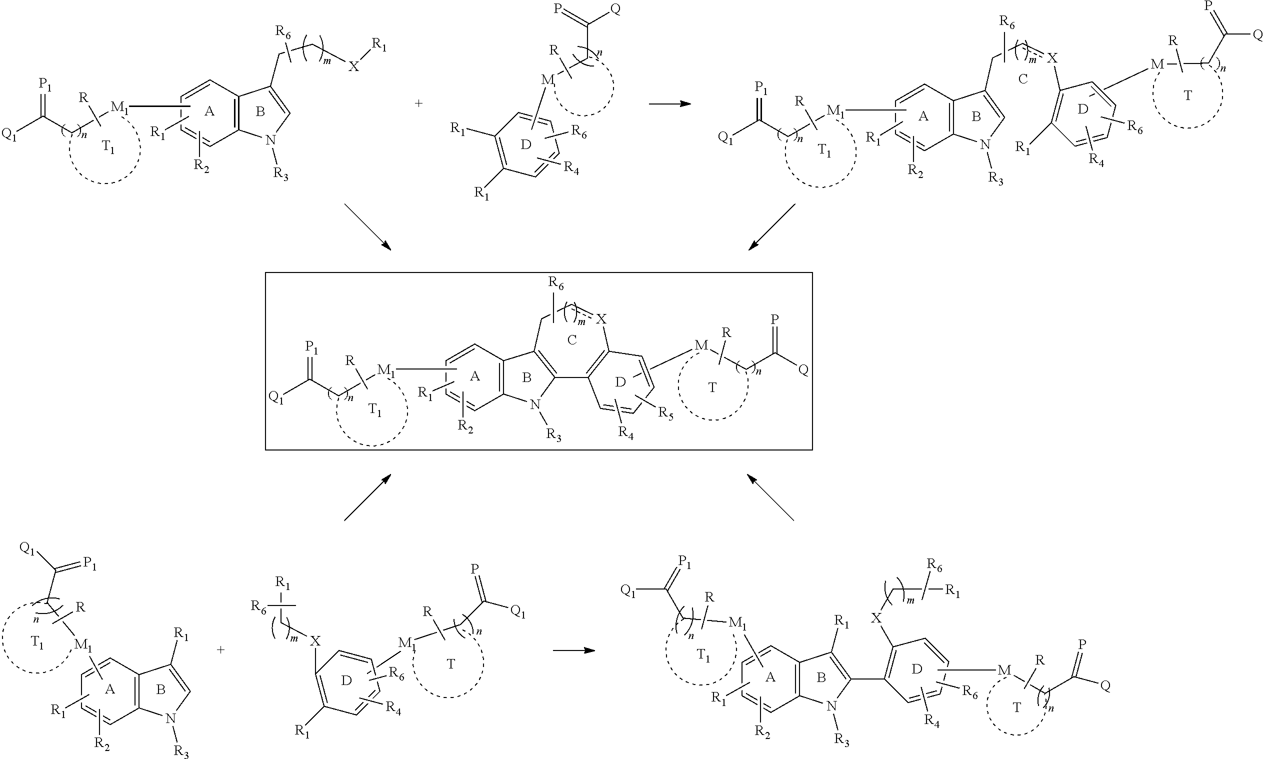

3—Condensation of a heterocyclic system substituted and/or substitutable as above containing the nucleus of the desired heterocyclic C-D which is activated as enamide and substituted with a Y substituted aryl (ring A is substituted and/or substitutable as above) [boronic acid B(OH)₂ or Si(Me)₃ or a mesityl-aryl-iodonium salt] to give an appropriate intermediate from which it will be cyclised with specific reagents to give indole junction ring B in the formation of heterocyclic system A-D substituted as desired for (I) and previously reported, General Scheme (Ic).

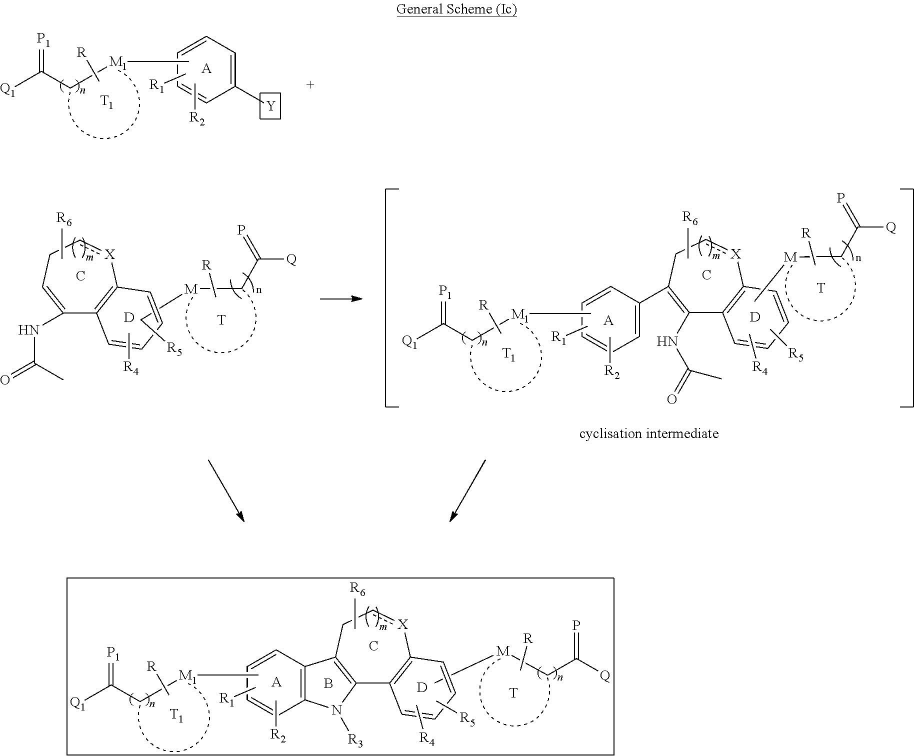

Many of the condensation methods described above to obtain heterocyclic intermediates A-B and/or C-D and the reactions of formation of the desired tetracyclic systems A-D from said systems are also reported in Chem. Rev. 2002, 102, 4303-4427, Eur. J. Org. Chem. 2006, 1379-1382, J. Heterocyclic Chem., 48, 1095 (2011), Adv. Synth. Catal. 2010, 352, 363-367, Chem. Eur. J. 2010, 16, 1124-1127, J. Am. Chem. Soc. 2006, 128, 581-590, J. Org. Chem. 2003, 68, 2807-2811, J. Org. Chem. 2014, 79, 7836-7843, Org. Lett., 2012, 14, 14, 3772-3775.

For example, the tetracyclic heterocycles A-D of structure (Xa-Xt), General Schemes (Id-Il), were obtained by following said methods.

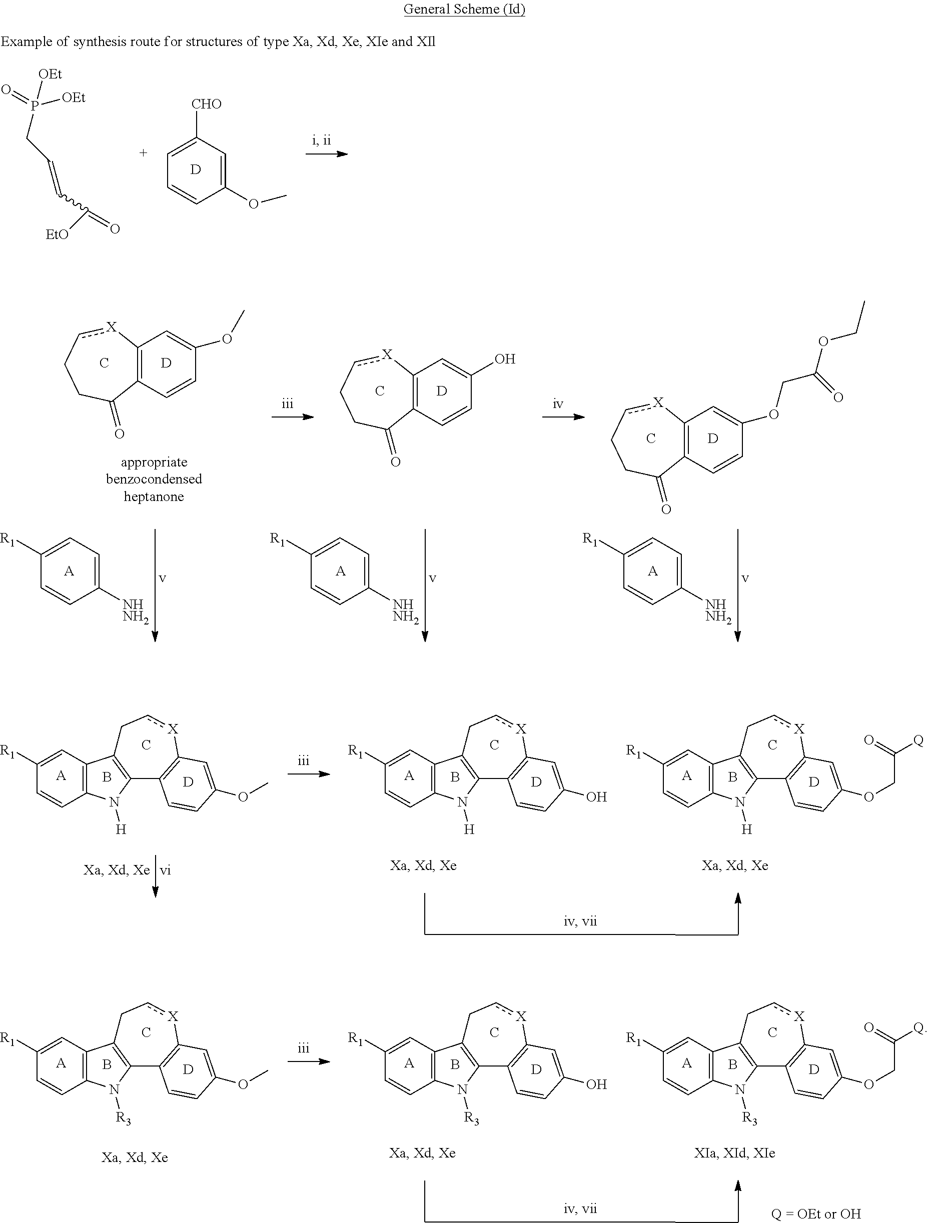

Reaction conditions—reagents, solvents and appropriate conditions according to type of reaction: i: an appropriately synthesised benzocondensed heptanone: NaH, THF or dioxane or another appropriate solvent, 3-OMe-benzaldehyde, THF or dioxane or an appropriate solvent and DMF; H₂/Pd—C 10% EtOH, or alternatively an appropriate commercial R₂-substituted-6,7,8,9-tetrahydro-benzocyclohepten-5-one; iii: 48% HBr, AcOH, 120° C., 12 h, or BBr₃, dichloromethane, −78° C. to −0°; iv: BrCH₂COOEt, K₂CO₃, acetone, reflux; v: 4-R₁-phenylhydrazine, or 4-R₁-phenylhydrazine hydrochloride or hydrobromide and appropriate acidic conditions (36% HCl reflux or AcOH, 120° C., 2-6 h, or microwave use in appropriate conditions 2-10 min., or BBr₃, dichloromethane, −78° C. to −0° C., 2 hours; vi: appropriate R₃—X halide (mainly but not only Br or Cl), NaH/anh. DMF, 80° C., 16 h, or microwaves in appropriate conditions 2-10 min.; vii: KOH/EtOH.

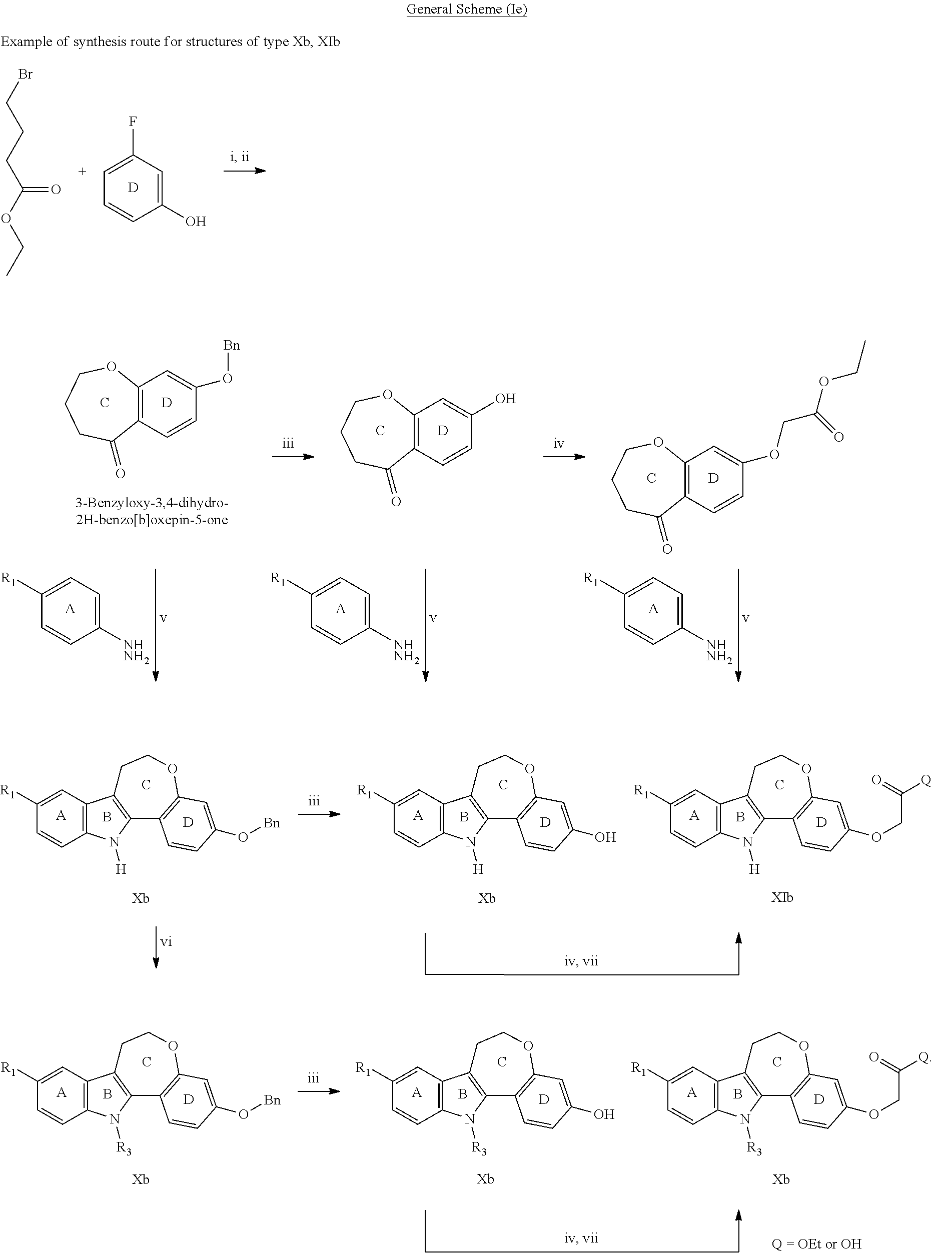

Reaction conditions—reagents, solvents and appropriate conditions according to type of reaction: i⁽¹⁾: a) 4-bromoethylbutyrate, KI, K₂CO₃, acetone, b) NaOH, EtOH; c) BnOH, K₂CO₃, DMF⁽¹⁾; ii⁽¹⁾: cyanuric chloride (C₃N₃Cl₃), dichloromethane, pyridine, 0° C., 30+30 min, r/t 3 hours, cooled to −60° C., followed by addition of solid AlCl₃ in portions, controlled t to 0° C. for 3-4 hours; iii: H₂/Pd—C 10% EtOH or AcOEt/EtOH; iv: BrCH₂COOEt, K₂CO₃, acetone, reflux; v: 4-R₁-phenylhydrazine, or 4-R₁-phenylhydrazine hydrochloride or hydrobromide and appropriate acidic conditions (36% HCl reflux or AcOH, 120° C., 2-6 h, or microwave use in appropriate conditions 2-10 min.; vi: appropriate R₃—X halide (mainly but not only Br or Cl), NaH/anh. DMF, 80° C., 16 h, or microwaves in appropriate conditions 2-10 min.; vii: KOH/EtOH; [⁽¹⁾ European Journal of Organic Chemistry (2014), 2014, (15), 3170-3181; Org. Lett., Vol. 10, No. 13, 2008.].

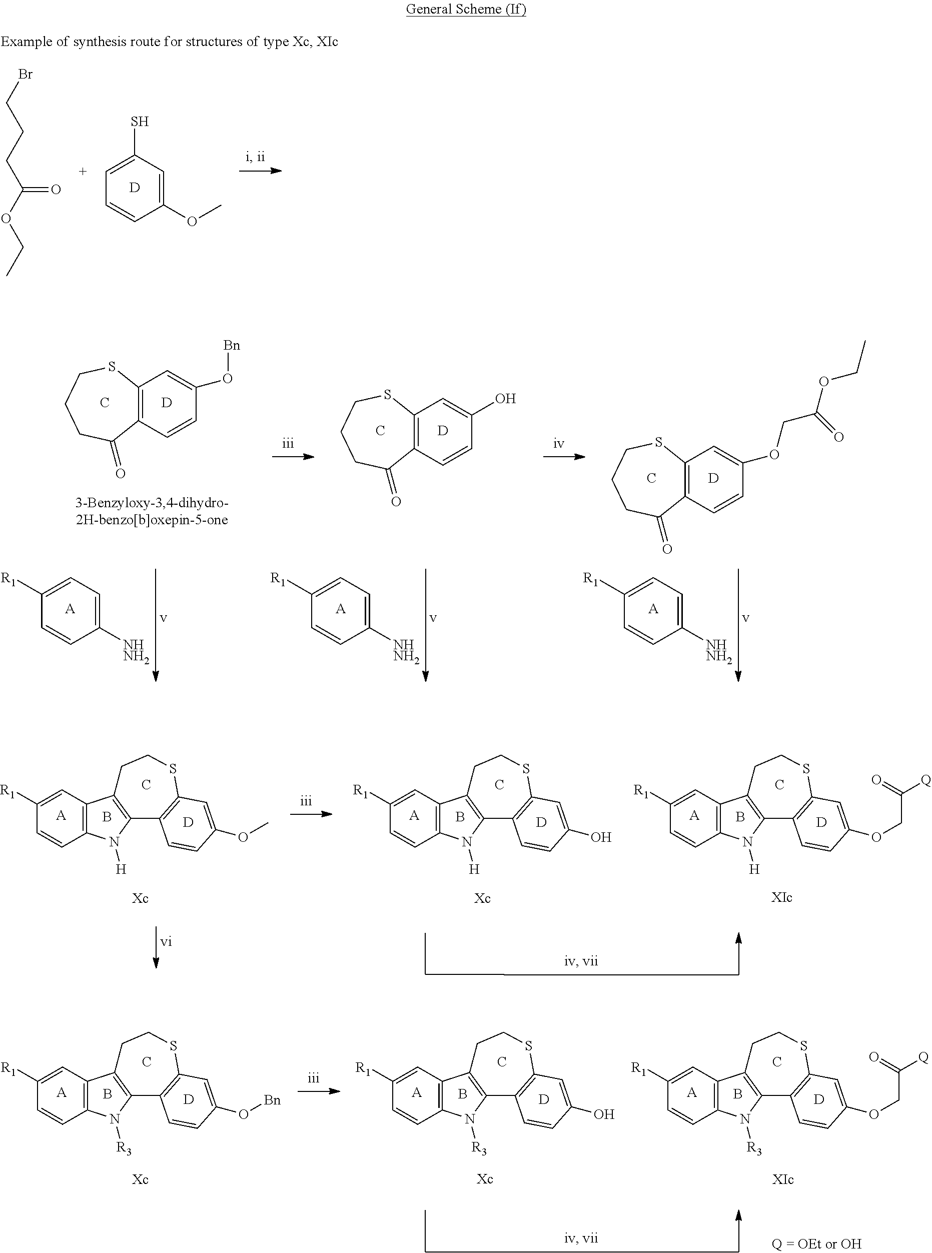

Reaction conditions—reagents, solvents and appropriate conditions according to type of reaction: i: a) 4-bromoethylbutyrate, KOH, EtOH, N₂ 10 days or microwaves 1 h, H₂O 5 hours, acidification pH=1 with 1N HCl; ii⁽ⁱ⁾: trichloroacetic anhydride, 70° C., until complete (TLC); iii: HBr 48%, AcOH, 120° C., 12 h., or BBr₃, dichloromethane, −78° C. to −0°; iv: BrCH₂COOEt, K₂CO₃, acetone, reflux; v: 4-R₁-phenylhydrazine, or 4-R₁-phenylhydrazine hydrochloride or hydrobromide and appropriate acidic conditions (36% HCl reflux or AcOH, 120° C., 2-6 h, or microwave use in appropriate conditions 2-10 min, 2 hours; vi: appropriate R₃—X halide (mainly but not only Br or Cl), NaH/anh. DMF, 80° C., 16 h, or microwaves in appropriate conditions 2-10 min.; vii: KOH/EtOH; [⁽¹⁾ Synthetic Communications 1, 39: 2664-2673, 2009.].

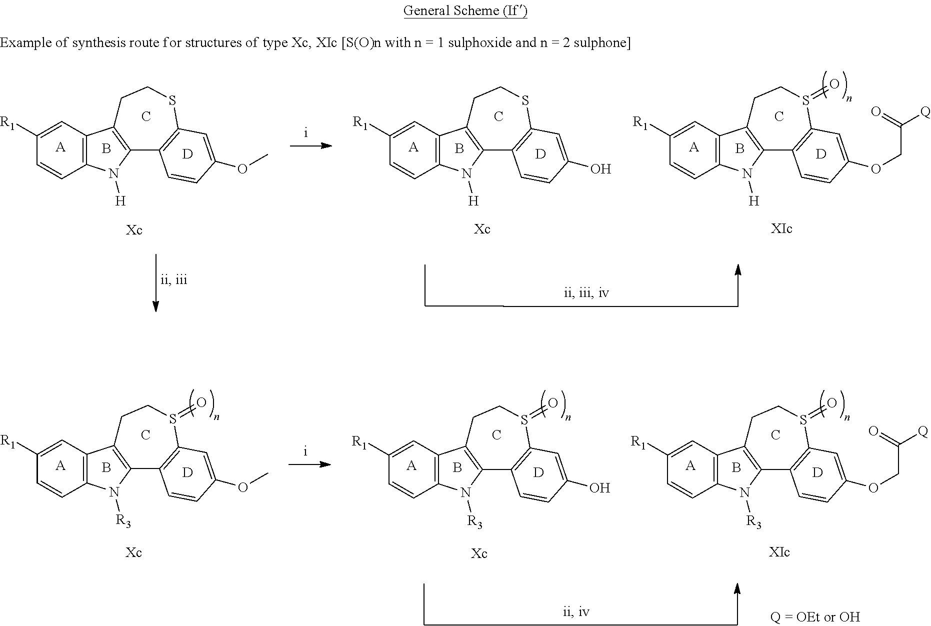

Reaction conditions—reagents, solvents and appropriate conditions according to type of reaction: i: HBr 48%, AcOH, 120° C., 12 h., or BBr₃, dichloromethane, −78° C. to −0° C.; MCPB, DCM r/t 2-6 hours or Ti(O-ᵢPr)₄, (R,R)-diethyltartrate, DCM, 15° C., 15-30 min., or Oxone®, THF/MeOH, r/t, 24-120 hours; iii: appropriate R₃—X halide (mainly but not only Br or CO, NaH/anh. DMF, 80° C., 16 h, or microwaves in appropriate conditions 2-10 iv: BrCH₂COOEt, K₂CO₃, acetone, reflux; iv: KOH/EtOH.

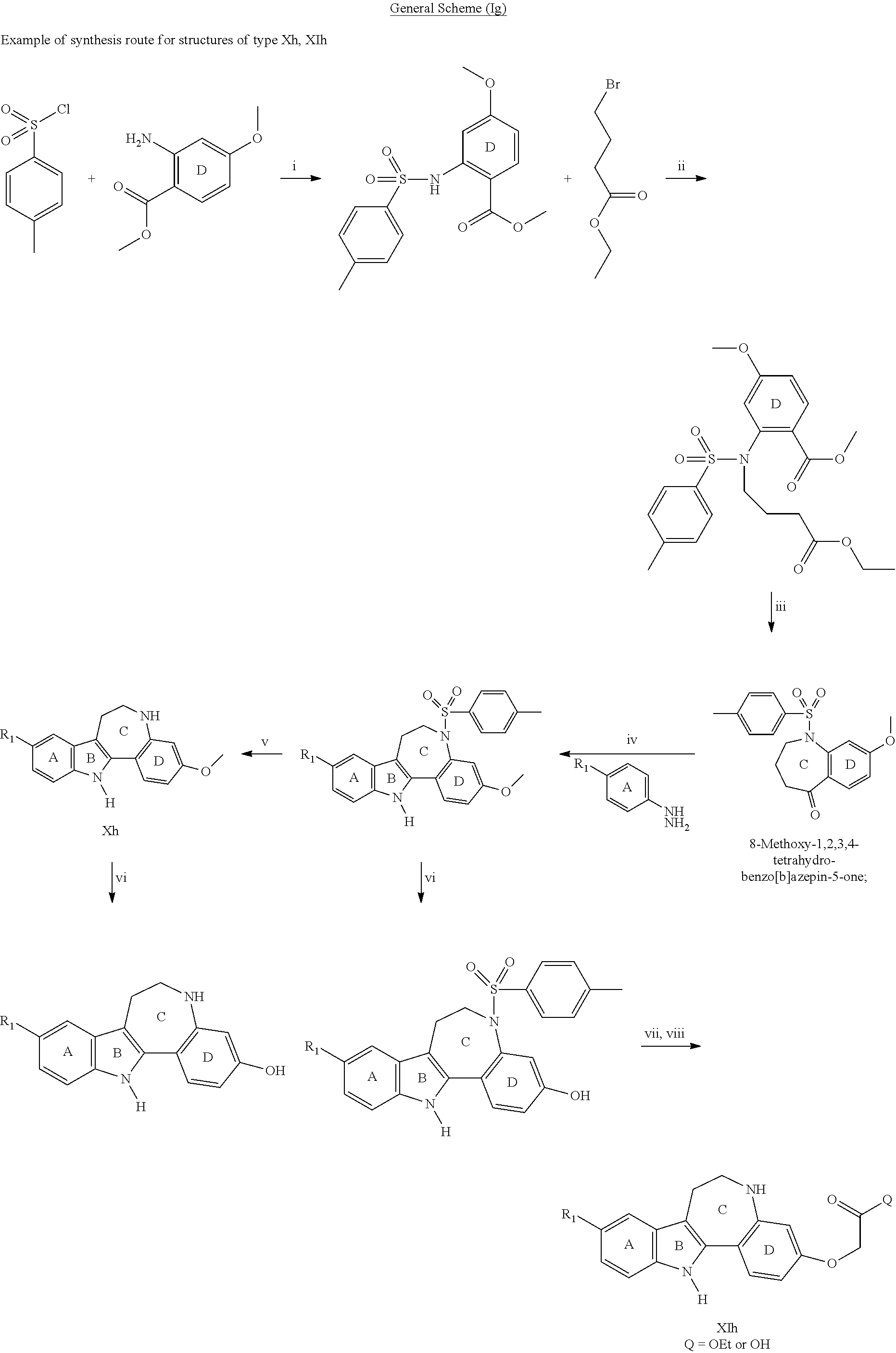

Reaction conditions—reagents, solvents and appropriate conditions according to type of reaction: i: pyridine, DCM, 4 hours r/t; K₂CO₃, MeCOEt, 12 hours r/t; t-BuOK, toluene, 12-18 hours r/t, then 6N HCl, AcOH, r/t, 12-18 h; iv: —R₁-phenylhydrazine, or 4-R₁-phenylhydrazine hydrochloride or hydrobromide and appropriate acidic conditions 36% HCl reflux or AcOH, 120° C., 2-6 h, or microwave use in appropriate conditions 2-10 min, 2 hours; v: polyphosphoric acid, 50-80° C., inert atmosphere (Ar or N₂), 1-2 hours, then NaOH/H₂O to pH 8-9, extraction; vi: HBr 48%, AcOH, 120° C., 12 h., or BBr₃, dichloromethane, −78° C. to −0°; vii: BrCH₂COOEt, K₂CO₃, acetone, reflux; iv: KOH/EtOH; viii: KOH/EtOH.

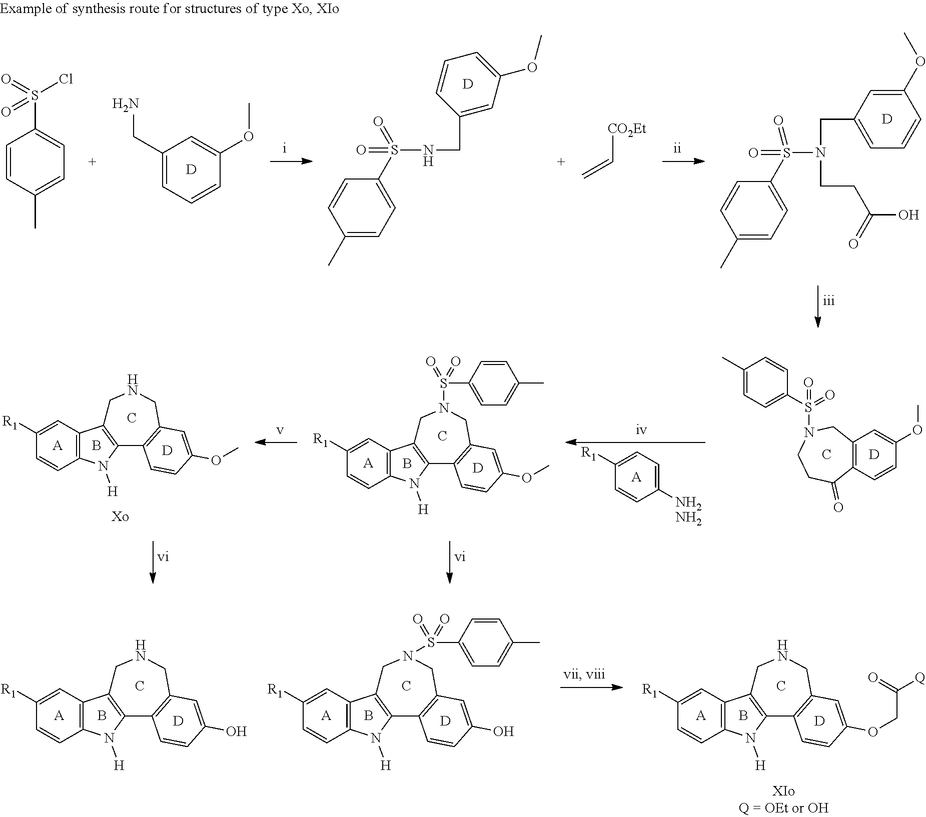

Reaction conditions—reagents, solvents and appropriate conditions according to type of reaction: i: NMM, DMAP (cat.), DCM or THF, 0-20° C., 4 hours, or Et₃N, H₂O, dioxane; K₂CO₃, DMF or CH₃CN, 12 hours r/t, KOH, EtOH, 12 hours, 10% HCl extraction; iii: t-BuOK, toluene, 12-18 hours r/t, then 6N HCl, AcOH, r/t, 12-18 h; iv: —R₁-phenylhydrazine, or 4-R₁-phenylhydrazine hydrochloride or hydrobromide and appropriate acidic conditions HCl 36% reflux or AcOH, 120° C., 2-6 h, or microwave use in appropriate conditions 2-10 min, 2 hours; v: polyphosphoric acid, 50-80° C., inert atmosphere (Ar or N₂), 1-2 hours, then NaOH/H₂O to pH 8-9, extraction; vi: HBr 48%, AcOH, 120° C., 12 h., or BBr₃, dichloromethane, −78° C. to −0°; vii: BrCH₂COOEt, K₂CO₃, acetone, reflux; iv: KOH/EtOH; viii: KOH/EtOH.

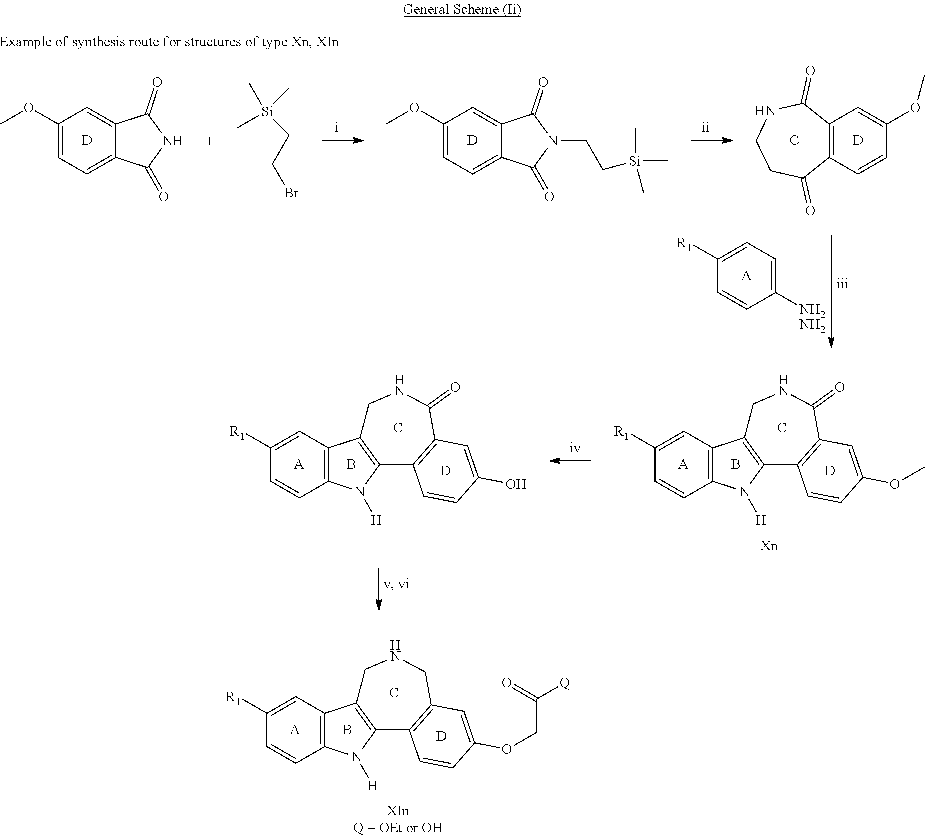

Reaction conditions—reagents, solvents and appropriate conditions according to type of reaction: i: CsCO₃, DMF, 1 hour, r/t; ii: irradiation at λ>250 in CH₃CN, [Tetrahedron Lett., 34, 37, 1993, 5855-58]; iii: —R₁-phenylhydrazine, or 4-R₁-phenylhydrazine hydrochloride or hydrobromide and appropriate acidic conditions 36% HCl reflux or AcOH, 120° C., 2-6 h, or microwave use in appropriate conditions 2-10 min, 2 hours; iv: HBr 48%, AcOH, 120° C., 12 h., or BBr₃, dichloromethane, −78° C. to −0°; v: BrCH₂COOEt, K₂CO₃, acetone, reflux; vi: KOH/EtOH.

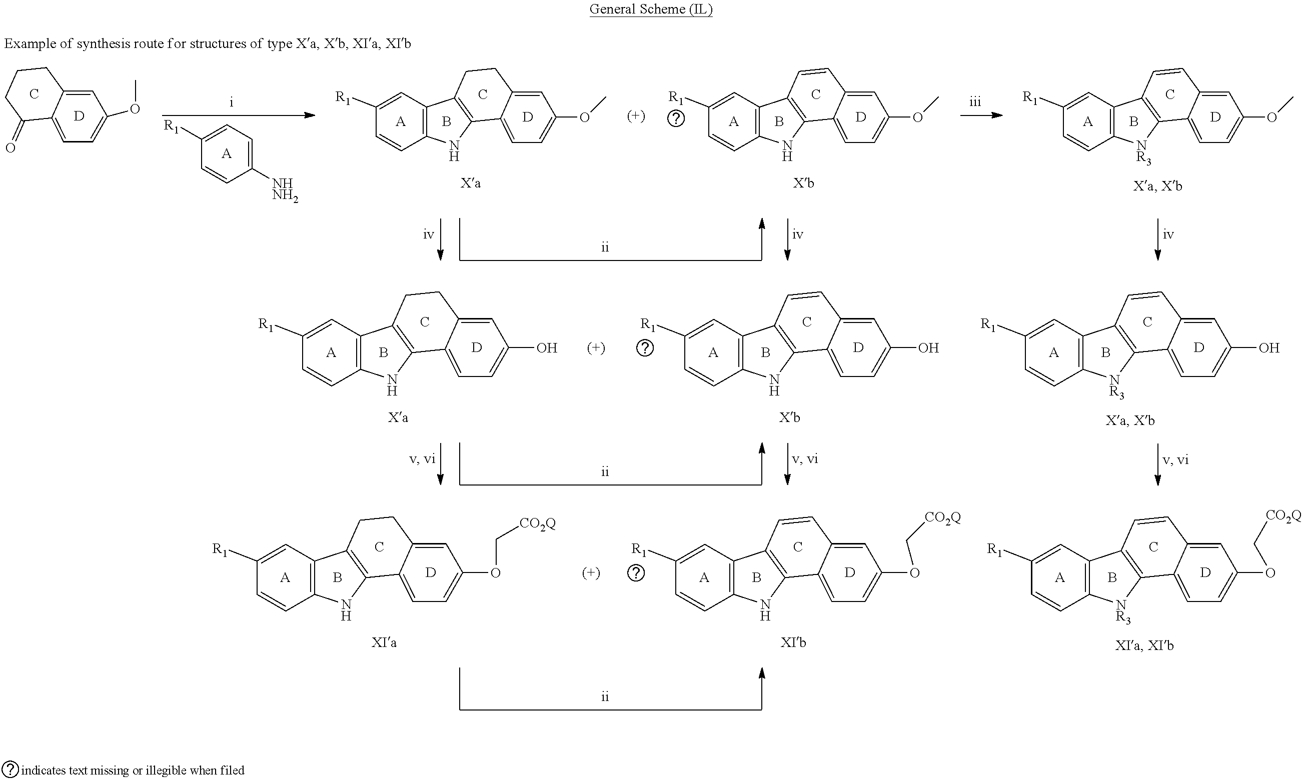

Reaction conditions—reagents, solvents and appropriate conditions according to type of reaction: i: —R₁-phenylhydrazine, or 4-R₁-phenylhydrazine hydrochloride or hydrobromide and appropriate acidic conditions 36% HCl reflux or AcOH, 120° C., from 2-6 h to 5 days, or microwave use in appropriate conditions 2-10 min, 2 hours, [(+)* according to conditions X′ and XI′ a and b mixed or a alone]; ii: AcOH, Pd/C 120° C., 20 hours or iia: (2,2,6,6-tetramethyl-piperidin-1-yl)oxyl (TEMPO)/HBF₄ 50%, 0° C., 10 min, iib: N-oxoammonium-TEMPO, CH₃CN, 0° C., 15 min; iii: appropriate R₃—X halide (mainly but not only Br or CO, NaH/anh. DMF, 80° C., 16 h, or microwaves in appropriate conditions 2-10 min.; iv: HBr 48%, AcOH, 120° C., 12 h, or BBr₃, dichloromethane, −78° C. to −0°; v: BrCH₂COOEt, K₂CO₃, acetone, reflux; vi: KOH/abs. EtOH, r/t, 20 hours/H⁺.

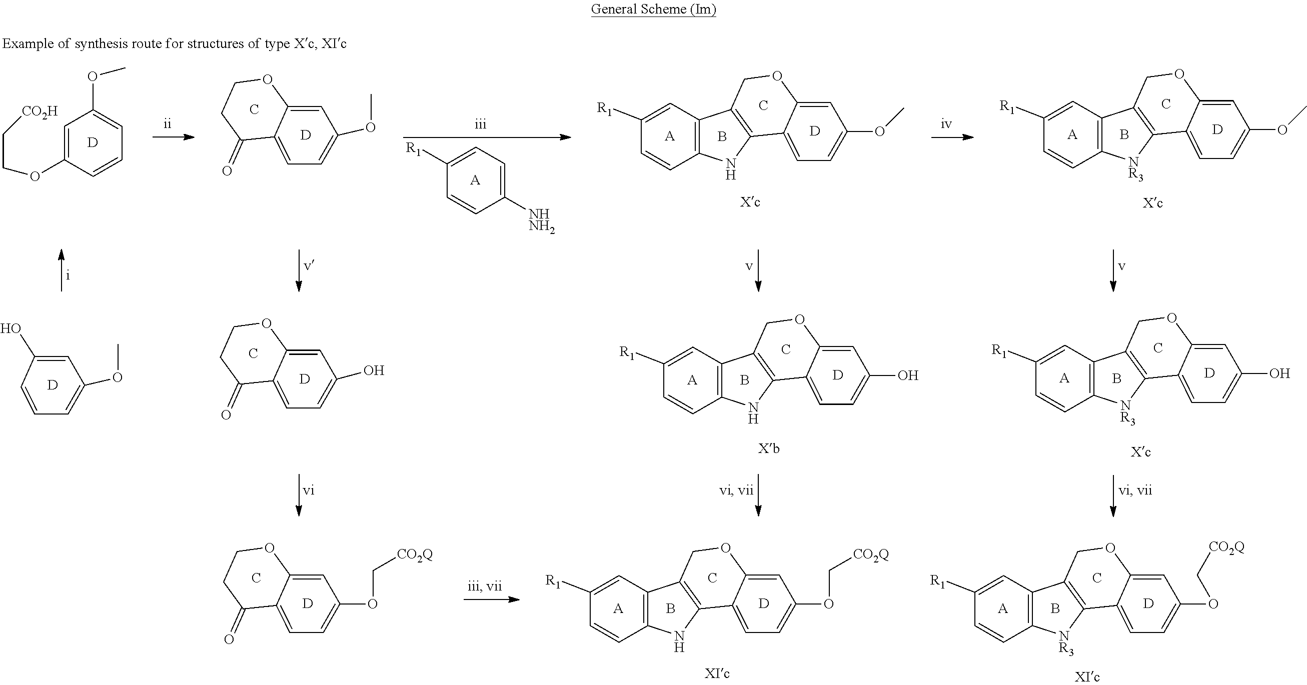

Reaction conditions—reagents, solvents and appropriate conditions according to type of reaction: i: 3-Cl-propionic acid, 20% KOH, 100° C., 3 days; ii: Polyphosphoric acid, 70° C., 1 hour; iii: —R₁-phenylhydrazine, or 4-R₁-phenylhydrazine hydrochloride or hydrobromide and appropriate acidic conditions 36% HCl reflux or AcOH, 120° C., from 2-6 h to 5 days, or polyphosphoric acid, 70-75° C., 1.5 hours, then 2 hours r/t, or microwave use in appropriate conditions 2-10 min, 2 hours; iv: appropriate R₃—X halide (mainly but not only Br or Cl), NaH/anh. DMF, 80° C., 16 h, or microwaves in appropriate conditions 2-10 min.; v: HBr 48%, AcOH, 120° C., 12 h, or BBr₃, dichloromethane, −78° C. to −0°; v′: pyridine hydrochloride 180-190° C., inert atmosphere (N₂ or Ar) 2 hours, or pyridine hydrochloride microwaves 5 min; vi: BrCH₂COOEt, K₂CO₃, acetone, reflux; vii: KOH/abs. EtOH, r/t, 20 hours/H⁺.

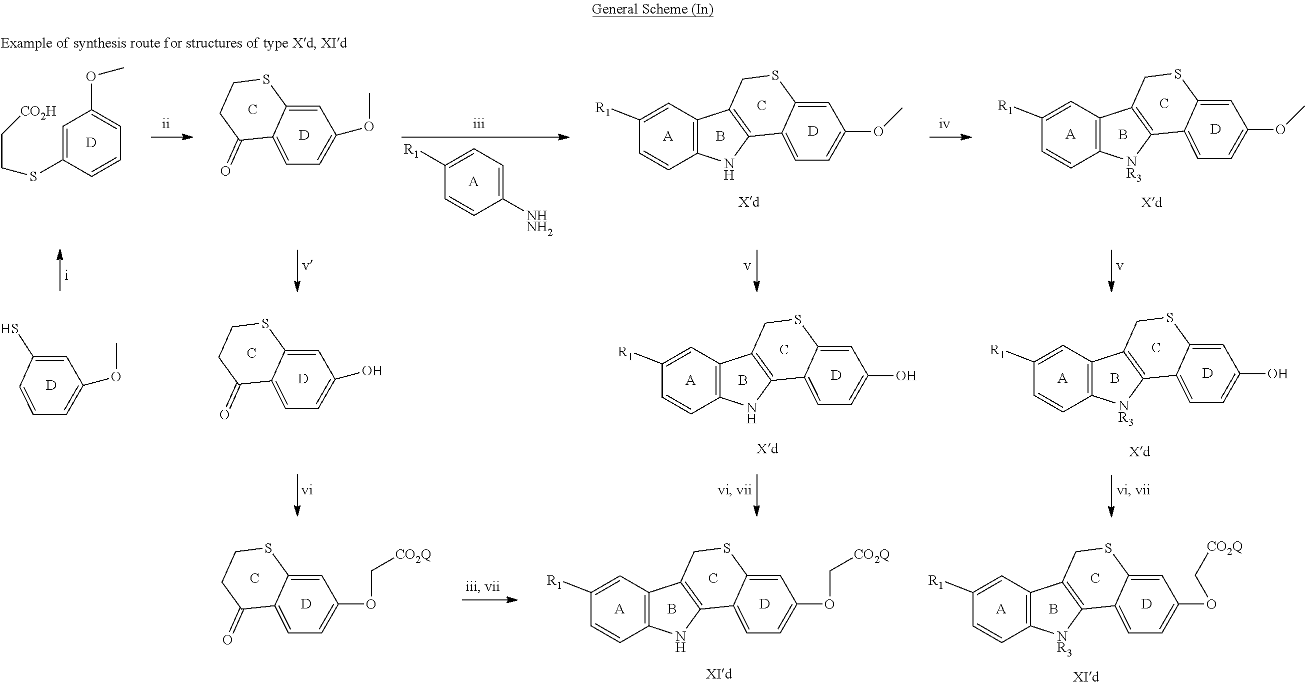

Reaction conditions—reagents, solvents and appropriate conditions according to type of reaction: i: 3-Cl-propionic acid, 20% NaOH, 100° C. for 2 hours then 60° C. for 12 hours, r/t/HCl 3 hours and extraction; ii: Polyphosphoric acid, 50° C., 4 hours; iii: —R₁-phenylhydrazine, or 4-R₁-phenylhydrazine hydrochloride or hydrobromide and appropriate acidic conditions 36% HCl reflux or AcOH, 120° C., from 2-6 h to 5 days, or microwave use in appropriate conditions 2-10 min, 2 hours; iv: appropriate R₃—X halide (mainly but not only Br or Cl), NaH/anh. DMF, 80° C., 16 h, or microwaves in appropriate conditions 2-10 min.; v: HBr 48%, AcOH, 120° C., 12 h, or BBr₃, dichloromethane, −78° C. to −0°; v′: pyridine hydrochloride 180-190° C., inert atmosphere (N₂ or Ar) 2 hours, or pyridine hydrochloride microwaves 5 min; vi: BrCH₂COOEt, K₂CO₃, acetone, reflux; vii: KOH/abs. EtOH, r/t, 20 hours/H⁺.

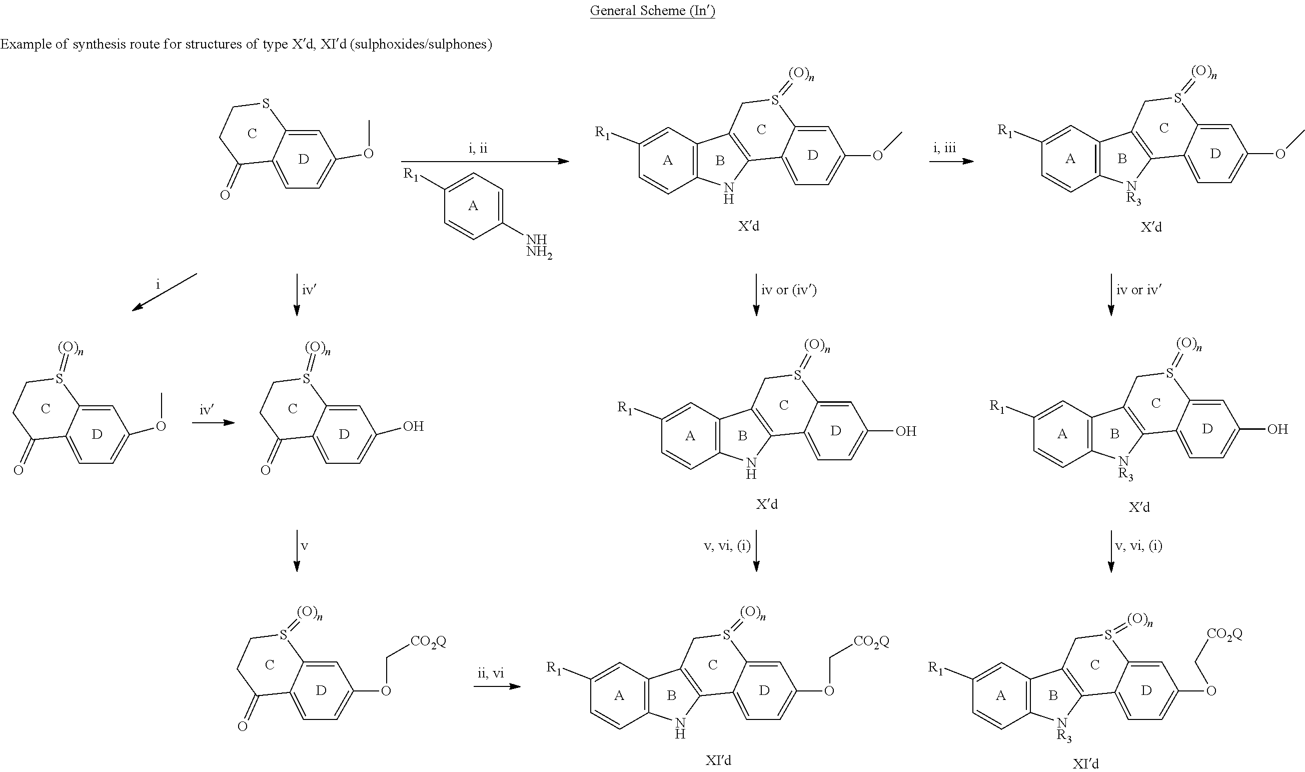

Reaction conditions—reagents, solvents and appropriate conditions according to type of reaction: i: R₁-phenylhydrazine, or 4-R₁-phenylhydrazine hydrochloride or hydrobromide and appropriate acidic conditions 36% HCl reflux or AcOH, 120° C., from 2-6 h to 5 days, or microwave use in appropriate conditions 2-10 min, 2 hours; ii: MCPB, DCM. r/t 2-6 hours or Ti(O-ᵢPr)₄, (R,R)-diethyltartrate, DCM, 15° C., 15-30 min. or Oxone®, THF/MeOH, r/t, 24-120 hours; iii: appropriate R₃—X halide (mainly but not only Br or Cl), NaH/anh. DMF, 80° C., 16 h, or microwaves in appropriate conditions 2-10 min.; iv: HBr 48%, AcOH, 120° C., 12 h, or BBr₃, dichloromethane, −78° C. to −0°; iv′: pyridine hydrochloride 180-190° C., inert atmosphere (N₂ or Ar) 2 hours, or pyridine hydrochloride microwaves 5 min; v: BrCH₂COOEt, K₂CO₃, acetone, reflux; vi: KOH/abs. EtOH, r/t, 20 hours/H⁺.

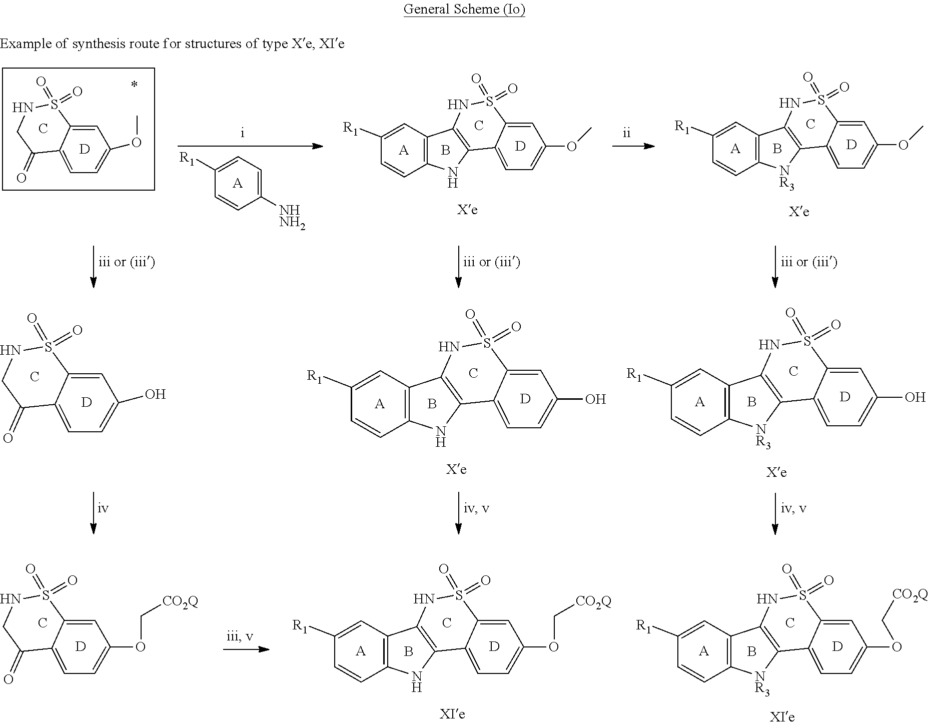

Reaction conditions—reagents, solvents and appropriate conditions according to type of reaction: i: R₁-phenylhydrazine, or 4-R₁-phenylhydrazine hydrochloride or hydrobromide and appropriate acidic conditions 36% HCl reflux or AcOH, 120° C., from 2-6 h to 5 days, or microwave use in appropriate conditions 2-10 min, 2 hours; ii: appropriate R₃—X halide (mainly but not only Br or Cl), NaH/anh. DMF, 80° C., 16 h, or microwaves in appropriate conditions 2-10 min.; HBr 48%, AcOH, 120° C., 12 h, or BBr₃, dichloromethane, −78° C. to −0°; iii′: pyridine hydrochloride 180-190° C., inert atmosphere (N₂ or Ar) 2 hours, or pyridine hydrochloride microwaves 5 min; iv: BrCH₂COOEt, KOH, EtOH 85%, r/t, 2 h, or BrCH₂COOEt, K₂CO₃, acetone or DMF, 70° C.; v: KOH/abs. EtOH, r/t, 20 hours/H⁺.

The intermediate 7-methoxy-1,1-dioxo-3,4-dihydro-2H-1,2-benzothiazin-4-one is prepared according to Scheme (Io′) below.

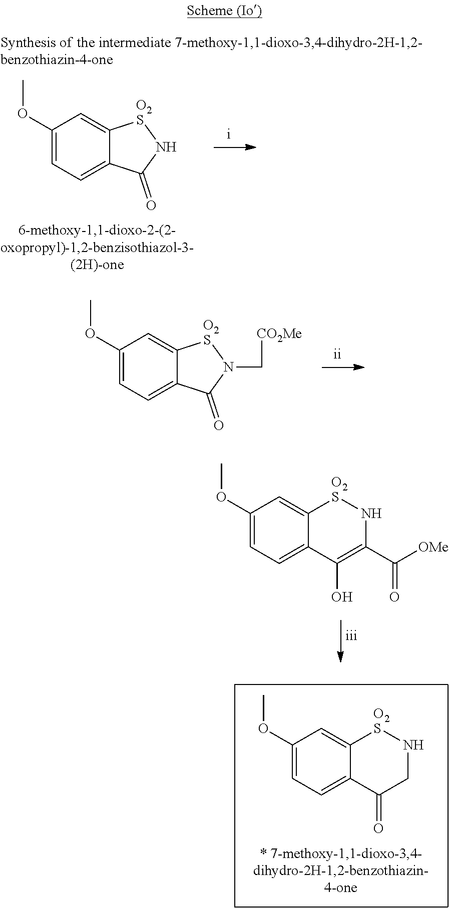

Reaction conditions—reagents, solvents and appropriate conditions according to type of reaction: i: NaH, DMF, 30 min r/t, methyl-chloroacetate 110° C.; ii: NaOMe, MeOH, 55° C., 30 min, HCl, 5° C. pH=3; iii: HCl/H₂O reflux.

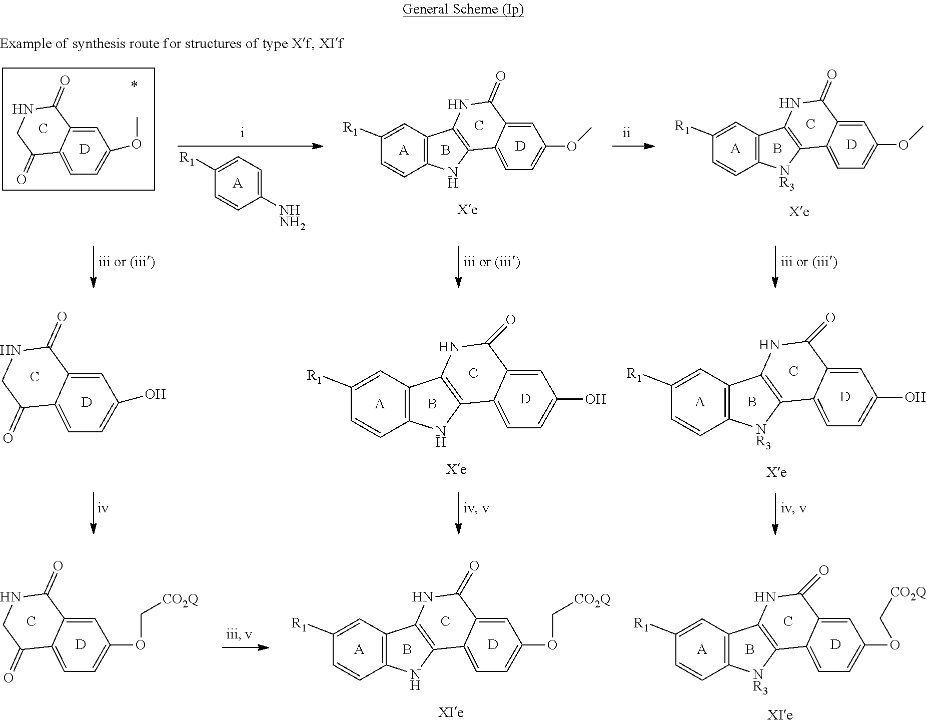

Reaction conditions—reagents, solvents and appropriate conditions according to type of reaction: i: R₁-phenylhydrazine, or 4-R₁-phenylhydrazine hydrochloride or hydrobromide and appropriate acidic conditions 36% HCl reflux or AcOH, 120° C., from 2-6 h to 5 days, or microwave use in appropriate conditions 2-10 min, 2 hours; ii: appropriate R₃—X halide (mainly but not only Br or Cl), NaH/anh. DMF, 80° C., 16 h, or microwaves in appropriate conditions 2-10 min.; HBr 48%, AcOH, 120° C., 12 h, or BBr₃, dichloromethane, −78° C. to −0°; iii′: pyridine hydrochloride 180-190° C., inert atmosphere (N₂ or Ar) 2 hours, or pyridine hydrochloride microwaves 5 min; iv: BrCH₂COOEt, KOH, EtOH 85%, r/t, 2 h or BrCH₂COOEt, K₂CO₃, acetone or DMF, 70° C.; v: KOH/abs. EtOH, r/t, 20 hours/H⁺.

The intermediate 7-methoxy-2,3-dihydro-isoquinolin-1,4-dione is prepared according to the following scheme (Scheme Ip′).

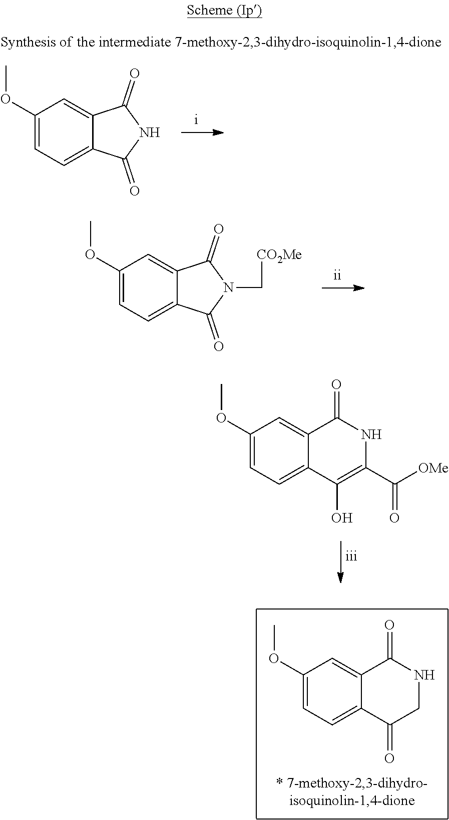

Reaction conditions—reagents, solvents and appropriate conditions according to type of reaction: i: NaH, DMF, 30 min r/t, methyl-chloroacetate 110° C.; ii: NaOMe, MeOH, 55° C., 30 min, HCl, 5° C. pH=3; iii: H₂SO₄/H₂O reflux, 1 hour.

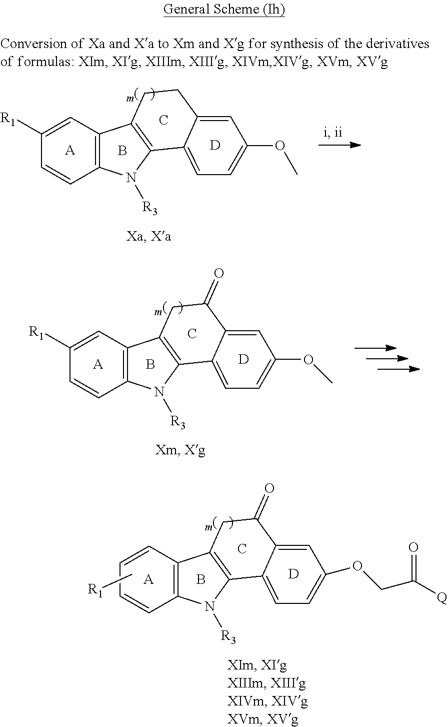

General reaction conditions for oxidation of methylenes (Xa and X′a) aryl-conjugated to carbonyl derivatives of type Xm and X′g for the synthesis of derivatives of formulas: XIm, XI′g, XIIm, XII′g, XIIIm, XIII′g, XIVm, XIV′g-reagents, solvents and appropriate conditions according to type of reaction: i: NaBH₄, Bi₂O₃, distilled H₂O r/t, forms a black precipitate washed with H₂O; ii: pyridine, AcOH, picolinic acid and t-BuOOH in H₂O (70). 30 min microwave heating at 100° C. for 16 h (glass vial), cool, dilute with DCM, filter through celite, evaporate at 1/p [Org. Lett., 7, 21, 4549-4552, 2005 and references cited therein].

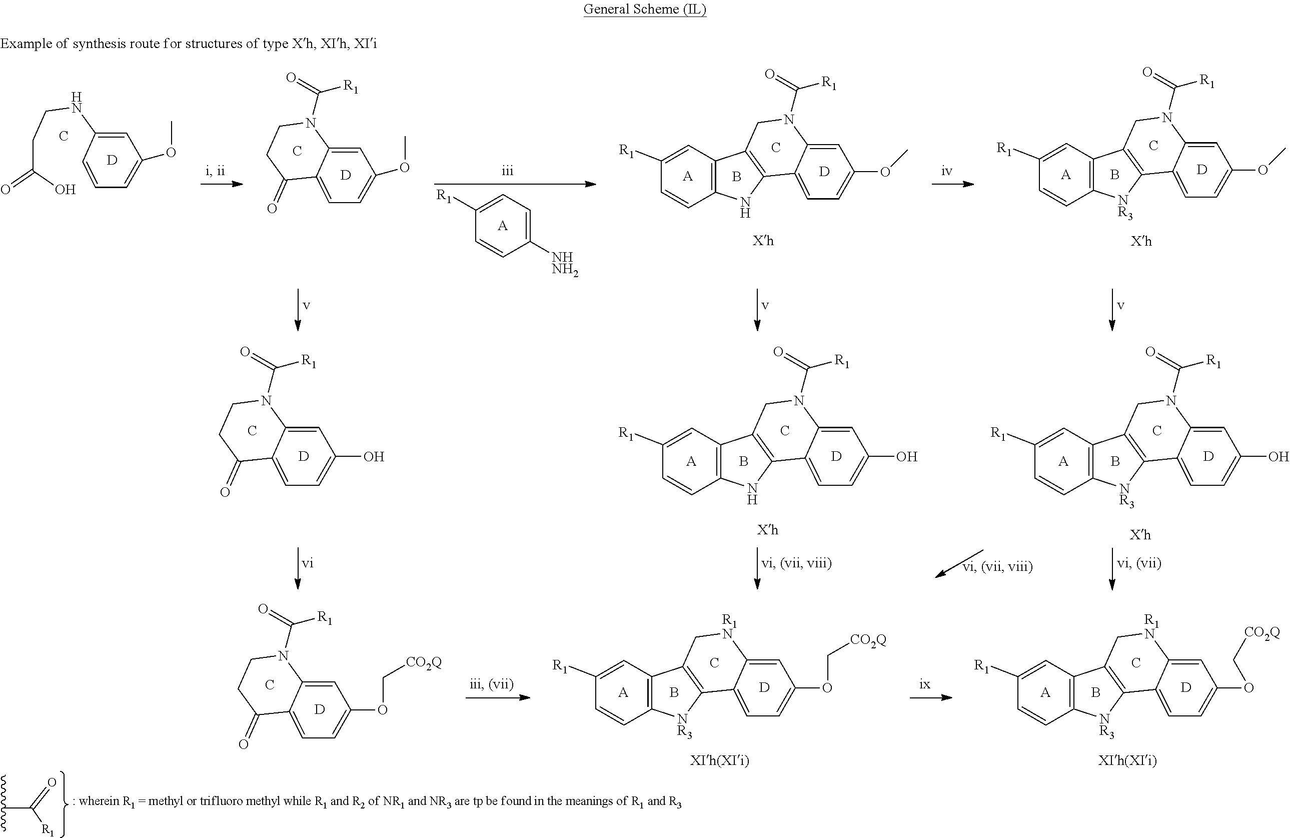

Reaction conditions—reagents, solvents and appropriate conditions according to type of reaction: i: [precursor synthesised as shown in JOC 67, 10, 3502-3505, 2002], polyphosphoric acid, 100° C., K₂CO₃/H₂O neutralisation, or propyl phosphonic acid, 90° C., 20 min, neutralisation and extraction; ii: Acetic anhydride or trifluoro acetic anhydride, 100° C., 16 hours, or acetyl chloride, toluene, pyridine, r/t, 12 hours; iii: R₁-phenylhydrazine, or 4-R₁-phenylhydrazine hydrochloride or hydrobromide and appropriate acidic conditions 36% HCl reflux or AcOH, 120° C., from 2-6 h to 5 days, or microwave use in appropriate conditions 2-10 min, 2 hours; iv: appropriate R₃—X halide (mainly but not only Br or Cl), NaH/anh. DMF, 80° C., 16 h, or microwaves in appropriate conditions 2-10 min.; v: BBr₃, dichloromethane, −78° C. to −0°, or: pyridine hydrochloride reflux 5 min, inert atmosphere (N₂ or Ar), H₂O, or pyridine hydrochloride microwaves 5 min, H₂O; vi: BrCH₂COOEt, KOH, EtOH 85%, r/t, 2 h or BrCH₂COOEt, K₂CO₃, acetone or DMF, 70° C.; vii: DBU, MeOH or CH₃CN, microwaves 20 min-4 hours or reflux 2-4 hours [Synth. Commun., 32(2), 265-272 (2002)]; or H₂SO₄/H₂O, MeOH or THF, reflux 5 min, then K₂CO₃/H₂O, r/t [Org. Lett. 16, 19, 5156-5159, 2014]; viii: appropriate R₁—X halide (mainly but not only Br or Cl), NaH/anh. DMF, 80° C., 16 h, or microwaves in appropriate conditions 2-10 min.; ix: KOH/abs. EtOH, r/t, 20 hours/H⁺.

The structures (X and X′) substituted in R₂ on the D ring as in (XI and XI′), which can preferably be acids (Q=OH), esters (Q=OR₁) or amides (Q=NHR₁), or in another preferential embodiment amides with a Q group consisting of a generic beta-lactam system as in general substructure XII from which amides with 3-amino-4-oxo azetidinones (XIII and XIII′) derive, or as in the case of amides with 7-amino-cephalosporin nuclei (XIV and XIV′), or as in the case of 6-amino-penicillin nuclei (XV and XV′), are obtainable either directly or by appropriate condensation according to General Scheme II:

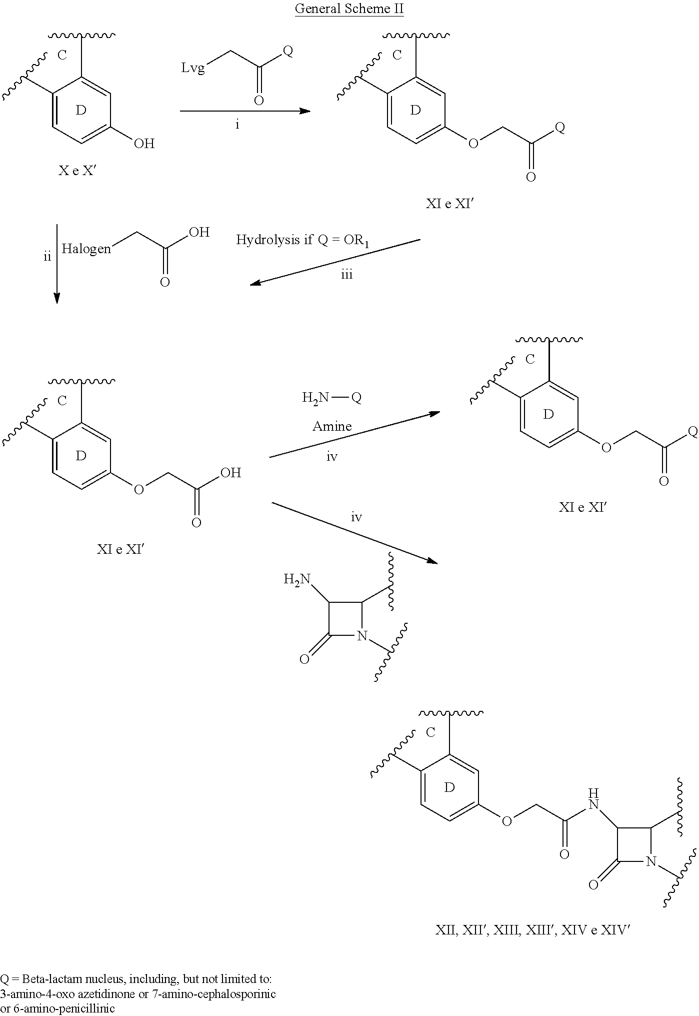

Reaction conditions—is Lvg=Halogen (Cl or Br or I) or MsO or TsO ester or OH; appropriate reagents, solvents and conditions according to the type of reaction; ii: (appropriate halide, Cl or Br or I), appropriate base (KOH or NaOH or K₂CO₃ or CsCO₃, appropriate solvents and conditions); iii: KOH (appropriate solvents and conditions); iv: acid activation: a) C₂O₂Cl₂, DMFcat, (solvents and appropriate conditions to form the suitable acid chloride of XI and XI′), or a′) ClCOOR, (solvents and appropriate conditions to form the suitable mixed anhydride of XI and XI′), or a″) activation as N-hydroxysuccinyl-ester, (solvents and appropriate conditions to form the suitable NOS ester of XI and XI′), or a′″) ECDI or other appropriate dehydrating agents (solvents and appropriate condensation conditions).

A further subject of the present invention is the use of the compounds of general formula (I) as described above for the treatment and diagnosis of degenerative disorders characterised by high cell proliferation and/or tissue degeneration.

The compounds of the invention can be used as therapeutic agents or diagnostic probes in degenerative disorders which may be endogenous or exogenous (ie. induced by infectious agents such as bacteria, viruses, protozoa or fungi), characterised by high cell proliferation and/or tissue degeneration.

The disorders which can be treated or diagnosed with the compounds of the invention are cancers of various kinds, and other disorders such as infectious disorders caused by pathogens wherein physiological homeostatic tissue control has been lost, and control of proliferative activity and cell apoptosis is important.

The following examples further illustrate the invention.

### EXAMPLES

Materials and Methods

The ¹H-NMR spectra were determined with a Varian Gemini 200 spectrometer operating at 200 MHz, or a Bruker Advance III HD 400 operating at 400 MHz. Chemical shifts (6) are expressed in parts per million (ppm). Coupling constants J are reported in Hertz. The following abbreviations have been used: singlet (s), doublet (d), triplet (t), broad singlet (br s) and multiplet (m). The preparatory Liquid Chromatography was conducted with flash chromatography, using pre-packed Isolute columns (Biotage) and glass columns containing silica gel 230-400 mesh. Thin-Layer Chromatography (TLC) was conducted with 60 F254 (MERCK) silica gel plates containing a fluorescent indicator. The various spots were highlighted with a UV lamp (256 nM). Evaporation was conducted under vacuum in a rotary evaporator, using anhydrous Na₂SO₄ as dehydrating agent.

The compounds of general formulas X′a to XV′m can be prepared, for example, as reported below for (XIV′a # RM37) and for the corresponding acid (XIV′a′ # RM53) (Scheme IIa).

### Example 1—Preparation of (6R, 7R)-tert-butyl-7-(2-(8-fluoro-6,11-dihydro-5H-benzo[a]carbazol-3-yloxy)acetamido)-3-(acetoxymethyl)-8-oxo-5-thia-1-azabicyclo[4.2.0]oct-2-ene-2-carboxylate (XIV′a # RM37) and its Corresponding Acid (6R,7R)-7-(2-[(8-fluoro-6,11-dihydro-5H-benzo[a]carbazol-3-yl)oxy]acetamido)-3-(acetoxymethyl)-8-oxy-5-thia-1-azabicyclo[4.2.0]oct-2-ene-2-carboxylic (XIV′a′ # RM53), Scheme IIa

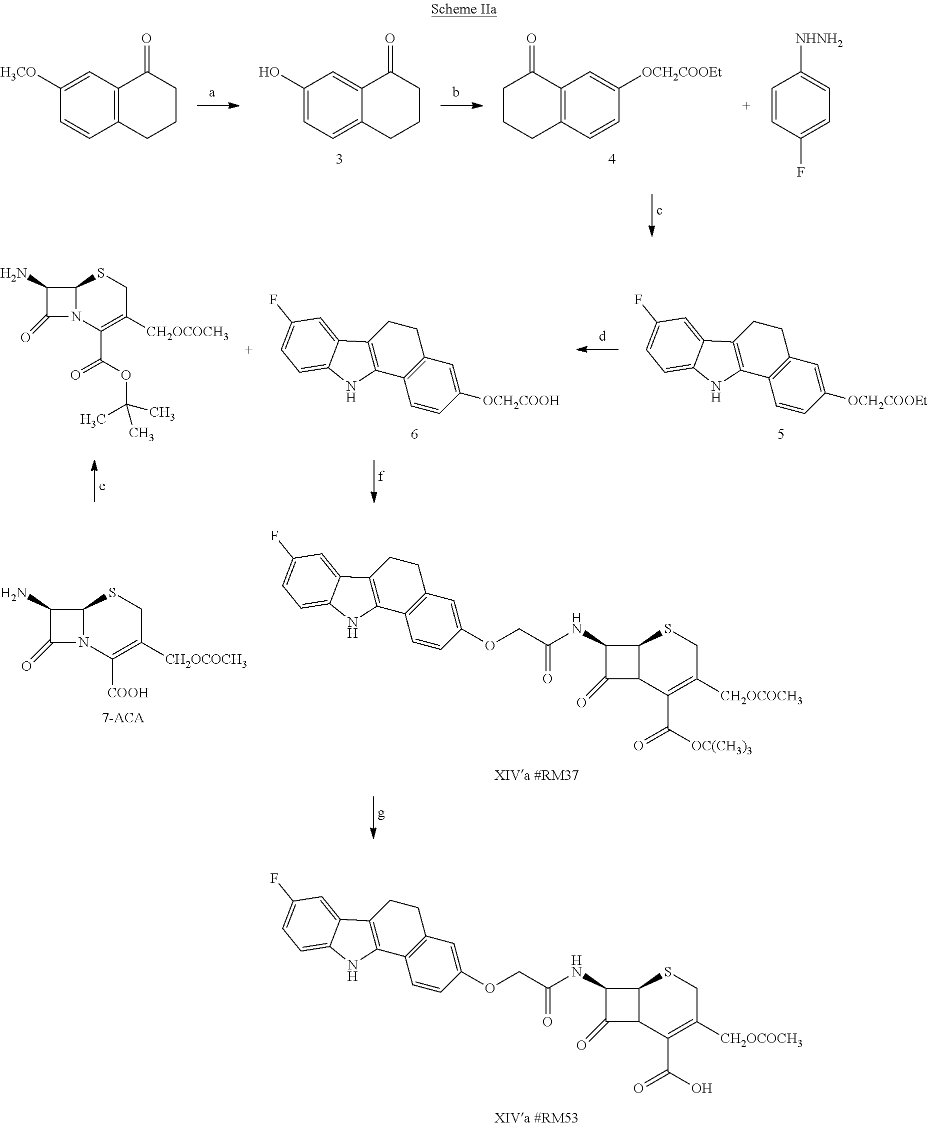

Reagents and conditions: a) HBr 48%, AcOH, 120° C., 12 h; b) BrCH₂COOEt, K₂CO₃, acetone, reflux; c) AcOH, 120° C., 6 h; d) KOH/EtOH; e) isobutene, H₂SO₄, dioxane; f) EDC, THF, 0° C.; g) TFA, anisole, CH₂Cl₂, 0° C.

### Synthesis of 7-hydroxy-3,4-dihydronaphthalen-1(2H)-one (3)

An aqueous solution of 48% HBr (62 mL) was added to a solution of 6-methoxy-1-tetralone (6 g, 34.05 mmols) in glacial AcOH (9 mL), and the reaction mixture was left at reflux at 120° C. under stirring overnight. The reaction was cooled and then evaporated under vacuum to give the crude compound 3 as a black semisolid. The residue was then purified by crushing with n-hexane to give 3 as a brown solid (6.76 g, 98% yield). ¹H-NMR (Acetone-d₆) δ: 1.98-2.18 (m, 4H); 2.80-2.95 (m, 2H); 6.70-6.82 (m, 2H); 7.80-7.90 (m, 1H).

### Synthesis of 2-(1,2,3,4-tetrahydro-1-oxonaphthalen-7-yloxy) ethyl acetate (4)

Potassium carbonate (17.120 g, 125.11 mmols) was added to a solution of hydroxy tetralone 3 (6.76 g, 41.70 mmols) in anhydrous acetone (200 mL) placed under nitrogen, and the mixture was left under stirring for 1 hour at room temperature. Ethyl bromoacetate (7 mL) was then added, and the reaction was left under stirring overnight at 57° C., then filtered to eliminate inorganic salts, and finally, the filtrate was evaporated at low pressure. The resulting residue was then taken up with AcOEt (200 mL) and washed with a saturated solution of NaHCO₃(2×100 mL) and brine (2×100 mL). The organic phase was then dried and evaporated to give a brown oil. Finally, the crude product was purified by flash chromatography on silica gel (n-hexane/AcOEt 5:1) to give the pure compound 4 as a yellow oil (6.67 g, 61% yield). ¹H-NMR (CDCl₃) δ: 1.30 (t, J=7.1 Hz, 3H); 2.05-2.17 (m, 2H); 2.61 (t, J=6.8 Hz, 2H); 2.92 (t, J=6.0 Hz, 2H); 4.3 (q, J=7.1 Hz, 2H); 4.67 (s, 2H); 6.71-6.84 (m, 2H); 8.0 (d, J=8.6 Hz, 1H).

### Synthesis of ethyl 2-[(8-fluoro-6,11-dihydro-5H-benzo[a]carbazol-3-yl)oxy]acetate (5)

A mixture of tetralone 4 (1.0 g, 3.80 mmols) and (4-fluorophenyl)hydrazine (0.643 g, 3.95 mmols) in glacial AcOH (5.3 mL) was placed at reflux at 120° C. for 6 hours under an inert atmosphere. The suspension was then cooled to room temperature and evaporated to give a solid yellow residue. The residue was taken up with AcOEt (150 mL), washed with H₂O (6×80 mL) and saturated solution of NaHCO₃ (1×80 mL), dried on Na₂SO₄ and evaporated at low pressure. The crude product obtained was then purified by crushing with n-hexane/Et₂O to give 5 as a yellow solid (0.920 g, 72% yield). ¹H-NMR (CDCl₃) δ: 1.32 (t, J=7.2 Hz, 3H); 2.85-3.09 (m, 4H); 4.22 (q, J=7.2 Hz, 2H); 4.65 (s, 2H); 6.72-6.72 (m, 6H); 7.08-7.31 (m, 3H); 8.18 (brs, 1H).

### Synthesis of 2-[(8-fluoro-6,11-dihydro-5H-benzo[a]carbazol-3-yl)oxy]acetic acid (6)

A mixture of carbazole 5 (0.920 g, 2.71 mmols) and KOH (0.182 g, 3.25 mmols) in absolute EtOH (65 mL) was left under stirring at room temperature for 2 days. The solvent was then evaporated and the resulting yellow residue was dissolved in H₂O. The aqueous solution was acidified with 10% HCl to pH 1 and extracted with AcOEt (4×100 mL). The organic phase, dried and evaporated, supplied carboxylic acid 6 as a yellow solid (0.767 g, 91% yield). ¹H-NMR (DMSO-d₆) δ: 2.75-2.90 (m, 2H); 2.92-3.05 (m, 2H); 4.70 (s, 2H); 6.79-6.84 (m, 1H); 6.86-6.96 (m, 2H); 7.14-7.24 (m, 1H); 7.26-7.38 (m, 1H); 7.51-7.63 (m, 1H); 11.42 (s, 1H).

### Synthesis of (6R, 7R)-tert-butyl-3-(acetoxymethyl)-7-amino-8-oxo-5-thia-1-aza-bicyclo[4.2.0]oct-2-ene-carboxylate (7)

Isobutene (32 mL) was added to a mixture of 7-aminocephalosporanic acid (7-ACA) (7.0 g, 25.71 mmols) and conc. H₂SO₄ (6.42 mL) in anhydrous dioxane (64 mL), placed at 0° C. The reaction mixture was stirred at room temperature for 2 hours. The mixture was then adjusted to an alkaline pH by adding a saturated solution of NaHCO₃, extracted with AcOEt (3×200 mL), washed with brine (5×100 mL), dried and evaporated to give a brown oil. The crude product was then purified by crushing with n-hexane to give pure 7 as a yellow solid (4.154 g, 50% yield). ¹H-NMR (CDCl₃) δ: 1.56 (s, 9H); 2.11 (s, 3H); 2.02-2.05 (brs, 2H); 3.40-3.57 (2d, J=18.2 Hz, 2H); 4.78-4.85 (m, 2H); 5.0 (d, J=5.1 Hz, 1H); 5.07 (d, J=13.2 Hz, 1H).

### Synthesis of (6R, 7R)-tert-butyl-7-(2-(8-fluoro-6,11-dihydro-5H-benzo[a]carbazol-3-yloxy)acetamido)-3-(acetoxymethyl)-8-oxo-5-thia-1-azabicyclo[4.2.0]oct-2-ene-2-carboxylate (XIV′a; # RA/137)

7-ACA tert-butyl ester 7 (0.211 g, 0.642 mmols) was added to a solution of carboxylic acid 6 (0.200 g, 0.642 mmols), in anhydrous THF (31 mL). The reaction mixture was cooled in an ice bath, and EDC (0.136 g, 0.642 mmols) was added gradually. The reaction was left under stirring in an inert atmosphere at room temperature for 24 hours. The solvent was evaporated at room temperature and the residue was taken up with CHCl₃ (60 mL), washed with H₂O (4×25 mL), dried on Na₂SO₄ and evaporated to give a yellow solid. The crude product was purified by flash chromatography (n-hexane/AcOEt 3:1) using an Isolute column (Si II) to give XIV′a as a yellow oil (0.267 g, 67% yield). ¹H-NMR (CDCl₃) δ: 1.55 (s, 9H); 2.07 (s, 3H); 2.87-2.94 (m, 2H); 2.99-3.06 (m, 2H); 3.31-3.58 (2d, J=18.4 Hz, 2H); 4.58 (s, 2H); 4.76-4.83 (m, 1H); 4.99-5.05 (m, 2H); 5.89-5.94 (q, J=4.9 Hz, 1H); 6.76-6.81 (m, 1H), 6.85-6.92 (m, 2H); 7.12-7.17 (m, 1H); 7.24-7.30 (m, 2H); 8.30 (s, 1H).

### Synthesis of (6R,7R)-7-(2-[(8-fluoro-6,11-dihydro-5H-benzo[a]carbazol-3-yl)oxy]acetamido)-3-(acetoxymethyl)-8-oxy-5-thia-1-azabicyclo[4.2.0]oct-2-ene-2-carboxylic acid (XIV′a′, # RM53)

Anisole (0.05 mL) and TFA (0.42 mL) were added to a solution of compound 1 (60 mg, 0,097 mmols) in anhydrous CH₂Cl₂ (1.0 mL) placed on ice. The reaction mixture was stirred at room temperature for 18 hours under a nitrogen atmosphere. The reaction was then evaporated at room temperature, and the resulting yellow solid was crushed with Et₂O to give compound XIV′a′ as a yellow solid (18 mg, 33% yield). ¹H-NMR (DMSO-d₆) δ: 2.04 (s, 3H); 2.81-2.88 (m, 2H); 2.94-3.0 (m, 2H); 3.48-3.69 (2d, J=18 Hz, 2H); 4.65-4.74 (m, 2H); 5.0 (d, J=12.8 Hz, 1H); 5.15 (d, J=4.8 Hz, 1H); 5.73-5.77 (q, J=4.8 Hz, 1H); 6.84-6.94 (m, 3H), 6.78-7.23 (m, 1H); 7.29-7.34 (m, 1H); 7.55-7.60 (m, 1H); 9.13 (d, J=8.4 Hz, 1H); 11.40 (s, 1H).

Example 2—by analogy with XIV′a; # RM37, compounds XIV′a (# GA09a, # GA09b, RM36, RM47) can be prepared, as reported below, and consequently, as for acid XIV′a′ # RM53, their corresponding acids of type XIV′a′ (# GA09a′, # GA09b′9) can be obtained (Scheme II b)

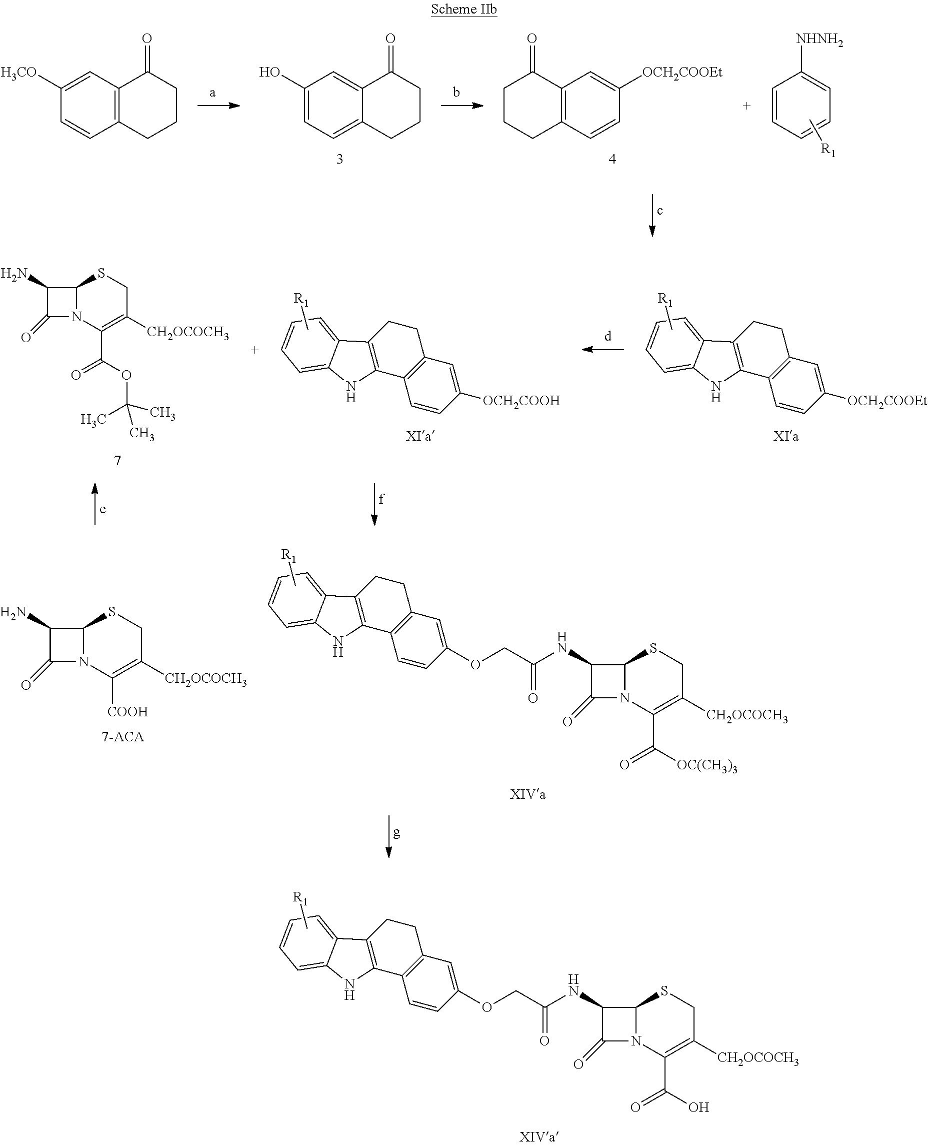

Reagents and conditions: a) HBr 48%, AcOH, 120° C., 12 h; b) BrCH₂COOEt, K₂CO₃, acetone, reflux; c) AcOH, 120° C., 6 h; d) KOH/EtOH; e) isobutene, H₂SO₄, dioxane; f) EDC, THF, 0° C.; g) TFA, anisole, CH₂Cl₂, 0° C.

### Example of Preparation of (6R, 7R)-tert-butyl-7-(2-(7-nitro-6,11-dihydro-5H-benzo[a]carbazol-3-yloxy)acetamido)-3-(acetoxymethyl)-8-oxo-5-thia-1-azabicyclo[4.2.0]oct-2-ene-2-carboxylate (XIV′a # GA09a), (6R, 7R)-tert-butyl-7-(2-(9-nitro-6,11-dihydro-5H-benzo[a]carbazol-3-yloxy)acetamido)-3-(acetoxymethyl)-8-oxo-5-thia-1-azabicyclo[4.2.0]oct-2-ene-2-carboxylate (XIV′a # GA09b) and their Corresponding Acids (6R,7R)-7-(2-[(7-nitro-6,11-dihydro-5H-benzo[a]carbazol-3-yl)oxy]acetamido)-3-(acetoxymethyl)-8-oxy-5-thia-1-azabicyclo[4.2.0]oct-2-ene-2-carboxylic acid (XIV′a′ # GA09a′) and (6R,7R)-7-(2-[(9-nitro-6,11-dihydro-5H-benzo[a]carbazol-3-yl)oxy]acetamido)-3-(acetoxymethyl)-8-oxy-5-thia-1-azabicyclo[4.2.0]oct-2-ene-2-carboxylic acid (XIV′a′ # GA09b′) Scheme IIb

### Example 3—General Method for the Preparation of 8-(R′)-5,6-dihydrobenzo[a]carbazoles (XI′a)

A suitable R′-phenylhydrazine (e.g. R′=4-MeO or 4-Cl or 4-Br or 3-NO₂) (8.39 mmols) is added to a tetralone solution 4 (8.05 mmols) in glacial acetic acid (11.4 ml), and the mixture is maintained at reflux and under stirring for 8 hours. After said time has elapsed, the reaction is cooled in an ice bath to facilitate the formation of a precipitate. The crystalline solid is separated from the reaction mixture by vacuum filtration. The crystalline solid obtained is washed 4/5 times with water to eliminate the excess glacial acetic acid. The precipitation mother liquors (acetic acid) are evaporated at 1/p, and the resulting residue is crystallised several times from EtOH/Et₂O and vacuum filtered.

XI′a (R′=8-MeO):

Yield: 17.21%. M.p.: 168-169. ¹HNMR (DMSO-d6) δ: 1.25 (t, J=7.2 Hz, 3H), 2.91 (m, 4H), 3.78 (s, 3H), 4.20 (q, J=7.2 Hz, 2H), 4.78 (s, 2H) 6.62-7.71 (m, 6H), 11.05 (s, H). MS m/e 352 (M+)

XI′a (R′=8-Cl):

Yield: 50.59%. M.p.: 147-148. ¹HNMR (DMSO-d6) δ: 1.25 (t, J=7.2 Hz, 3H), 2.92 (m, 4H), 4.20 (q, J=7.2 Hz, 2H), 4.79 (s; 2H), 6.81-7.78 (m, 6H), 11.45 (s, H). MS m/e 356 (M+)

XI′a (R′=8-Br):

Yield: 39.99%. M.p.: 154-155. ¹HNMR (DMSO-d6) δ: 1.25 (t, J=7.2 Hz, 3H), 2.91 (m, 4H), 4.20 (q, J=7.2 Hz, 2H), 4.79 (s, 2H) 6.81-7.71 (m, 6H), 11.47 (s, H). MS m/e 400 (M+)

### Example 4—General Method for the Preparation of 7-(R′)-5,6-dihydrobenzo[a]carbazoles and 9-(R′)-5,6-dihydrobenzo[a]carbazoles (XI′a)

The appropriate 3-R′ phenyl hydrazine (e.g. R′=3-NO₂) (11.36 mmols) is added to a solution of the appropriate tetralone 3 (11.0 mmols) in glacial acetic acid (15.5 ml), and the mixture is heated to reflux under stirring for 6 hours. When that time has elapsed, the solvent (acetic acid) is removed by evaporation at 1/p and the semisolid residue obtained, dissolved in THF and preabsorbed on silica gel 60 (Merck 70-230 mesh, using a mixture/silica weight ratio of 1/7), is MPLC chromatographed on a silica gel 60 (Merck 230-400 mesh) column (d: 4 cm, h: 45 cm), using a hexane-ethyl acetate 2:1 mixture as eluent and collecting fractions of about 20 ml.

The two regioisomers 7-(NO₂)-5,6-dihydrobenzo[a]carbazole (XI′a, R′=7-NO₂) and 9-(NO₂)-5,6-dihydrobenzo[a]carbazole (XI′a, R′=9-NO₂) are separated by the chromatography (present in the crude reaction product at the ratio of 1:1 demonstrated by 1HMNR).

XI′a (R′=7-NO₂):

Yield: 10.6% M.p.: 221-222° C. ¹HNMR (DMSO-d6): δ 1.24 (t, J=7.2 Hz, 3H), 2.85-3.15 (m, 2H), 4.19 (q, J=7.2 Hz, 2H), 4.80 (s, 2H), 6.50-8.60 (m, 6H), 11.52 (s, H).

XI′a (R′=9-NO₂):

Yield: 29.7% M.p.: 179-180° C. ¹HNMR (DMSO-d6): δ 1.23 (t, J=7.2 Hz, 3H), 1.77-2.01 and 2.42-2.80 (2m, 4H), 4.18 (q, J=7.2 Hz, 2H), 4.77 (s, 2H) 6.74-6.88, 6.62-7.00 and 7.82-8.21 (3m, 6H), 10.05 (s, H).

### Example 5

Similarly to the compounds of type X′a, benzyl esters 15a and 15c can be prepared as pure from tetralones 16-18; for example, 5,6-dihydrobenzo[a]carbazoles (XI′a) 19-24 are prepared.

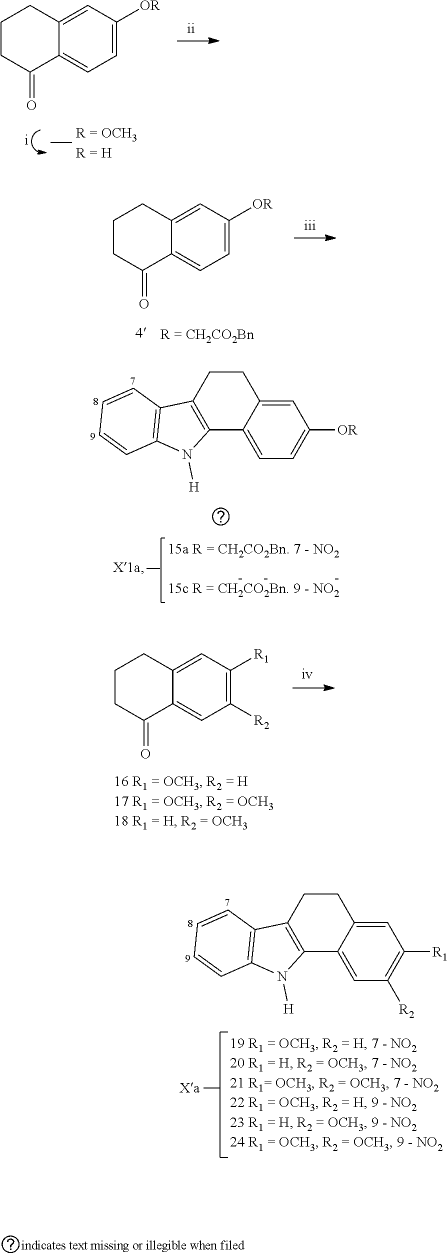

Conditions—i: HBr, CH3CO2H, reflux, 12 h; RBr, acetone, K2CO3, reflux, 12 h; iii: 12 or 13, 3-NO2-PhNHNH2 or 4-NO2-PhNHNH2, CH3CO2H, reflux, 6 h; iv: 16-18, 3-NO2-PhNHNH2, CH3CO2H, reflux, 6 h.

### Synthesis of benzyl 2-(1,2,3,4-tetrahydro-1-oxonaphthalen-7-yloxy) acetate (4′)

The preparation is similar to that of the synthesis of ethyl 2-(1,2,3,4-tetrahydro-1-oxonaphthalen-7-yloxy) acetate (4). In this case, benzyl bromoacetate is used as reagent instead of ethyl bromoacetate. As in the case of 4, 4′ also presents as a transparent oil. (93% yield). 1HNMR (CDCl3): δ 2.14 (m, 2H), 2.63 (t, J=5.6 Hz, 2H), 2.90 (t, J=6.4 Hz, 2H), 4.72 (s, 2H), 5.25 (s, 2H), 6.65-8.12 (m, 8H).

### Synthesis of 5,6-dihydrobenzo[a]carbazoles (XI′a) 15a and 15c (benzyl esters)

Similarly to the ethyl esters of 5,6-dihydrobenzo[a]carbazoles (XI′a) previously described using tetralone 4′, benzyl esters 15a and 15c were obtained.

15a (R′=7-NO₂): Yield: 14.4%; m.p.: 185-186° C.; ¹HNMR (DMSO-d6): δ 2.85-3.15 (m, 4H), 4.91 (s, 2H), 5.21 (s, 2H), 6.90-7.85 (m, 11H), 12.25 (s, H).

15c (R′=9-NO₂): Yield: 56.0%; m.p.: 205-206° C.; ¹HNMR (DMSO-d6): δ 2.85-3.05 (m, 4H), 4.91 (s, 2H), 5.21 (s, 2H), 6.85-8.45 (m, 11H), 12.18 (s, H).

### Synthesis of 5,6-dihydrobenzo[a]carbazoles (X′a) 19-24

Using tetralones 16-18 as described in the synthesis of 5 with tetralone 3,6-dihydrobenzo[a]carbazoles (X′a) 19-24 were obtained.

19: Yield: 73.2% ¹HNMR (DMSO-d6): δ 2.60-3.10 (m, 4H), 3.63 (s, 3H), 6.43-7.96 (m, 6H), 12.20 (s, H).

20: Yield: 83% ¹HNMR (DMSO-d6): δ 2.51-2.76 (m, 4H), 3.50 (s, 3H), 6.03-7.53 (m, 6H), 12.20 (s, H).

21: Yield: 75% ¹HNMR (DMSO-d6): δ 2.40-2.96 (m, 4H), 3.86 (s, 3H), 3.93 (s, 3H), 6.60-8.11 (m, 5H).

22: Yield: 74% ¹HNMR (DMSO-d6): δ 2.58-3.11 (m, 4H), 3.61 (s, 3H), 6.44-7.90 (m, 6H), 12.21 (s, H).

23: Yield: 78% ¹HNMR (DMSO-d6): δ 2.50-2.72 (m, 4H), 3.54 (s, 3H), 6.13-7.53 (m, 6H), 12.20 (s, H).

24: Yield: 72% ¹HNMR (DMSO-d6): δ 2.38-2.89 (m, 4H), 3.82 (s, 3H), 3.73 (s, 3H), 6.48-8.01 (m, 5H).

### Example 6—Synthesis Scheme for 5,6-dihydrobenzo[a]carbazoles type X′a (39, 42, 43, 46, 49, 59-63, 67, 71, 72)

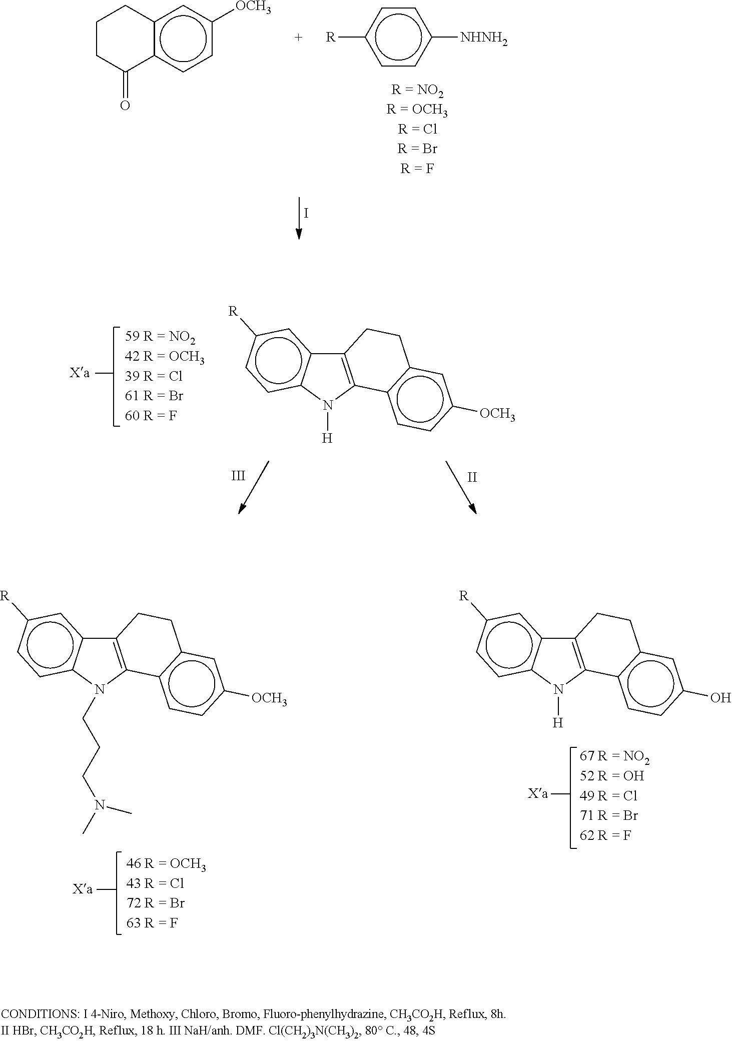

### Synthesis of 5,6-dihydrobenzo[a]carbazoles X′a (39, 42, 59-61)

The appropriate 4-R′-phenylhydrazine (4-NO₂; 4-MeO; 4-Cl; 4-Br; 4-F) (59.01 mmols) is added to a solution of 6-methoxy-1-tetralone (56.74 mmols) in glacial acetic acid (80.32 ml) and the mixture is maintained at reflux, under stirring, for 8 hours. After said time, a crystalline solid is separated from the reaction mixture by cooling in an ice bath, vacuum filtered and washed several times with H₂O to eliminate the excess glacial AcOH. The mother liquor (acetic acid), still containing aliquots of the desired carbazole (39, 42, 59-61) mixed with 6-methoxy-1-tetralone, is evaporated at 1/p, and the residue is crystallised several times with EtOH. The precipitate, purified by crystallisation, is vacuum filtered, supplying other pure aliquots of the corresponding carbazoles.

39: Yield: 82%. M.p.: 170-171. ¹HNMR (CDCl₃): δ 2.89-3.09 (m, 4H), 3.80 (s, 3H), 6.69-7.41 (m, 6H), 8.06 (s, H).

MS m/e 283 (M+)

42: Yield: 33%. M.p.: 186-187. ¹HNMR (CDCl₃): δ 2.91-3.00 (m, 4H), 3.78 (s, 3H), 3.88 (s, 3H), 6.61-8.42 (m, 6H), 11.03 (s, H). MS m/e 279 (M⁺)

59: Yield: 10%. M.p.: 197-198. ¹HNMR (CDCl₃): δ 2.55-2.90 (m, 4H), 3.78 (s, 3H), 6.68-8.15 (m, 6H), 10.20 (s, H).

60: Yield: 89%. M.p.: 175-176. ¹HNMR (CDCl3): δ 2.89-3.00 (m, 4H), 3.81 (s, 3H), 6.69-7.28 (m, 6H), 8.06 (s, H).

MS m/e 267 (M⁺)

61: Yield: 97%. M.p.: 166-167. ¹HNMR (CDCl₃): δ 0.73-3.09 (m, 4H), 3.81 (s, 3H), 6.66-8.12 (m, 6H), 8.76 (s, H).

MS m/e 328 (M⁺)

### Synthesis of 5,6-dihydrobenzo[a]carbazoles X′a (49, 52, 62, 67, 71)

HBr 48% (0.64 ml) is added to a solution of the appropriate carbazole (39, 42, 59, 60, 61) (0.35 mmols) in glacial acetic acid (0.09 ml). The mixture is maintained at reflux and under stirring at 120° C. for 18 hours. After said time, the solvent is evaporated at 1/p to obtain a solid consisting of (49, 52, 62, 67, 71).

49: Yield: 82%. M.p.: 215-216. ¹HNMR (DMSO-d6): δ 2.51-2.87 (m, 4H), 6.63-7.51 (m, 6H), 11.39 (s, H).

MS m/e 269 (M⁺)

52: Yield: 98%. M.p.: >330. ¹HNMR (DMSO-d6): δ 2.49-2.81 (m, 4H), 6.42-7.33 (m, 6H), 10.79 (s, H).

MS m/e 251 (M⁺)

62: Yield: 96%. M.p.: 183-184. ¹HNMR (DMSO-d6): δ 2.51-2.86 (m, 4H), 6.53-7.31 (m, 6H), 11.21 (s, H).

MS m/e 253 (M⁺)

67: Yield: 91%. M.p.: 293-294. ¹HNMR (DMSO-d6): δ 2.48-2.52 (m, 4H), 7.10-8.50 (m, 6H), 11.39 (s, H).

71: Yield: 48%. M.p.>330. ¹HNMR (DMSO-d6): δ 2.33-2.56 (m, 4H), 6.43-8.52 (m, 6H), 11.90 (s, H).

### General Method for N-alkylation of 5,6-dihydrobenzo[a]carbazoles X′a (39, 42, 59-61)

The appropriate carbazole to be alkylated (39, 42, 59-61) (3.5 mmols) is added in portions to a solution of 60% NaH (15.5 mmols) in anh. DMF (7 ml) over a period of 30 minutes. The appropriate R₃X alkyl halide (7.04 mmols) [in the example R₃X═(N,N-dimethyl amino)-propyl chloride.HCl], dissolved in anh. DMF (5 ml), is then dripped in, and the mixture is left under stirring at 80° C. for 48 hours. The solvent is evaporated at 1/p, and the residue is taken up with MeOH HPLC, placed in an ice bath and salified with Et₂O.HCl and anh. Et₂O. The resulting precipitate (43, 46, 63, 72) is vacuum filtered.

43: Yield: 95%. M.p.>330. ¹HNMR (DMSO-d6): δ 2.70-2.98 (m, 8H), 3.35 (s, 6H), 3.81 (s, 3H), 4.52 (t, 2H, J=6.8 Hz), 6.84-7.69 (m, 6H). MS m/e 368 (M⁺)

46: Yield: 32%. M.p.: 202-203. ¹HNMR (DMSO-d6): δ 2.70-2.84 (m, 8H), 3.80 (s, 6H), 3.90 (s, 3H), 3.95 (s, 3H), 4.39 (t, 2H, J=6.8 Hz), 6.85-7.79 (m, 61-1). MS m/e 364 (M⁺)

63: Yield: 63%. M.p.: 207-208. ¹HNMR (DMSO-d6): δ 2.67-2.82 (m, 8H), 3.57 (s, 3H), 3.62 (s, 3H), 3.79 (s, 3H), 4.48 (t, 2H, J=7.2 Hz), 6.80-7.64 (m, 6H). MS m/e 352 (M⁺)

72: Yield: 91%. M.p.: 234-235. ¹HNMR (DMSO-d6): δ 2.70-2.84 (m, 8H), 3.80 (s, 6H), 3.95 (s, 3H), 4.35 (t, 2H, J=6.8 Hz), 6.98-8.44 (m, 6H). MS m/e 413 (M⁺)

### Example 7

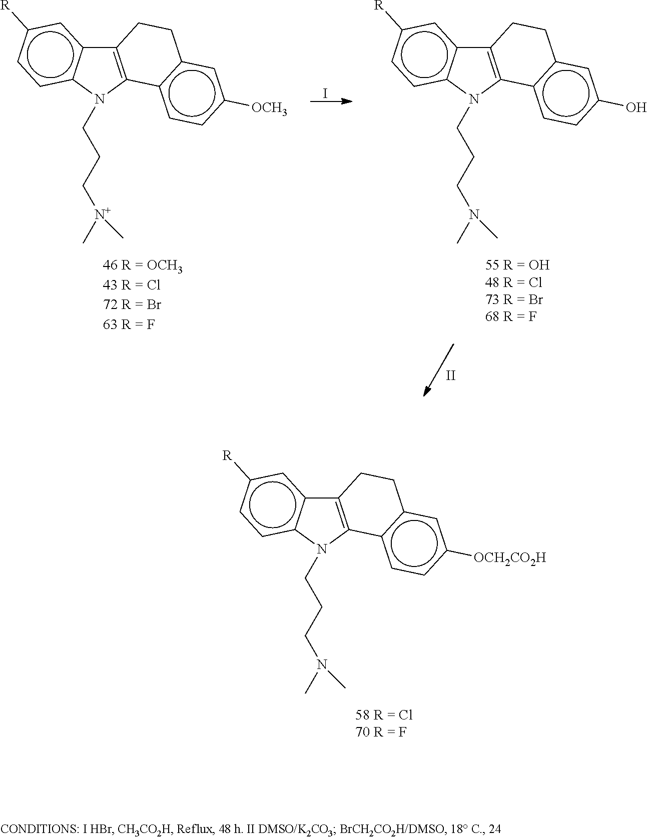

### General Method for N-alkylation of 5,6-dihydrobenzo[a]carbazoles X′a. (48, 55, 68, 73)

48% HBr (0.5 ml) is added to a solution of N-alkylated carbazole 43, 46, 63, 72 (0.25 mmols) in glacial acetic acid (0.07 ml). The mixture is maintained at reflux and under stirring at 120° C. for 48 hours. After said time, the solvent is evaporated at 1/p, and carbazoles 48 and 73 are recovered pure, directly from the residue obtained. Conversely, for 68 and 55, purification by crystallisation and precipitation of their hydrochlorides is required. MeOH is added to the crude residue, the mixture is cooled in an ice bath, and Et₂OxHCl and anh. Et₂O are added. The resulting precipitate consists of pure hydrochlorides of 55 and 68 which are recovered by vacuum filtration.

48: Yield: 67%. M.p.: 228-229. ¹HNMR (DMSO-d6): δ 2.63-2.80 (m, 8H), 3.40 (s, 6H), 4.86 (t, 2H, J=8 Hz), 7.29-8.25 (m, 6H).

MS m/e 354 (M⁺)

55: Yield: 86%. M.p.: 139-140. ¹HNMR (DMSO-d6): δ 2.74-2.80 (m, 8H), 3.35 (s, 6H), 4.87 (t, 2H, J=8 Hz), 6.69-8.44 (m, 6H). MS m/e 336 (M⁺)

68: Yield: 53%. M.p.>330. ¹HNMR (DMSO-d6): δ 2.68-2.79 (m, 8H), 3.39 (s, 6H), 4.70 (t, 2H, J=8 Hz), 6.55-7.11 (m, 6H). MS m/e 337 (M⁺)

73: Yield: 95%. M.p.>330. ¹HNMR (DMSO-d6): δ 2.75-2.90 (m, 8H), 3.14 (s, 6H), 4.90 (t, 2H, J=8 Hz), 7.10-8.43 (m, 6H).

### General Synthesis Method for Acids of Type XI′a

### Synthesis of 11-(3-dimethylamino-propyl)-8-chloro-5,11-dihydro-6H-benzo[a]carbazol-3-yloxy]-acetic acid 58 and 11-(3-dimethylamino-propyl)-8-fluoro-5,11-dihydro-6H-benzo[a]carbazol-3-yloxy]-acetic acid 70

K₂CO₃ (23 mmols) is added to a solution of the appropriate 5,6-dihydrobenzo[a]carbazole intermediate X′a (in example 48 or 68) (5.7 mmols) in DMSO (10 ml) until complete solubilisation. A solution of BrCH₂COOH (5.7 mmols) in DMSO (3 ml) is then dripped in at 18° C. After 24 hours, H₂O, ice and CHCl₃ are added to the reaction mixture. The solution is acidified to pH 3, and the resulting precipitate is vacuum filtered. The filtrate is taken up several times with Et0H and dried each time, until the desired acids 58 and 70 are obtained.

58: Yield: 47%. M.p.: 189-190° C. ¹HNMR (DMSO-d6): δ 3.14-3.23 (m, 8H), 3.61 (s, 6H), 3.94 (s, 2H), 4.86 (t, 2H, J=8 Hz), 7.02-8.21 (m, 6H). MS m/e 412 (M⁺)

70: Yield: 14%. M.p.: 22° C. ¹HNMR (DMSO-d6): δ 3.23-3.33 (m, 8H), 3.72 (s, 6H), 3.97 (s, 2H), 4.86 (t, 2H, J=8 Hz), 6.56-8.67 (m, 6H).

### Example 8

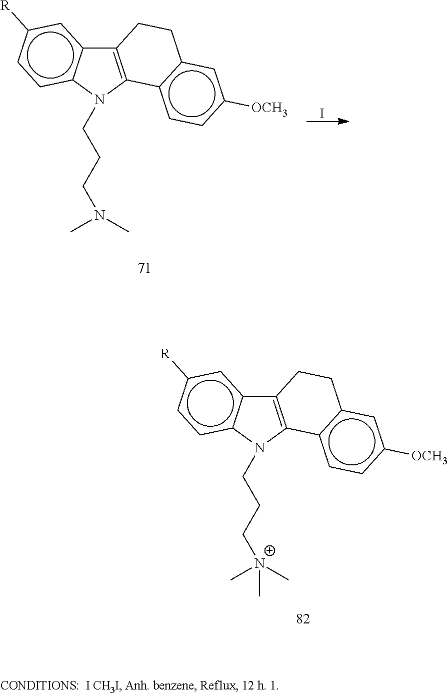

General method of quaternisation of amino groups in R₃ of 5,6-dihydrobenzo[a]carbazoles of general formula X-XII

### Synthesis of [3-(8-bromo-3-methoxy-5,6-dihydro-benzo[a]carbazol-11-yl)-propyl]-trimethyl-ammonium iodide 82

A suspension of the appropriate 5,6-dihydrobenzo[a]carbazole intermediate of general formula X-XII (in the example 5,6-dihydrobenzo[a]carbazole 71 xHCl) in saturated solution of NaHCO₃ is extracted several times with AcOEt. The separated organic solvent is dried with MgSO₄ and evaporated at lip to obtain a semisolid residue consisting of 71 free base.

CH₃I (53 mmols) is added to a solution of 71 free base (7.5 mmols) in anhydrous benzene (76 ml), and the mixture is left under stirring and at reflux for 12 hours. Under these conditions an insoluble solid consisting of the desired quaternary ammonium iodide 82 separates in the reaction solvent, which is vacuum filtered.

82: Yield: 77%. M.p.: 223-224. ¹HNMR (DMSO-d6): δ 2.74-2.90 (m, 8H), 3.81 (s, 9H), 3.94 (s, 3H), 4.47 (t, 3H, J=7.2 Hz),

6.89-8.56 (m, 6H)

### Example 9

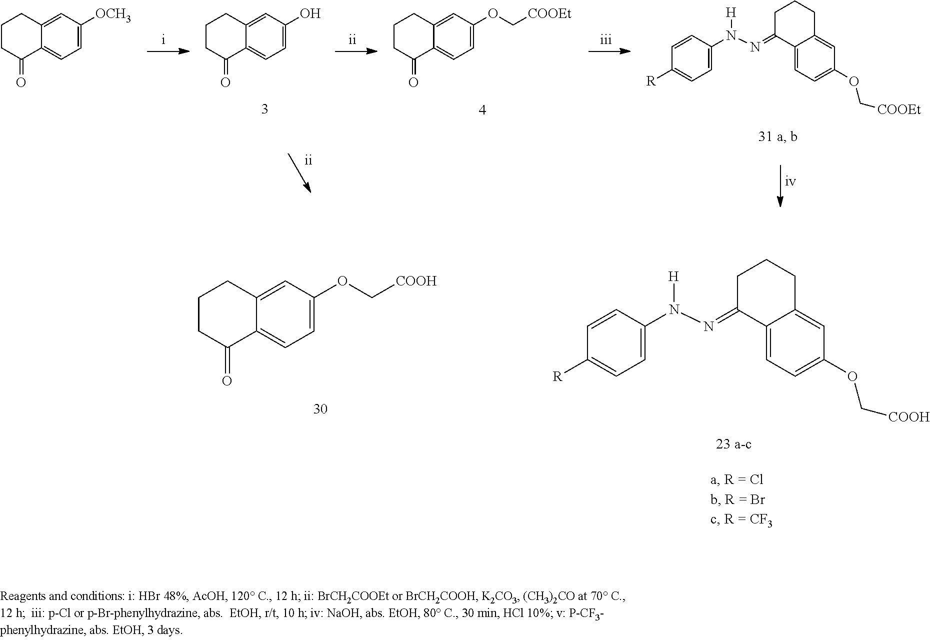

### Synthesis of 6-hydroxy-tetralone (3)

A 48% solution of hydrobromic acid (103 mL) is added to a solution of 6-methoxy-1-tetralone (10.0 g, 56.8 mmols) in glacial acetic acid (15 mL). The mixture is maintained at reflux at 120° C. and under stirring for 12 hours. After said time the solvent is evaporated at 1/p to obtain a crystalline solid essentially consisting of 3.

3: Yield: 98%. ¹HNMR (DMSO-d6): δ 1.98-3.20 (3m, 6H), 6.5-7.6 and 7.8-8.1 (2m, 3H)

Similarly to the preparation of 4, acid 30 can be directly obtained by following the method reported below:

### Synthesis of 2-(1,2,3,4-tetrahydro-1-oxonaphthalen-7-yloxy) acetic acid (30)

Potassium carbonate (141.8 mmols) and alpha-bromoacetic acid (49.1 mmols) are added in succession to a solution of 6-hydroxy-1-tetralone 3 (32.7 mmols) in anhydrous acetone (215 mL). The reaction mixture is maintained at reflux and under stirring at 60° C. for 20 hours, after which it is evaporated at 1/p and the solid residue is taken up with H₂0 and acidified to pH 4-5 with 10% HCl. The resulting mixture is then extracted 3 times with AcOEt, and the combined organic phases are dried on MgSO₄ and evaporated at 1/p to give the desired acid 30.

30: Yield: 63%. ¹HNMR (DMSO-d6): δ 1.97-2.06 (m, 2H), 2.49-2.56 (m, 2H), 2.86-2.92 (t, 2H), 4.77 (s, 2H), 6.85-6.90 (x, 2H), 7.79-7.83 (d, 1H).

### Preparation Method of the Hydrazone Intermediates 31 a, b

The appropriate R-substituted phenylhydrazine (10 mmols) is added to a solution of the appropriate tetralone 4 or 30 (8 mmols) in absolute ethanol (100 mL). The mixture is maintained at room temperature under stirring for 10 hours. After said time the solvent is removed by evaporation at low pressure, and the resulting solid is crystallised from chloroform/hexane to give, depending on the phenylhydrazine used, the desired hydrazones 23a-c or 31a, b.

31a: Yield: 47%. ¹HNMR (DMSO-d6) δ: 1.22 (t, 0.1=7.1 Hz, 3H), 1.82-1.87 (m, 2H), 2.57-2.69 (m, 4H), 4.17 (q, 0.1=7.1 Hz, 2H), 4.77 (s, 2H), 6.71 (d, J=2.6 Hz, 1H), 6.80 (dd, J=8.7, 2.6 Hz, 1H), 7.22 (s, 4H), 7.96 (d, J=8.7 Hz, 1H), 10.16 (bs, 1H).

31b: Yield: 53%. ¹HNMR (DMSO-d6) δ: 1.22 (t, 0.1=7.1 Hz, 3H), 1.83-1.88 (m, 2H), 2.56-2.72 (m, 4H), 4.17 (q, 0.1=7.1 Hz, 2H), 4.77 (s, 2H), 6.71 (d, J=2.5 Hz, 1H), 5.80 (dd, J=8.7, 2.5 Hz, 1H), 7.16 (d, J=8.8 Hz, 2H), 7.34 (d, J=8.8 Hz, 2H), 7.96 (d, J=8.7 Hz, 1H), 9.27 (bs, 1H).

### Method of Preparation of Acid Hydrazones 23a, b

3.3 mL of a 1N aqueous solution of NaOH (3.3 mmols) is added to a solution of the appropriate ethyl ester (31a or 31b) (1.9 mmols) in ethanol (1.2 mL). The resulting mixture is then heated to reflux for 1 h. The reaction mixture is cooled to room temperature, poured into 0.4 mL of concentrated HCl (3.6 mmols) and cooled in ice, and the solid that precipitates is collected by filtration and dried under vacuum.

23a: Yield: 63%. M.p.: 212-213° C. ¹HNMR (DMSO-d6) δ: 1.81-1.85 (m, 2H), 2.55-2.68 (m, 4H), 4.45 (s, 2H), 6.62 (d, J=2.2 Hz, 1H), 6.75 (dd, J=8.6, 2.2 Hz, 1H), 7.21 (s, 4H), 7.93 (d, J=8.6 Hz, 1H), 9.23 (bs, 1H).

23b: Yield: 49%. M.p.>330° C. ¹HNMR (DMSO-d6) δ: 1.81-1.85 (m, 2H), 2.55-2.67 (m, 4H), 4.42 (s, 2H), 3.62 (d, J=1.7 Hz, 1H), 6.74 (dd, J=8.8, 1.7 Hz, 1H), 7.15 (d, J=8.2 Hz, 2H), 7.32 (d, J=8.2 Hz, 2H), 7.92 (d, J=8.8 Hz, 1H), 9.24 (bs, 1H).

### Direct Synthesis from Tetralone 30 of Acid Hydrazone 23c

4-trifluoromethyl)phenylhydrazine (0.95 g) is added to a solution of acid 30 (0.10 g, 0.45 mmols) in absolute ethanol (30 mL). The mixture is maintained at room temperature under stirring for 3 days. After said time the solvent is removed by evaporation at 1/p, and the resulting solid is crystallised from chloroform/hexane to give 0.091 g of the desired acid 23c.

23c: Yield: 53%. M.p.: 196-197° C.: ¹H NMR (DMSO-d6) δ: 1.86 (m, 2H), 2.64-2.72 (m, 4H), 4.69 (s, 2H), 6.72-1H), 6.81 (d, J=8.9 Hz, 1H), 7.34 (d, J=8.4 Hz, 2H), 7.52 (d, J=8.4 Hz,), 8.00 (d, J=8.9 Hz, 1H), 9.60 (bs, 1H).

### Example 10

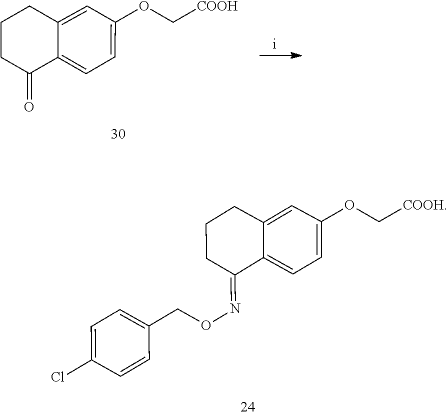 [SMILES: O=C(O)COc1ccc2c(c1)CCCC2=O ; O=C(O)COc1ccc2c(c1)CCC/C2=N\OCc1ccc(Cl)cc1]

Reagents and conditions. i: p-Cl-Benzyloxyamine hydrochloride, CH₃CN r/t, 6 days.

### Synthesis of [5-(4-chloro-benzyloximino)-5,6,7,8-tetrahydro-naphthalen-2-yloxy]-acetic acid (oxime ether 24)

A solution of tetralone 30 (0.44 g, 2 mmols) and of p-chloro-benzylhydroxy-amine (0.39 g, 2 mmols) in CH₃CN (60 mL) is maintained under stirring at room temperature for 6 days. After said time, the solvent is evaporated at 1/p and the solid crude product obtained is purified by silica-gel chromatography, eluting with a hexane/AcOEt 1:1 mixture containing 2% AcOH. From the main fractions collected by eliminating the solvent mixture, a solid crystallises which essentially consists of the desired oxime ether 24.

24: Yield: 45%. M.p.: 143-144° C.: ¹HNMR (CDCl₃) δ: 0.82 (m, 2H), 2.74 (m, 4H), 4.67 (brs, H exchange=_), 5.15 (s, 2H), 6.60-6.80 (m, 2H), 7.20-7.40 (m, 4H), 7.90 (d, J=8.6 Hz, 1H).

### Example 11

Alternative methods a) and b) for preparation of the following acids: 8-R-(5,11-dihydro-6H-benzo[a]carbazol-3-yloxy)-acetic acids 25a-c of type XIII′a and 8-R-(11H-benzo[a]carbazol-3-yloxy)-acetic acids of type XIII′b and their corresponding ethyl esters 32a, b (type XIII′a) and 33a, b (type XIII′b)

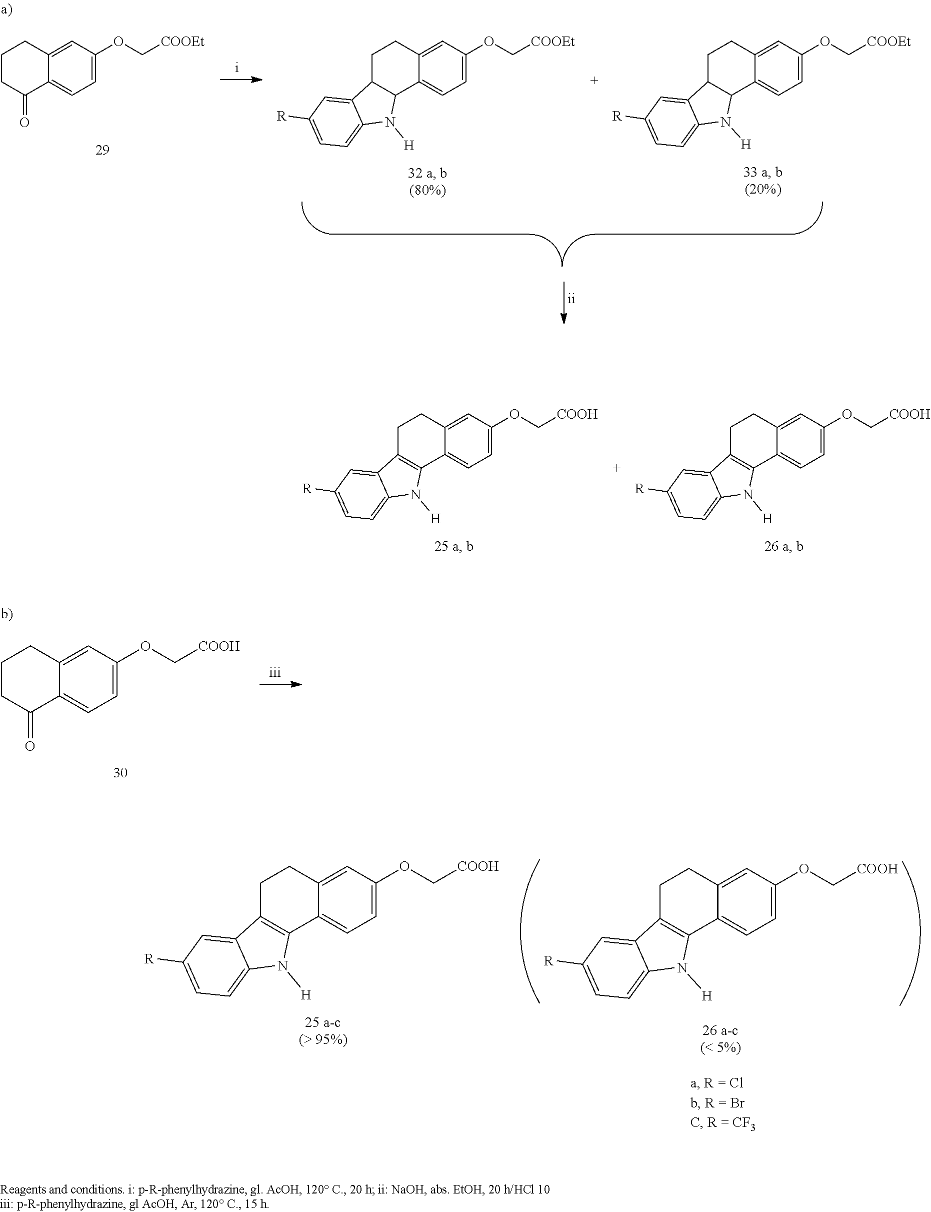

Method a)

### Synthesis of ethyl esters 32a, b (type XIII′a) and 33a, b (type XIII′b)

The appropriate 4-R-phenylhydrazine (8.4 moles) is added to a solution of tetralone 29 (2.00 g, 8.05 mmols) in glacial acetic acid (11 mL), and the mixture is maintained at reflux under stirring for 20 hours. After said time the solvent is evaporated at 1/p, and the resulting solid is dissolved in the minimum quantity of Et₂0 and washed several times with H₂0 and a saturated solution of NaHCO₃. The ether solution is then evaporated at 1/p, supplying the desired ethyl esters (32a,b) and (33a,b) in admixture. ¹H-NMR analysis reveals a % composition in admixture of the two carbazoles of type XIII′a (32a) and XIII′b (33a) at the ratio of 84/16, and that of the admixture of 32b (type XIII′a) and 33b (type XIII′b) at the ratio of 80/20.

32a: ¹HNMR (DMSO-d6) δ: 1.22 (t, 0.1=7.1 Hz, 3H), 2.90 (m, 4H), 4.18 (q, 0.1=7.1-Hz, 2H), 4.81 (s, 2H), 6.80-7.15 (m, 3H), 7.25-8.62 (m, 3H), 11.55 (s, 1H).

33a: ¹HNMR (DMSO-d6) δ: 1.22 (t, 0.1=7.1 Hz, 3H), 4.18 (q, 0.1=7.1 Hz, 2H), −0.90 (s, 2H), 8.15-8.45 (m, 3H), 12.23 (s, 1H).

32: ¹HNMR (DMSO-d6) δ: 1.22 (t, 0.1=7.1 Hz, 3H), 2.89 (m, 4H), 4.18 (q, . . . ; =7.1 Hz, 2H), 4.80 (s, 2H), 6.81-7.63 (m, 6H), 11.55 (s, 1H).

33b: ¹HNMR (DMSO-d6) δ: 1.23 (t, 0.1=7.1 HZ, 3H), 4.18 (q, 0.1=7.1 Hz, 2H), −0.49 (s, 2H), 7.10-7.60 and 8.10-8.50 (m, 8H), 12.28 (s, 1H).

### Acid Synthesis: 8-R-(5,11-dihydro-6H-benzo[a]carbazol-3-yloxy)-acetic acids 25a-c of type XIII′a and 8-R-(11H-benzo[a]carbazol-3-yloxy)-acetic acids of type XIII′b

KOH (3.6 mmols) is added to a solution of the appropriate ester 32a, b (3 mmols) and absolute EtOH (108 mL), and the mixture is left under stirring at r/t for 20 hours. After said time, the solvent is evaporated at 1/p. The resulting solid is dissolved in the minimum quantity of H₂O and several washes are performed with AcOEt, after which a 10% HCl solution is added until pH 4-5 is reached. The result is a solid which is vacuum filtered. Said solid is then crystallised from EtOH to obtain the acids of type XIII′a 25a and 25b still about 10% contaminated with the corresponding aromatic carbazoles type XIII′b (26a, b).

25a: Yield: 12% ¹HNMR (DMSO-d6) δ: 2.89 (m, 4H), 4.70 (s, 2H), 6.80-7.07 (m, 3H), 7.25-−0.75 (m, 3H), 11.54 (s, 1H).

25b: Yield: 10% ¹HNMR (DMSO-d6) δ: 2.86 (m, 4H), 4.84 (s, 2H), 6.70-6.90 (m, 2H), 7.05-−0.40 (m, 2H), 7.50-7.70 (m, 2H), 11.63 (s, 1H).

Method b)

### Acid synthesis: 8-R-(5,11-dihydro-6H-benzo[a]carbazol-3-yloxy)-acetic acids 25a-c

A solution of tetralone 30 (1.4 mmols) and the appropriate substituted 4-R-phenylhydrazine (1.5 mmols) in glacial acetic acid (2 mL), previously degassed with a stream of Ar, is heated to reflux overnight under an inert atmosphere of Ar. The mixture is then concentrated at 1/p and the residue is purified by flash chromatography on silica gel (eluent hexane/ethyl acetate 1:1+2% acetic acid). The resulting solid is crystallised from EtOH to give practically pure acids of type XIII′ a (25a-c).

25a: Yield: 83%. M.p.: 146-148° C.: ¹HNMR (DMSO-d6) δ: 2.89 (m, 4H), 4.70 (s, 2H), 6.80-7.07 (m, 3H), 7.25-−0.75 (m, 3H), 11.54 (s, 1H).

25b: Yield: 53%. M.p.: 153-155° C.: ¹HNMR (DMSO-d6) δ: 2.86 (m, 4H), 4.84 (s, 2H), 6.70-6.90 (m, 2H), 7.05-−0.40 (m, 2H), 7.50-7.70 (m, 2H), 11.63 (s, 1H).

25c: Yield: 63%. M.p.: 243-245° C.: ¹HNMR (DMSO-d6) δ: 2.92-2.98 (m, 4H), 4.71 (s, 2H), 6.91 (dd, J=8.4, 2.5 Hz, 1H), 6.93 (d, J=2.5 Hz, 1H), 7.33 (d, J=8.6 Hz, 1H), 7.52 (d, J=3.6 Hz, 1H), 7.60 (d, J=8.4 Hz, 1H), 7.84 (s, 1H), 11.84 (s, 1H).

### Example 12—Methods of Aromatising the C Ring of Nuclei Type X′a, XI′a, XIII′a, XIV′a and XV′a

This type of oxidative conversion typical of 5,11-dihydro-6H-benzo[a]carbazoles of type X′a, XI′a, XIII′a, XIV′a and XV′a to 11H-benzo[a]carbazoles of type X′b, XI′b, XIII′b, XIV′b and XV′b can be obtained as required by methods a)-c) reported below by way of example for oxidation to the 11H-benzo[a]carbazoles 26a, b and 37 of 8-(Cl)-5,6-dihydrobenzo[a]carbazoles 9a and 36 or 8-(Br)-5,6-dihydrobenzo[a]carbazole 9b.

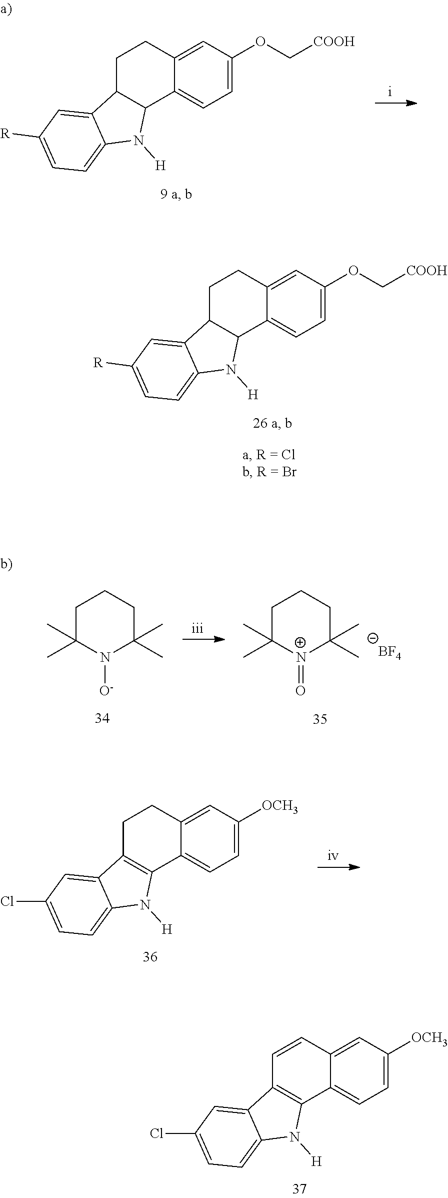

Reagents and conditions. i: AcOH, 120° C., 5 days; ii: AcOH, Pd—C, 120° C., 20 h iii: HBF₄ 50%, 0° C., 10 min; iv: CH₃CN, TEMPO, 0° C., 15 min.

Method a)

### Synthesis of (8-bromo-11H-benzo[a]carbazol-3-yloxy)-acetic acid 26a and (8-bromo-11H-benzo[a]carbazol-3-yloxy)-acetic acid 26b

A solution of the appropriate carbazole (25a, b) (3 mmols) in glacial acetic acid (20 mL) is left at reflux in air for 5 days. After said time the solution is concentrated and the precipitated solid collected by vacuum filtration. The precipitate consisting of the desired products of oxidation (26a, b) is washed with small aliquots of CHCl₃.

26a: Yield: 73%. M.p.: 250-253° C. ¹H NMR (DMSO-d6) δ: 4.81 (s; 2H), 7.28-7.47 (m, 3H), 7.50-7.62 (m, 2H), 8.14-8.22 (m, 1H), 8.39-8.44 (d, 1H).

26b: Yield: 66%. M.p.: 228-231° C. ¹H NMR (DMSO-d6) δ: 4.39 (s, 2H), 6.76 (x, 1H), 7.20-7.7 (m, 5H), 8.14-8.6 (m, 3H), 12.25 (s, 1H).

Method b)

### Preparation of 2,2,6,6-tetramethylpiperidin-1-oxonium tetrafluoroborate (35)

A 48% solution of tetrafluoroboric acid (9 mL, 0.06 mmols) is added drop by drop to a solution of TEMPO (2,2,6,6-tetramethyl-1-oxy-piperidine) (34) (5 g, 32.00 mmols) in Et₂0 (20 mL). During dripping, the solution is maintained under stirring at the temperature of 0° C. At the end of the addition, the temperature is maintained at 16° C. for 10 minutes. The yellow precipitate that forms during this time, which is collected by filtration, washed with cold ether and dried, consists solely of 35 (3.3 g).

35: Yield 41%. M.p.: 164-165° C.

### Synthesis of 8-chloro-3-methoxy-11H-benzo[a]carbazole (37)

A solution of TEMPO (0.17 g, 0.66 mmols) in acetonitrile (1.20 mL) is added by slow dripping to a solution of 8-chloro-3-methoxy-5,11-dihydro-6H-benzo[a]carbazole 36 (0.20 g, 0.70 mmols) in anhydrous acetonitrile (7 mL). The solution is maintained under stirring in an ice bath for 15 minutes, after which the resulting solid is collected by vacuum filtration.

37: Yield 99%. M.p.: 165-167° C. ¹HNMR (DMSO-d6) δ: 3.91 (s, 3H), 7.29-7.58 (m, 5H), 8.15-8.43 (m, 3H), −2.25 (s, 1H).

### Example 13—Example of Reduction of an NO₂ Group in R₁ on the a Ring of Compounds of Type I-XIII′ (where Applicable)

Reduction of 3-methoxy-9-nitro-5,11-dihydro-6H-benzo[a]carbazole 40 to the amine 3-methoxy-11H-benzo[a]carbazol-9-yl 41

Ni-Raney (66 mg) is added as catalyst under inert atmosphere (Ar) to a solution of 40 (2.00 mmols) in absolute EtOH (37 mL) plus 64% hydrazine hydrate (5 mL). The mixture is left at reflux, under stirring, hydrazine (9.3 mmols) is added drop by drop, and the reflux continues for a further 2 hours. After said time the mixture is filtered through celite under an inert atmosphere (Ar). The filtrate is evaporated at lip, and the solid residue essentially consists of the desired product 41.

41: Yield 94%. M.p.: 182-184. ¹H NMR (DMSO-d6) δ: 2.70-3.88 (m, 4H), 4.76 (s, 2H), 6.34-6.54 (m, 2H), 6.77-6.84 (m, 2H), 7.07-7.11 (d, 1H), 7.38-7.42 (d, 1H), 10.67 (s, 1H).

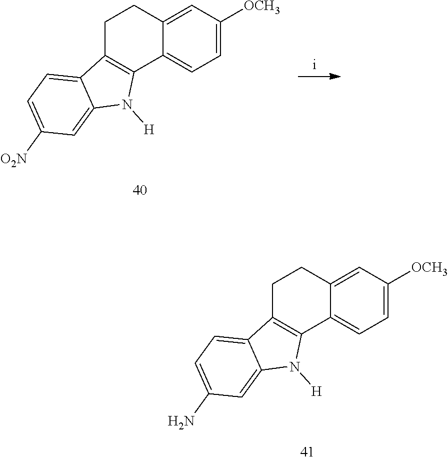 [SMILES: COc1ccc2c(c1)CCc1c-2[nH]c2cc([N+](=O)[O-])ccc12 ; COc1ccc2c(c1)CCc1c-2[nH]c2cc(N)ccc12]

### Reagents and Conditions. i: N₂H₄H₂O, M-Raney, Abs. EtOH, 80° C., 1.5 l

### Example 14—Example of Use of TEMPO 35 in the Oxidation of a Nucleus of Type 5,6,7,12-tetrahydro-benzo[6,7]cyclohepta[1,2-b]indole (type Xa, XIa, XIIIa, XIVa, XVa)

### Example of oxidation of 3,4-dimethoxy-10-nitro-5,6,7,12-tetrahydro-benzo[6,7]cyclohepta[1,2-b]indole 42 to 3,4-dimethoxy-10-nitro-5,12-dihydro-6H-benzo[6,7]cyclohepta[1,2-b]indol-7-one 43

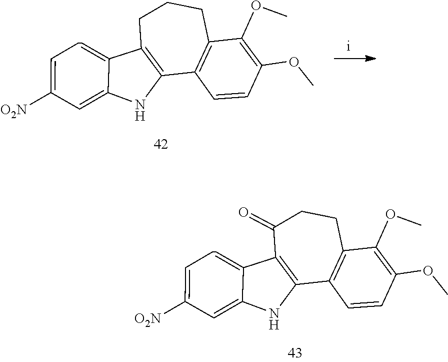

### Reagents and Conditions: TEMPO (35), THF, r/t, 6 Days

### Synthesis of 3,4-dimethoxy-10-nitro-5,12-dihydro-6H-benzo[6,7]cyclohepta[1,2-b]indol-7-one 43

A solution of TEMPO (35) (0.23 g, 0.89 mmol) in anh. acetonitrile (2 mL) is added by slow dripping to a solution of 3,4-dimethoxy-10-nitro-5,6,7,12-tetrahydro-benzo[6,7]cyclohepta[1,2-b]indole 42 [obtained as above for the analogues of type X′a as pure XI′a, namely by reacting an appropriate R₂-substituted 6,7,8,9-tetrahydro-benzocyclohepten-5-one with the appropriate phenylhydrazine, scheme Id, same conditions for X′a and XIa] (0.3 g, 0.9 mmol) in anhydrous acetonitrile (50 mL), under stirring at 0° C. in an inert atmosphere. The solution is stirred at r/t for 6 days, the solvent is removed by evaporation at lip, and the solid residue obtained is purified by flash chromatography on silica gel (hexane/ethyl acetate 1:1), supplying the desired pure ketone 43 as a glassy yellow oil.

43: Yield 28%. ¹H NMR (DMSO-d6) δ: 2.49 (s, 3H), 2.50 (s, 3H), 3.75-3.90 (m, 4H), 7.50-8.50 (m, 5H), 10.1 (s, 1H).

### Example 15—Biopharmacological Activity Tests

Measurements of biological activity conducted by the following methods:

1) Costa, B; Grillone, A F; Salvetti, A; Rocchiccioli, S; Iacopetti, P; Daniele, S; Da Pozzo, E; Campiglia, P; Novellino, E; Martini, C; Rossi, L An antibody-free strategy for screening putative HDM2 inhibitors using crude bacterial lysates expressing GST-HDM2 recombinant protein Drug Testing and Analysis (2013), 5, 7, 596-601.

2) Costa B, Bendinelli S, Gabelloni P, Da Pozzo E, Daniele S, et al. Human Glioblastoma Multiforme: p53 Reactivation by a Novel MDM2 Inhibitor. PLoS ONE (2013) 8(8): e72281. doi:10.1371/journal.pone.0072281.

Results:

1) Inhibitory effect on p53/MDM2 complex binding (IC50 nM/% inhibition at 10 uM) of some ligands of general formula I and of nutlin (Nut-3 used as reference). Test performed on cell lysate of human glioblastoma (GBM) cells U343MG (1,2). The results are set out in Table 2.

TABLE 2

| # | # | | |
|---|---|---|---|
| substructure | Code | Structure | IC50 nM |
| Comparator Medicament | Nut-3 | 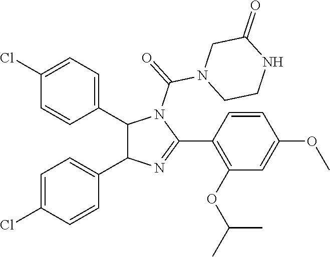 [SMILES: COc1ccc(C2=NC(c3ccc(Cl)cc3)C(c3ccc(Cl)cc3)N2C(=O)N2CCNC(=O)C2)c(OC(C)C)c1] | 296 ± 15 |
| XIc XIl | RA1 | 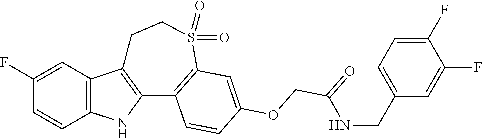 [SMILES: O=C(COc1ccc2c(c1)S(=O)(=O)CCc1c-2[nH]c2ccc(F)cc12)NCc1ccc(F)c(F)c1] | 920 ± 20 |
| XIa | RA2 | 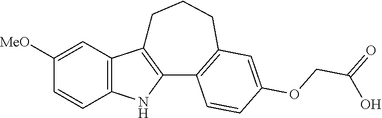 [SMILES: COc1ccc2[nH]c3c(c2c1)CCCc1cc(OCC(=O)O)ccc1-3] | 2900 ± 120 |
| XIr | RA3 | 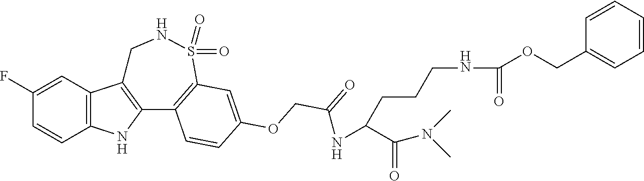 [SMILES: CN(C)C(=O)C(CCCNC(=O)OCc1ccccc1)NC(=O)COc1ccc2c(c1)S(=O)(=O)NCc1c-2[nH]c2ccc(F)cc12] | 620 ± 50 |
| Xa | GA17M8 | 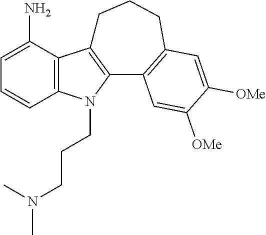 [SMILES: COc1cc2c(cc1OC)-c1c(c3c(N)cccc3n1CCCN(C)C)CCC2] | 12700 ± 1500 |
| XIb | RM66 | 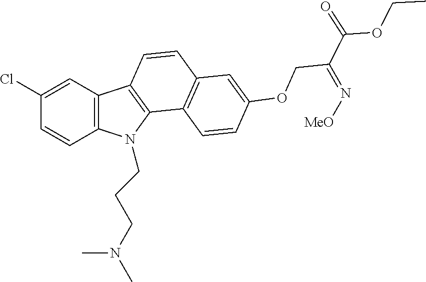 [SMILES: CCOC(=O)/C(COc1ccc2c(ccc3c4cc(Cl)ccc4n(CCCN(C)C)c23)c1)=N/OC] | 2700 ± 120 |
| XIb | RM58 | 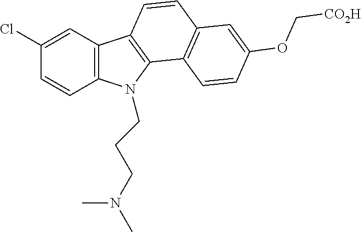 [SMILES: CN(C)CCCn1c2ccc(Cl)cc2c2ccc3cc(OCC(=O)O)ccc3c21] | (58% a 10 uM) |
| XIb | RM85 | 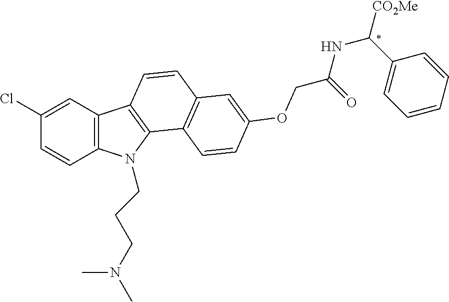 [SMILES: COC(=O)C(NC(=O)COc1ccc2c(ccc3c4cc(Cl)ccc4n(CCCN(C)C)c23)c1)c1ccccc1] | (82% a 10 uM) |
| XI'd | RA6 | 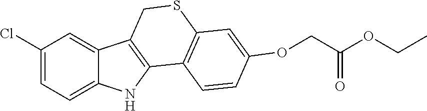 [SMILES: CCOC(=O)COc1ccc2c(c1)SCc1c-2[nH]c2ccc(Cl)cc12] | (18% a 10 uM) |
| XI'd | RA7 | 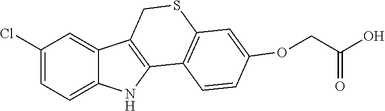 [SMILES: O=C(O)COc1ccc2c(c1)SCc1c-2[nH]c2ccc(Cl)cc12] | n.a. |
| XI'd | RA9 | 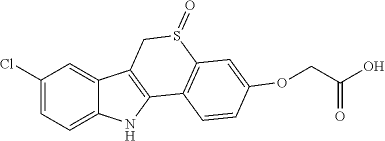 [SMILES: O=C(O)COc1ccc2c(c1)S(=O)Cc1c-2[nH]c2ccc(Cl)cc12] | n.a. |
| XI'd | RA11 | 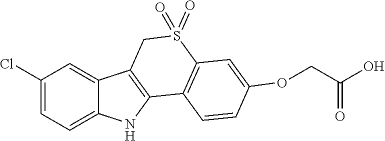 [SMILES: O=C(O)COc1ccc2c(c1)S(=O)(=O)Cc1c-2[nH]c2ccc(Cl)cc12] | n.a. |
| XI'd | RA12 | 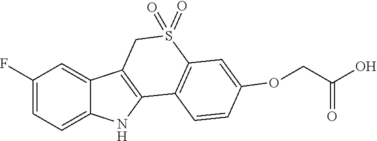 [SMILES: O=C(O)COc1ccc2c(c1)S(=O)(=O)Cc1c-2[nH]c2ccc(F)cc12] | n.a. |
| XI'd | RA13 | 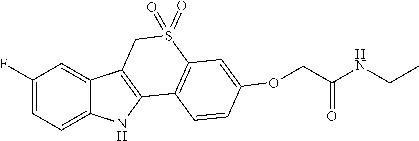 [SMILES: CCNC(=O)COc1ccc2c(c1)S(=O)(=O)Cc1c-2[nH]c2ccc(F)cc12] | (22% a 10 uM) |
| XI'g | RA14 | 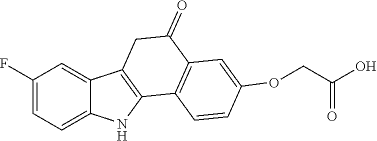 [SMILES: O=C(O)COc1ccc2c(c1)C(=O)Cc1c-2[nH]c2ccc(F)cc12] | n.a. |
| X'c | RA15 | 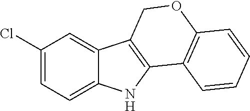 [SMILES: Clc1ccc2[nH]c3c(c2c1)COc1ccccc1-3] | (<5% a 10 uM) |
| X'c | RA16 | 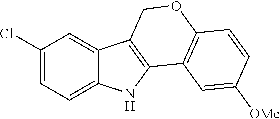 [SMILES: COc1ccc2c(c1)-c1[nH]c3ccc(Cl)cc3c1CO2] | (<5% a 10 uM) |
| XI'c | RA17 | 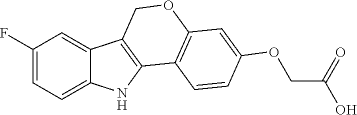 [SMILES: O=C(O)COc1ccc2c(c1)OCc1c-2[nH]c2ccc(F)cc12] | n.a. |
| XI'c | RA18 | 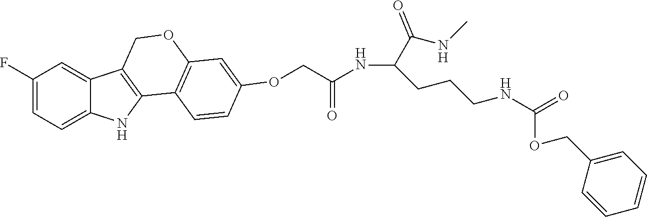 [SMILES: CNC(=O)C(CCCNC(=O)OCc1ccccc1)NC(=O)COc1ccc2c(c1)OCc1c-2[nH]c2ccc(F)cc12] | 1350 ± 150 |
| XI'c | RA19 | 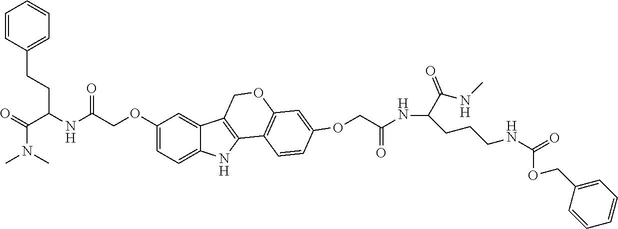 [SMILES: CNC(=O)C(CCCNC(=O)OCc1ccccc1)NC(=O)COc1ccc2c(c1)OCc1c-2[nH]c2ccc(OCC(=O)NC(CCc3ccccc3)C(=O)N(C)C)cc12] | 450 ± 15 |
| XI'h | RA20 | 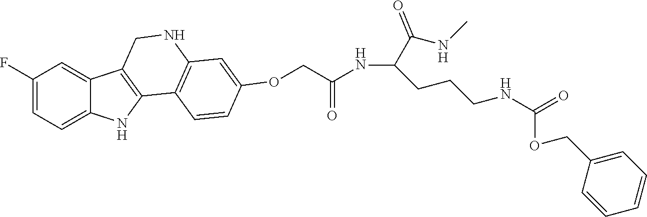 [SMILES: CNC(=O)C(CCCNC(=O)OCc1ccccc1)NC(=O)COc1ccc2c(c1)NCc1c-2[nH]c2ccc(F)cc12] | 1550 ± 250 |
| XIV'a | GA09a | 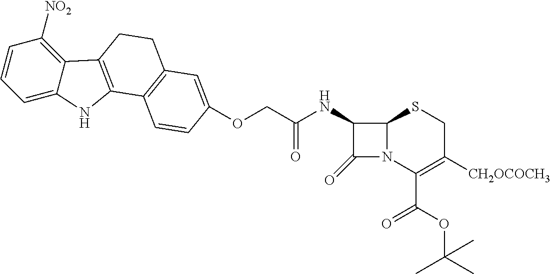 | 650 ± 50 |
| XIV'a' | GA09a' | 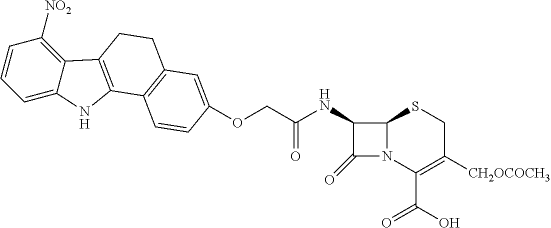 | n.a. |
| XIV'a | GA09b | 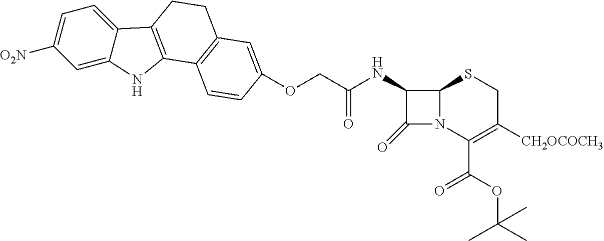 | 225 ± 20 |
| XIV'a' | GA09b' | 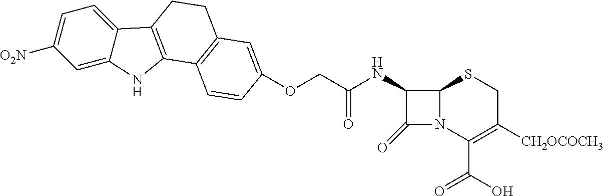 | n.a. |
| XIV'a | RM36 | 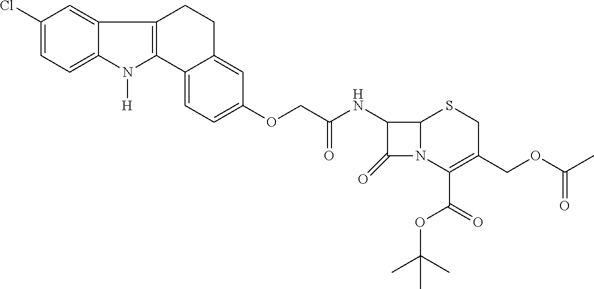 [SMILES: CC(=O)OCC1=C(C(=O)OC(C)(C)C)N2C(=O)C(NC(=O)COc3ccc4c(c3)CCc3c-4[nH]c4ccc(Cl)cc34)C2SC1] | 280 ± 20 |
| XIV'a | RM37 | 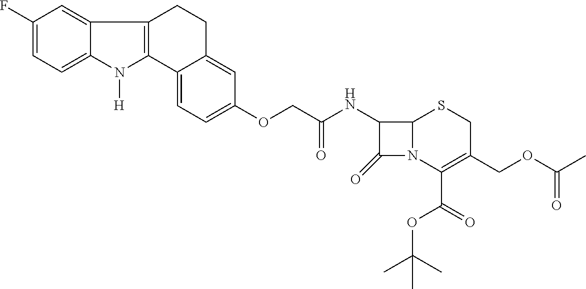 [SMILES: CC(=O)OCC1=C(C(=O)OC(C)(C)C)N2C(=O)C(NC(=O)COc3ccc4c(c3)CCc3c-4[nH]c4ccc(F)cc34)C2SC1] | 222 ± 44 |
| XIV'a' | RM53 | 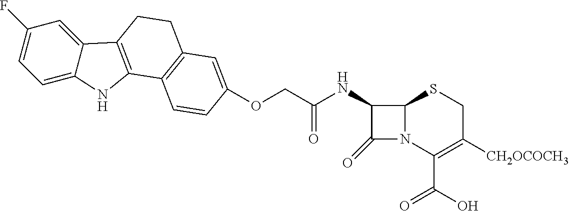 | (<5% a 10 uM) |
| XIV'a | RM47 | 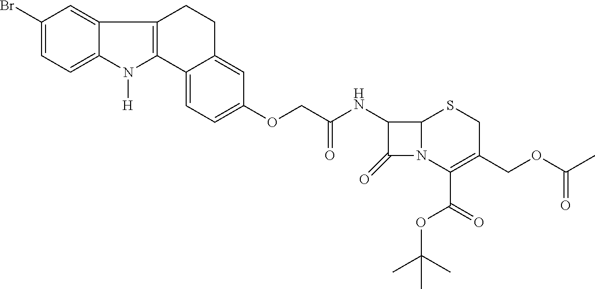 [SMILES: CC(=O)OCC1=C(C(=O)OC(C)(C)C)N2C(=O)C(NC(=O)COc3ccc4c(c3)CCc3c-4[nH]c4ccc(Br)cc34)C2SC1] | 195 ± 22 |
| XIV'd | RA25 | 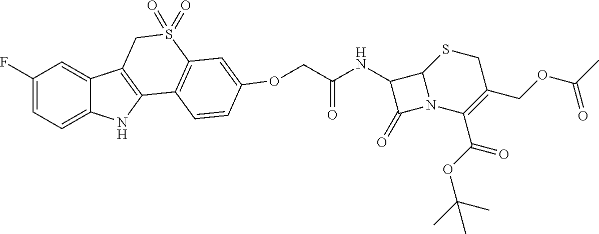 [SMILES: CC(=O)OCC1=C(C(=O)OC(C)(C)C)N2C(=O)C(NC(=O)COc3ccc4c(c3)S(=O)(=O)Cc3c-4[nH]c4ccc(F)cc34)C2SC1] | 143 ± 16 |
| XV'e | RA26 | 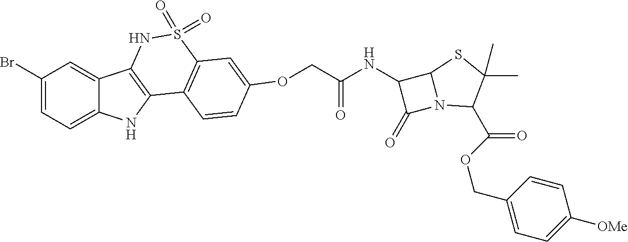 [SMILES: COc1ccc(COC(=O)C2N3C(=O)C(NC(=O)COc4ccc5c(c4)S(=O)(=O)Nc4c-5[nH]c5ccc(Br)cc45)C3SC2(C)C)cc1] | 120 ± 90 |
| XV'g' | RA29 | 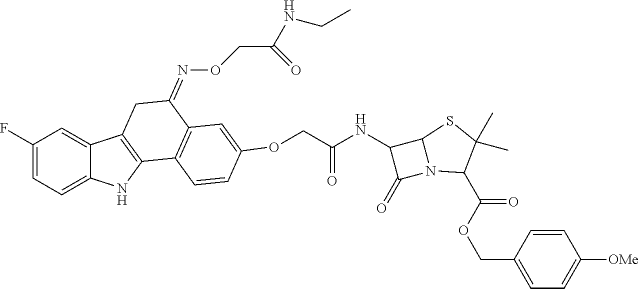 [SMILES: CCNC(=O)CO/N=C1/Cc2c([nH]c3ccc(F)cc23)-c2ccc(OCC(=O)NC3C(=O)N4C3SC(C)(C)C4C(=O)OCc3ccc(OC)cc3)cc21] | 100 ± 30 |
| XIIId | RA30 | 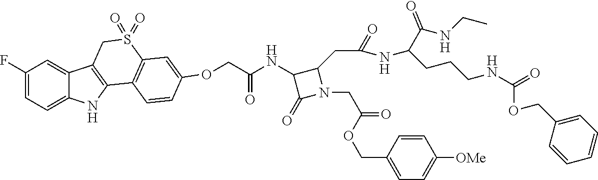 [SMILES: CCNC(=O)C(CCCNC(=O)OCc1ccccc1)NC(=O)CC1C(NC(=O)COc2ccc3c(c2)S(=O)(=O)Cc2c-3[nH]c3ccc(F)cc23)C(=O)N1CC(=O)OCc1ccc(OC)cc1] | 150 ± 10 |
| XIV'c | RA31 | 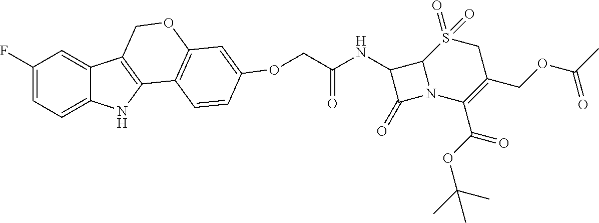 [SMILES: CC(=O)OCC1=C(C(=O)OC(C)(C)C)N2C(=O)C(NC(=O)COc3ccc4c(c3)OCc3c-4[nH]c4ccc(F)cc34)C2S(=O)(=O)C1] | 120 ± 45 |
| XV'c | RA35 | 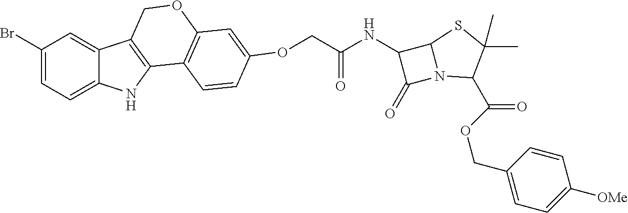 [SMILES: COc1ccc(COC(=O)C2N3C(=O)C(NC(=O)COc4ccc5c(c4)OCc4c-5[nH]c5ccc(Br)cc45)C3SC2(C)C)cc1] | 375 ± 22 |
| XIV'e | RA36 | 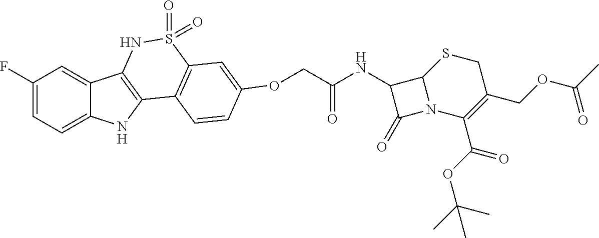 [SMILES: CC(=O)OCC1=C(C(=O)OC(C)(C)C)N2C(=O)C(NC(=O)COc3ccc4c(c3)S(=O)(=O)Nc3c-4[nH]c4ccc(F)cc34)C2SC1] | 220 ± 100 |
| XIV'g' | RA37 | 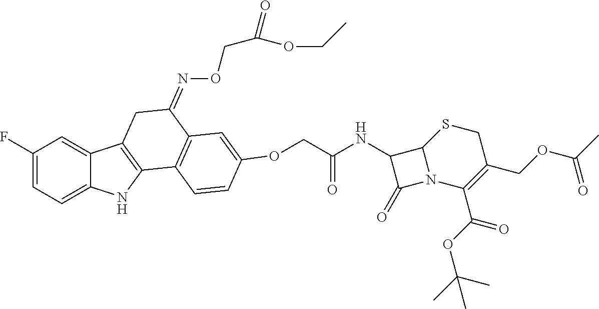 [SMILES: CCOC(=O)CO/N=C1/Cc2c([nH]c3ccc(F)cc23)-c2ccc(OCC(=O)NC3C(=O)N4C(C(=O)OC(C)(C)C)=C(COC(C)=O)CSC34)cc21] | 180 ± 150 |
| XIV'g' | RM38 | 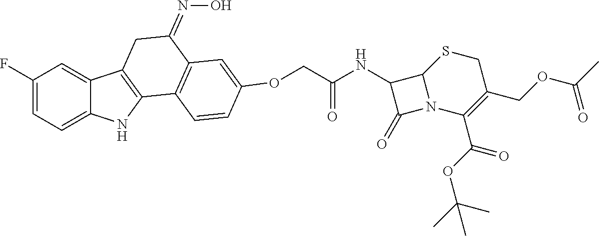 [SMILES: CC(=O)OCC1=C(C(=O)OC(C)(C)C)N2C(=O)C(NC(=O)COc3ccc4c(c3)/C(=N\O)Cc3c-4[nH]c4ccc(F)cc34)C2SC1] | 222 ± 210 |
| XIV'g | RM39 | 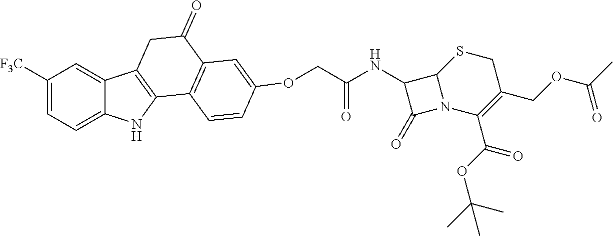 [SMILES: CC(=O)OCC1=C(C(=O)OC(C)(C)C)N2C(=O)C(NC(=O)COc3ccc4c(c3)C(=O)Cc3c-4[nH]c4ccc(C(F)(F)F)cc34)C2SC1] | 232 ± 200 |
| XIVr | RA40 | 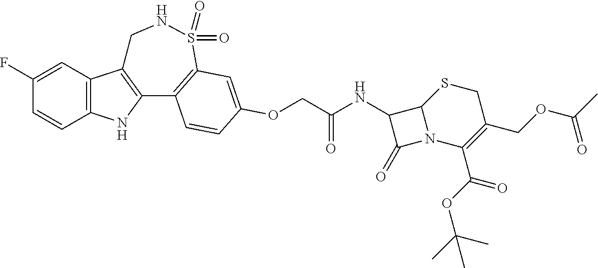 [SMILES: CC(=O)OCC1=C(C(=O)OC(C)(C)C)N2C(=O)C(NC(=O)COc3ccc4c(c3)S(=O)(=O)NCc3c-4[nH]c4ccc(F)cc34)C2SC1] | 197 ± 124 |
| XVr | RA42 | 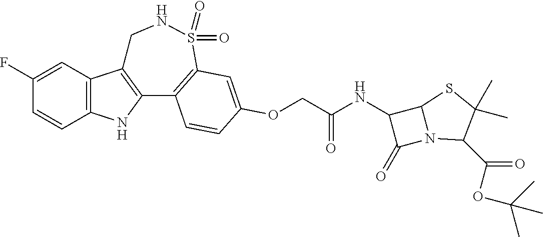 [SMILES: CC(C)(C)OC(=O)C1N2C(=O)C(NC(=O)COc3ccc4c(c3)S(=O)(=O)NCc3c-4[nH]c4ccc(F)cc34)C2SC1(C)C] | 292 ± 25 |
| XIVc | RM43 | 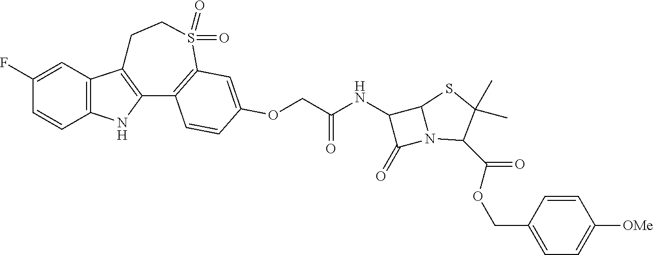 [SMILES: COc1ccc(COC(=O)C2N3C(=O)C(NC(=O)COc4ccc5c(c4)S(=O)(=O)CCc4c-5[nH]c5ccc(F)cc45)C3SC2(C)C)cc1] | 228 ± 16 |
| XIIIr | RA44 | 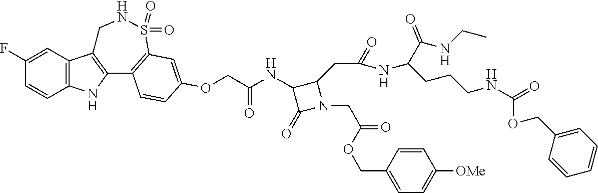 [SMILES: CCNC(=O)C(CCCNC(=O)OCc1ccccc1)NC(=O)CC1C(NC(=O)COc2ccc3c(c2)S(=O)(=O)NCc2c-3[nH]c3ccc(F)cc23)C(=O)N1CC(=O)OCc1ccc(OC)cc1] | 223 ± 22 |

2) Stabilisation of p53 (U343MG, 1, 2).

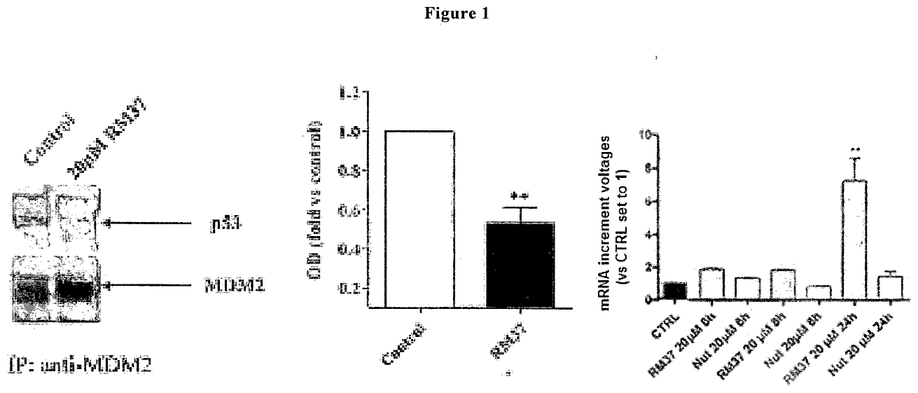

The stabilisation of p53 in the presence of RM37 or Nut-3 is due to the reduction of p53 bonded to MDM2, not induction from scratch of mRNA synthesis of p53 (1,2). The results are set out in FIG. 1.

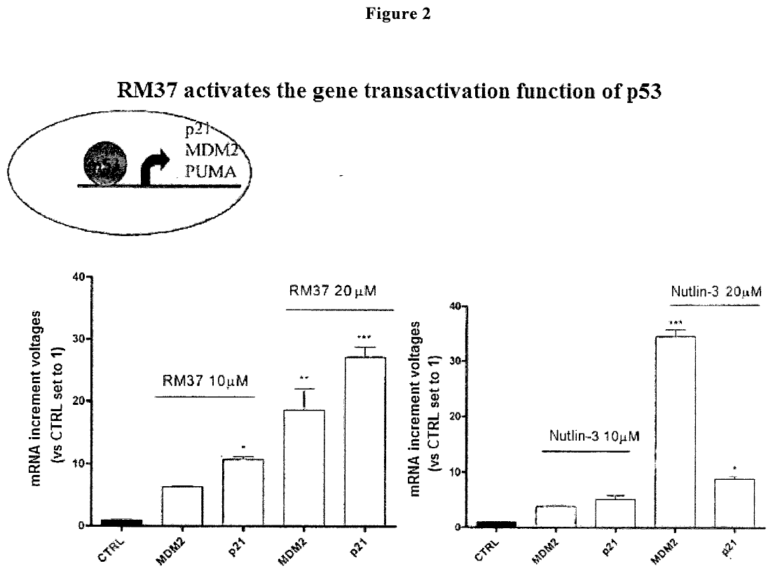

3) Effect of RM37 and Nut-3 on activation of the gene transactivation function of p53 (U343MG; 1,2). The results are set out in FIG. 2.

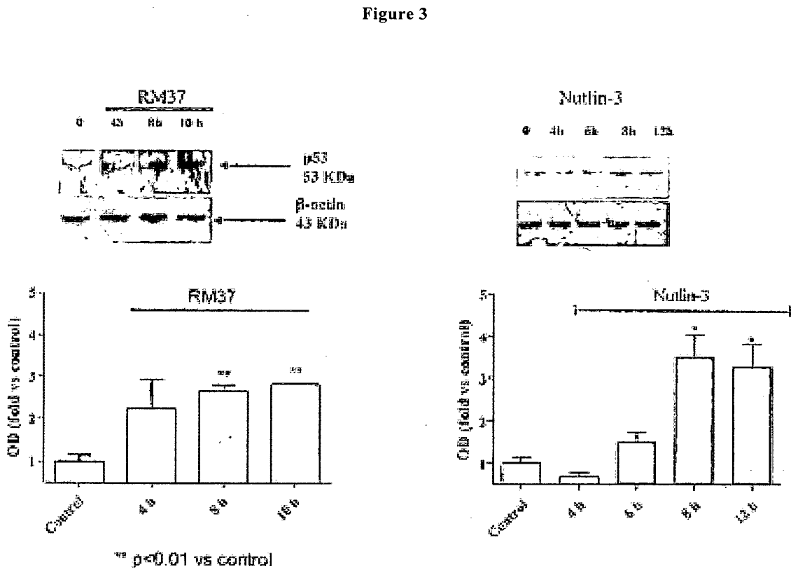

4) RM37 stabilises the intracellular levels of protein p53 (343MG; 1,2). The results are set out in FIG. 3.

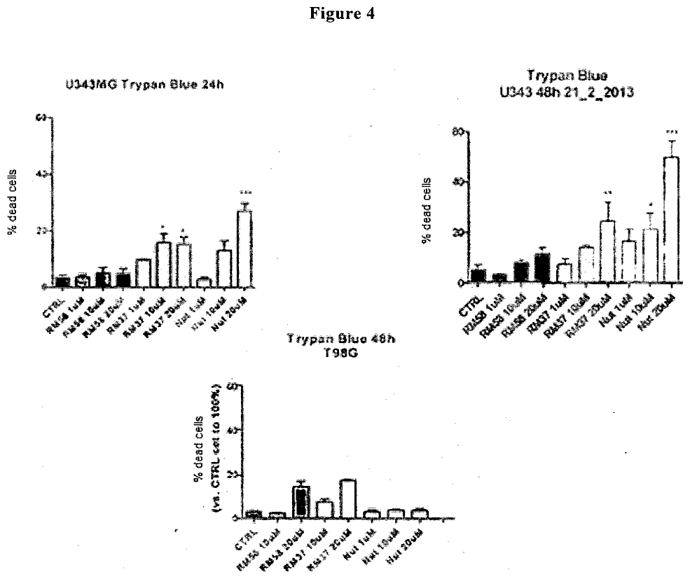

5) Antitumoral effect in vitro (RM37 vs Nut-3): dead cell count (U343MG; 1,2). The results are set out in FIG. 4.

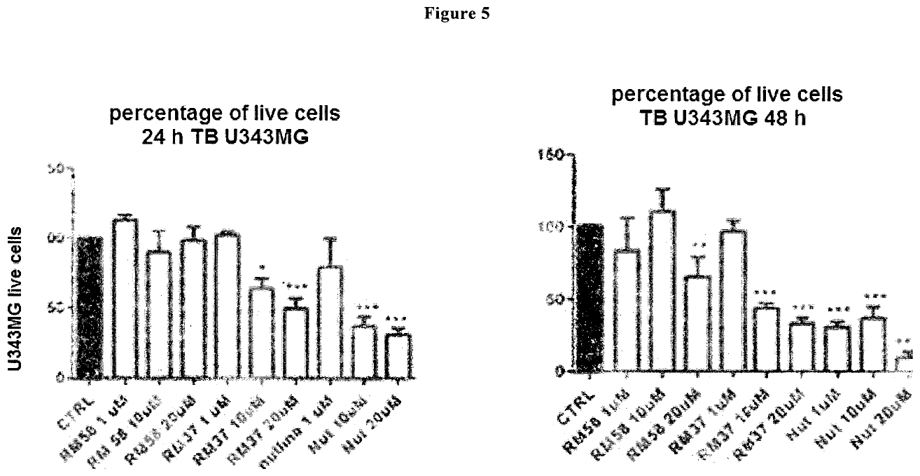

6) Antitumoral effect in vitro (RM37 vs Nut-3): live cell count (U343MG; 1,2). The results are set out in FIG. 5.

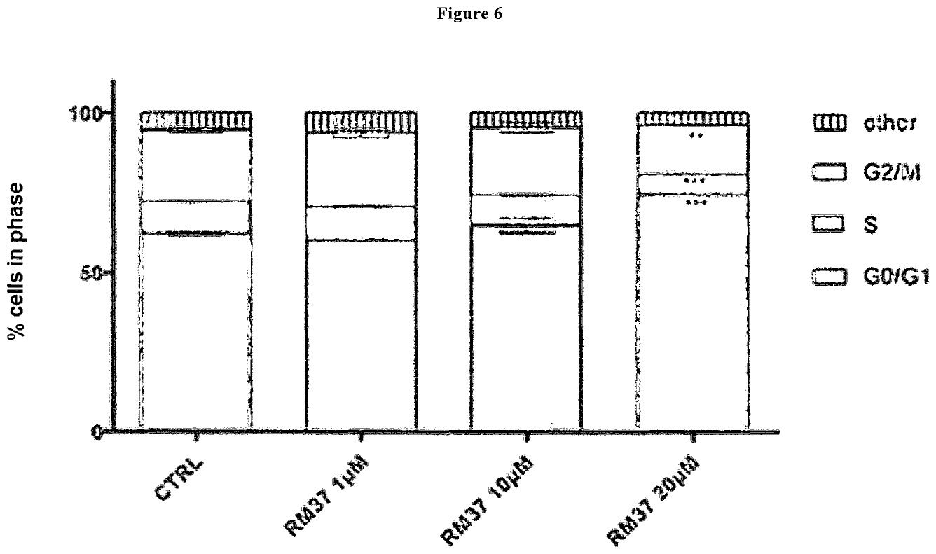

7) Effect of RM37 on cell cycle (U343MG; 1,2). RM37 blocks the cell cycle in G1. The results are set out in FIG. 6.

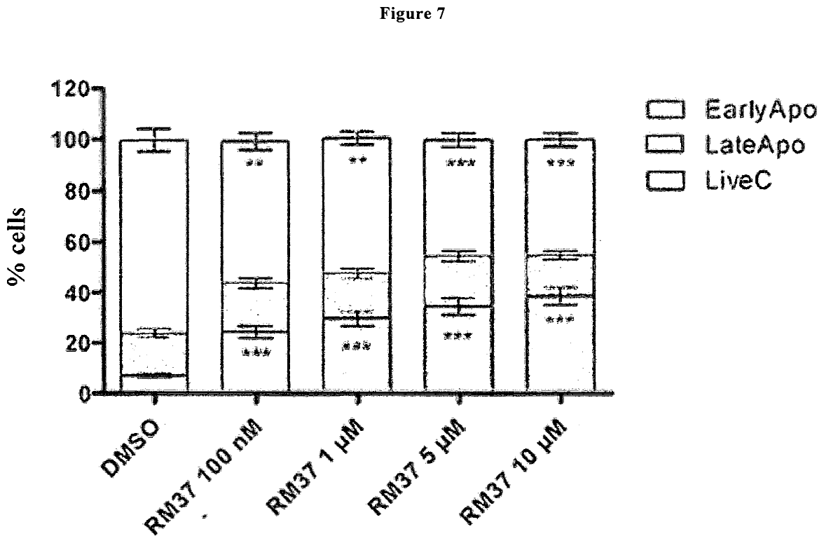

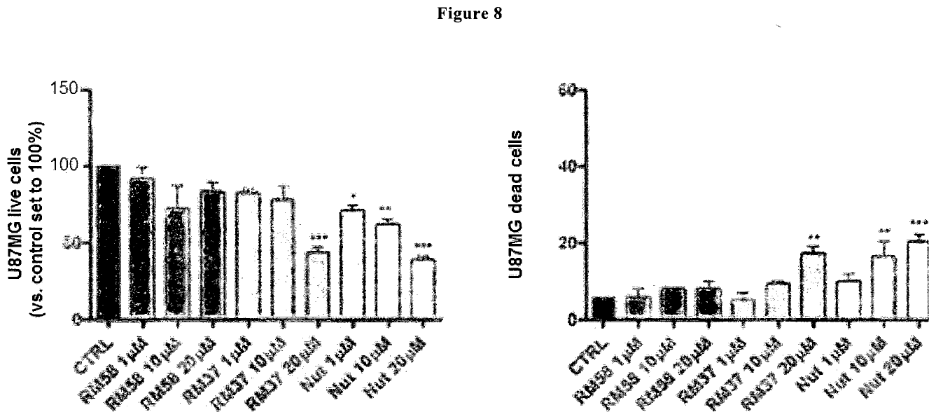

8) Effect of RM37 on cell apoptosis (U343MG; 1,2). RM37 induces cell apoptosis (U343MG). The results are set out in FIG. 7 and FIG. 8.

1. Compounds of general formula (I): 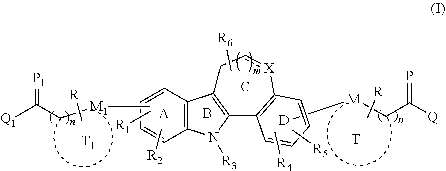
wherein
n is an integer ranging from 0-12;
m is an integer ranging from 0-2;
the phenyl rings A e D condensed in the tetracyclic system A-D are optionally substituted, both or only the ring A o only the ring D, in any of the substitutable, by a chain of formula II o II′: 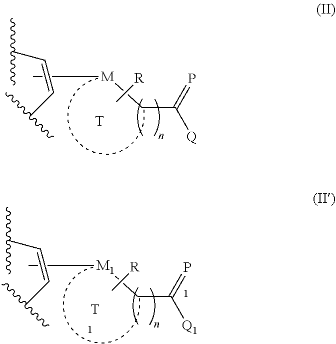
wherein the chain of formula II may be represented by an amino acid, natural o non-natural, of straight type of formula III (with T absent) or cyclic type of formula IV (with T present); or by a peptide sequence, containing amino acids natural and/or non-natural of type (III) and (IV), of formula (V); o by a peptide sequence of formula (VI) ending with an amino acid of type (III) or (IV); or by a sequence of formula (VII) or (VIII): 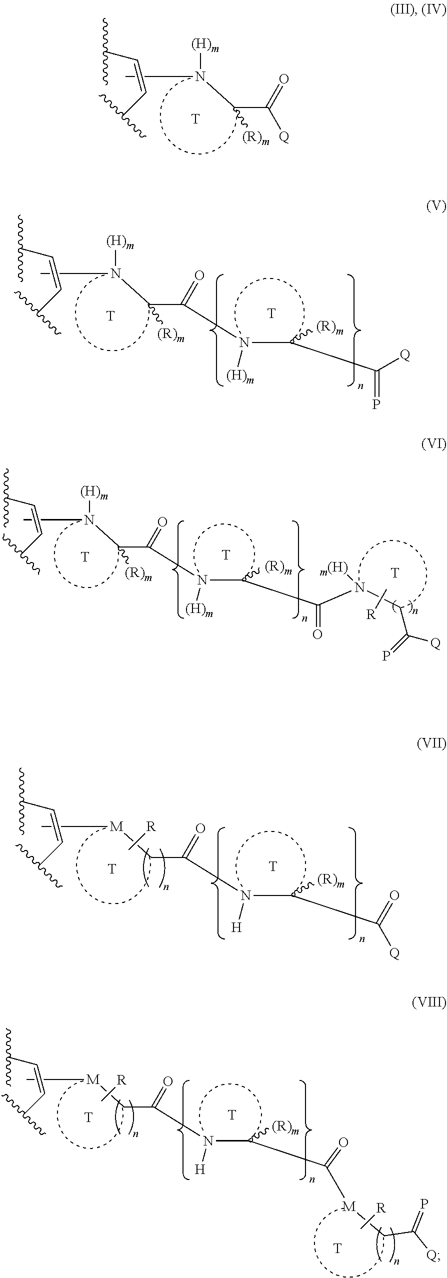
R is selected from the group consisting of hydrogen or a group R₁ or a group G wherein G is a carbon atom also halogenated or poly-halogenated with atoms of F or Cl or Br or I, or a carbon atom of a monocyclic o bicyclic aryl, or a carbon or a nitrogen atom being part of an aromatic or non-aromatic heterocyclic system selected from the group consisting of pyrrole, pyrrolidine, 3-pyrroline, 2H-pyrrole, 2-pyrroline, indole, isoindole, 3H-indole, indolizine, indoline, furan, benzofuran, isobenzofuran, 2H-pyran, 4H-pyran, benzo[b]thiophene, thiophene, pyridine, piperidine, 4H-quinolizine, isoquinoline, quinoline, tetrahydroquinoline, 1,8-naphthyridine, acridine, oxazole, isoxazole, benzoxazole, benzothiazole, isothiazole, triazole, imidazole, 2-imidazole, imidazolidine, tetrazole, 1,2,3-triazole, 1,2,4-triazole, 1,2,3-oxadiazole, benzimidazole, purine, 1,4-dioxane, 1,3-dioxolane, 1,3-dithiane, 1,4-dithiane, 1,3,5-trithiane, morpholine, thiomorpholine, phenothiazine, pyrazole, 2-pyrazoline, pyrazolidine, quinazoline, cinnoline, pyrimidine, pyrazine, pteridine, phthalazine, 1,2,4-triazine, 1,3,5-triazine, pyridazine, piperazine, quinoxaline, phenazine, 1H-indazole; or R is a C₁-C₁₀ carbon atoms chain, saturated or unsaturated, straight or branched, optionally substituted with a substituent selected from R₁, G, a hydroxyl, —O-alkyl, —ONO₂, —O-G, phenyl, substituted phenyl, heteroaryl, substituted heteroaryl, —NH-G, —NG₂, —NR₇R₈, ═N—O-G, —NH—O-G, —COOH, —(CH₂)ₚ—COOH, —(CHR₂)ₚ—COOH, —(CH₂)ₚ—CO—NHR′, —(CHR₂)ₚ—CO—NHR′, wherein p can be a number from 0 to 12 and wherein R₇ and R₈ are independently selected from hydrogen, all the meanings of R₁, all the meanings of R₂;
or R is a C₁-C₆ carbon atoms chain, saturated or unsaturated, straight or branched, optionally substituted with aryl, —CO— alkyl, —CO-aryl, —CO-heteroaryl; —CONH-alkyl, —CONH-alkyl-ONO₂; —CONH-acyl; —CONH-acyl-ONO₂, —CONH-aryl, —SO₂-alkyl, —SO₂NH₂, —SO₂NH-alkyl, —SO₂NH-aryl, wherein aryl may be phenyl, substituted, phenyl, heteroaryl or substituted heteroaryl, and wherein R′ is selected from alkyl, alkyl-O—NO₂, aryl, acyl e acylaryl, wherein aryl may be phenyl, substituted, phenyl, heteroaryl or substituted heteroaryl, or as indicated in the meanings of G, when m=2 the groups R are 2 and they are 2 substituents, the same or different, selected from the meanings of R; in some cases two groups R can form together a ring which has as closure the bearing carbon of R with the proviso that when m is 2, T is absent;
R₁ is selected from H, F, Cl, Br, I, -aryl, -heteroaryl, —R, —NO₂, —NH₂, —NHCH₃, —NH-alkyl, —NH-aryl, —NH-heteroaryl, —NH—R, —N(R)₂, —NRR₁, —N(CH₂)₂, —N(CH₂)₃, —N(CH₂)₄, —N(CH₂)₅, N(CH₂)₄O, —N(CH₂)₄S(O)ₘ, —N(CH₂)₄N—R, —N(CH₂)₄N—R₁, NHCOCH₃, —NHCO-alkyl, —NH—CO-cycloalkyl, —NHCO-aryl, —NHCO— heteroaryl, —NHCO—R, —NHSO₂CH₃, —NHSO₂-alkyl, —NHSO₂-cycloalkyl, —NHSO₂-aryl, —NHSO₂-heteroaryl, —NHSO₂—R, —SCH₃, —SR, —SO₂CH₃, —SO₂-alkyl, —SO₂-cycloalkyl, —SO₂-aryl, —SO₂-heteroaryl, —SO₂—R, —SO₂NH₂, —SO₂NHCH₃, —SO₂NH-alkyl, —SO₂NH-cycloalkyl, —SO₂NH-aryl, —SO₂NH-heteroaryl, —SO₂NH—R, —SO₂NHCOCH3, —SO₂NHCO-alkyl, —SO₂NHCO-aryl, —SO₂NHCO-heteroaryl, —SO₂NHCO-cycloalkyl, —C(CH)₄N (e.g. 2-pyridine, 3-pyridine, 4-pyridine), —C(CH)₃O (e.g. 2-furan, 3-furan), —C(CH)₃S (e.g. 2-thiophene, 3-thiophene), —CH(CH₂)O (e.g. oxirane), —CH(CH₂)S (e.g. thiiran), —CO₂H, —CO₂CH₃, —CO₂-alkyl, —CO₂-aryl, —CO₂-heteroaryl, —CO₂-cycloalkyl, —CO₂R, —CONHCH₃, —CONH-alkyl, —CONH-aryl, —CONH-heteroaryl, —CONH-cycloalkyl, —CONH—R, —CONRCH₃, —CONRR, —CONHSO₂CH₃, —CONHSO₂-alkyl, —O—CH₂-alkyl, —O—(CH₂)n-R, —O-acyl, —O-aryl, —O-heteroaryl, —O-cycloalkyl, —O—R, —O—CH₂OCH₃, —O—CH₂OCH₂CH₃, —O—CH₂(OCH₂CH₂)ₙ—OCH3, —O—(CH₂)ₙ—NH-alkyl, —O—(CH₂)ₙ—N-(alkyl)2, —O—(CH₂)ₙ—NH-cycloalkyl with ring from 4 to 6 carbon atoms, —O—(CH₂)ₙ—R with the R group as defined above, —O—CH₂ (NH CH₂CH₂)n-OCH₃, —O—CH₂(CH₂)ₙCO(NHCH₂CH₂)ₙ—OCH₃, —O—CH₂(CH₂)ₙSO₂(NHCH₂CH₂)ₙ—OCH₃, —N(CH₂CH₂)₂N—(CH₂CH₂)ₙ—NH, CH₃N(CH₂CH₂)₂N—(CH₂CH₂)ₙ—, CH₃ (CH₂)ₙCO—N(CH₂CH₂)₂N—(CH₂CH₂)ₙ, CH₃ (CH₂)ₙSO₂—N(CH₂CH₂)₂N—(CH₂CH₂)ₙ—, O(CH₂CH₂)₂N—(CH₂CH₂)ₙ—, monocyclic or bicyclic aryl, or an aromatic or non-aromatic heterocyclic system selected from the meanings of R;
M is selected from O, CH₂, CH—R₂, C═CH—R₂, C(R₂)ₘ, —CH—OH, —CH—O—R₂, C═CH—O—R₂, C═O, C═ONR₂, NH, N—R₂, N—OH, N—O—R₂, NR₂CO—, S, S═O, S(═O)₂, wherein R₂ is independently selected from the meanings of R, or R₁, R₂, R₃, R₄, R₅, R₆, R₇ and R₈;
M₁ is independently selected from the meanings of M, as defined above, or absent;
R₂, R₃, R₄, R₅, R₆, R₇, R₈, the same or different, are independently selected from the meanings of R, or R′, R₁;
P is selected from O, Q, CH₂, CH—R₂, C— (R₂)ₘ, CH—OH, CH—O—R₂, CH—O-acyl, NH, N-acyl, N—R₂, N—OH, N—O—R₂, N—O-acyl, wherein R₂ is independently selected from the meanings of R, or R′, R₁, R₂, R₃, R₄, R₅, R₆, R₇ and R₈;
P₁ is independently selected from the meanings of P, as defined above, or absent;
Q is selected from H, R₂, O—R₂, O-acyl, NH₂, NH—R₂, N— (R₂)ₘ, NH-acyl, NHCOO—CH₂Bn, NHCOO-t-Bu, NHCOO—R, NH—OH, NH—O—R₂, N(R₂)—O—R₂, NH—O-acyl, wherein R₂ is independently selected from the meanings of R, or R′, R″, R₁, R₂, R₃, R₄, R₅, R₆, R₇ and R₈; or Q is a beta-lactam structure of formula (IX′), (IX″), (IX′″): 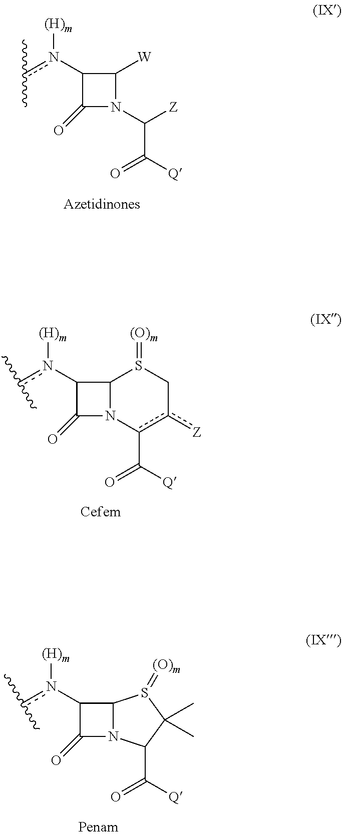
wherein Q′ is selected from H, R₂, O—R₂, O-acyl, NH₂, NH—R₂, N—(R₂)ₘ, NH-acyl, NHCOO—CH₂Bn, NHCOO-t-Bu, NHCOO—R, NH—OH, NH—O—R₂, N(R₂)—O—R₂, NH—O-acyl, wherein R₂ is independently selected from the meanings of R, or R′, R″, R₁, R₂, R₃, R₄, R₅, R₆, R₇ and R₈;
Q₁ is independently selected from the meanings of Q, as defined above, or absent;
T is a saturated or containing unsaturation cyclic ring, consisting of 3 to 8 atoms linked together and containing carbon atoms and/or also N, O or S(O)ₘ or also substituted, as in the defined meanings, by R, R′, R″, R₁, R₂, R₃, R₄, R₅, R₆, R₇ and R₈ or by X, M, P, W or Z or as defined in the meanings of the cyclic systems of G for R and/or R₁;
T₁ is independently selected from the meanings as T, as defined above, or absent;
X is selected from groups —CH₂—, —CH(R″)—, ═C(R″)—, —O—, —S(═O)ₘ—, ═N—, —N(R″)—, —C(═O)—, —C(═P)—, —N(R″) CO, —CON(R″)—, wherein R″ is H is one of the meanings defined for R, R′ and for R₁, R₂, R₃, R₄, R₅, R₆, R₇ and R₈;
W is selected from H, or from the meanings of T or Z or R, or also of R′, R″, R₁, R₂, R₃, R₄, R₅, R₆, R₇ and R₈;
Z is selected from H, CH₃, CH₂OCOCH₃, or, taken together with the carbon atom to which it is linked, Z is selected from ═CH₂, ═CH(R₂), C(═O), C(═P); or Z is selected from the meanings of R, or of R′, R″, R₁, R₂, R₃, R₄, R₅, R₆, R₇ and R₈, or also from the meanings of M, P.

2. Compounds according to claim 1, wherein the compounds of formula (I) are characterized by a substructure of formula (X): 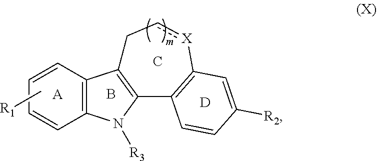
wherein m, X, R₁, R₂, R₃ are as defined in claim 1.

3. Compounds according to claim 2, wherein the compounds of formula (X) are characterized by a substructure of formula (XI) or (XII): 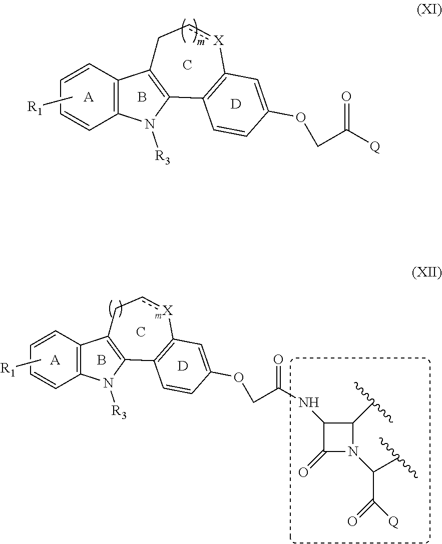
wherein 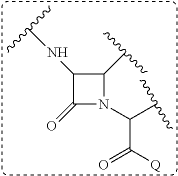
is a substituent of formula IX′, IX″ or IX′″.

4. Compounds according to claim 3, wherein the compounds of formula (XII) are characterized by a substructure of formula (XIII), (XIV) or (XV):

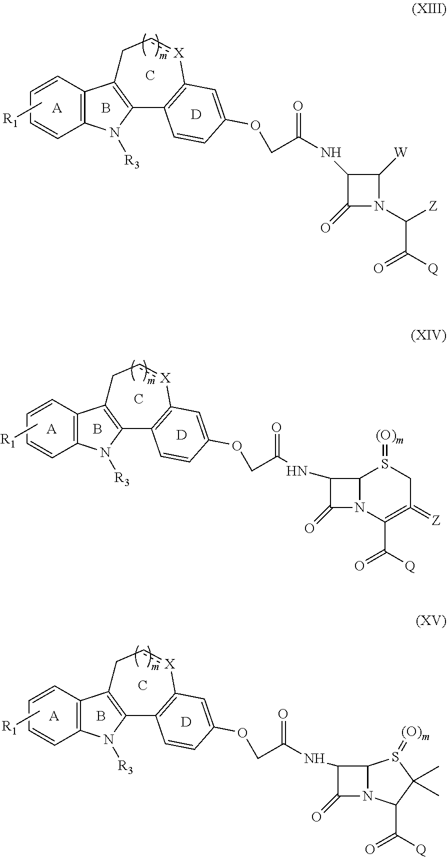

5. Compounds according to claim 2, wherein the compounds of formula (X)
have a formula selected from the following:

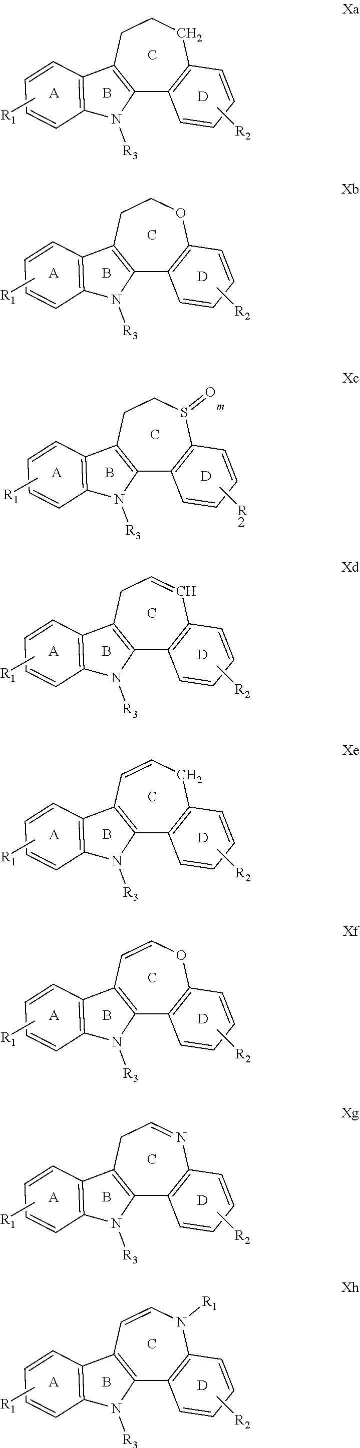

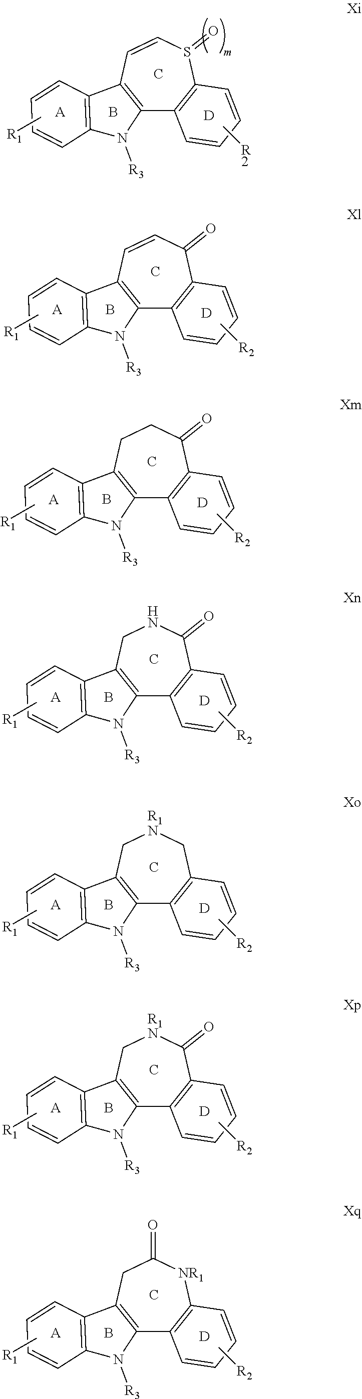

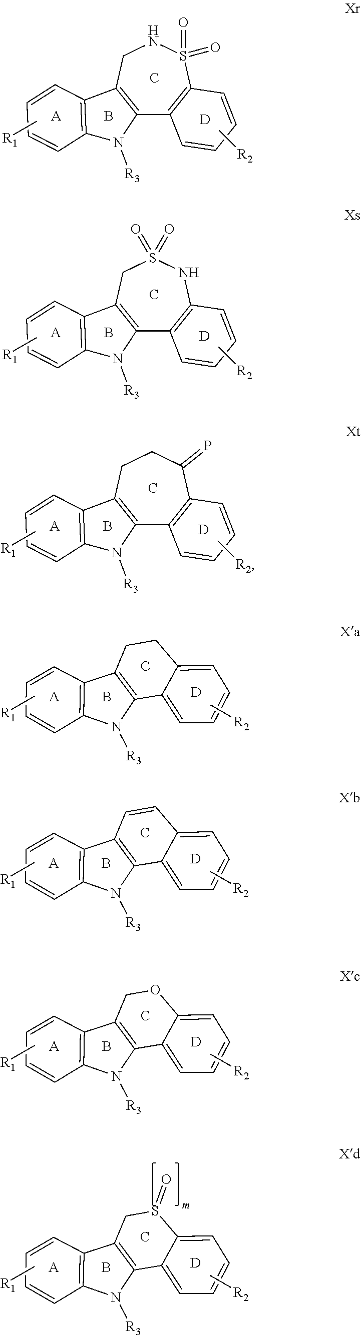

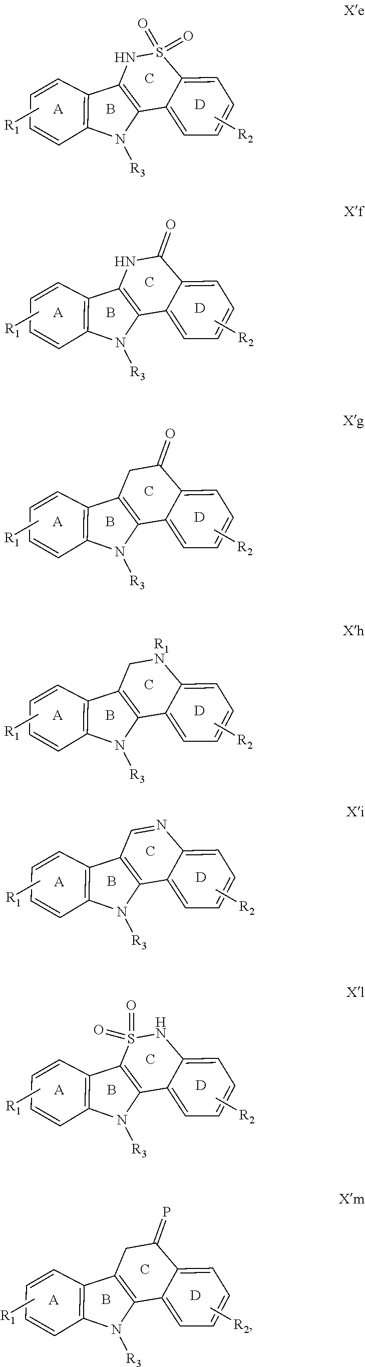

6. Compounds according to claim 3, wherein the compounds of formula (XI) have a formula selected from the following:

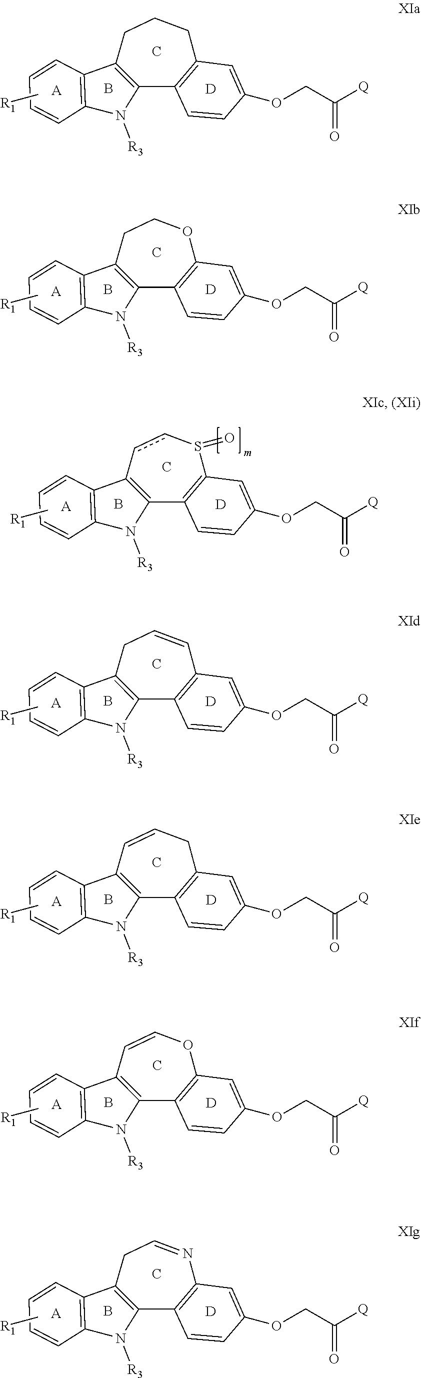

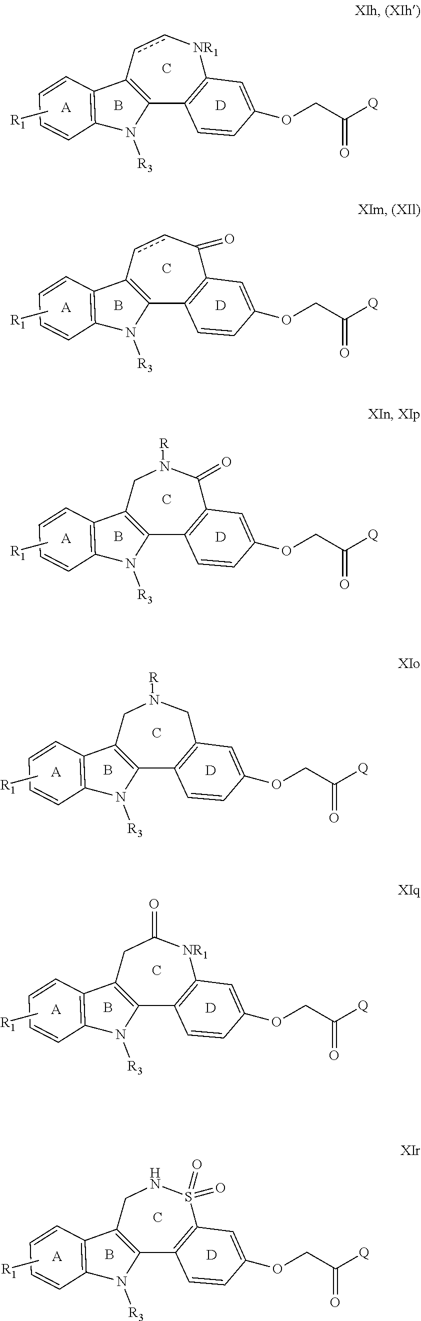

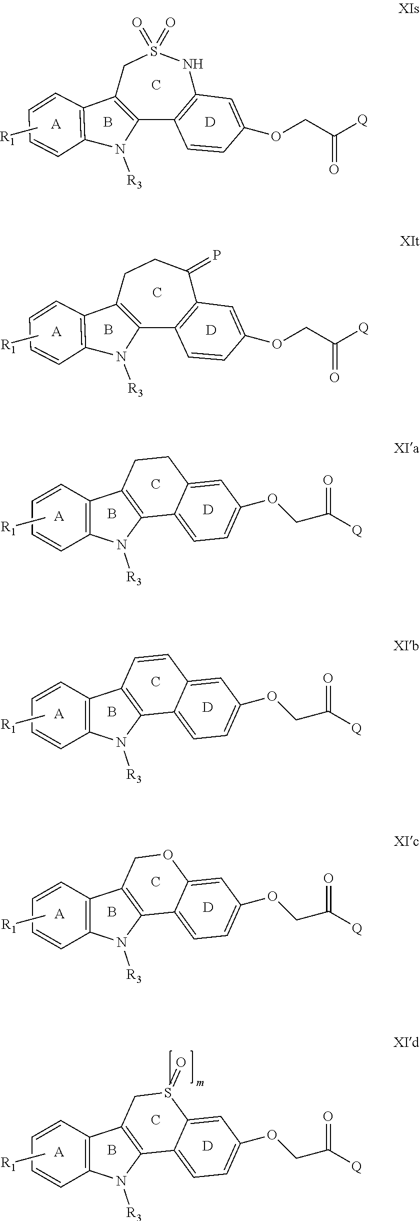

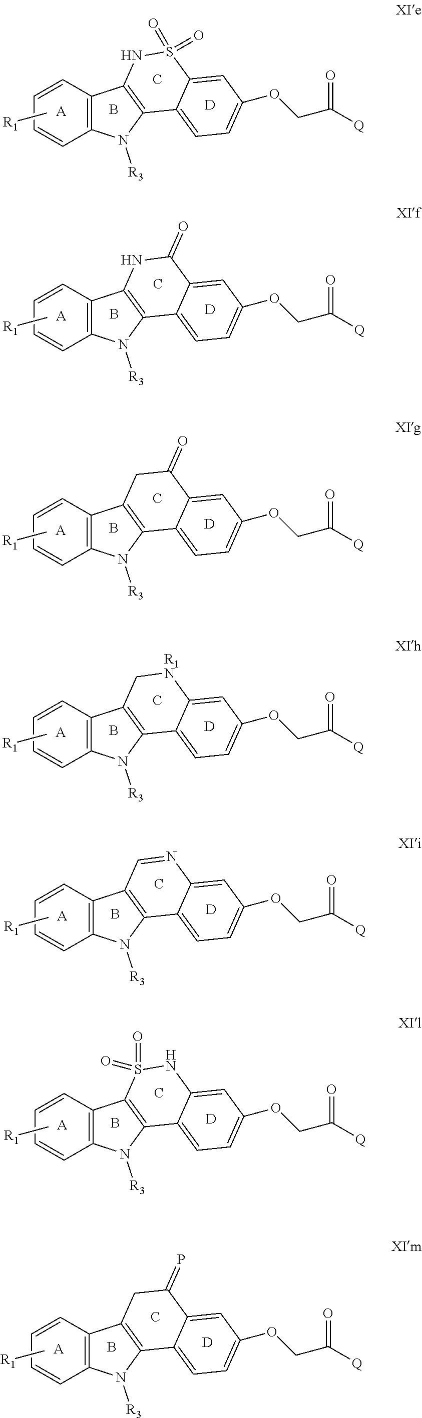

7. Compounds according to claim 4, wherein the compounds of formula (XIII) have a formula selected from the following:

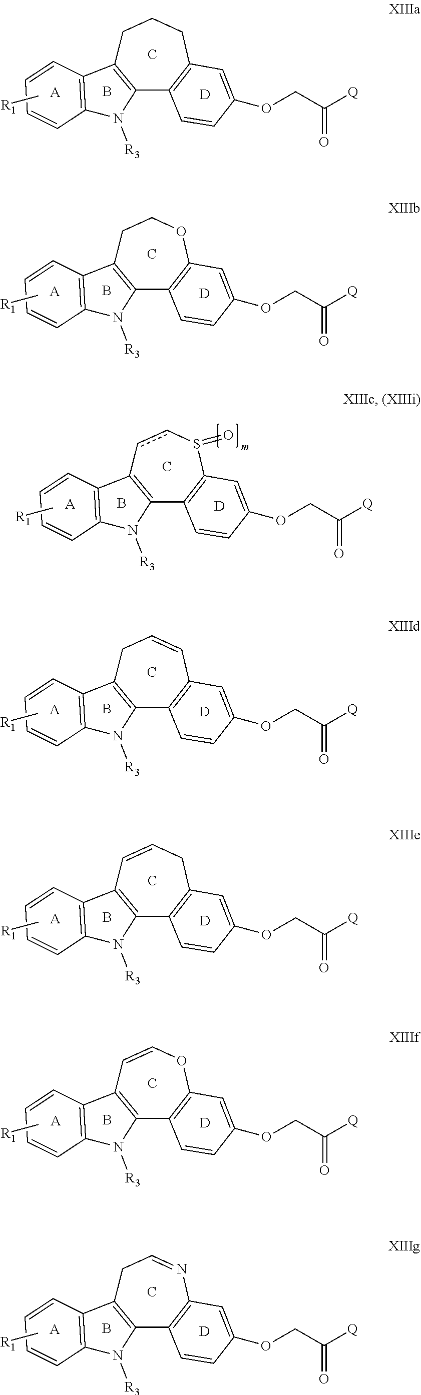

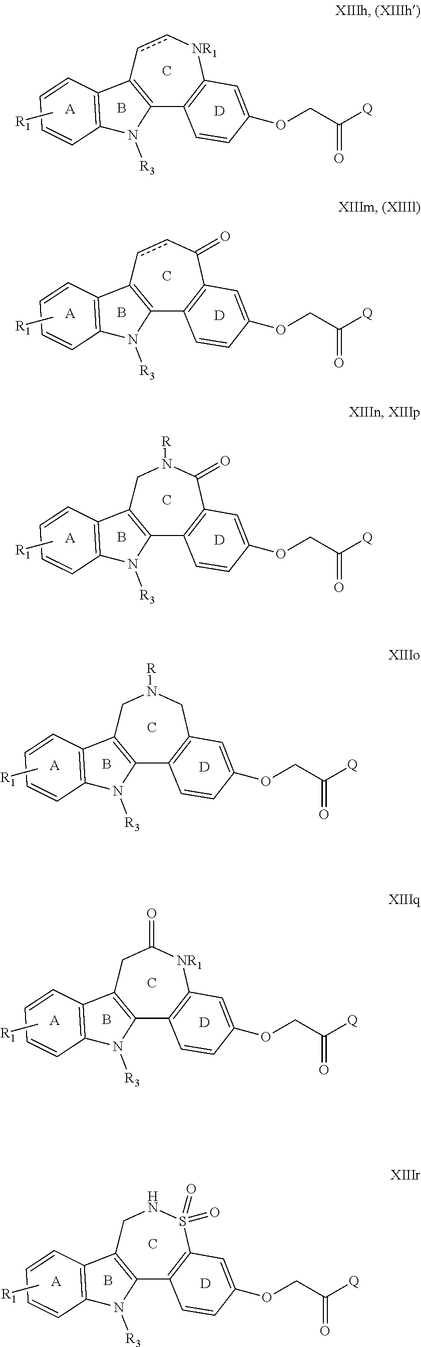

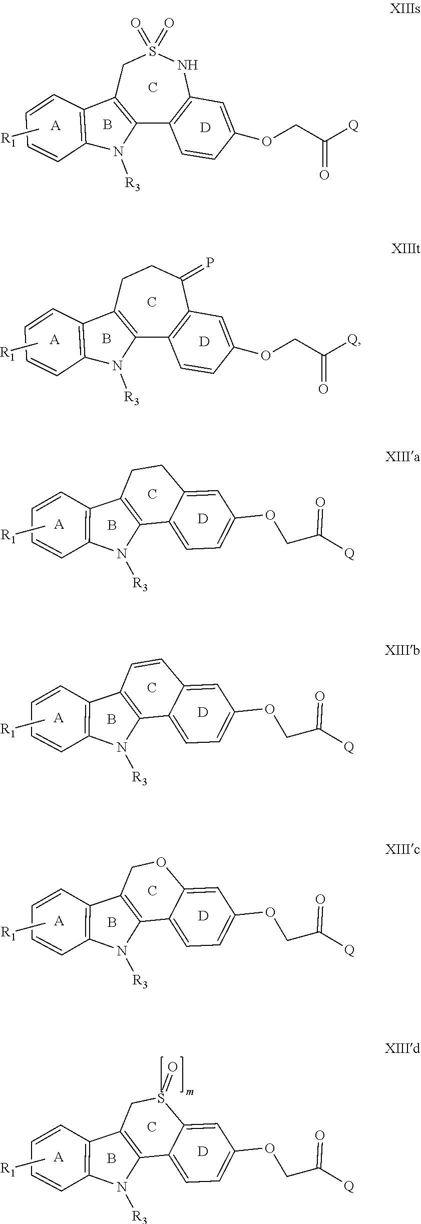

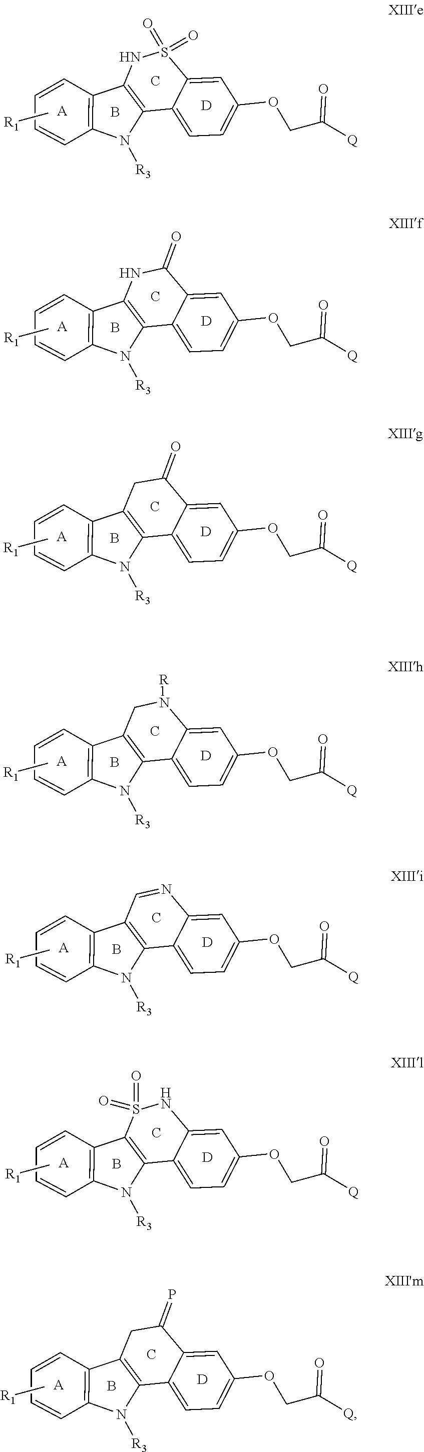

8. Compounds according to claim 4, wherein the compounds of formula (XIV) have a formula selected from the following:

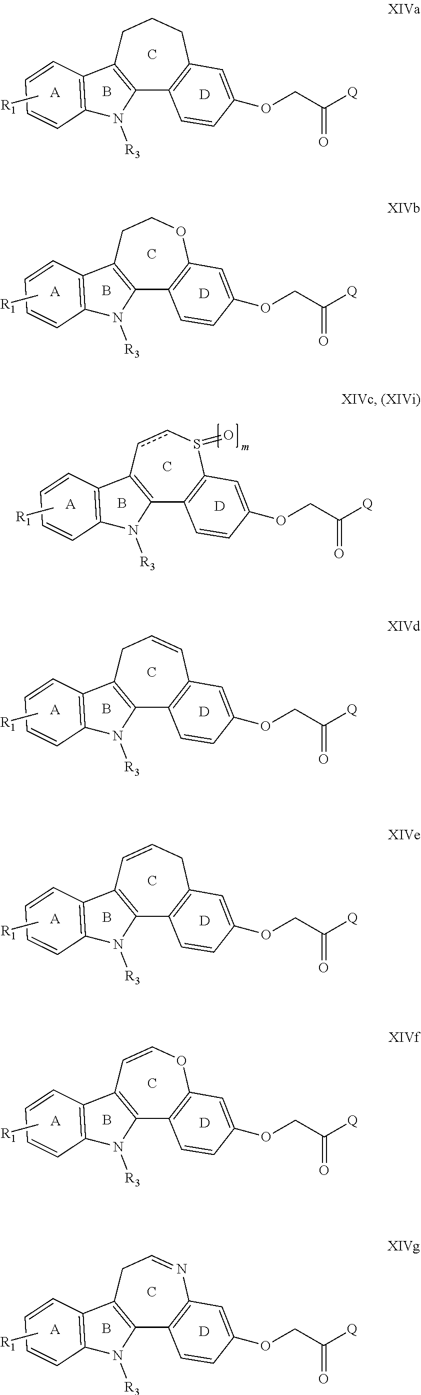

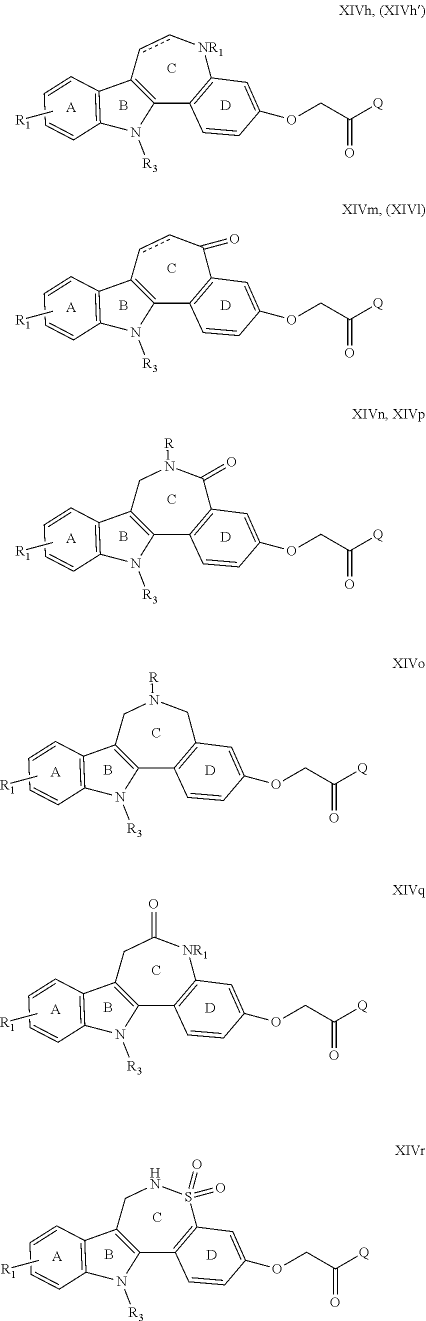

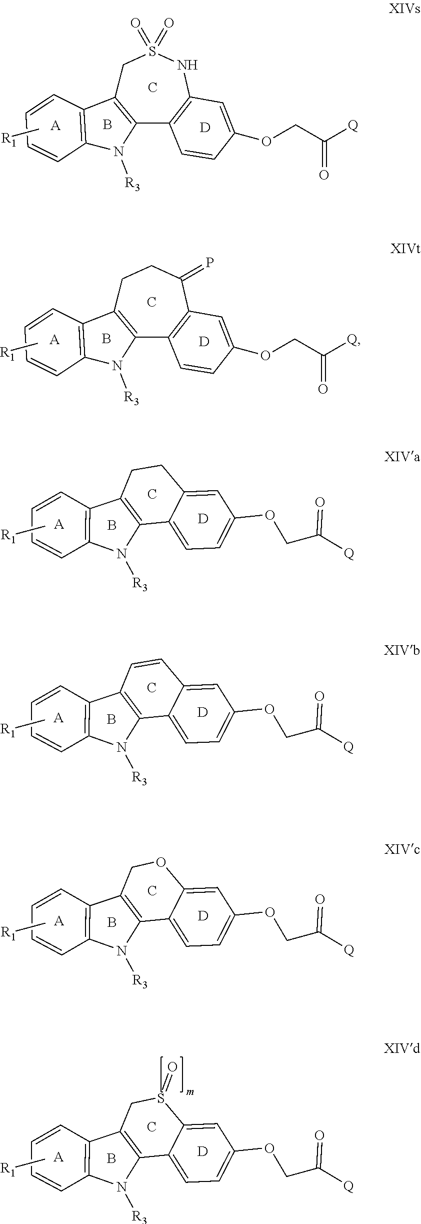

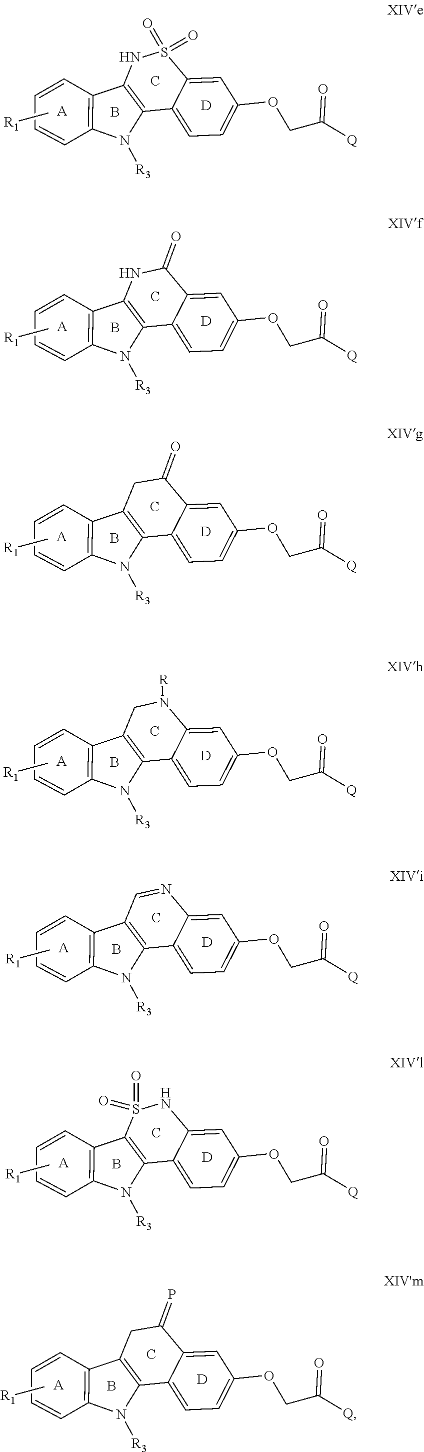

9. Compounds according to claim 4, wherein the compounds of formula (XV) have a formula selected from the following:

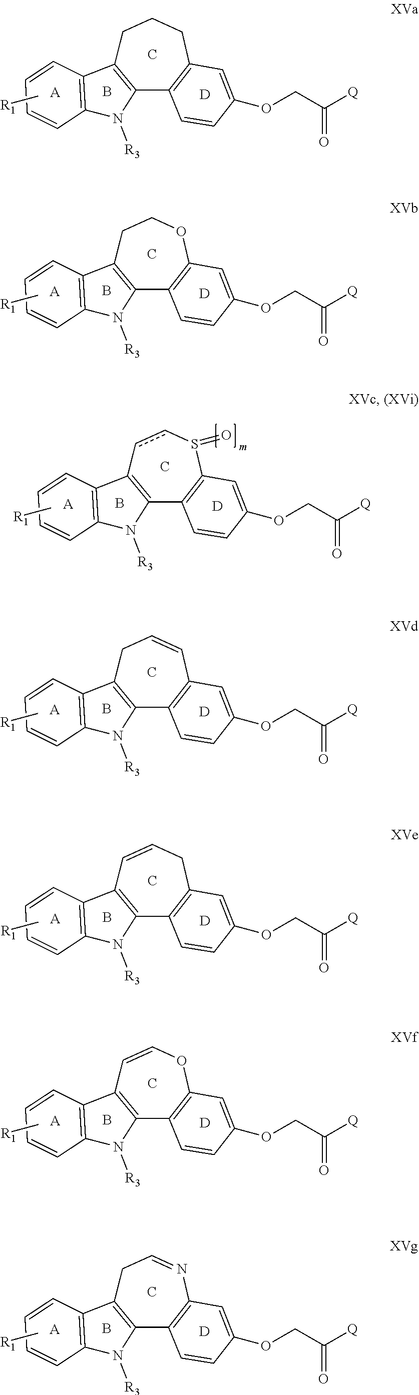

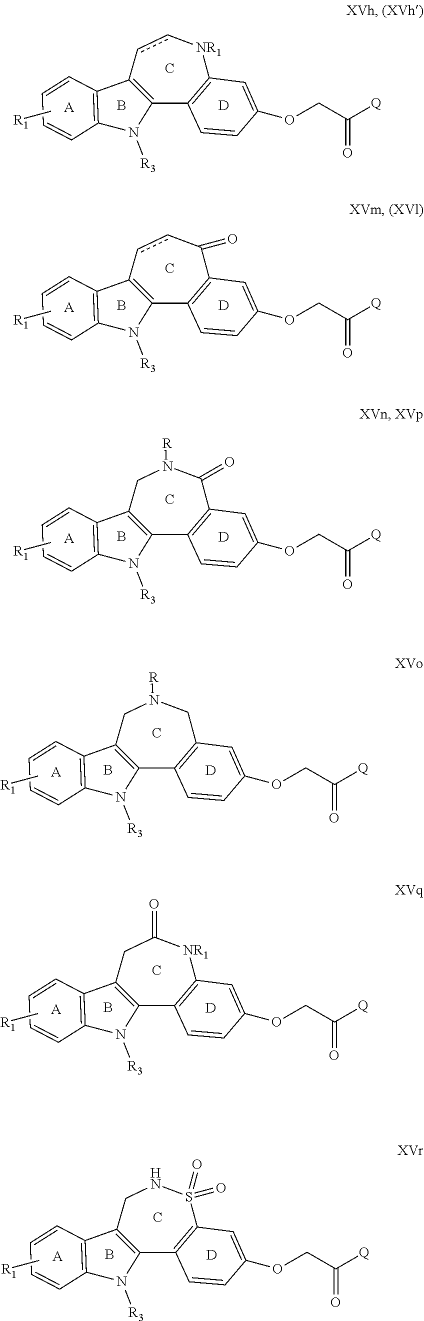

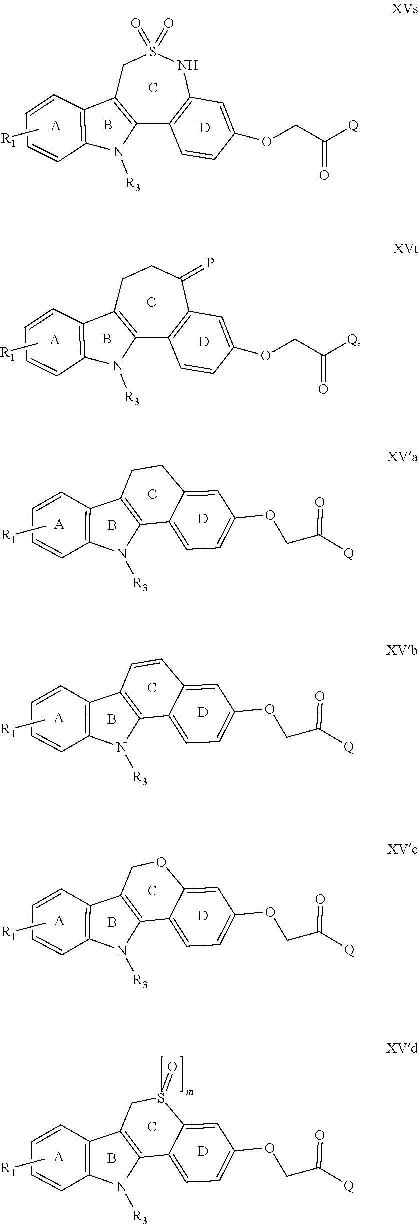

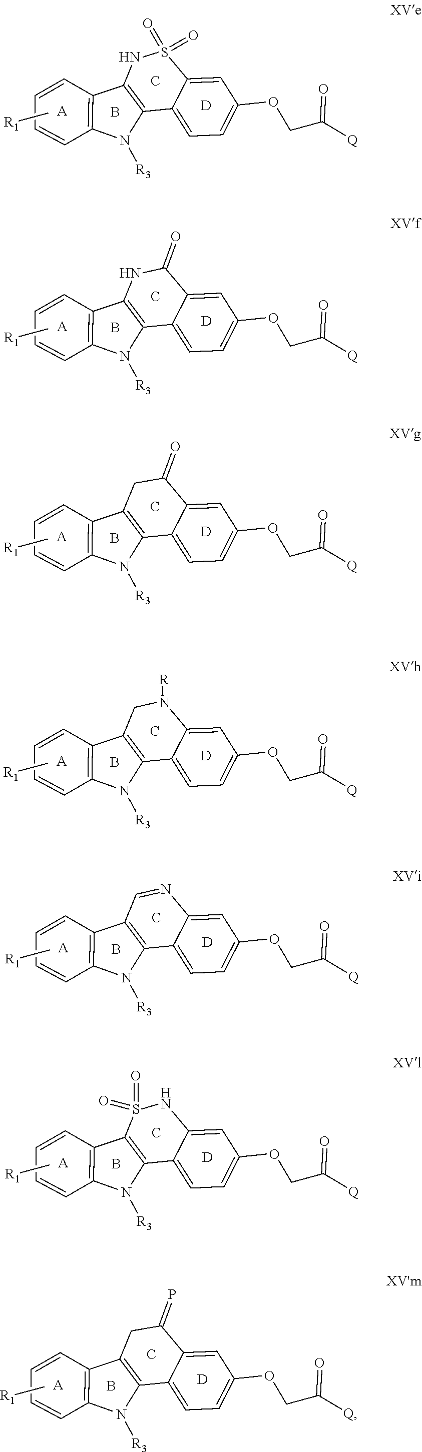

10. Compounds according to claim 1, wherein the compounds are selected from the group consisting of:

| # | # | |
|---|---|---|
| substructure | Abbreviation | Structure |
| XIc XIl | RA1 | 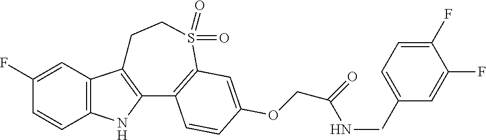 |
| XIa | RA2 | 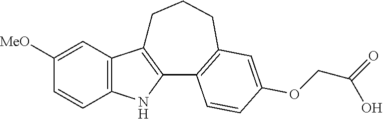 [SMILES: COc1ccc2[nH]c3c(c2c1)CCCc1cc(OCC(=O)O)ccc1-3] |
| XIr | RA3 | 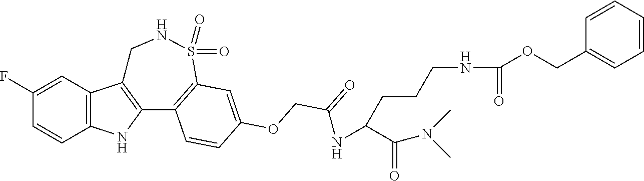 [SMILES: CN(C)C(=O)C(CCCNC(=O)OCc1ccccc1)NC(=O)COc1ccc2c(c1)S(=O)(=O)NCc1c-2[nH]c2ccc(F)cc12] |
| Xa | GA17M8 | 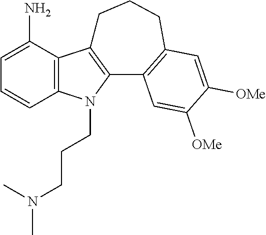 [SMILES: COc1cc2c(cc1OC)-c1c(c3c(N)cccc3n1CCCN(C)C)CCC2] |
| Xa | GA17M10 | 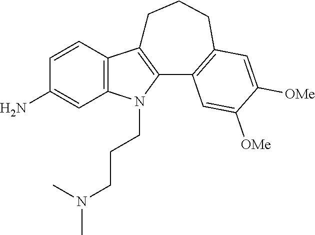 [SMILES: COc1cc2c(cc1OC)-c1c(c3ccc(N)cc3n1CCCN(C)C)CCC2] |
| XIb | GA11S | 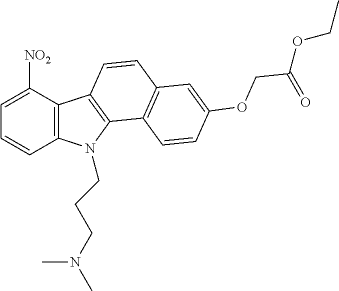 [SMILES: CCOC(=O)COc1ccc2c(ccc3c4c([N+](=O)[O-])cccc4n(CCCN(C)C)c23)c1] |
| XIb | RM66 | 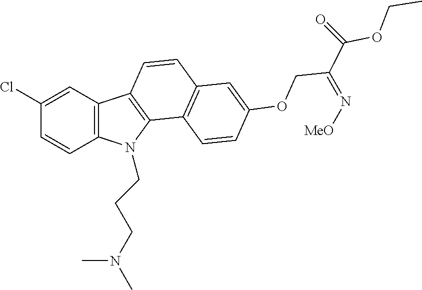 [SMILES: CCOC(=O)/C(COc1ccc2c(ccc3c4cc(Cl)ccc4n(CCCN(C)C)c23)c1)=N/OC] |
| XIb | RM58 | 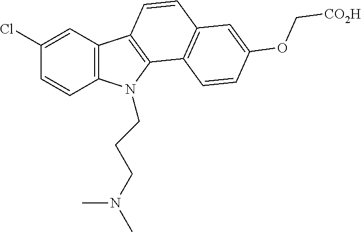 [SMILES: CN(C)CCCn1c2ccc(Cl)cc2c2ccc3cc(OCC(=O)O)ccc3c21] |
| XIb | RM70 | 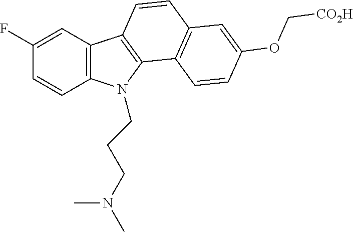 [SMILES: CN(C)CCCn1c2ccc(F)cc2c2ccc3cc(OCC(=O)O)ccc3c21] |
| XIb | RM85 | 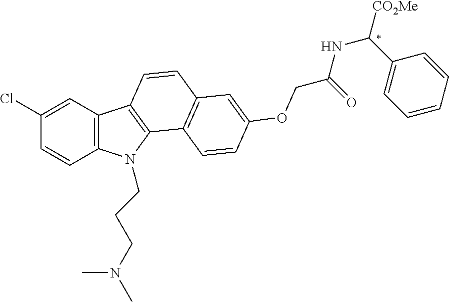 [SMILES: COC(=O)C(NC(=O)COc1ccc2c(ccc3c4cc(Cl)ccc4n(CCCN(C)C)c23)c1)c1ccccc1] |
| XI'd | RA6 | 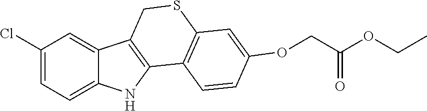 [SMILES: CCOC(=O)COc1ccc2c(c1)SCc1c-2[nH]c2ccc(Cl)cc12] |
| XI'd | RA7 | 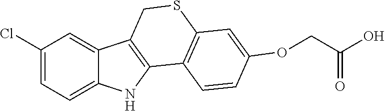 [SMILES: O=C(O)COc1ccc2c(c1)SCc1c-2[nH]c2ccc(Cl)cc12] |
| XI'd | RA9 | 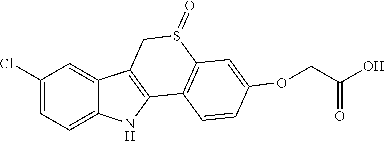 [SMILES: O=C(O)COc1ccc2c(c1)S(=O)Cc1c-2[nH]c2ccc(Cl)cc12] |
| XI'd | RA11 | 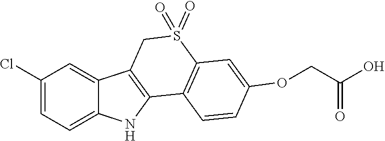 [SMILES: O=C(O)COc1ccc2c(c1)S(=O)(=O)Cc1c-2[nH]c2ccc(Cl)cc12] |
| XI'd | RA12 | 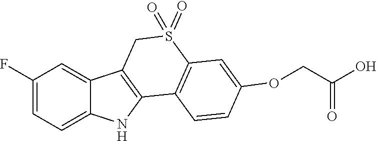 [SMILES: O=C(O)COc1ccc2c(c1)S(=O)(=O)Cc1c-2[nH]c2ccc(F)cc12] |
| XI'd | RA13 | 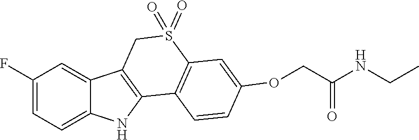 [SMILES: CCNC(=O)COc1ccc2c(c1)S(=O)(=O)Cc1c-2[nH]c2ccc(F)cc12] |
| XI'g | RA14 | 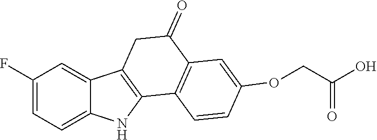 [SMILES: O=C(O)COc1ccc2c(c1)C(=O)Cc1c-2[nH]c2ccc(F)cc12] |
| X'c | RA15 | 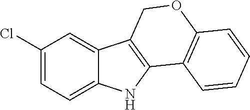 [SMILES: Clc1ccc2[nH]c3c(c2c1)COc1ccccc1-3] |
| X'c | RA16 | 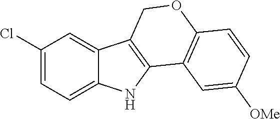 [SMILES: COc1ccc2c(c1)-c1[nH]c3ccc(Cl)cc3c1CO2] |
| XI'c | RA17 | 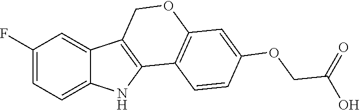 [SMILES: O=C(O)COc1ccc2c(c1)OCc1c-2[nH]c2ccc(F)cc12] |
| XI'c | RA18 | 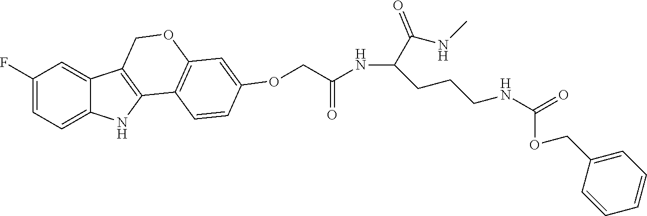 [SMILES: CNC(=O)C(CCCNC(=O)OCc1ccccc1)NC(=O)COc1ccc2c(c1)OCc1c-2[nH]c2ccc(F)cc12] |
| XI'c | RA19 | 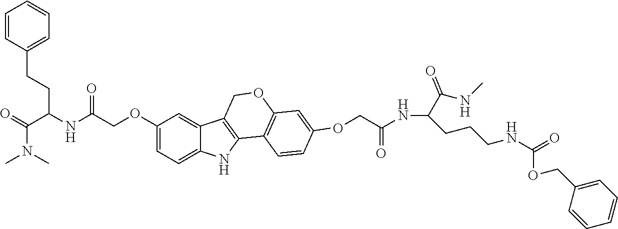 [SMILES: CNC(=O)C(CCCNC(=O)OCc1ccccc1)NC(=O)COc1ccc2c(c1)OCc1c-2[nH]c2ccc(OCC(=O)NC(CCc3ccccc3)C(=O)N(C)C)cc12] |
| XI'h | RA20 | 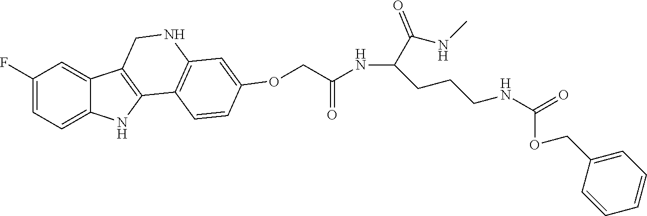 [SMILES: CNC(=O)C(CCCNC(=O)OCc1ccccc1)NC(=O)COc1ccc2c(c1)NCc1c-2[nH]c2ccc(F)cc12] |
| X'e | RA21 | 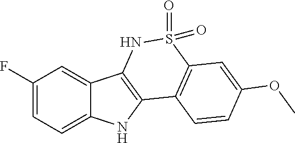 [SMILES: COc1ccc2c(c1)S(=O)(=O)Nc1c-2[nH]c2ccc(F)cc12] |
| XI'e | RA22 | 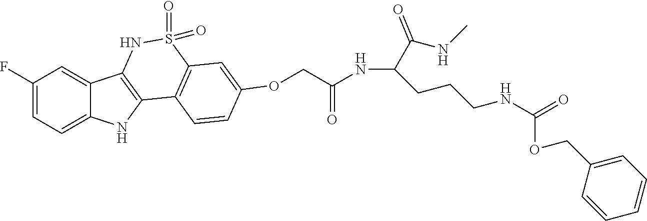 [SMILES: CNC(=O)C(CCCNC(=O)OCc1ccccc1)NC(=O)COc1ccc2c(c1)S(=O)(=O)Nc1c-2[nH]c2ccc(F)cc12] |
| XIV'a | GA09a | 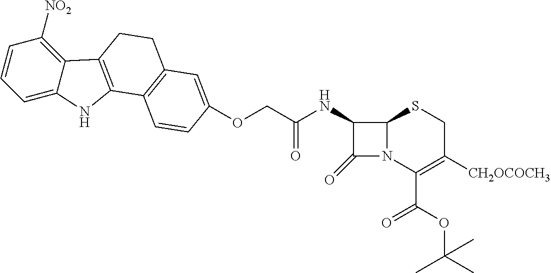 |
| XIV'a' | GA09a' | 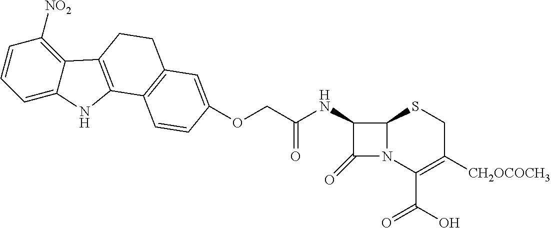 |
| XIV'a | GA09b | 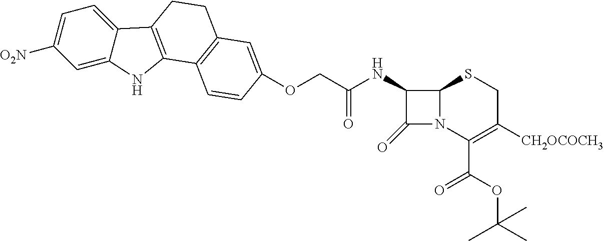 |
| XIV'a' | GA09b' | 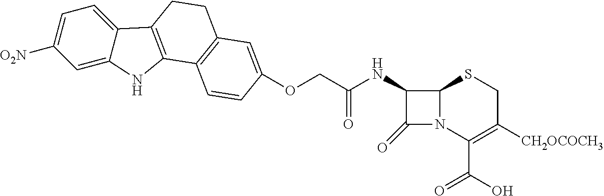 |
| XIV'a | RM36 | 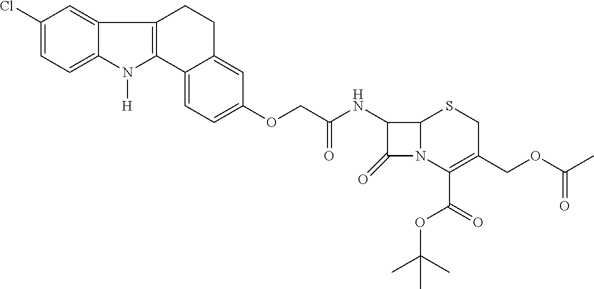 [SMILES: CC(=O)OCC1=C(C(=O)OC(C)(C)C)N2C(=O)C(NC(=O)COc3ccc4c(c3)CCc3c-4[nH]c4ccc(Cl)cc34)C2SC1] |
| XIV'a | RM37 | 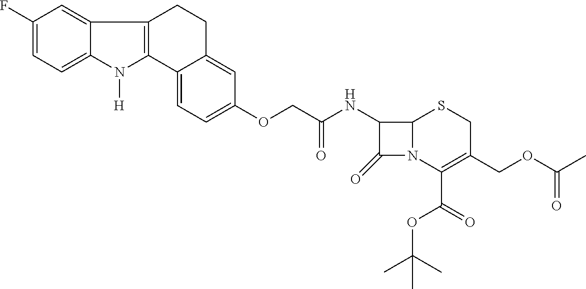 [SMILES: CC(=O)OCC1=C(C(=O)OC(C)(C)C)N2C(=O)C(NC(=O)COc3ccc4c(c3)CCc3c-4[nH]c4ccc(F)cc34)C2SC1] |
| XIV'a' | RM53 | 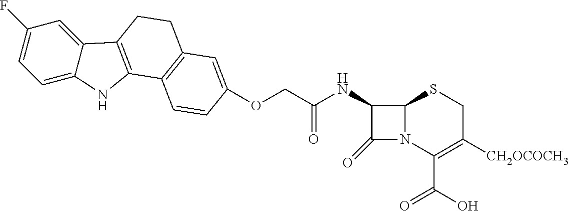 |
| XIV'a | RM47 | 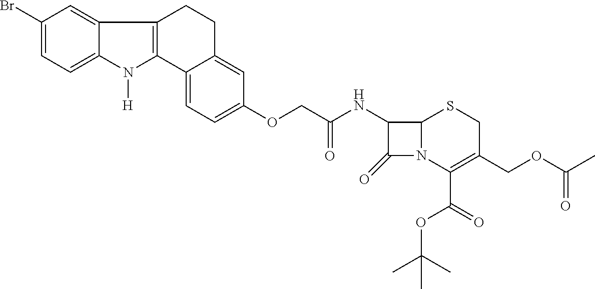 [SMILES: CC(=O)OCC1=C(C(=O)OC(C)(C)C)N2C(=O)C(NC(=O)COc3ccc4c(c3)CCc3c-4[nH]c4ccc(Br)cc34)C2SC1] |
| XIV'd | RA23 | 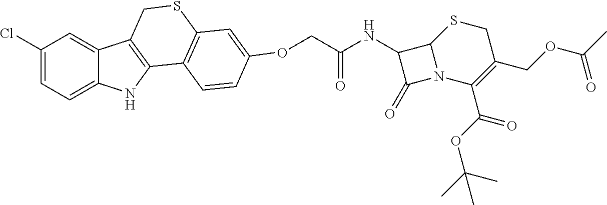 [SMILES: CC(=O)OCC1=C(C(=O)OC(C)(C)C)N2C(=O)C(NC(=O)COc3ccc4c(c3)SCc3c-4[nH]c4ccc(Cl)cc34)C2SC1] |
| XIV'd | RA24 | 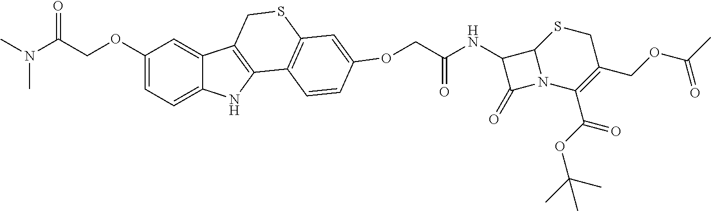 [SMILES: CC(=O)OCC1=C(C(=O)OC(C)(C)C)N2C(=O)C(NC(=O)COc3ccc4c(c3)SCc3c-4[nH]c4ccc(OCC(=O)N(C)C)cc34)C2SC1] |
| XIV'd | RA25 | 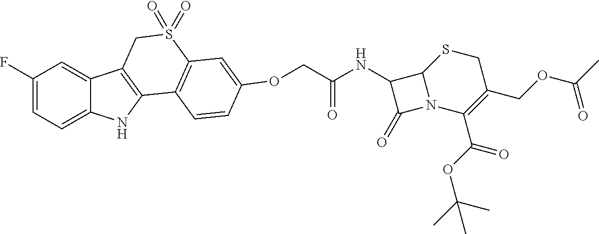 [SMILES: CC(=O)OCC1=C(C(=O)OC(C)(C)C)N2C(=O)C(NC(=O)COc3ccc4c(c3)S(=O)(=O)Cc3c-4[nH]c4ccc(F)cc34)C2SC1] |
| XV'e | RA26 | 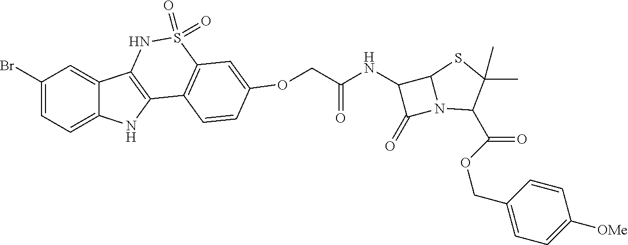 [SMILES: COc1ccc(COC(=O)C2N3C(=O)C(NC(=O)COc4ccc5c(c4)S(=O)(=O)Nc4c-5[nH]c5ccc(Br)cc45)C3SC2(C)C)cc1] |
| XV'd | RA27 | 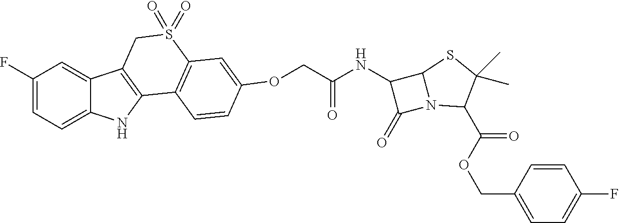 [SMILES: CC1(C)SC2C(NC(=O)COc3ccc4c(c3)S(=O)(=O)Cc3c-4[nH]c4ccc(F)cc34)C(=O)N2C1C(=O)OCc1ccc(F)cc1] |
| XV'd | RA28 | 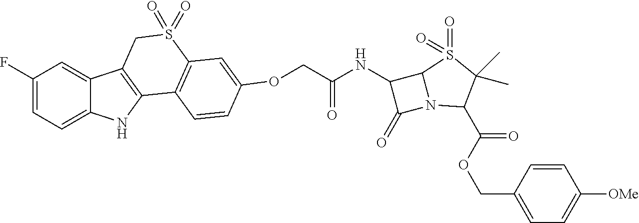 [SMILES: COc1ccc(COC(=O)C2N3C(=O)C(NC(=O)COc4ccc5c(c4)S(=O)(=O)Cc4c-5[nH]c5ccc(F)cc45)C3S(=O)(=O)C2(C)C)cc1] |
| XV'g' | RA29 | 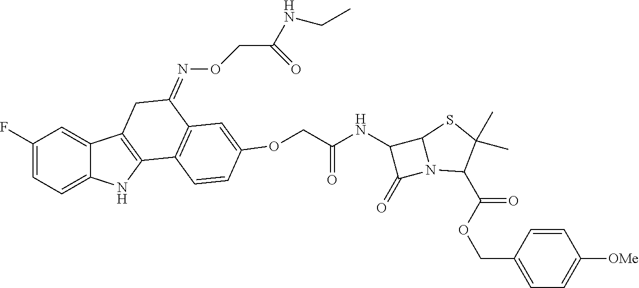 [SMILES: CCNC(=O)CO/N=C1/Cc2c([nH]c3ccc(F)cc23)-c2ccc(OCC(=O)NC3C(=O)N4C3SC(C)(C)C4C(=O)OCc3ccc(OC)cc3)cc21] |
| XIIId | RA30 | 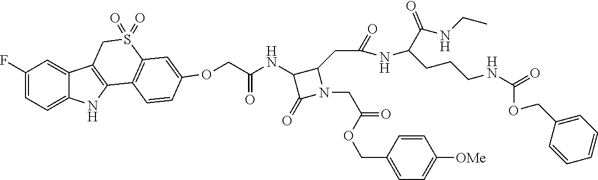 [SMILES: CCNC(=O)C(CCCNC(=O)OCc1ccccc1)NC(=O)CC1C(NC(=O)COc2ccc3c(c2)S(=O)(=O)Cc2c-3[nH]c3ccc(F)cc23)C(=O)N1CC(=O)OCc1ccc(OC)cc1] |
| XIV'c | RA31 | 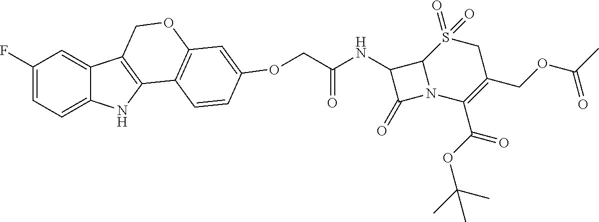 [SMILES: CC(=O)OCC1=C(C(=O)OC(C)(C)C)N2C(=O)C(NC(=O)COc3ccc4c(c3)OCc3c-4[nH]c4ccc(F)cc34)C2S(=O)(=O)C1] |
| XIV'c | RA32 | 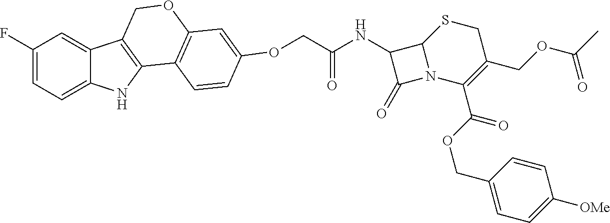 [SMILES: COc1ccc(COC(=O)C2=C(COC(C)=O)CSC3C(NC(=O)COc4ccc5c(c4)OCc4c-5[nH]c5ccc(F)cc45)C(=O)N23)cc1] |
| XIV'c | RA33 | 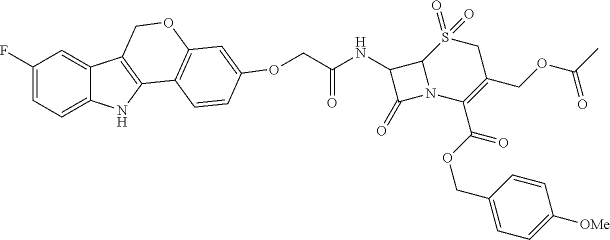 [SMILES: COc1ccc(COC(=O)C2=C(COC(C)=O)CS(=O)(=O)C3C(NC(=O)COc4ccc5c(c4)OCc4c-5[nH]c5ccc(F)cc45)C(=O)N23)cc1] |
| XIII'c | RA34 | 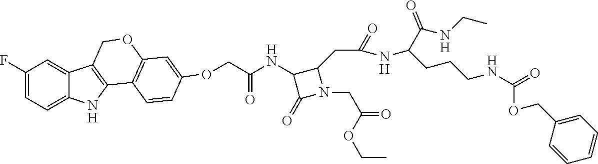 [SMILES: CCNC(=O)C(CCCNC(=O)OCc1ccccc1)NC(=O)CC1C(NC(=O)COc2ccc3c(c2)OCc2c-3[nH]c3ccc(F)cc23)C(=O)N1CC(=O)OCC] |
| XV'c | RA35 | 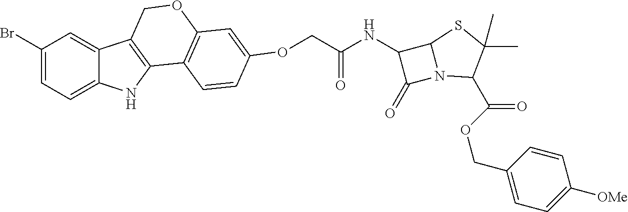 [SMILES: COc1ccc(COC(=O)C2N3C(=O)C(NC(=O)COc4ccc5c(c4)OCc4c-5[nH]c5ccc(Br)cc45)C3SC2(C)C)cc1] |
| XIV'e | RA36 | 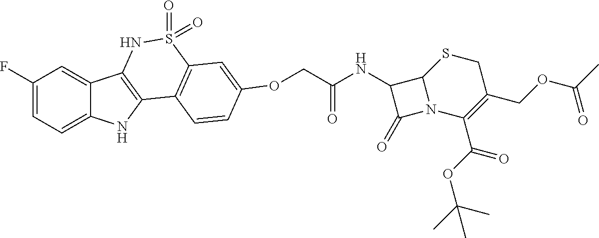 [SMILES: CC(=O)OCC1=C(C(=O)OC(C)(C)C)N2C(=O)C(NC(=O)COc3ccc4c(c3)S(=O)(=O)Nc3c-4[nH]c4ccc(F)cc34)C2SC1] |
| XIV'g' | RA37 | 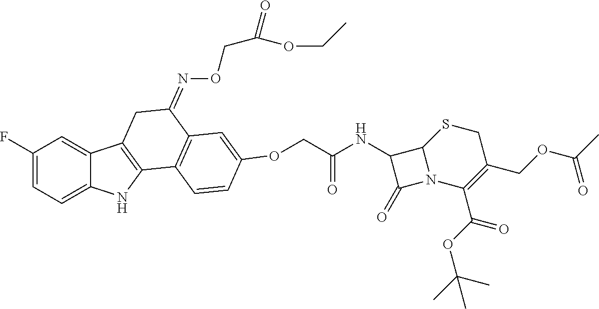 [SMILES: CCOC(=O)CO/N=C1/Cc2c([nH]c3ccc(F)cc23)-c2ccc(OCC(=O)NC3C(=O)N4C(C(=O)OC(C)(C)C)=C(COC(C)=O)CSC34)cc21] |
| XIV'g' | RA38 | 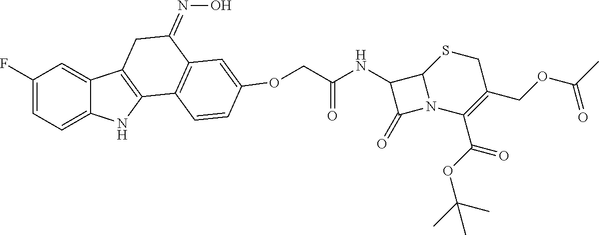 [SMILES: CC(=O)OCC1=C(C(=O)OC(C)(C)C)N2C(=O)C(NC(=O)COc3ccc4c(c3)/C(=N\O)Cc3c-4[nH]c4ccc(F)cc34)C2SC1] |
| XIV'g | RA39 | 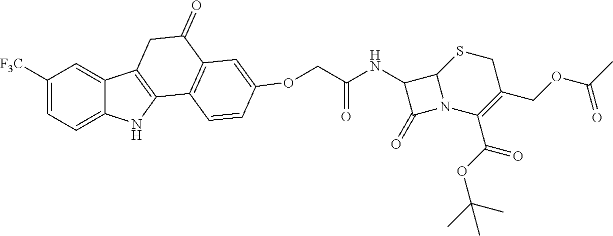 [SMILES: CC(=O)OCC1=C(C(=O)OC(C)(C)C)N2C(=O)C(NC(=O)COc3ccc4c(c3)C(=O)Cc3c-4[nH]c4ccc(C(F)(F)F)cc34)C2SC1] |
| XIVr | RA40 | 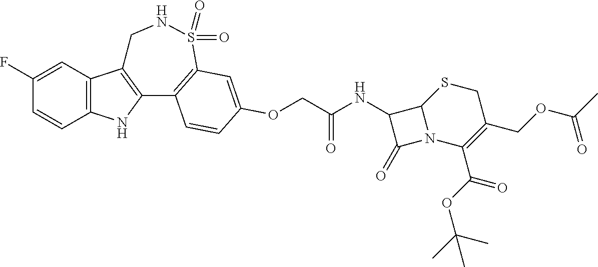 [SMILES: CC(=O)OCC1=C(C(=O)OC(C)(C)C)N2C(=O)C(NC(=O)COc3ccc4c(c3)S(=O)(=O)NCc3c-4[nH]c4ccc(F)cc34)C2SC1] |
| XIVc | RA41 | 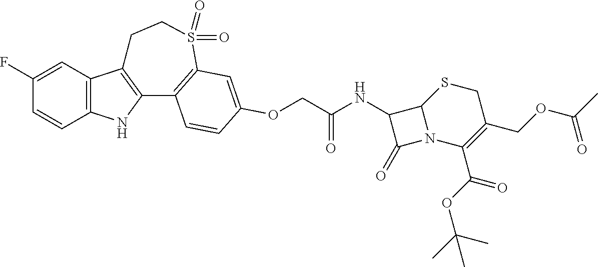 [SMILES: CC(=O)OCC1=C(C(=O)OC(C)(C)C)N2C(=O)C(NC(=O)COc3ccc4c(c3)S(=O)(=O)CCc3c-4[nH]c4ccc(F)cc34)C2SC1] |
| XVr | RA42 | 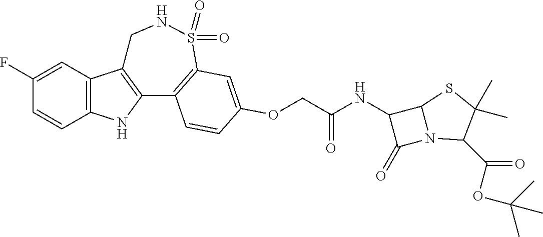 [SMILES: CC(C)(C)OC(=O)C1N2C(=O)C(NC(=O)COc3ccc4c(c3)S(=O)(=O)NCc3c-4[nH]c4ccc(F)cc34)C2SC1(C)C] |
| XIVc | RA43 | 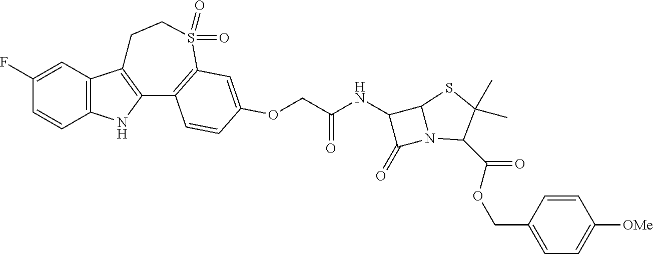 [SMILES: COc1ccc(COC(=O)C2N3C(=O)C(NC(=O)COc4ccc5c(c4)S(=O)(=O)CCc4c-5[nH]c5ccc(F)cc45)C3SC2(C)C)cc1] |
| XIIIr | RA44 | 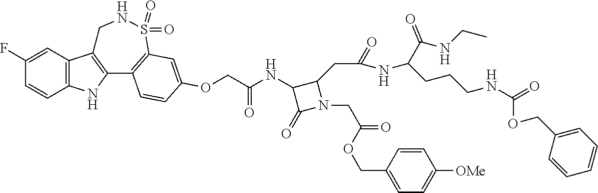 [SMILES: CCNC(=O)C(CCCNC(=O)OCc1ccccc1)NC(=O)CC1C(NC(=O)COc2ccc3c(c2)S(=O)(=O)NCc2c-3[nH]c3ccc(F)cc23)C(=O)N1CC(=O)OCc1ccc(OC)cc1] |

11. A method for treatment or diagnosis of degenerative pathologies characterized by high cell proliferation and/or tissue degeneration, comprising applying an effective amount of the compound of claim 1.

12. Compounds according to claim 2, wherein the compounds are selected from the group consisting of:

| # | # | |
|---|---|---|
| substructure | Abbreviation | Structure |
| XIc XII | RA1 | 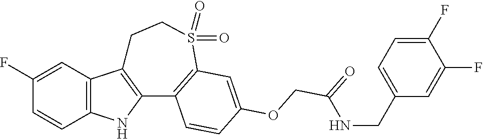 [SMILES: O=C(COc1ccc2c(c1)S(=O)(=O)CCc1c-2[nH]c2ccc(F)cc12)NCc1ccc(F)c(F)c1] |
| XIa | RA2 | 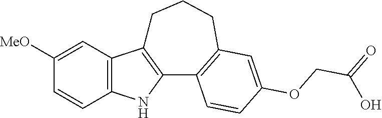 [SMILES: COc1ccc2[nH]c3c(c2c1)CCCc1cc(OCC(=O)O)ccc1-3] |
| XIr | RA3 | 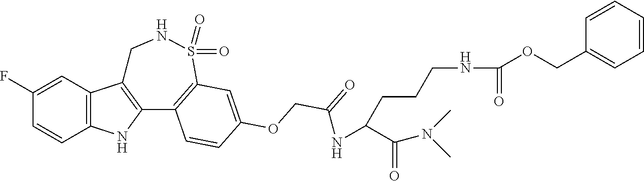 [SMILES: CN(C)C(=O)C(CCCNC(=O)OCc1ccccc1)NC(=O)COc1ccc2c(c1)S(=O)(=O)NCc1c-2[nH]c2ccc(F)cc12] |
| Xa | GA17M8 | 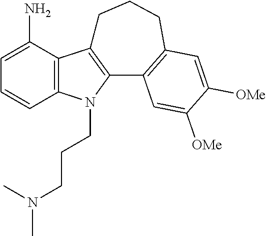 [SMILES: COc1cc2c(cc1OC)-c1c(c3c(N)cccc3n1CCCN(C)C)CCC2] |
| Xa | GA17M10 | 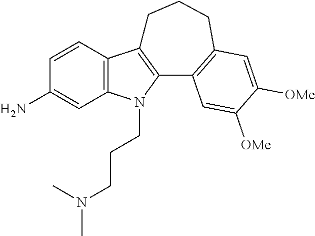 [SMILES: COc1cc2c(cc1OC)-c1c(c3ccc(N)cc3n1CCCN(C)C)CCC2] |
| XIb | GA11S | 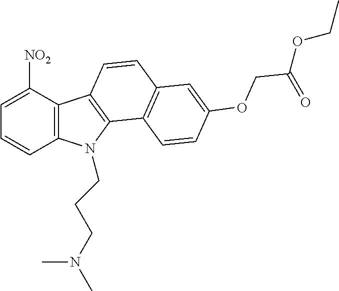 [SMILES: CCOC(=O)COc1ccc2c(ccc3c4c([N+](=O)[O-])cccc4n(CCCN(C)C)c23)c1] |
| XIb | RM66 | 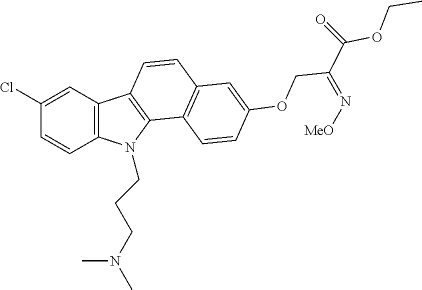 [SMILES: CCOC(=O)/C(COc1ccc2c(ccc3c4cc(Cl)ccc4n(CCCN(C)C)c23)c1)=N/OC] |
| XIb | RM58 | 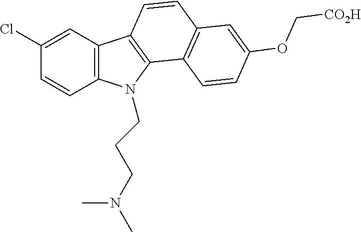 [SMILES: CN(C)CCCn1c2ccc(Cl)cc2c2ccc3cc(OCC(=O)O)ccc3c21] |
| XIb | RM70 | 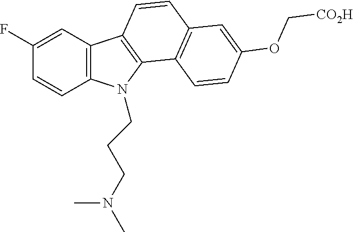 [SMILES: CN(C)CCCn1c2ccc(F)cc2c2ccc3cc(OCC(=O)O)ccc3c21] |
| XIb | RM85 | 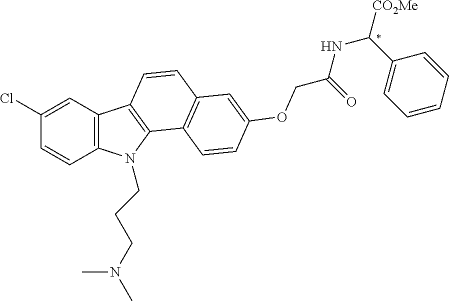 [SMILES: COC(=O)C(NC(=O)COc1ccc2c(ccc3c4cc(Cl)ccc4n(CCCN(C)C)c23)c1)c1ccccc1] |
| XI'd | RA6 | 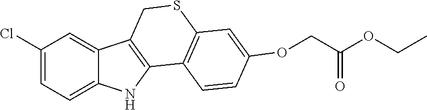 [SMILES: CCOC(=O)COc1ccc2c(c1)SCc1c-2[nH]c2ccc(Cl)cc12] |
| XI'd | RA7 | 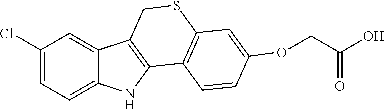 [SMILES: O=C(O)COc1ccc2c(c1)SCc1c-2[nH]c2ccc(Cl)cc12] |
| XI'd | RA9 | 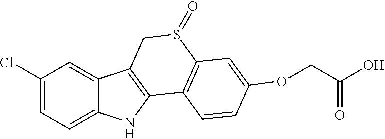 [SMILES: O=C(O)COc1ccc2c(c1)S(=O)Cc1c-2[nH]c2ccc(Cl)cc12] |
| XI'd | RA11 | 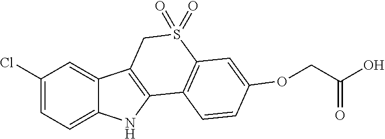 [SMILES: O=C(O)COc1ccc2c(c1)S(=O)(=O)Cc1c-2[nH]c2ccc(Cl)cc12] |
| XI'd | RA12 | 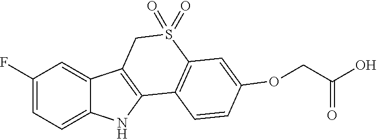 [SMILES: O=C(O)COc1ccc2c(c1)S(=O)(=O)Cc1c-2[nH]c2ccc(F)cc12] |
| XI'd | RA13 | 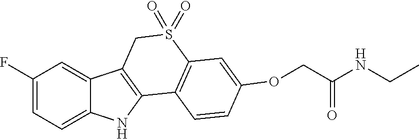 [SMILES: CCNC(=O)COc1ccc2c(c1)S(=O)(=O)Cc1c-2[nH]c2ccc(F)cc12] |
| XI'g | RA14 | 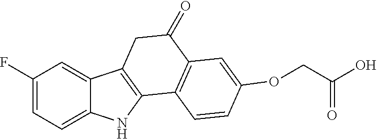 [SMILES: O=C(O)COc1ccc2c(c1)C(=O)Cc1c-2[nH]c2ccc(F)cc12] |
| X'c | RA15 | 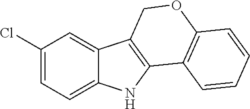 [SMILES: Clc1ccc2[nH]c3c(c2c1)COc1ccccc1-3] |
| X'c | RA16 | 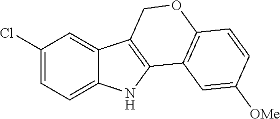 [SMILES: COc1ccc2c(c1)-c1[nH]c3ccc(Cl)cc3c1CO2] |
| XI'c | RA17 | 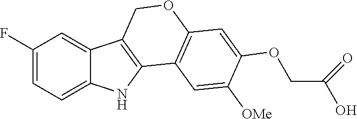 [SMILES: COc1cc2c(cc1OCC(=O)O)OCc1c-2[nH]c2ccc(F)cc12] |
| XI'c | RA18 | 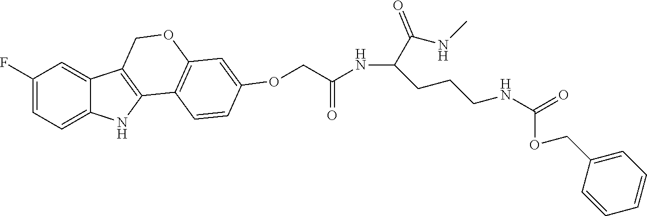 [SMILES: CNC(=O)C(CCCNC(=O)OCc1ccccc1)NC(=O)COc1ccc2c(c1)OCc1c-2[nH]c2ccc(F)cc12] |
| XI'c | RA19 | 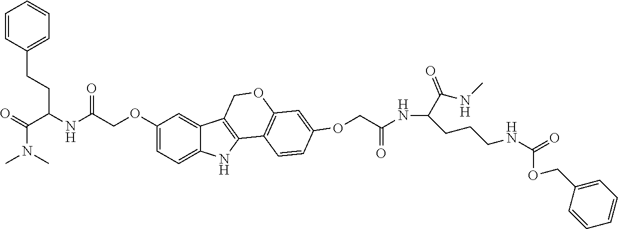 [SMILES: CNC(=O)C(CCCNC(=O)OCc1ccccc1)NC(=O)COc1ccc2c(c1)OCc1c-2[nH]c2ccc(OCC(=O)NC(CCc3ccccc3)C(=O)N(C)C)cc12] |
| XI'h | RA20 | 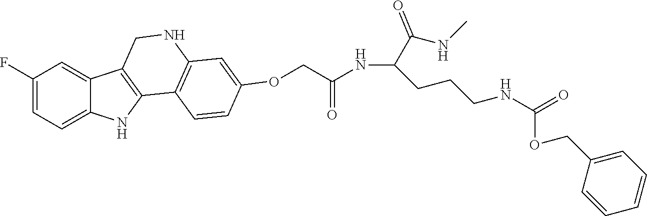 [SMILES: CNC(=O)C(CCCNC(=O)OCc1ccccc1)NC(=O)COc1ccc2c(c1)NCc1c-2[nH]c2ccc(F)cc12] |
| X'e | RA21 | 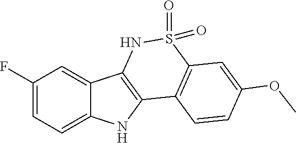 [SMILES: COc1ccc2c(c1)S(=O)(=O)Nc1c-2[nH]c2ccc(F)cc12] |
| XI'e | RA22 | 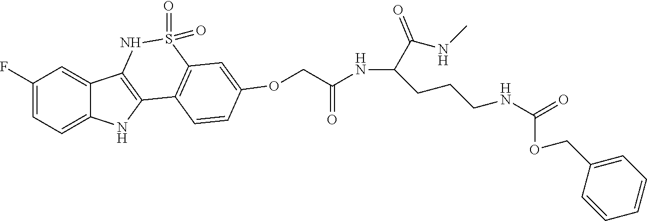 [SMILES: CNC(=O)C(CCCNC(=O)OCc1ccccc1)NC(=O)COc1ccc2c(c1)S(=O)(=O)Nc1c-2[nH]c2ccc(F)cc12] |
| XIV'a | GA09a | 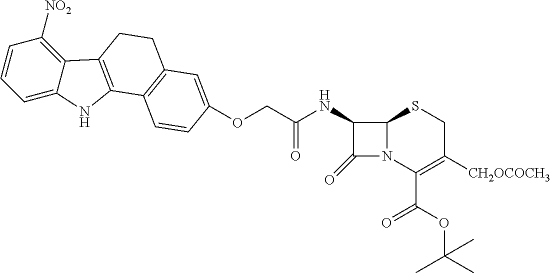 |
| XIV'a' | GA09a' | 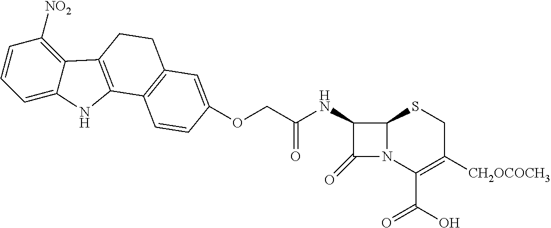 |
| XIV'a | GA09b | 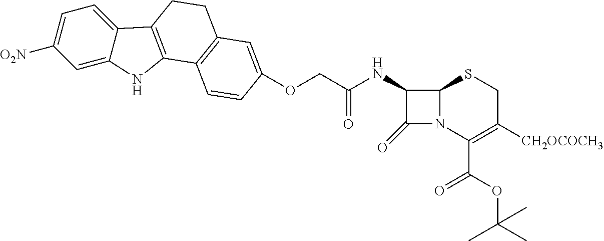 |
| XIV'a' | GA09b' | 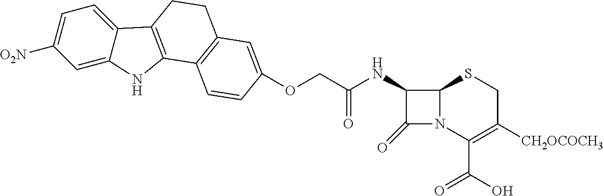 |
| XIV'a | RM36 | 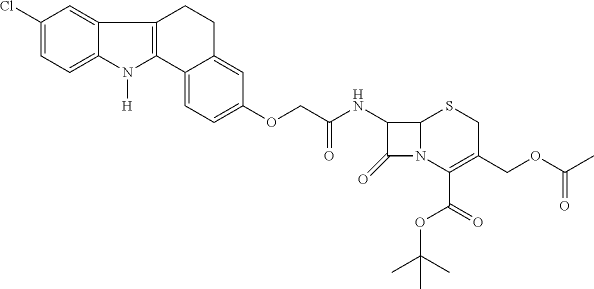 [SMILES: CC(=O)OCC1=C(C(=O)OC(C)(C)C)N2C(=O)C(NC(=O)COc3ccc4c(c3)CCc3c-4[nH]c4ccc(Cl)cc34)C2SC1] |
| XIV'a | RM37 | 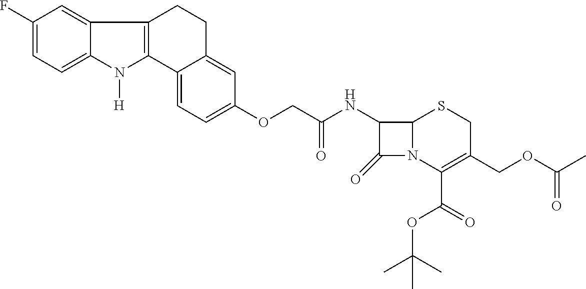 [SMILES: CC(=O)OCC1=C(C(=O)OC(C)(C)C)N2C(=O)C(NC(=O)COc3ccc4c(c3)CCc3c-4[nH]c4ccc(F)cc34)C2SC1] |
| XIV'a' | RM53 | 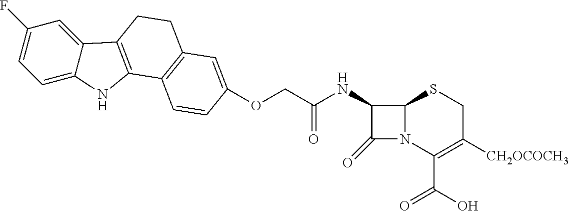 |
| XIV'a | RM47 | 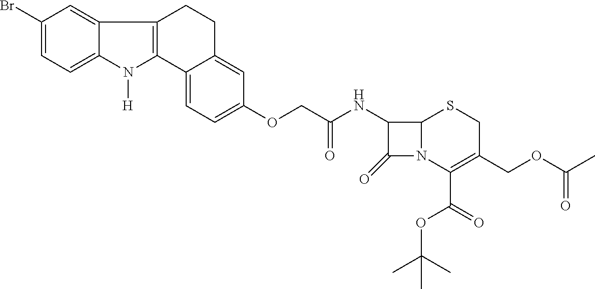 [SMILES: CC(=O)OCC1=C(C(=O)OC(C)(C)C)N2C(=O)C(NC(=O)COc3ccc4c(c3)CCc3c-4[nH]c4ccc(Br)cc34)C2SC1] |
| XIV'd | RA23 | 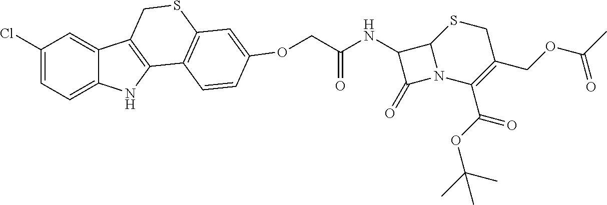 [SMILES: CC(=O)OCC1=C(C(=O)OC(C)(C)C)N2C(=O)C(NC(=O)COc3ccc4c(c3)SCc3c-4[nH]c4ccc(Cl)cc34)C2SC1] |
| XIV'd | RA24 | 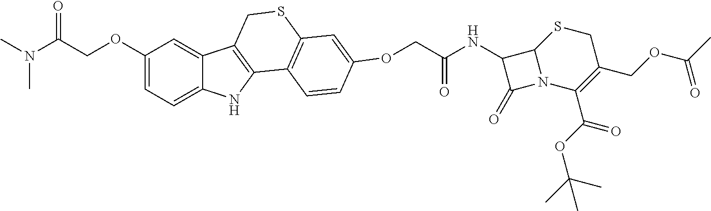 [SMILES: CC(=O)OCC1=C(C(=O)OC(C)(C)C)N2C(=O)C(NC(=O)COc3ccc4c(c3)SCc3c-4[nH]c4ccc(OCC(=O)N(C)C)cc34)C2SC1] |
| XIV'd | RA25 | 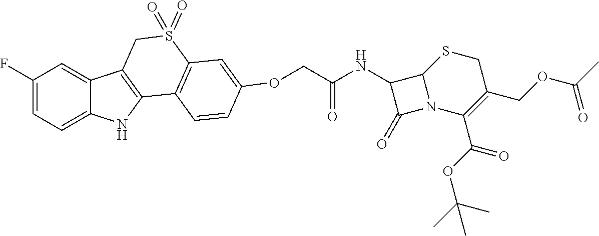 [SMILES: CC(=O)OCC1=C(C(=O)OC(C)(C)C)N2C(=O)C(NC(=O)COc3ccc4c(c3)S(=O)(=O)Cc3c-4[nH]c4ccc(F)cc34)C2SC1] |
| XV'e | RA26 | 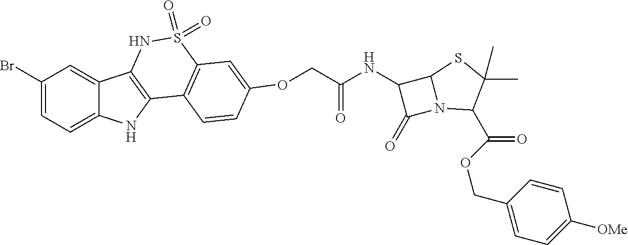 [SMILES: COc1ccc(COC(=O)C2N3C(=O)C(NC(=O)COc4ccc5c(c4)S(=O)(=O)Nc4c-5[nH]c5ccc(Br)cc45)C3SC2(C)C)cc1] |
| XV'd | RA27 | 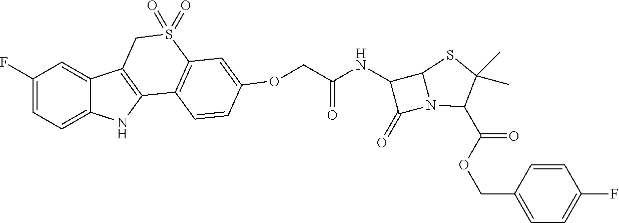 [SMILES: CC1(C)SC2C(NC(=O)COc3ccc4c(c3)S(=O)(=O)Cc3c-4[nH]c4ccc(F)cc34)C(=O)N2C1C(=O)OCc1ccc(F)cc1] |
| XV'd | RA28 | 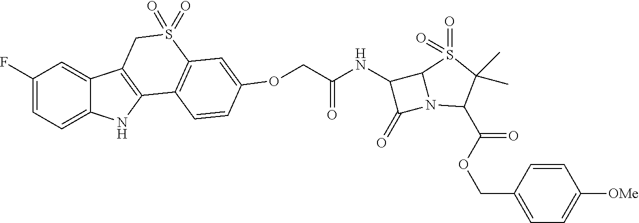 [SMILES: COc1ccc(COC(=O)C2N3C(=O)C(NC(=O)COc4ccc5c(c4)S(=O)(=O)Cc4c-5[nH]c5ccc(F)cc45)C3S(=O)(=O)C2(C)C)cc1] |
| XV'g' | RA29 | 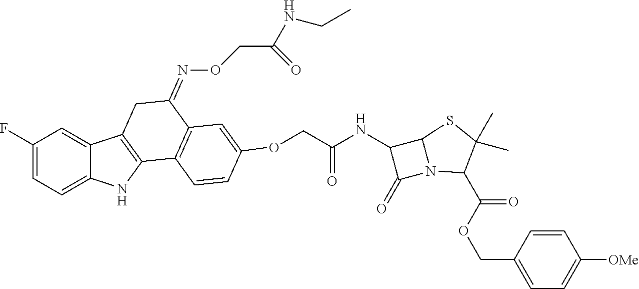 [SMILES: CCNC(=O)CO/N=C1/Cc2c([nH]c3ccc(F)cc23)-c2ccc(OCC(=O)NC3C(=O)N4C3SC(C)(C)C4C(=O)OCc3ccc(OC)cc3)cc21] |
| XIIId | RA30 | 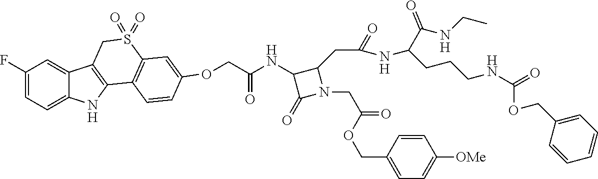 [SMILES: CCNC(=O)C(CCCNC(=O)OCc1ccccc1)NC(=O)CC1C(NC(=O)COc2ccc3c(c2)S(=O)(=O)Cc2c-3[nH]c3ccc(F)cc23)C(=O)N1CC(=O)OCc1ccc(OC)cc1] |
| XIV'c | RA31 | 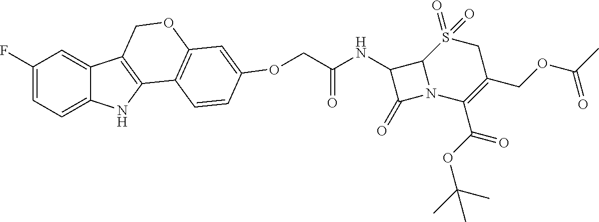 [SMILES: CC(=O)OCC1=C(C(=O)OC(C)(C)C)N2C(=O)C(NC(=O)COc3ccc4c(c3)OCc3c-4[nH]c4ccc(F)cc34)C2S(=O)(=O)C1] |
| XIV'c | RA32 | 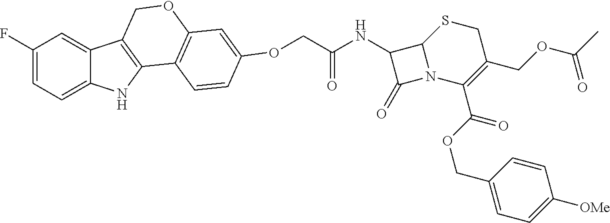 [SMILES: COc1ccc(COC(=O)C2=C(COC(C)=O)CSC3C(NC(=O)COc4ccc5c(c4)OCc4c-5[nH]c5ccc(F)cc45)C(=O)N23)cc1] |
| XIV'c | RA33 | 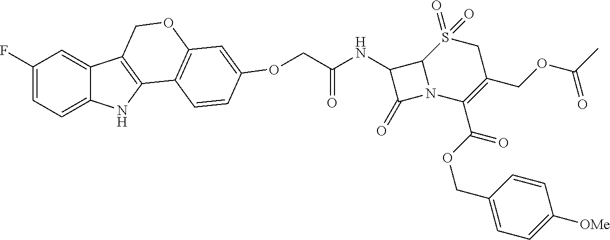 [SMILES: COc1ccc(COC(=O)C2=C(COC(C)=O)CS(=O)(=O)C3C(NC(=O)COc4ccc5c(c4)OCc4c-5[nH]c5ccc(F)cc45)C(=O)N23)cc1] |
| XIII'c | RA34 | 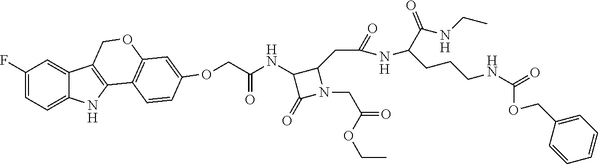 [SMILES: CCNC(=O)C(CCCNC(=O)OCc1ccccc1)NC(=O)CC1C(NC(=O)COc2ccc3c(c2)OCc2c-3[nH]c3ccc(F)cc23)C(=O)N1CC(=O)OCC] |
| XV'c | RA35 | 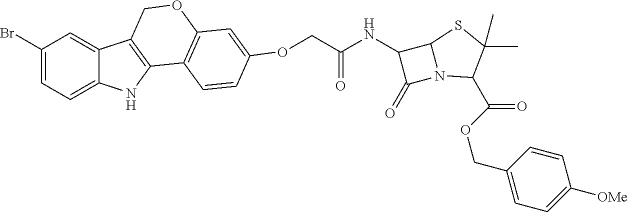 [SMILES: COc1ccc(COC(=O)C2N3C(=O)C(NC(=O)COc4ccc5c(c4)OCc4c-5[nH]c5ccc(Br)cc45)C3SC2(C)C)cc1] |
| XIV'e | RA36 | 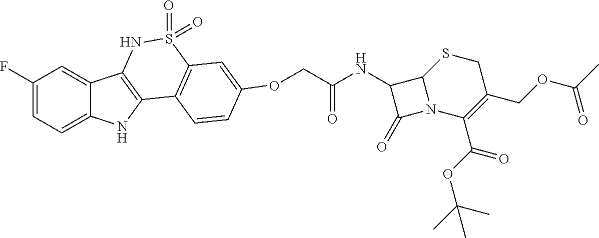 [SMILES: CC(=O)OCC1=C(C(=O)OC(C)(C)C)N2C(=O)C(NC(=O)COc3ccc4c(c3)S(=O)(=O)Nc3c-4[nH]c4ccc(F)cc34)C2SC1] |
| XIV'g' | RA37 | 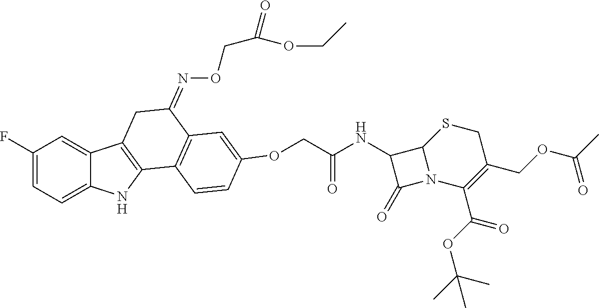 [SMILES: CCOC(=O)CO/N=C1/Cc2c([nH]c3ccc(F)cc23)-c2ccc(OCC(=O)NC3C(=O)N4C(C(=O)OC(C)(C)C)=C(COC(C)=O)CSC34)cc21] |
| XIV'g' | RA38 | 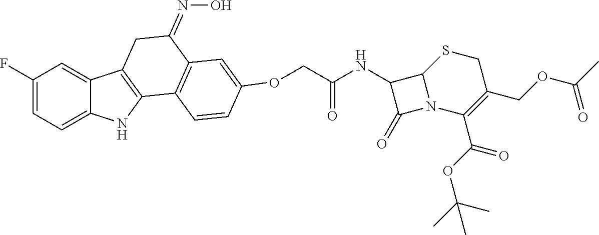 [SMILES: CC(=O)OCC1=C(C(=O)OC(C)(C)C)N2C(=O)C(NC(=O)COc3ccc4c(c3)/C(=N\O)Cc3c-4[nH]c4ccc(F)cc34)C2SC1] |
| XIV'g | RA39 | 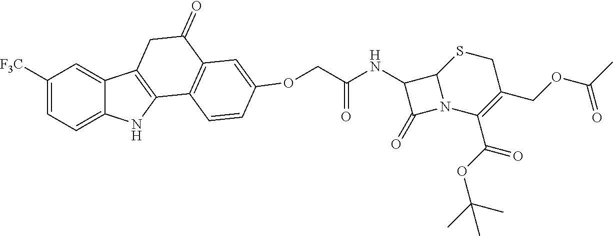 [SMILES: CC(=O)OCC1=C(C(=O)OC(C)(C)C)N2C(=O)C(NC(=O)COc3ccc4c(c3)C(=O)Cc3c-4[nH]c4ccc(C(F)(F)F)cc34)C2SC1] |
| XIVr | RA40 | 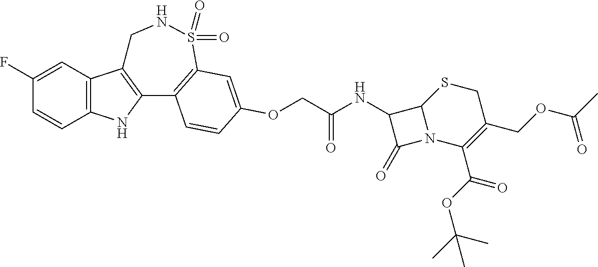 [SMILES: CC(=O)OCC1=C(C(=O)OC(C)(C)C)N2C(=O)C(NC(=O)COc3ccc4c(c3)S(=O)(=O)NCc3c-4[nH]c4ccc(F)cc34)C2SC1] |
| XIVc | RA41 | 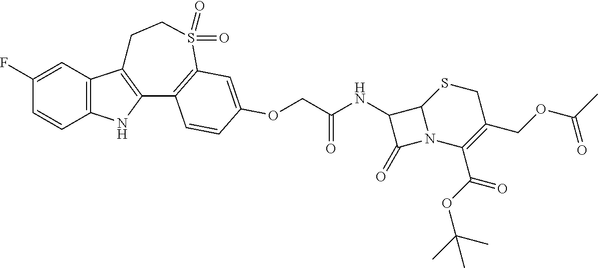 [SMILES: CC(=O)OCC1=C(C(=O)OC(C)(C)C)N2C(=O)C(NC(=O)COc3ccc4c(c3)S(=O)(=O)CCc3c-4[nH]c4ccc(F)cc34)C2SC1] |
| XVr | RA42 | 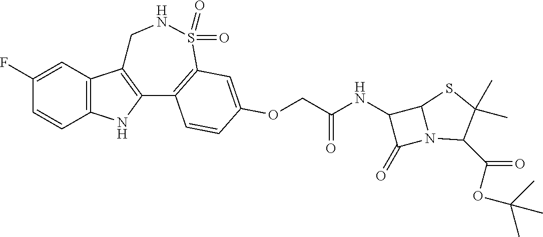 [SMILES: CC(C)(C)OC(=O)C1N2C(=O)C(NC(=O)COc3ccc4c(c3)S(=O)(=O)NCc3c-4[nH]c4ccc(F)cc34)C2SC1(C)C] |
| XIVc | RA43 | 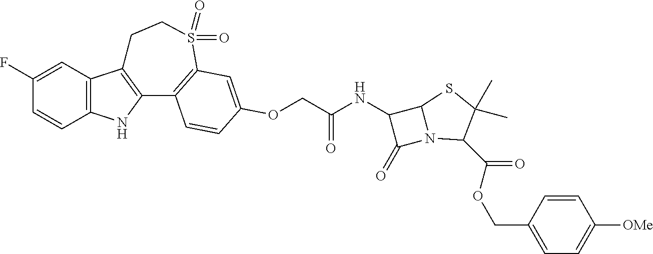 [SMILES: COc1ccc(COC(=O)C2N3C(=O)C(NC(=O)COc4ccc5c(c4)S(=O)(=O)CCc4c-5[nH]c5ccc(F)cc45)C3SC2(C)C)cc1] |
| XIIIr | RA44 | 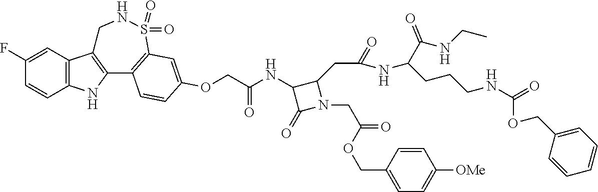 [SMILES: CCNC(=O)C(CCCNC(=O)OCc1ccccc1)NC(=O)CC1C(NC(=O)COc2ccc3c(c2)S(=O)(=O)NCc2c-3[nH]c3ccc(F)cc23)C(=O)N1CC(=O)OCc1ccc(OC)cc1] |

13. Compounds according to claim 3, wherein the compounds are selected from the group consisting of:

| # | # | |
|---|---|---|
| substructure | Abbreviation | Structure |
| XIc XII | RA1 | 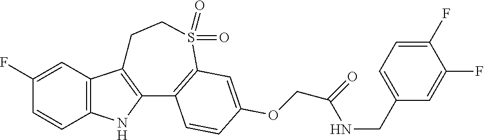 [SMILES: O=C(COc1ccc2c(c1)S(=O)(=O)CCc1c-2[nH]c2ccc(F)cc12)NCc1ccc(F)c(F)c1] |
| XIa | RA2 | 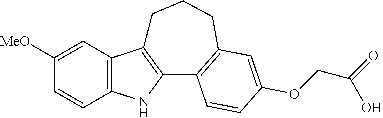 [SMILES: COc1ccc2[nH]c3c(c2c1)CCCc1cc(OCC(=O)O)ccc1-3] |
| XIr | RA3 | 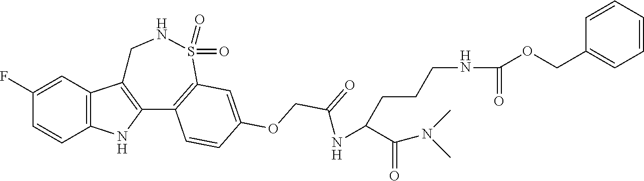 [SMILES: CN(C)C(=O)C(CCCNC(=O)OCc1ccccc1)NC(=O)COc1ccc2c(c1)S(=O)(=O)NCc1c-2[nH]c2ccc(F)cc12] |
| Xa | GA17M8 | 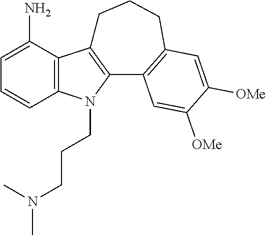 [SMILES: COc1cc2c(cc1OC)-c1c(c3c(N)cccc3n1CCCN(C)C)CCC2] |
| Xa | GA17M10 | 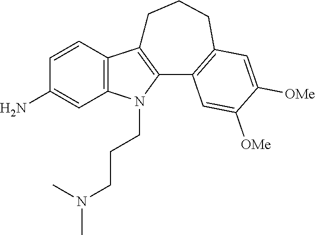 [SMILES: COc1cc2c(cc1OC)-c1c(c3ccc(N)cc3n1CCCN(C)C)CCC2] |
| XIb | GA11S | 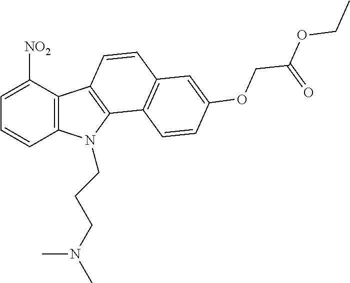 [SMILES: CCOC(=O)COc1ccc2c(ccc3c4c([N+](=O)[O-])cccc4n(CCCN(C)C)c23)c1] |
| XIb | RM66 | 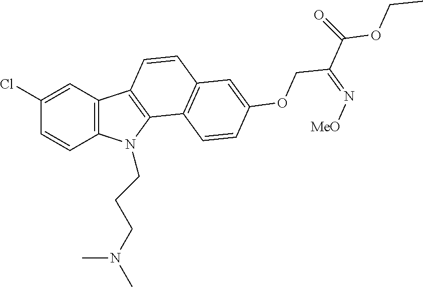 [SMILES: CCOC(=O)/C(COc1ccc2c(ccc3c4cc(Cl)ccc4n(CCCN(C)C)c23)c1)=N/OC] |
| XIb | RM58 | 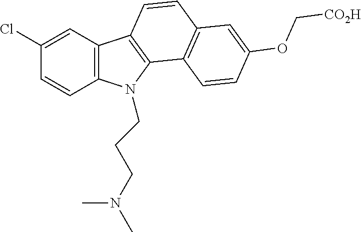 [SMILES: CN(C)CCCn1c2ccc(Cl)cc2c2ccc3cc(OCC(=O)O)ccc3c21] |
| XIb | RM70 | 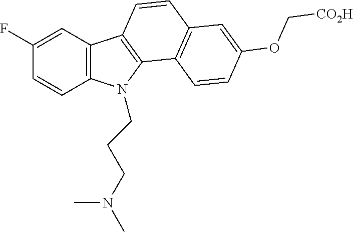 [SMILES: CN(C)CCCn1c2ccc(F)cc2c2ccc3cc(OCC(=O)O)ccc3c21] |
| XIb | RM85 | 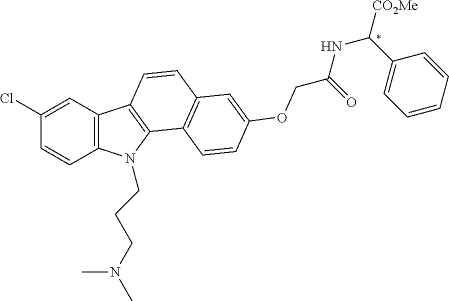 [SMILES: COC(=O)C(NC(=O)COc1ccc2c(ccc3c4cc(Cl)ccc4n(CCCN(C)C)c23)c1)c1ccccc1] |
| XI'd | RA6 | 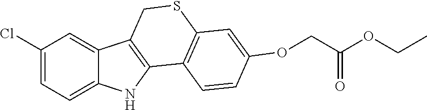 [SMILES: CCOC(=O)COc1ccc2c(c1)SCc1c-2[nH]c2ccc(Cl)cc12] |
| XI'd | RA7 | 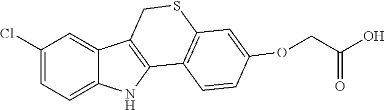 [SMILES: O=C(O)COc1ccc2c(c1)SCc1c-2[nH]c2ccc(Cl)cc12] |
| XI'd | RA9 | 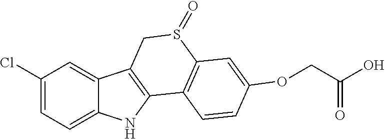 [SMILES: O=C(O)COc1ccc2c(c1)S(=O)Cc1c-2[nH]c2ccc(Cl)cc12] |
| XI'd | RA11 | 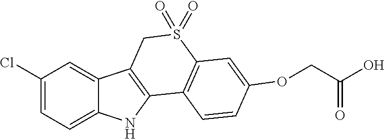 [SMILES: O=C(O)COc1ccc2c(c1)S(=O)(=O)Cc1c-2[nH]c2ccc(Cl)cc12] |
| XI'd | RA12 | 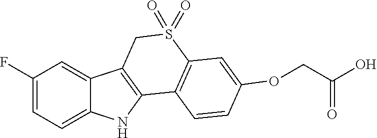 [SMILES: O=C(O)COc1ccc2c(c1)S(=O)(=O)Cc1c-2[nH]c2ccc(F)cc12] |
| XI'd | RA13 | 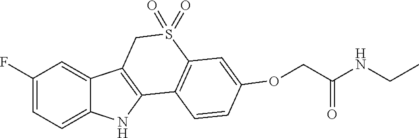 [SMILES: CCNC(=O)COc1ccc2c(c1)S(=O)(=O)Cc1c-2[nH]c2ccc(F)cc12] |
| XI'g | RA14 | 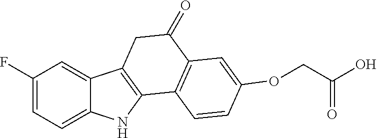 [SMILES: O=C(O)COc1ccc2c(c1)C(=O)Cc1c-2[nH]c2ccc(F)cc12] |
| X'c | RA15 | 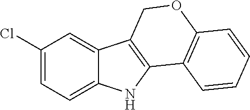 [SMILES: Clc1ccc2[nH]c3c(c2c1)COc1ccccc1-3] |
| X'c | RA16 | 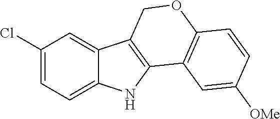 [SMILES: COc1ccc2c(c1)-c1[nH]c3ccc(Cl)cc3c1CO2] |
| XI'c | RA17 | 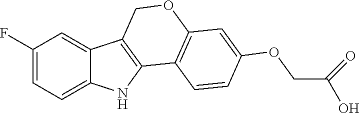 [SMILES: O=C(O)COc1ccc2c(c1)OCc1c-2[nH]c2ccc(F)cc12] |
| XI'c | RA18 | 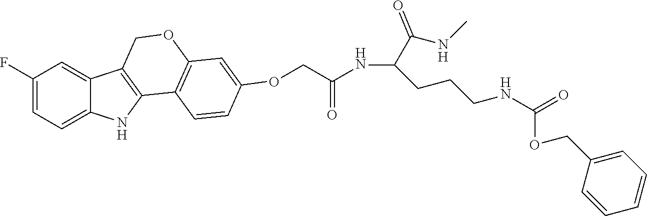 [SMILES: CNC(=O)C(CCCNC(=O)OCc1ccccc1)NC(=O)COc1ccc2c(c1)OCc1c-2[nH]c2ccc(F)cc12] |
| XI'c | RA19 | 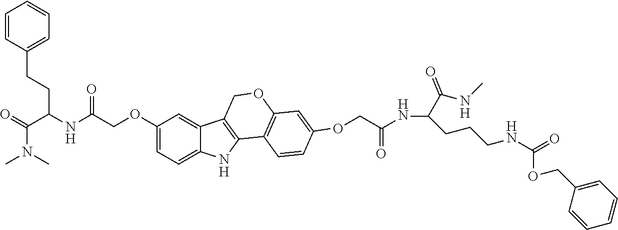 [SMILES: CNC(=O)C(CCCNC(=O)OCc1ccccc1)NC(=O)COc1ccc2c(c1)OCc1c-2[nH]c2ccc(OCC(=O)NC(CCc3ccccc3)C(=O)N(C)C)cc12] |
| XI'h | RA20 | 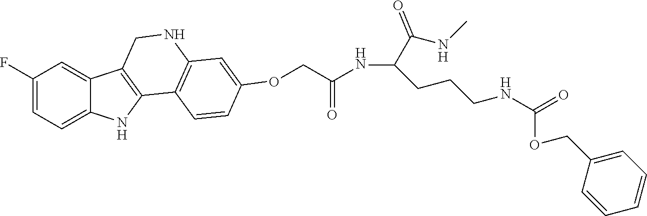 [SMILES: CNC(=O)C(CCCNC(=O)OCc1ccccc1)NC(=O)COc1ccc2c(c1)NCc1c-2[nH]c2ccc(F)cc12] |
| X'e | RA21 | 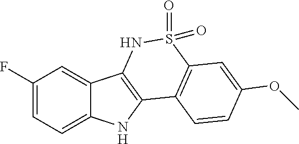 [SMILES: COc1ccc2c(c1)S(=O)(=O)Nc1c-2[nH]c2ccc(F)cc12] |
| XI'e | RA22 | 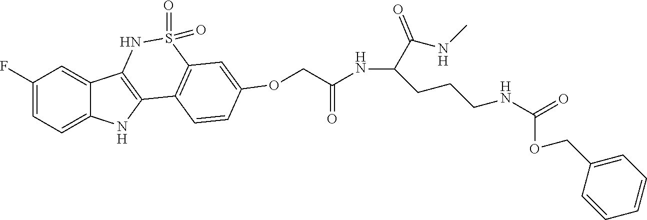 [SMILES: CNC(=O)C(CCCNC(=O)OCc1ccccc1)NC(=O)COc1ccc2c(c1)S(=O)(=O)Nc1c-2[nH]c2ccc(F)cc12] |
| XIV'a | GA09a | 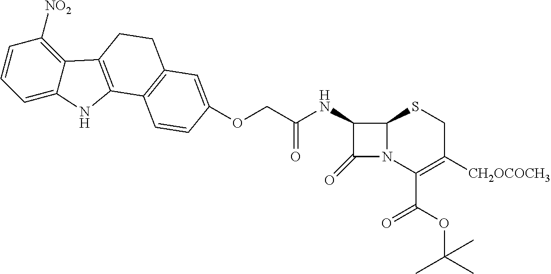 |
| XIV'a' | GA09a' | 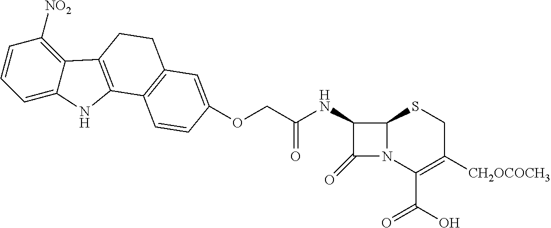 |
| XIV'a | GA09b | 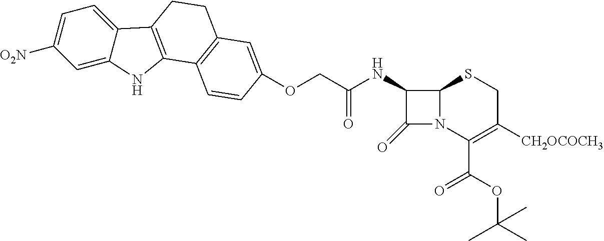 |
| XIV'a' | GA09b' | 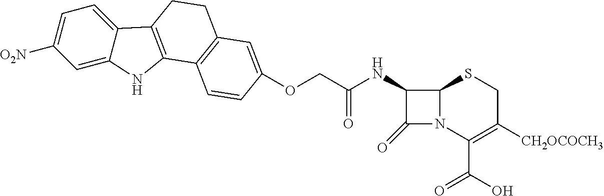 |
| XIV'a | RM36 | 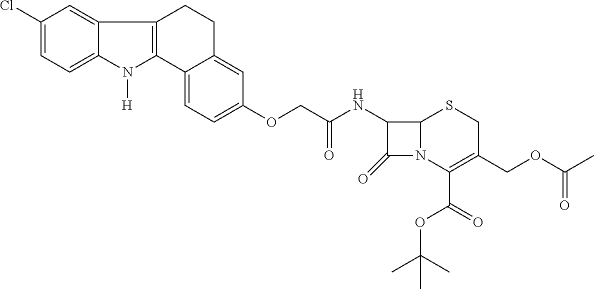 [SMILES: CC(=O)OCC1=C(C(=O)OC(C)(C)C)N2C(=O)C(NC(=O)COc3ccc4c(c3)CCc3c-4[nH]c4ccc(Cl)cc34)C2SC1] |
| XIV'a | RM37 | 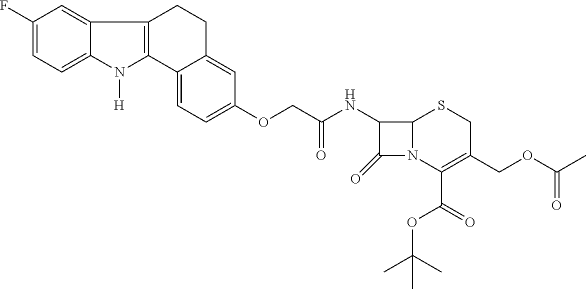 [SMILES: CC(=O)OCC1=C(C(=O)OC(C)(C)C)N2C(=O)C(NC(=O)COc3ccc4c(c3)CCc3c-4[nH]c4ccc(F)cc34)C2SC1] |
| XIV'a' | RM53 | 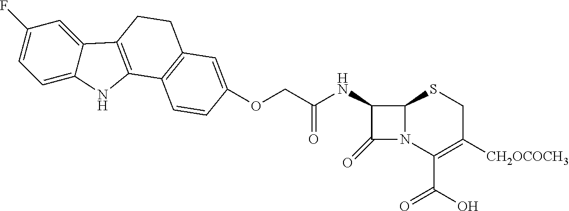 |
| XIV'a | RM47 | 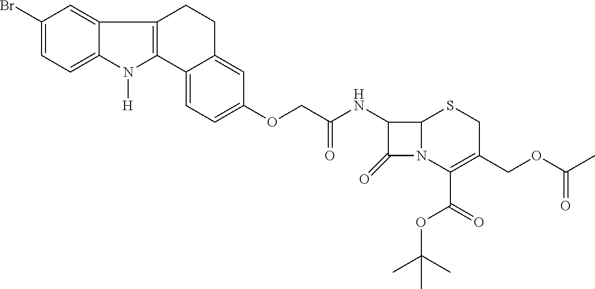 [SMILES: CC(=O)OCC1=C(C(=O)OC(C)(C)C)N2C(=O)C(NC(=O)COc3ccc4c(c3)CCc3c-4[nH]c4ccc(Br)cc34)C2SC1] |
| XIV'd | RA23 | 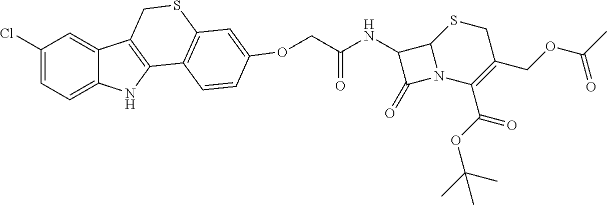 [SMILES: CC(=O)OCC1=C(C(=O)OC(C)(C)C)N2C(=O)C(NC(=O)COc3ccc4c(c3)SCc3c-4[nH]c4ccc(Cl)cc34)C2SC1] |
| XIV'd | RA24 | 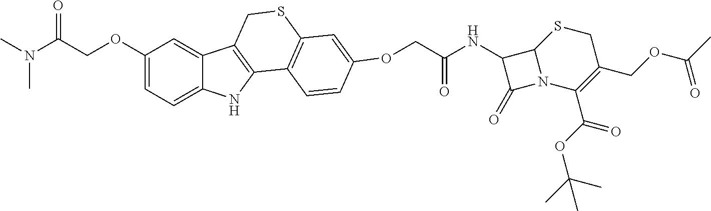 [SMILES: CC(=O)OCC1=C(C(=O)OC(C)(C)C)N2C(=O)C(NC(=O)COc3ccc4c(c3)SCc3c-4[nH]c4ccc(OCC(=O)N(C)C)cc34)C2SC1] |
| XIV'd | RA25 | 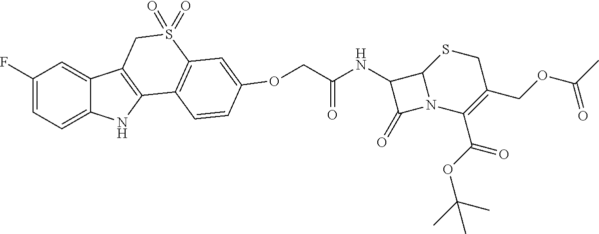 [SMILES: CC(=O)OCC1=C(C(=O)OC(C)(C)C)N2C(=O)C(NC(=O)COc3ccc4c(c3)S(=O)(=O)Cc3c-4[nH]c4ccc(F)cc34)C2SC1] |
| XV'e | RA26 | 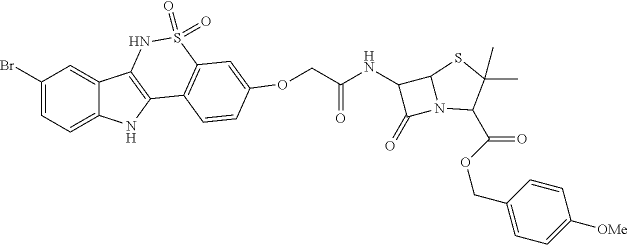 [SMILES: COc1ccc(COC(=O)C2N3C(=O)C(NC(=O)COc4ccc5c(c4)S(=O)(=O)Nc4c-5[nH]c5ccc(Br)cc45)C3SC2(C)C)cc1] |
| XV'd | RA27 | 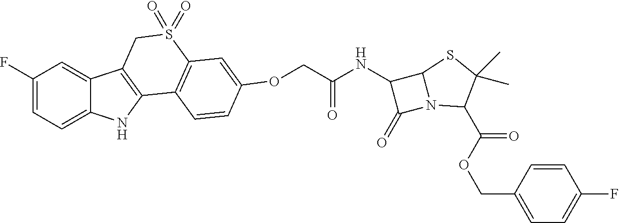 [SMILES: CC1(C)SC2C(NC(=O)COc3ccc4c(c3)S(=O)(=O)Cc3c-4[nH]c4ccc(F)cc34)C(=O)N2C1C(=O)OCc1ccc(F)cc1] |
| XV'd | RA28 | 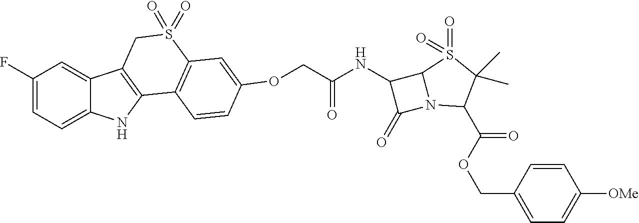 [SMILES: COc1ccc(COC(=O)C2N3C(=O)C(NC(=O)COc4ccc5c(c4)S(=O)(=O)Cc4c-5[nH]c5ccc(F)cc45)C3S(=O)(=O)C2(C)C)cc1] |
| XV'g' | RA29 | 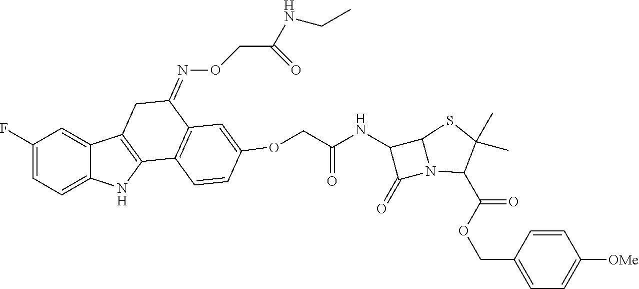 [SMILES: CCNC(=O)CO/N=C1/Cc2c([nH]c3ccc(F)cc23)-c2ccc(OCC(=O)NC3C(=O)N4C3SC(C)(C)C4C(=O)OCc3ccc(OC)cc3)cc21] |
| XIIId | RA30 | 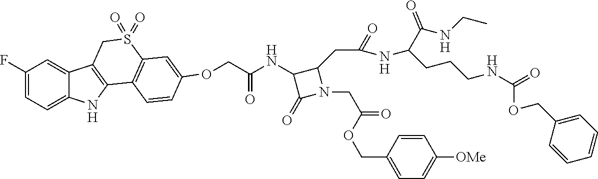 [SMILES: CCNC(=O)C(CCCNC(=O)OCc1ccccc1)NC(=O)CC1C(NC(=O)COc2ccc3c(c2)S(=O)(=O)Cc2c-3[nH]c3ccc(F)cc23)C(=O)N1CC(=O)OCc1ccc(OC)cc1] |
| XIV'c | RA31 | 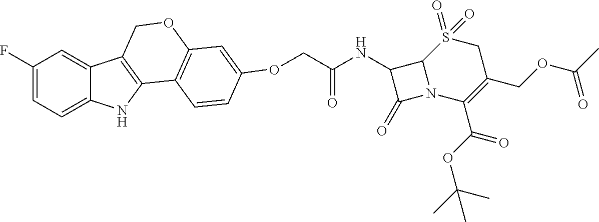 [SMILES: CC(=O)OCC1=C(C(=O)OC(C)(C)C)N2C(=O)C(NC(=O)COc3ccc4c(c3)OCc3c-4[nH]c4ccc(F)cc34)C2S(=O)(=O)C1] |
| XIV'c | RA32 | 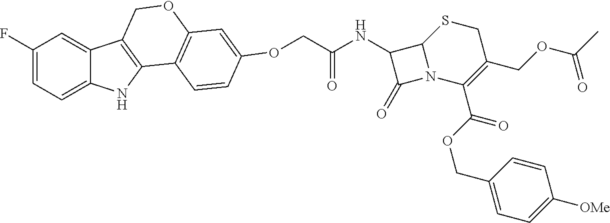 [SMILES: COc1ccc(COC(=O)C2=C(COC(C)=O)CSC3C(NC(=O)COc4ccc5c(c4)OCc4c-5[nH]c5ccc(F)cc45)C(=O)N23)cc1] |
| XIV'c | RA33 | 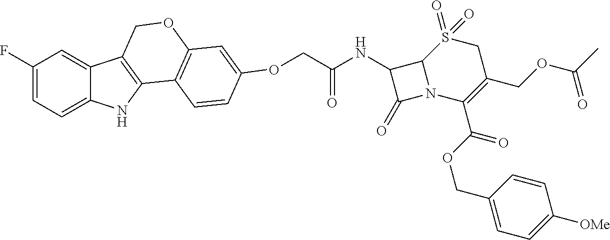 [SMILES: COc1ccc(COC(=O)C2=C(COC(C)=O)CS(=O)(=O)C3C(NC(=O)COc4ccc5c(c4)OCc4c-5[nH]c5ccc(F)cc45)C(=O)N23)cc1] |
| XIII'c | RA34 | 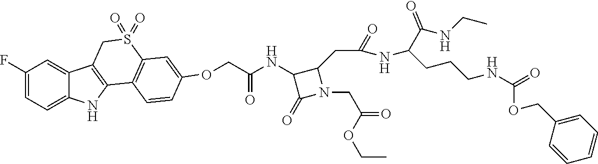 [SMILES: CCNC(=O)C(CCCNC(=O)OCc1ccccc1)NC(=O)CC1C(NC(=O)COc2ccc3c(c2)S(=O)(=O)Cc2c-3[nH]c3ccc(F)cc23)C(=O)N1CC(=O)OCC] |
| XV'c | RA35 | 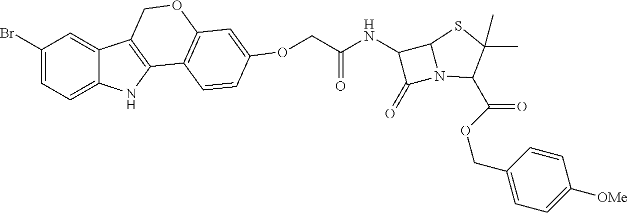 [SMILES: COc1ccc(COC(=O)C2N3C(=O)C(NC(=O)COc4ccc5c(c4)OCc4c-5[nH]c5ccc(Br)cc45)C3SC2(C)C)cc1] |
| XIV'e | RA36 | 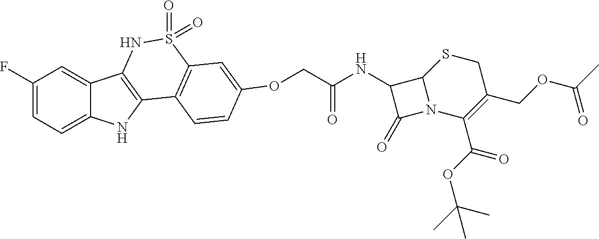 [SMILES: CC(=O)OCC1=C(C(=O)OC(C)(C)C)N2C(=O)C(NC(=O)COc3ccc4c(c3)S(=O)(=O)Nc3c-4[nH]c4ccc(F)cc34)C2SC1] |
| XIV'g' | RA37 | 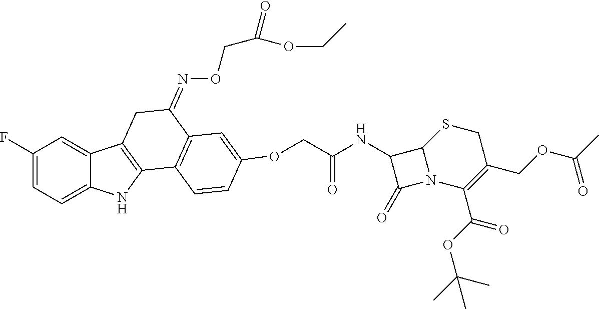 [SMILES: CCOC(=O)CO/N=C1/Cc2c([nH]c3ccc(F)cc23)-c2ccc(OCC(=O)NC3C(=O)N4C(C(=O)OC(C)(C)C)=C(COC(C)=O)CSC34)cc21] |
| XIV'g' | RA38 | 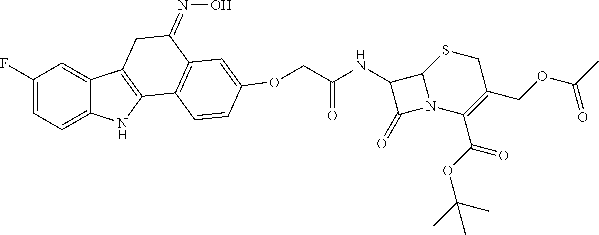 [SMILES: CC(=O)OCC1=C(C(=O)OC(C)(C)C)N2C(=O)C(NC(=O)COc3ccc4c(c3)/C(=N\O)Cc3c-4[nH]c4ccc(F)cc34)C2SC1] |
| XIV'g | RA39 | 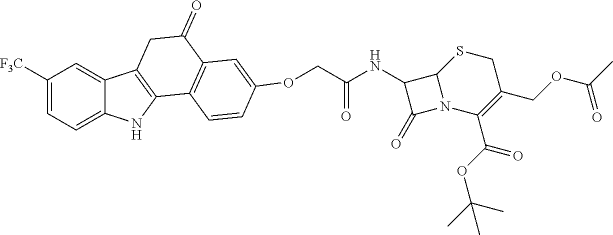 [SMILES: CC(=O)OCC1=C(C(=O)OC(C)(C)C)N2C(=O)C(NC(=O)COc3ccc4c(c3)C(=O)Cc3c-4[nH]c4ccc(C(F)(F)F)cc34)C2SC1] |
| XIVr | RA40 | 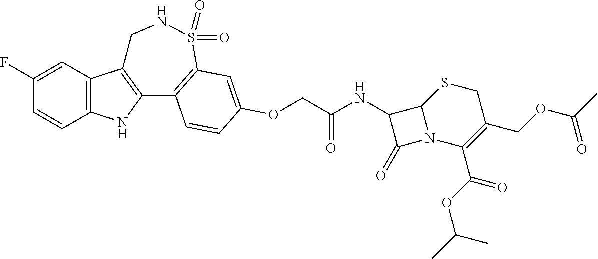 [SMILES: CC(=O)OCC1=C(C(=O)OC(C)C)N2C(=O)C(NC(=O)COc3ccc4c(c3)S(=O)(=O)NCc3c-4[nH]c4ccc(F)cc34)C2SC1] |
| XIVc | RA41 | 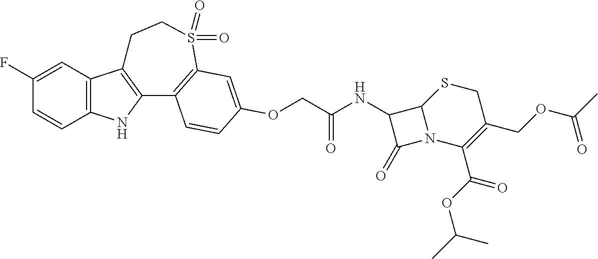 [SMILES: CC(=O)OCC1=C(C(=O)OC(C)C)N2C(=O)C(NC(=O)COc3ccc4c(c3)S(=O)(=O)CCc3c-4[nH]c4ccc(F)cc34)C2SC1] |
| XVr | RA42 | 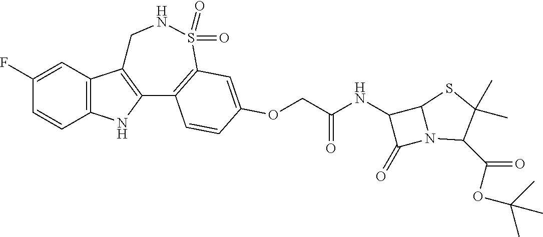 [SMILES: CC(C)(C)OC(=O)C1N2C(=O)C(NC(=O)COc3ccc4c(c3)S(=O)(=O)NCc3c-4[nH]c4ccc(F)cc34)C2SC1(C)C] |
| XIVc | RA43 | 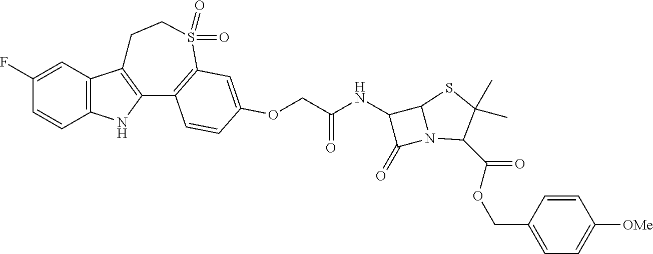 [SMILES: COc1ccc(COC(=O)C2N3C(=O)C(NC(=O)COc4ccc5c(c4)S(=O)(=O)CCc4c-5[nH]c5ccc(F)cc45)C3SC2(C)C)cc1] |
| XIIIr | RA44 | 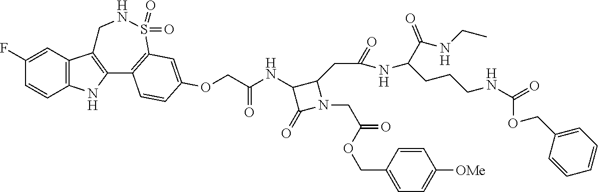 [SMILES: CCNC(=O)C(CCCNC(=O)OCc1ccccc1)NC(=O)CC1C(NC(=O)COc2ccc3c(c2)S(=O)(=O)NCc2c-3[nH]c3ccc(F)cc23)C(=O)N1CC(=O)OCc1ccc(OC)cc1] |

14. Compounds according to claim 4, wherein the compounds are selected from the group consisting of:

| # | # | |
|---|---|---|
| substructure | Abbreviation | Structure |
| XIc XII | RA1 | 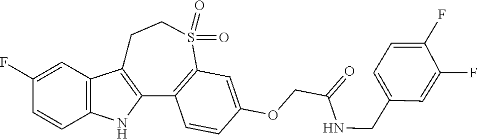 [SMILES: O=C(COc1ccc2c(c1)S(=O)(=O)CCc1c-2[nH]c2ccc(F)cc12)NCc1ccc(F)c(F)c1] |
| XIa | RA2 | 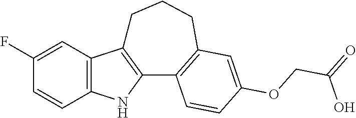 [SMILES: O=C(O)COc1ccc2c(c1)CCCc1c-2[nH]c2ccc(F)cc12] |
| XIr | RA3 | 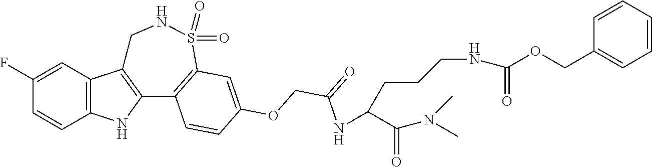 [SMILES: CN(C)C(=O)C(CCCNC(=O)OCc1ccccc1)NC(=O)COc1ccc2c(c1)S(=O)(=O)NCc1c-2[nH]c2ccc(F)cc12] |
| Xa | GA17M8 | 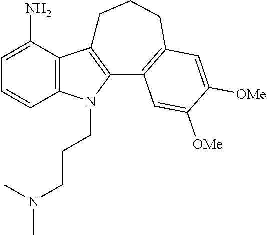 [SMILES: COc1cc2c(cc1OC)-c1c(c3c(N)cccc3n1CCCN(C)C)CCC2] |
| Xa | GA17M10 | 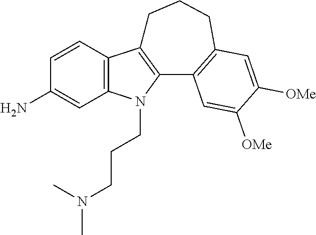 [SMILES: COc1cc2c(cc1OC)-c1c(c3ccc(N)cc3n1CCCN(C)C)CCC2] |
| XIb | GA11S | 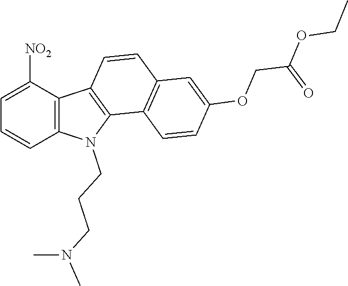 [SMILES: CCOC(=O)COc1ccc2c(ccc3c4c([N+](=O)[O-])cccc4n(CCCN(C)C)c23)c1] |
| XIb | RM66 | 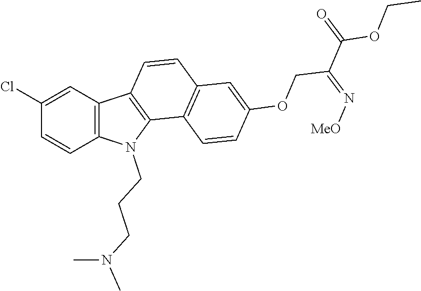 [SMILES: CCOC(=O)/C(COc1ccc2c(ccc3c4cc(Cl)ccc4n(CCCN(C)C)c23)c1)=N/OC] |
| XIb | RM58 | 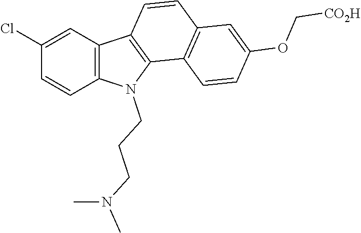 [SMILES: CN(C)CCCn1c2ccc(Cl)cc2c2ccc3cc(OCC(=O)O)ccc3c21] |
| XIb | RM70 | 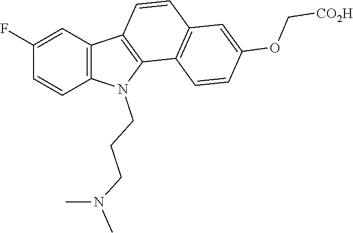 [SMILES: CN(C)CCCn1c2ccc(F)cc2c2ccc3cc(OCC(=O)O)ccc3c21] |
| XIb | RM85 | 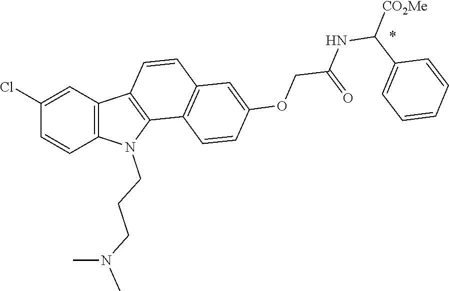 [SMILES: COC(=O)C(NC(=O)COc1ccc2c(ccc3c4cc(Cl)ccc4n(CCCN(C)C)c23)c1)c1ccccc1] |
| XI′d | RA6 | 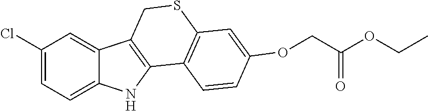 [SMILES: CCOC(=O)COc1ccc2c(c1)SCc1c-2[nH]c2ccc(Cl)cc12] |
| XI′d | RA7 | 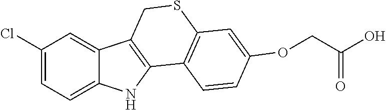 [SMILES: O=C(O)COc1ccc2c(c1)SCc1c-2[nH]c2ccc(Cl)cc12] |
| XI′d | RA9 | 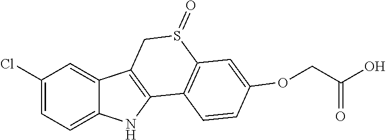 [SMILES: O=C(O)COc1ccc2c(c1)S(=O)Cc1c-2[nH]c2ccc(Cl)cc12] |
| XI′d | RA11 | 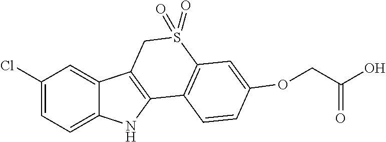 [SMILES: O=C(O)COc1ccc2c(c1)S(=O)(=O)Cc1c-2[nH]c2ccc(Cl)cc12] |
| XI′d | RA12 | 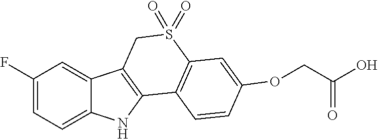 [SMILES: O=C(O)COc1ccc2c(c1)S(=O)(=O)Cc1c-2[nH]c2ccc(F)cc12] |
| XI′d | RA13 | 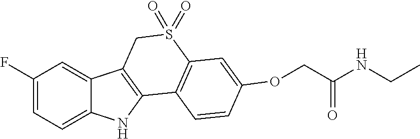 [SMILES: CCNC(=O)COc1ccc2c(c1)S(=O)(=O)Cc1c-2[nH]c2ccc(F)cc12] |
| XI′g | RA14 | 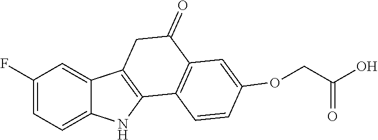 [SMILES: O=C(O)COc1ccc2c(c1)C(=O)Cc1c-2[nH]c2ccc(F)cc12] |
| X′c | RA15 | 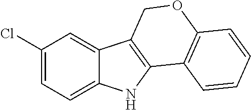 [SMILES: Clc1ccc2[nH]c3c(c2c1)COc1ccccc1-3] |
| X′c | RA16 | 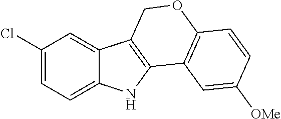 [SMILES: COc1ccc2c(c1)-c1[nH]c3ccc(Cl)cc3c1CO2] |
| XI′c | RA17 | 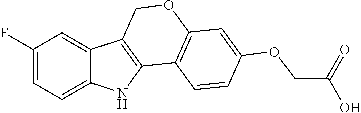 [SMILES: O=C(O)COc1ccc2c(c1)OCc1c-2[nH]c2ccc(F)cc12] |
| XI′c | RA18 | 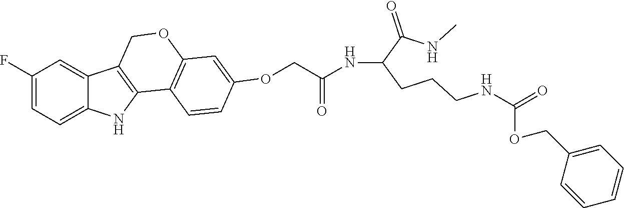 [SMILES: CNC(=O)C(CCCNC(=O)OCc1ccccc1)NC(=O)COc1ccc2c(c1)OCc1c-2[nH]c2ccc(F)cc12] |
| XI′c | RA19 | 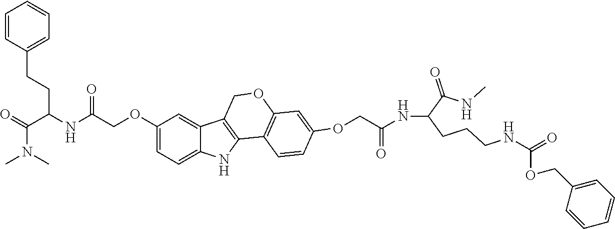 [SMILES: CNC(=O)C(CCCNC(=O)OCc1ccccc1)NC(=O)COc1ccc2c(c1)OCc1c-2[nH]c2ccc(OCC(=O)NC(CCc3ccccc3)C(=O)N(C)C)cc12] |
| XI′h | RA20 | 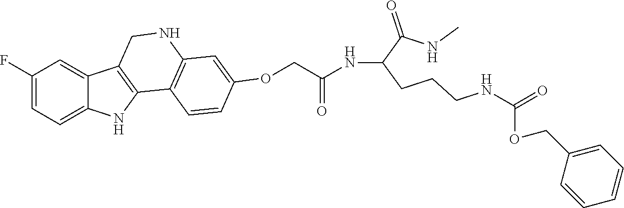 [SMILES: CNC(=O)C(CCCNC(=O)OCc1ccccc1)NC(=O)COc1ccc2c(c1)NCc1c-2[nH]c2ccc(F)cc12] |
| X′e | RA21 | 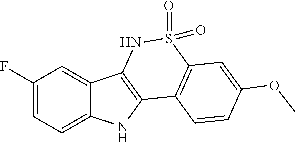 [SMILES: COc1ccc2c(c1)S(=O)(=O)Nc1c-2[nH]c2ccc(F)cc12] |
| XI′e | RA22 | 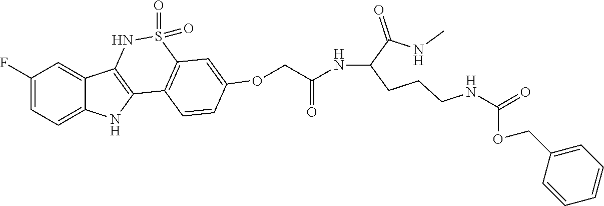 [SMILES: CNC(=O)C(CCCNC(=O)OCc1ccccc1)NC(=O)COc1ccc2c(c1)S(=O)(=O)Nc1c-2[nH]c2ccc(F)cc12] |
| XIV′a | GA09a | 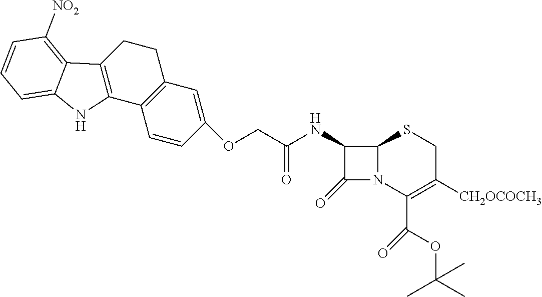 |
| XIV′a′ | GA09a′ | 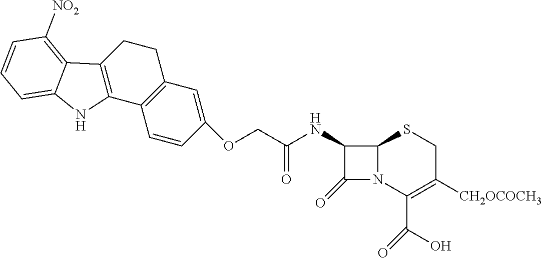 |
| XIV′a | GA09b | 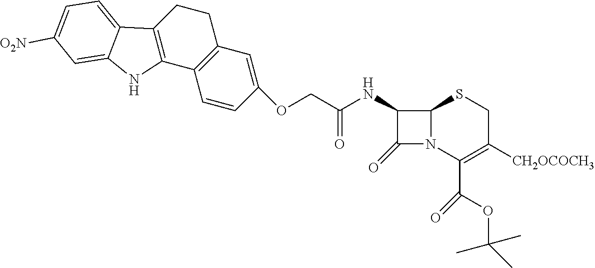 |
| XIV′a′ | GA09b | 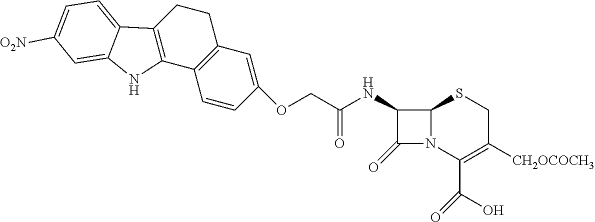 |
| XIV′a | RM36 | 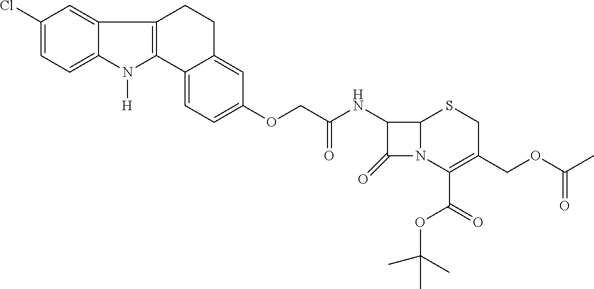 [SMILES: CC(=O)OCC1=C(C(=O)OC(C)(C)C)N2C(=O)C(NC(=O)COc3ccc4c(c3)CCc3c-4[nH]c4ccc(Cl)cc34)C2SC1] |
| XIV′a | RM37 | 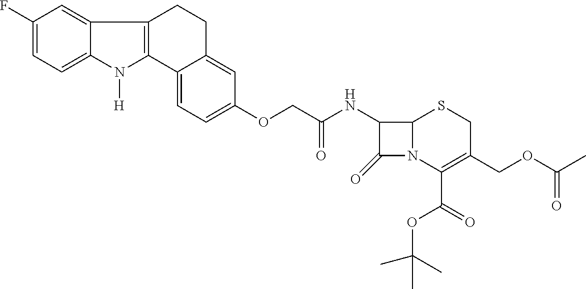 [SMILES: CC(=O)OCC1=C(C(=O)OC(C)(C)C)N2C(=O)C(NC(=O)COc3ccc4c(c3)CCc3c-4[nH]c4ccc(F)cc34)C2SC1] |
| XIV′a′ | RM53 | 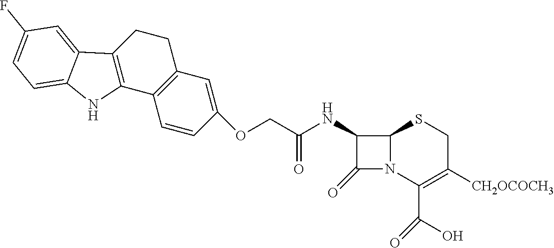 |
| XIV′a | RM47 | 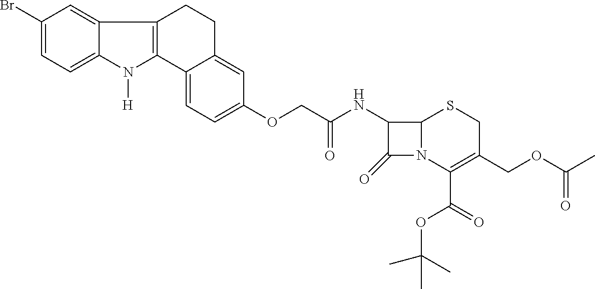 [SMILES: CC(=O)OCC1=C(C(=O)OC(C)(C)C)N2C(=O)C(NC(=O)COc3ccc4c(c3)CCc3c-4[nH]c4ccc(Br)cc34)C2SC1] |
| XIV′d | RA23 | 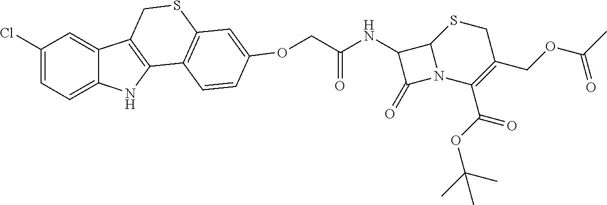 [SMILES: CC(=O)OCC1=C(C(=O)OC(C)(C)C)N2C(=O)C(NC(=O)COc3ccc4c(c3)SCc3c-4[nH]c4ccc(Cl)cc34)C2SC1] |
| XIV′d | RA24 | 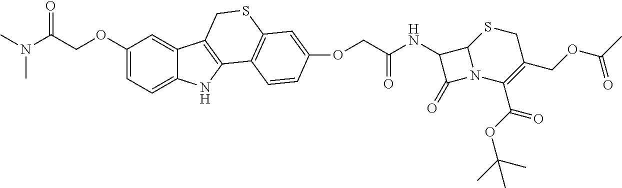 [SMILES: CC(=O)OCC1=C(C(=O)OC(C)(C)C)N2C(=O)C(NC(=O)COc3ccc4c(c3)SCc3c-4[nH]c4ccc(OCC(=O)N(C)C)cc34)C2SC1] |
| XIV′d | RA25 | 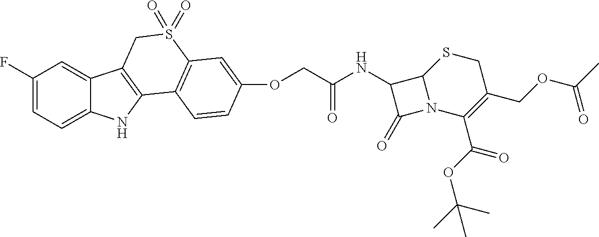 [SMILES: CC(=O)OCC1=C(C(=O)OC(C)(C)C)N2C(=O)C(NC(=O)COc3ccc4c(c3)S(=O)(=O)Cc3c-4[nH]c4ccc(F)cc34)C2SC1] |
| XV′e | RA26 | 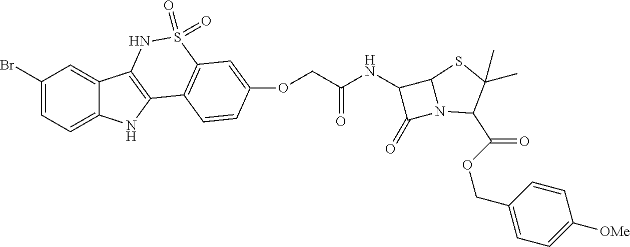 [SMILES: COc1ccc(COC(=O)C2N3C(=O)C(NC(=O)COc4ccc5c(c4)S(=O)(=O)Nc4c-5[nH]c5ccc(Br)cc45)C3SC2(C)C)cc1] |
| XV′d | RA27 | 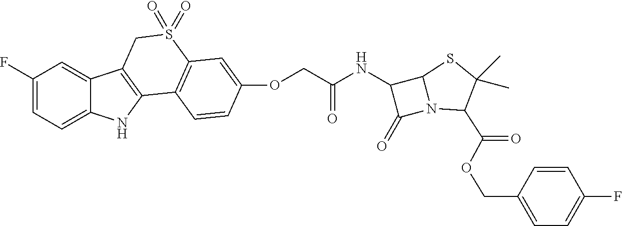 [SMILES: CC1(C)SC2C(NC(=O)COc3ccc4c(c3)S(=O)(=O)Cc3c-4[nH]c4ccc(F)cc34)C(=O)N2C1C(=O)OCc1ccc(F)cc1] |
| XV′d | RA28 | 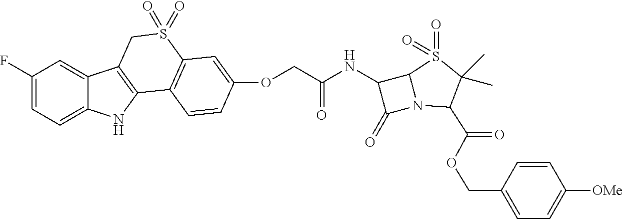 [SMILES: COc1ccc(COC(=O)C2N3C(=O)C(NC(=O)COc4ccc5c(c4)S(=O)(=O)Cc4c-5[nH]c5ccc(F)cc45)C3S(=O)(=O)C2(C)C)cc1] |
| XV′g′ | RA29 | 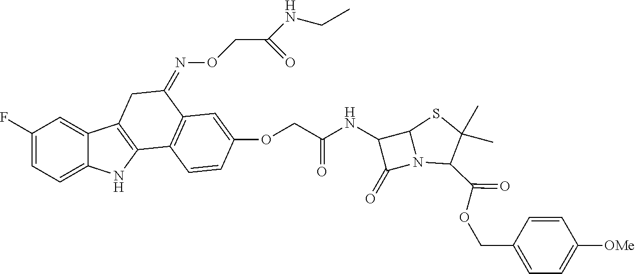 [SMILES: CCNC(=O)CO/N=C1/Cc2c([nH]c3ccc(F)cc23)-c2ccc(OCC(=O)NC3C(=O)N4C3SC(C)(C)C4C(=O)OCc3ccc(OC)cc3)cc21] |
| XIIId | RA30 | 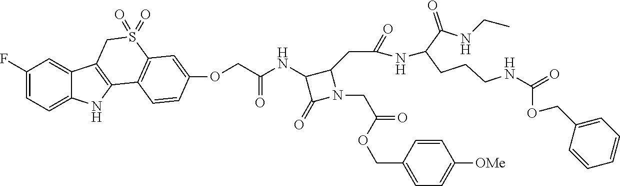 [SMILES: CCNC(=O)C(CCCNC(=O)OCc1ccccc1)NC(=O)CC1C(NC(=O)COc2ccc3c(c2)S(=O)(=O)Cc2c-3[nH]c3ccc(F)cc23)C(=O)N1CC(=O)OCc1ccc(OC)cc1] |
| XIV′c | RA31 | 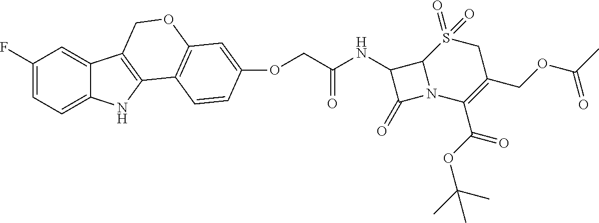 [SMILES: CC(=O)OCC1=C(C(=O)OC(C)(C)C)N2C(=O)C(NC(=O)COc3ccc4c(c3)OCc3c-4[nH]c4ccc(F)cc34)C2S(=O)(=O)C1] |
| XIV′c | RA32 | 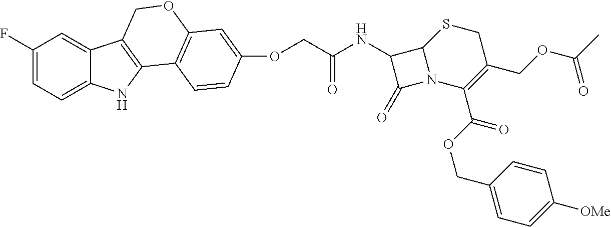 [SMILES: COc1ccc(COC(=O)C2=C(COC(C)=O)CSC3C(NC(=O)COc4ccc5c(c4)OCc4c-5[nH]c5ccc(F)cc45)C(=O)N23)cc1] |
| XIV′c | RA33 | 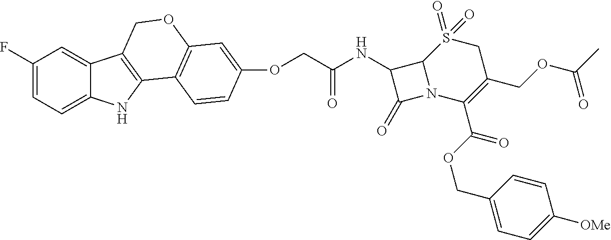 [SMILES: COc1ccc(COC(=O)C2=C(COC(C)=O)CS(=O)(=O)C3C(NC(=O)COc4ccc5c(c4)OCc4c-5[nH]c5ccc(F)cc45)C(=O)N23)cc1] |
| XIII′c | RA34 | 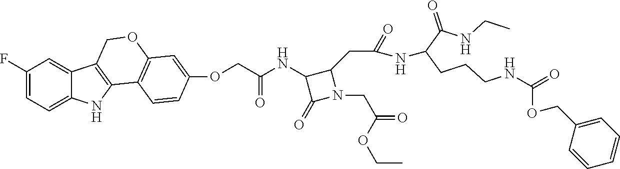 [SMILES: CCNC(=O)C(CCCNC(=O)OCc1ccccc1)NC(=O)CC1C(NC(=O)COc2ccc3c(c2)OCc2c-3[nH]c3ccc(F)cc23)C(=O)N1CC(=O)OCC] |
| XV′c | RA35 | 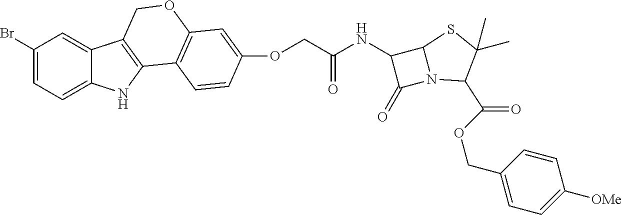 [SMILES: COc1ccc(COC(=O)C2N3C(=O)C(NC(=O)COc4ccc5c(c4)OCc4c-5[nH]c5ccc(Br)cc45)C3SC2(C)C)cc1] |
| XIV′e | RA36 | 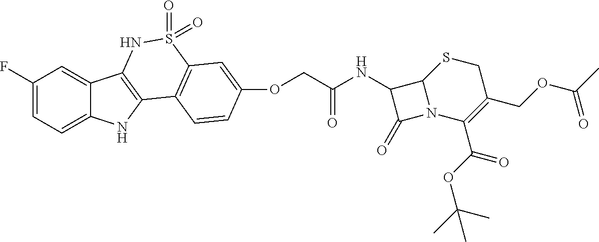 [SMILES: CC(=O)OCC1=C(C(=O)OC(C)(C)C)N2C(=O)C(NC(=O)COc3ccc4c(c3)S(=O)(=O)Nc3c-4[nH]c4ccc(F)cc34)C2SC1] |
| XIV′g′ | RA37 | 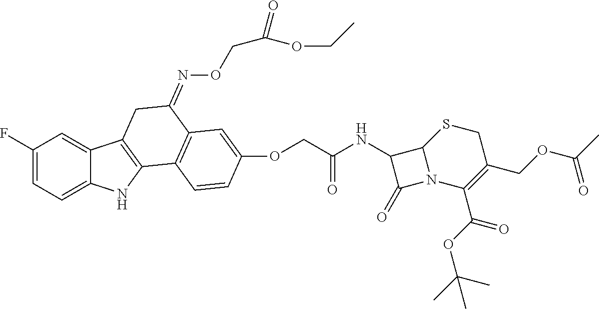 [SMILES: CCOC(=O)CO/N=C1/Cc2c([nH]c3ccc(F)cc23)-c2ccc(OCC(=O)NC3C(=O)N4C(C(=O)OC(C)(C)C)=C(COC(C)=O)CSC34)cc21] |
| XIV′g′ | RA38 | 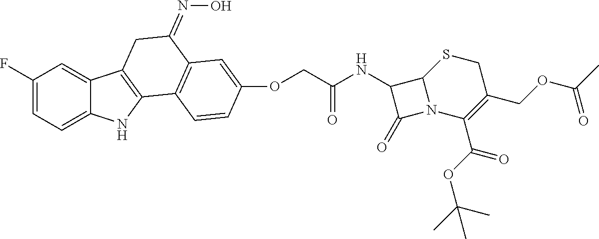 [SMILES: CC(=O)OCC1=C(C(=O)OC(C)(C)C)N2C(=O)C(NC(=O)COc3ccc4c(c3)/C(=N\O)Cc3c-4[nH]c4ccc(F)cc34)C2SC1] |
| XIV′g | RA39 | 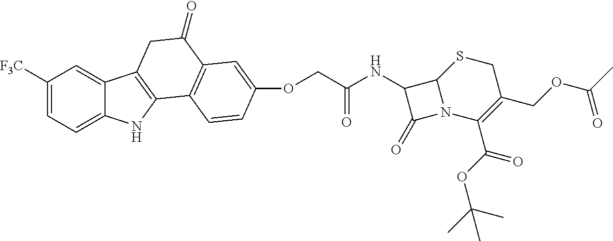 [SMILES: CC(=O)OCC1=C(C(=O)OC(C)(C)C)N2C(=O)C(NC(=O)COc3ccc4c(c3)C(=O)Cc3c-4[nH]c4ccc(C(F)(F)F)cc34)C2SC1] |
| XIVr | RA40 | 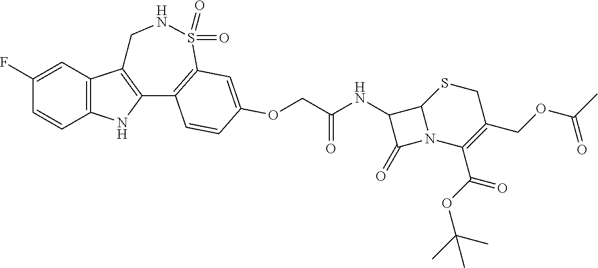 [SMILES: CC(=O)OCC1=C(C(=O)OC(C)(C)C)N2C(=O)C(NC(=O)COc3ccc4c(c3)S(=O)(=O)NCc3c-4[nH]c4ccc(F)cc34)C2SC1] |
| XIVc | RA41 | 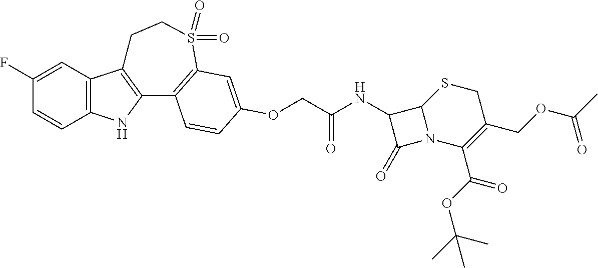 [SMILES: CC(=O)OCC1=C(C(=O)OC(C)(C)C)N2C(=O)C(NC(=O)COc3ccc4c(c3)S(=O)(=O)CCc3c-4[nH]c4ccc(F)cc34)C2SC1] |
| XVr | RA42 | 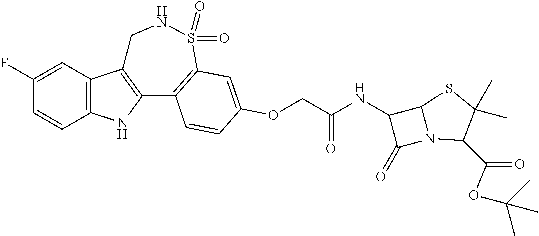 [SMILES: CC(C)(C)OC(=O)C1N2C(=O)C(NC(=O)COc3ccc4c(c3)S(=O)(=O)NCc3c-4[nH]c4ccc(F)cc34)C2SC1(C)C] |
| XIVc | RA43 | 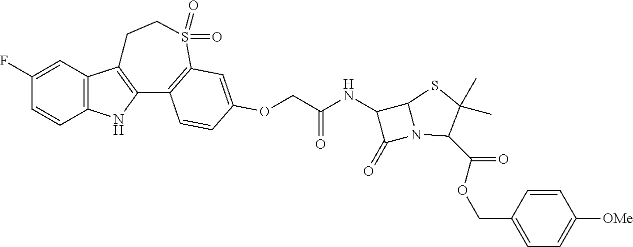 [SMILES: COc1ccc(COC(=O)C2N3C(=O)C(NC(=O)COc4ccc5c(c4)S(=O)(=O)CCc4c-5[nH]c5ccc(F)cc45)C3SC2(C)C)cc1] |
| XIIIr | RA44 | 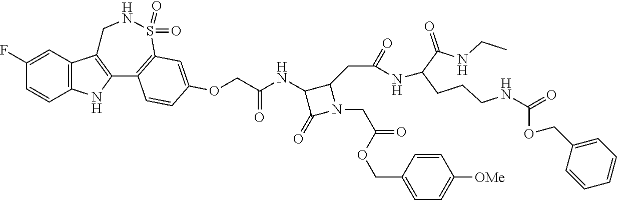 [SMILES: CCNC(=O)C(CCCNC(=O)OCc1ccccc1)NC(=O)CC1C(NC(=O)COc2ccc3c(c2)S(=O)(=O)NCc2c-3[nH]c3ccc(F)cc23)C(=O)N1CC(=O)OCc1ccc(OC)cc1] |

15. Compounds according to claim 5, wherein the compounds are selected from the group consisting of:

| # | # | |
|---|---|---|
| substructure | Abbreviation | Structure |
| XIc XII | RA1 | 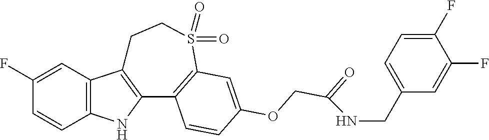 [SMILES: O=C(COc1ccc2c(c1)S(=O)(=O)CCc1c-2[nH]c2ccc(F)cc12)NCc1ccc(F)c(F)c1] |
| XIa | RA2 | 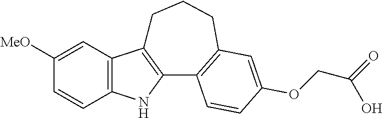 [SMILES: COc1ccc2[nH]c3c(c2c1)CCCc1cc(OCC(=O)O)ccc1-3] |
| XIr | RA3 | 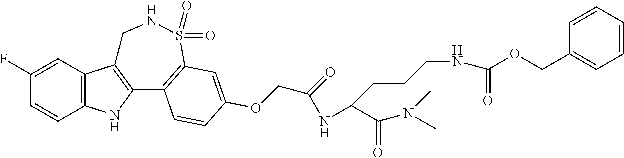 [SMILES: CN(C)C(=O)C(CCCNC(=O)OCc1ccccc1)NC(=O)COc1ccc2c(c1)S(=O)(=O)NCc1c-2[nH]c2ccc(F)cc12] |
| Xa | GA17M8 | 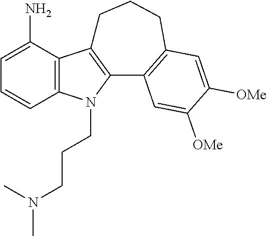 [SMILES: COc1cc2c(cc1OC)-c1c(c3c(N)cccc3n1CCCN(C)C)CCC2] |
| Xa | GA17M10 | 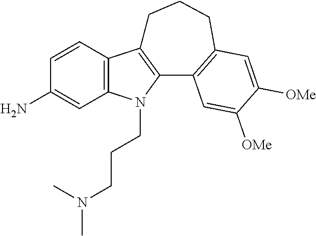 [SMILES: COc1cc2c(cc1OC)-c1c(c3ccc(N)cc3n1CCCN(C)C)CCC2] |
| XIb | GA11S | 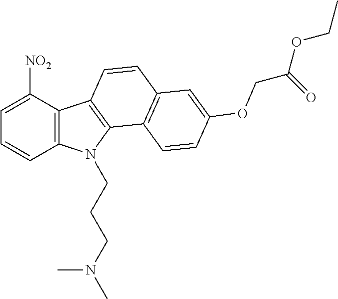 [SMILES: CCOC(=O)COc1ccc2c(ccc3c4c([N+](=O)[O-])cccc4n(CCCN(C)C)c23)c1] |
| XIb | RM66 | 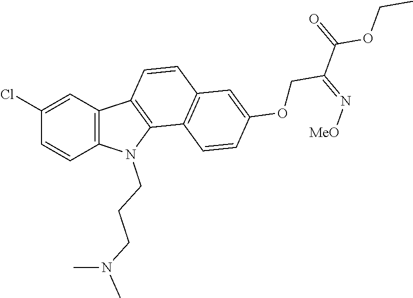 [SMILES: CCOC(=O)/C(COc1ccc2c(ccc3c4cc(Cl)ccc4n(CCCN(C)C)c23)c1)=N/OC] |
| XIb | RM58 | 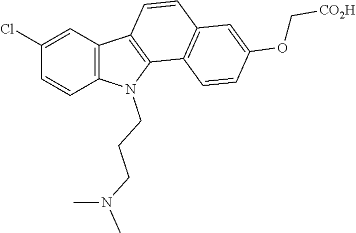 [SMILES: CN(C)CCCn1c2ccc(Cl)cc2c2ccc3cc(OCC(=O)O)ccc3c21] |
| XIb | RM70 | 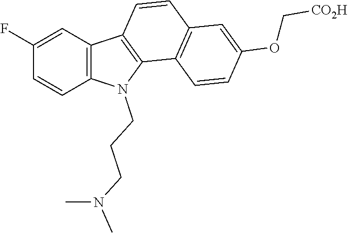 [SMILES: CN(C)CCCn1c2ccc(F)cc2c2ccc3cc(OCC(=O)O)ccc3c21] |
| XIb | RM85 | 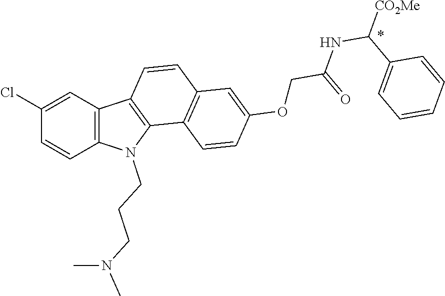 [SMILES: COC(=O)C(NC(=O)COc1ccc2c(ccc3c4cc(Cl)ccc4n(CCCN(C)C)c23)c1)c1ccccc1] |
| XI′d | RA6 | 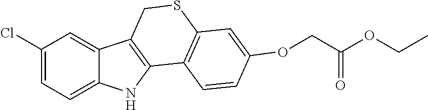 [SMILES: CCOC(=O)COc1ccc2c(c1)SCc1c-2[nH]c2ccc(Cl)cc12] |
| XI′d | RA7 | 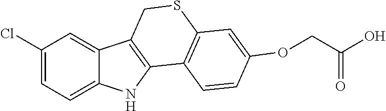 [SMILES: O=C(O)COc1ccc2c(c1)SCc1c-2[nH]c2ccc(Cl)cc12] |
| XI′d | RA9 | 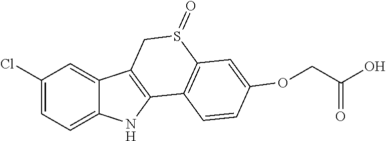 [SMILES: O=C(O)COc1ccc2c(c1)S(=O)Cc1c-2[nH]c2ccc(Cl)cc12] |
| XI′d | RA11 | 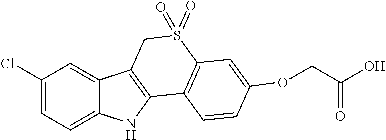 [SMILES: O=C(O)COc1ccc2c(c1)S(=O)(=O)Cc1c-2[nH]c2ccc(Cl)cc12] |
| XI′d | RA12 | 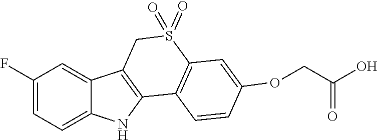 [SMILES: O=C(O)COc1ccc2c(c1)S(=O)(=O)Cc1c-2[nH]c2ccc(F)cc12] |
| XI′d | RA13 | 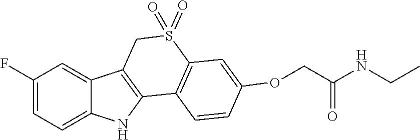 [SMILES: CCNC(=O)COc1ccc2c(c1)S(=O)(=O)Cc1c-2[nH]c2ccc(F)cc12] |
| XI′g | RA14 | 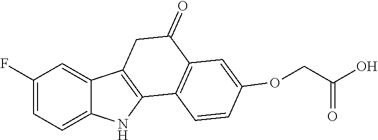 [SMILES: O=C(O)COc1ccc2c(c1)C(=O)Cc1c-2[nH]c2ccc(F)cc12] |
| X′c | RA15 | 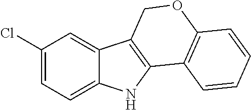 [SMILES: Clc1ccc2[nH]c3c(c2c1)COc1ccccc1-3] |
| X′c | RA16 | 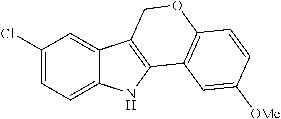 [SMILES: COc1ccc2c(c1)-c1[nH]c3ccc(Cl)cc3c1CO2] |
| XI′c | RA17 | 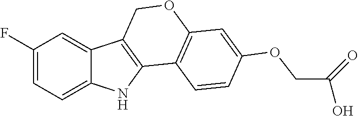 [SMILES: O=C(O)COc1ccc2c(c1)OCc1c-2[nH]c2ccc(F)cc12] |
| XI′c | RA18 | 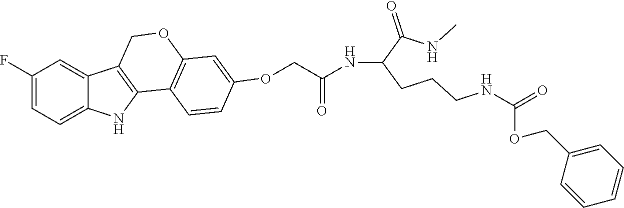 [SMILES: CNC(=O)C(CCCNC(=O)OCc1ccccc1)NC(=O)COc1ccc2c(c1)OCc1c-2[nH]c2ccc(F)cc12] |
| XI′c | RA19 | 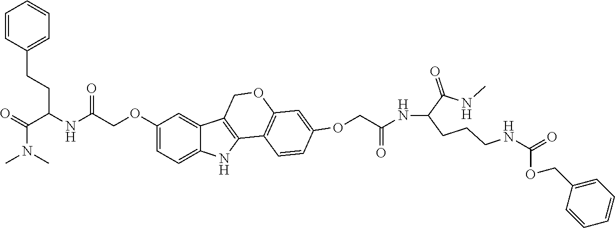 [SMILES: CNC(=O)C(CCCNC(=O)OCc1ccccc1)NC(=O)COc1ccc2c(c1)OCc1c-2[nH]c2ccc(OCC(=O)NC(CCc3ccccc3)C(=O)N(C)C)cc12] |
| XI′h | RA20 | 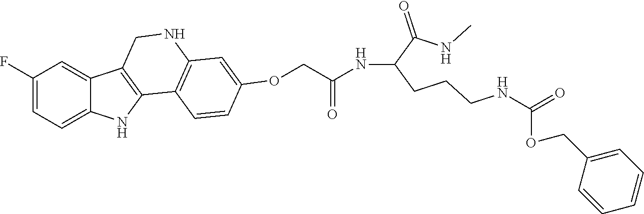 [SMILES: CNC(=O)C(CCCNC(=O)OCc1ccccc1)NC(=O)COc1ccc2c(c1)NCc1c-2[nH]c2ccc(F)cc12] |
| X′e | RA21 | 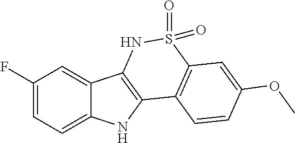 [SMILES: COc1ccc2c(c1)S(=O)(=O)Nc1c-2[nH]c2ccc(F)cc12] |
| XI′e | RA22 | 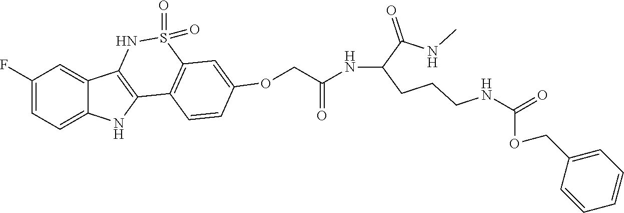 [SMILES: CNC(=O)C(CCCNC(=O)OCc1ccccc1)NC(=O)COc1ccc2c(c1)S(=O)(=O)Nc1c-2[nH]c2ccc(F)cc12] |
| XIV′a | GA09a | 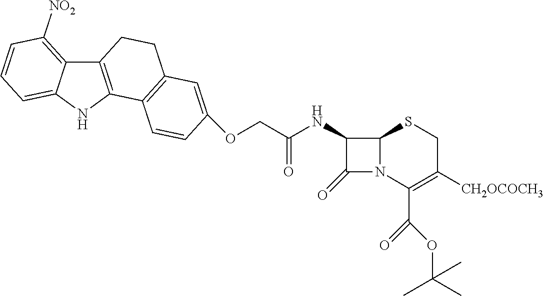 |
| XIV′a′ | GA09a′ | 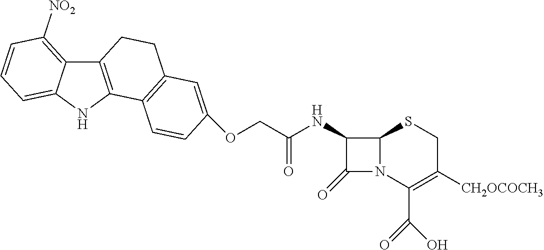 |
| XIV′a | GA09b | 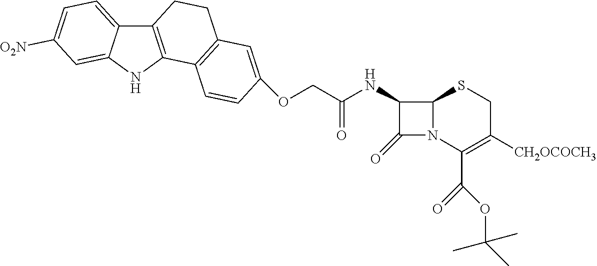 |
| XIV′a′ | GA09b′ | 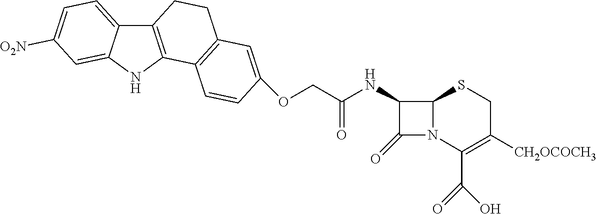 |
| XIV′a | RM36 | 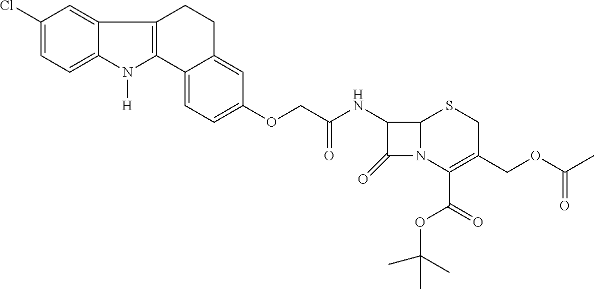 [SMILES: CC(=O)OCC1=C(C(=O)OC(C)(C)C)N2C(=O)C(NC(=O)COc3ccc4c(c3)CCc3c-4[nH]c4ccc(Cl)cc34)C2SC1] |
| XIV′a | RM37 | 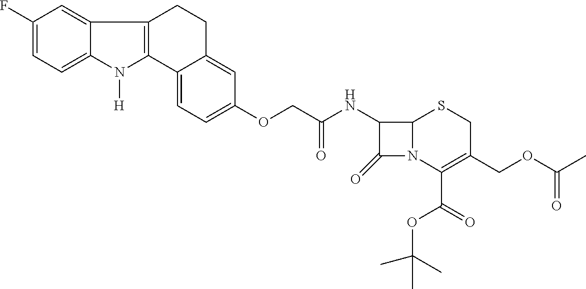 [SMILES: CC(=O)OCC1=C(C(=O)OC(C)(C)C)N2C(=O)C(NC(=O)COc3ccc4c(c3)CCc3c-4[nH]c4ccc(F)cc34)C2SC1] |
| XIV′a′ | RM53 | 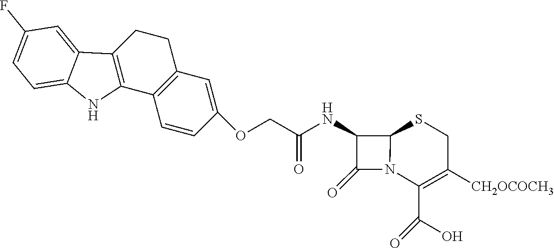 |
| XIV′a | RM47 | 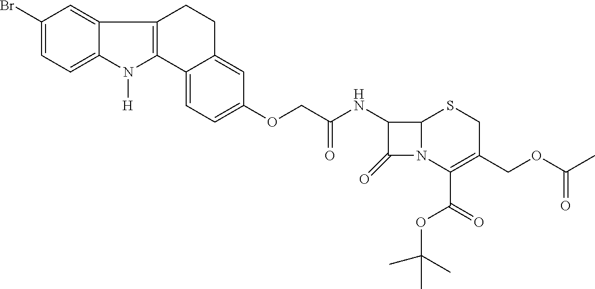 [SMILES: CC(=O)OCC1=C(C(=O)OC(C)(C)C)N2C(=O)C(NC(=O)COc3ccc4c(c3)CCc3c-4[nH]c4ccc(Br)cc34)C2SC1] |
| XIV′d | RA23 | 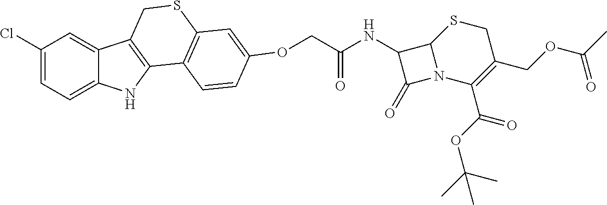 [SMILES: CC(=O)OCC1=C(C(=O)OC(C)(C)C)N2C(=O)C(NC(=O)COc3ccc4c(c3)SCc3c-4[nH]c4ccc(Cl)cc34)C2SC1] |
| XIV′d | RA24 | 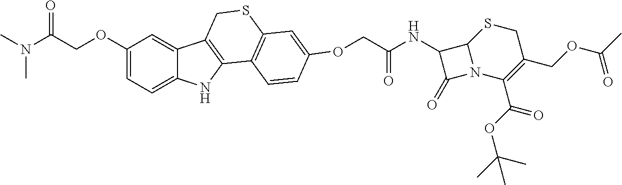 [SMILES: CC(=O)OCC1=C(C(=O)OC(C)(C)C)N2C(=O)C(NC(=O)COc3ccc4c(c3)SCc3c-4[nH]c4ccc(OCC(=O)N(C)C)cc34)C2SC1] |
| XIV′d | RA25 | 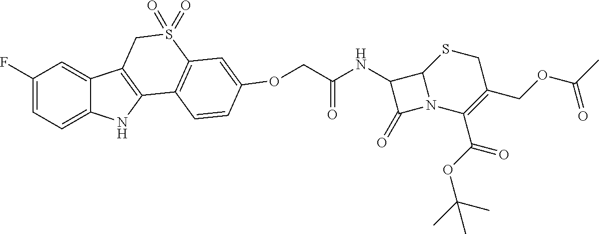 [SMILES: CC(=O)OCC1=C(C(=O)OC(C)(C)C)N2C(=O)C(NC(=O)COc3ccc4c(c3)S(=O)(=O)Cc3c-4[nH]c4ccc(F)cc34)C2SC1] |
| XV′e | RA26 | 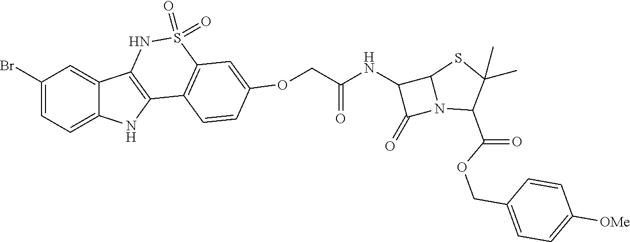 [SMILES: COc1ccc(COC(=O)C2N3C(=O)C(NC(=O)COc4ccc5c(c4)S(=O)(=O)Nc4c-5[nH]c5ccc(Br)cc45)C3SC2(C)C)cc1] |
| XV′d | RA27 | 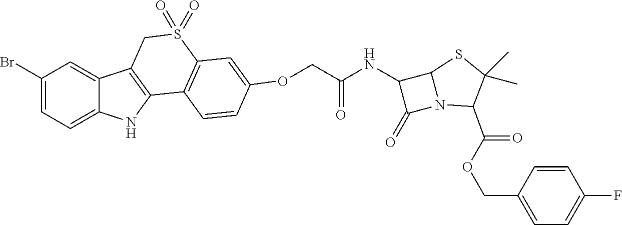 [SMILES: CC1(C)SC2C(NC(=O)COc3ccc4c(c3)S(=O)(=O)Cc3c-4[nH]c4ccc(Br)cc34)C(=O)N2C1C(=O)OCc1ccc(F)cc1] |
| XV′d | RA28 | 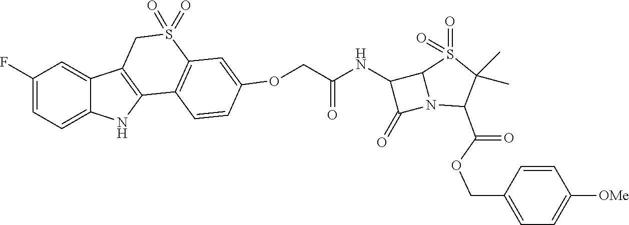 [SMILES: COc1ccc(COC(=O)C2N3C(=O)C(NC(=O)COc4ccc5c(c4)S(=O)(=O)Cc4c-5[nH]c5ccc(F)cc45)C3S(=O)(=O)C2(C)C)cc1] |
| XV′g′ | RA29 | 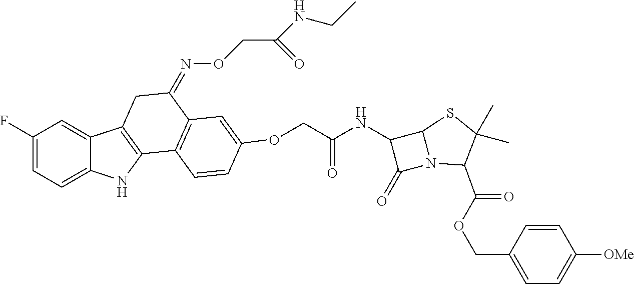 [SMILES: CCNC(=O)CO/N=C1/Cc2c([nH]c3ccc(F)cc23)-c2ccc(OCC(=O)NC3C(=O)N4C3SC(C)(C)C4C(=O)OCc3ccc(OC)cc3)cc21] |
| XIIId | RA30 | 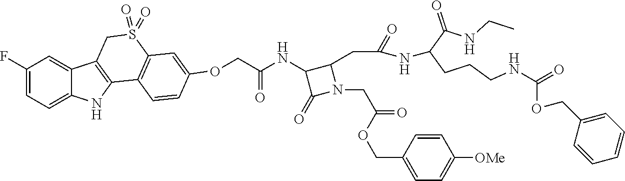 [SMILES: CCNC(=O)C(CCCNC(=O)OCc1ccccc1)NC(=O)CC1C(NC(=O)COc2ccc3c(c2)S(=O)(=O)Cc2c-3[nH]c3ccc(F)cc23)C(=O)N1CC(=O)OCc1ccc(OC)cc1] |
| XIV′c | RA31 | 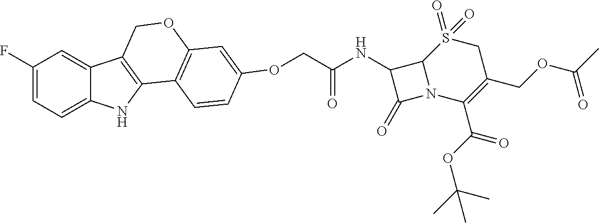 [SMILES: CC(=O)OCC1=C(C(=O)OC(C)(C)C)N2C(=O)C(NC(=O)COc3ccc4c(c3)OCc3c-4[nH]c4ccc(F)cc34)C2S(=O)(=O)C1] |
| XIV′c | RA32 | 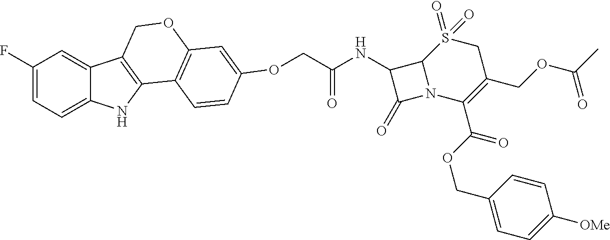 [SMILES: COc1ccc(COC(=O)C2=C(COC(C)=O)CS(=O)(=O)C3C(NC(=O)COc4ccc5c(c4)OCc4c-5[nH]c5ccc(F)cc45)C(=O)N23)cc1] |
| XIV′c | RA33 | 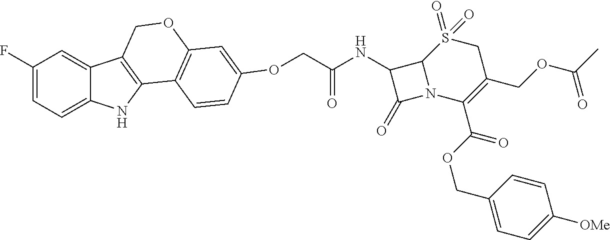 [SMILES: COc1ccc(COC(=O)C2=C(COC(C)=O)CS(=O)(=O)C3C(NC(=O)COc4ccc5c(c4)OCc4c-5[nH]c5ccc(F)cc45)C(=O)N23)cc1] |
| XIII′c | RA34 | 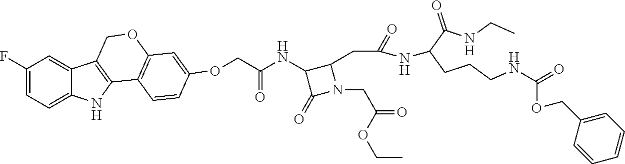 [SMILES: CCNC(=O)C(CCCNC(=O)OCc1ccccc1)NC(=O)CC1C(NC(=O)COc2ccc3c(c2)OCc2c-3[nH]c3ccc(F)cc23)C(=O)N1CC(=O)OCC] |
| XV′c | RA35 | 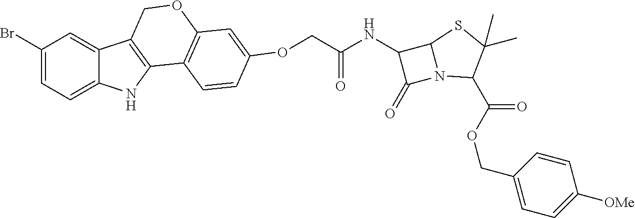 [SMILES: COc1ccc(COC(=O)C2N3C(=O)C(NC(=O)COc4ccc5c(c4)OCc4c-5[nH]c5ccc(Br)cc45)C3SC2(C)C)cc1] |
| XIV′e | RA36 | 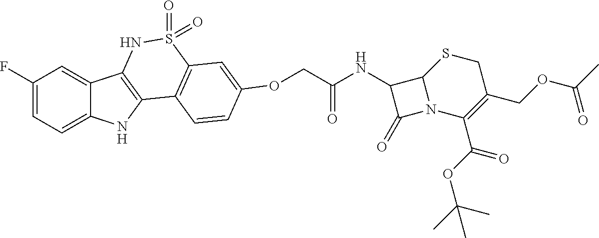 [SMILES: CC(=O)OCC1=C(C(=O)OC(C)(C)C)N2C(=O)C(NC(=O)COc3ccc4c(c3)S(=O)(=O)Nc3c-4[nH]c4ccc(F)cc34)C2SC1] |
| XIV′g′ | RA37 | 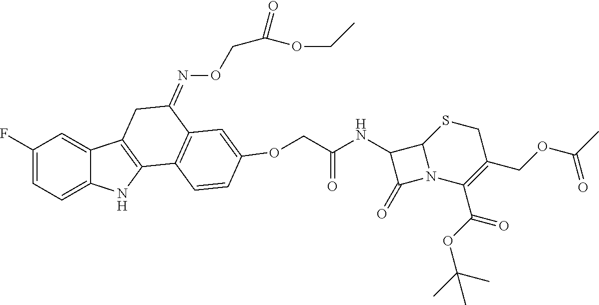 [SMILES: CCOC(=O)CO/N=C1/Cc2c([nH]c3ccc(F)cc23)-c2ccc(OCC(=O)NC3C(=O)N4C(C(=O)OC(C)(C)C)=C(COC(C)=O)CSC34)cc21] |
| XIV′g′ | RA38 | 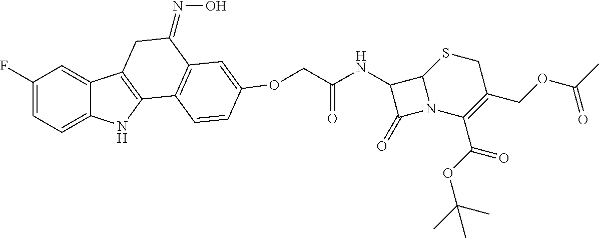 [SMILES: CC(=O)OCC1=C(C(=O)OC(C)(C)C)N2C(=O)C(NC(=O)COc3ccc4c(c3)/C(=N\O)Cc3c-4[nH]c4ccc(F)cc34)C2SC1] |
| XIV′g | RA39 | 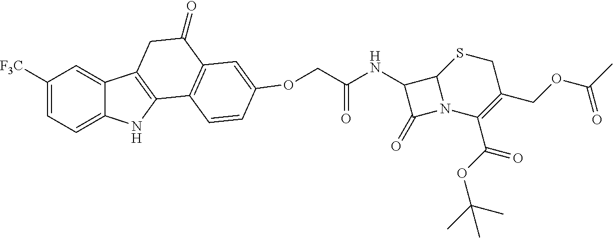 [SMILES: CC(=O)OCC1=C(C(=O)OC(C)(C)C)N2C(=O)C(NC(=O)COc3ccc4c(c3)C(=O)Cc3c-4[nH]c4ccc(C(F)(F)F)cc34)C2SC1] |
| XIVr | RA40 | 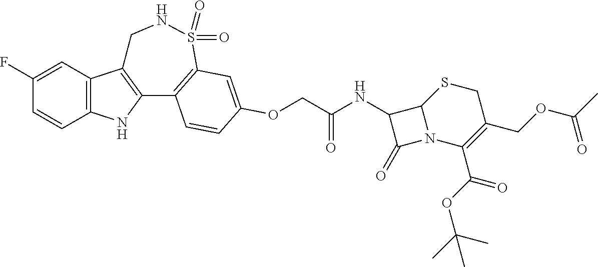 [SMILES: CC(=O)OCC1=C(C(=O)OC(C)(C)C)N2C(=O)C(NC(=O)COc3ccc4c(c3)S(=O)(=O)NCc3c-4[nH]c4ccc(F)cc34)C2SC1] |
| XIVc | RA41 | 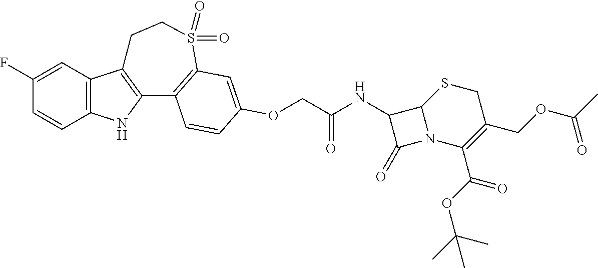 [SMILES: CC(=O)OCC1=C(C(=O)OC(C)(C)C)N2C(=O)C(NC(=O)COc3ccc4c(c3)S(=O)(=O)CCc3c-4[nH]c4ccc(F)cc34)C2SC1] |
| XVr | RA42 | 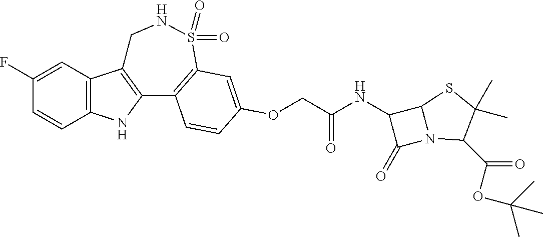 [SMILES: CC(C)(C)OC(=O)C1N2C(=O)C(NC(=O)COc3ccc4c(c3)S(=O)(=O)NCc3c-4[nH]c4ccc(F)cc34)C2SC1(C)C] |
| XIVc | RA43 | 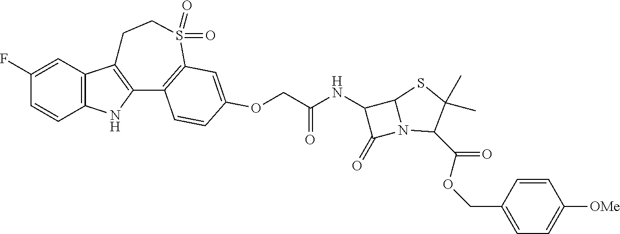 [SMILES: COc1ccc(COC(=O)C2N3C(=O)C(NC(=O)COc4ccc5c(c4)S(=O)(=O)CCc4c-5[nH]c5ccc(F)cc45)C3SC2(C)C)cc1] |
| XIIIr | RA44 | 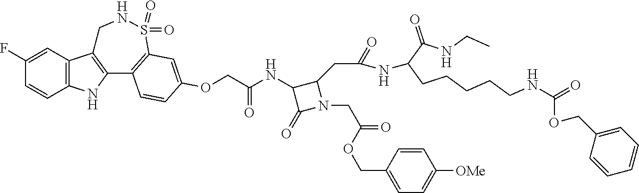 [SMILES: CCNC(=O)C(CCCCCNC(=O)OCc1ccccc1)NC(=O)CC1C(NC(=O)COc2ccc3c(c2)S(=O)(=O)NCc2c-3[nH]c3ccc(F)cc23)C(=O)N1CC(=O)OCc1ccc(OC)cc1] |

16. Compounds according to claim 6, wherein the compounds are selected from the group consisting of:

| # | # | |
|---|---|---|
| substructure | Abbreviation | Structure |
| XIc XII | RA1 | 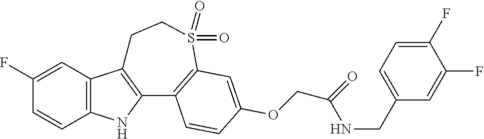 [SMILES: O=C(COc1ccc2c(c1)S(=O)(=O)CCc1c-2[nH]c2ccc(F)cc12)NCc1ccc(F)c(F)c1] |
| XIa | RA2 | 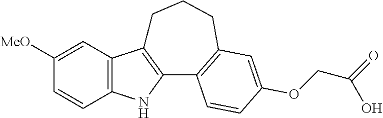 [SMILES: COc1ccc2[nH]c3c(c2c1)CCCc1cc(OCC(=O)O)ccc1-3] |
| XIr | RA3 | 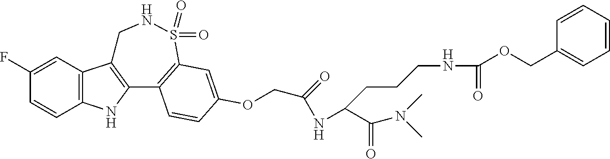 [SMILES: CN(C)C(=O)C(CCCNC(=O)OCc1ccccc1)NC(=O)COc1ccc2c(c1)S(=O)(=O)NCc1c-2[nH]c2ccc(F)cc12] |
| Xa | GA17M8 | 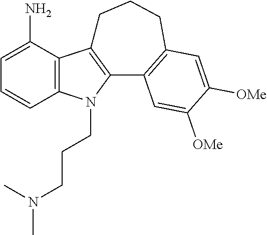 [SMILES: COc1cc2c(cc1OC)-c1c(c3c(N)cccc3n1CCCN(C)C)CCC2] |
| Xa | GA17M10 | 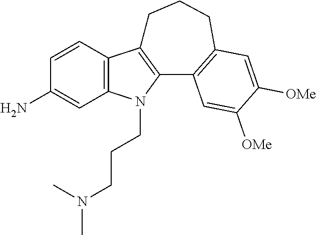 [SMILES: COc1cc2c(cc1OC)-c1c(c3ccc(N)cc3n1CCCN(C)C)CCC2] |
| XIb | GA11S | 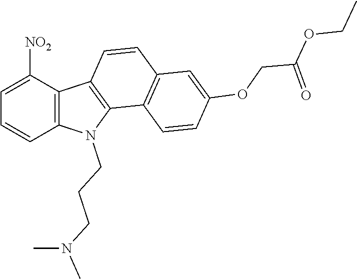 [SMILES: CCOC(=O)COc1ccc2c(ccc3c4c([N+](=O)[O-])cccc4n(CCCN(C)C)c23)c1] |
| XIb | RM66 | 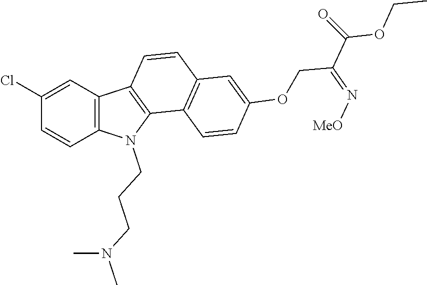 [SMILES: CCOC(=O)/C(COc1ccc2c(ccc3c4cc(Cl)ccc4n(CCCN(C)C)c23)c1)=N/OC] |
| XIb | RM58 | 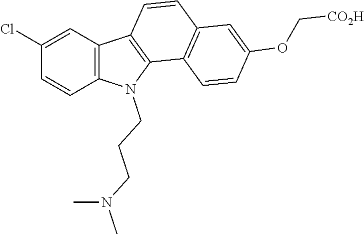 [SMILES: CN(C)CCCn1c2ccc(Cl)cc2c2ccc3cc(OCC(=O)O)ccc3c21] |
| XIb | RM70 | 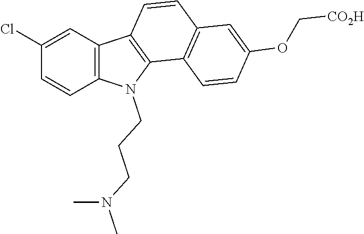 [SMILES: CN(C)CCCn1c2ccc(Cl)cc2c2ccc3cc(OCC(=O)O)ccc3c21] |
| XIb | RM85 | 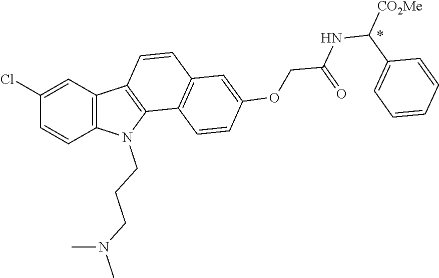 [SMILES: COC(=O)C(NC(=O)COc1ccc2c(ccc3c4cc(Cl)ccc4n(CCCN(C)C)c23)c1)c1ccccc1] |
| XI′d | RA6 | 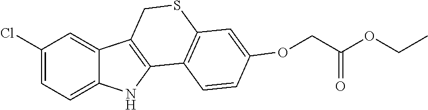 [SMILES: CCOC(=O)COc1ccc2c(c1)SCc1c-2[nH]c2ccc(Cl)cc12] |
| XI′d | RA7 | 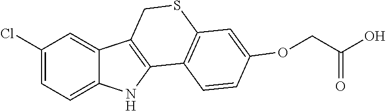 [SMILES: O=C(O)COc1ccc2c(c1)SCc1c-2[nH]c2ccc(Cl)cc12] |
| XI′d | RA9 | 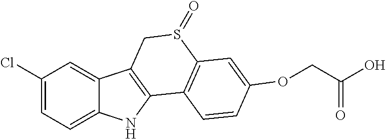 [SMILES: O=C(O)COc1ccc2c(c1)S(=O)Cc1c-2[nH]c2ccc(Cl)cc12] |
| XI′d | RA11 | 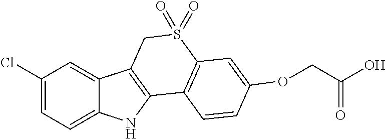 [SMILES: O=C(O)COc1ccc2c(c1)S(=O)(=O)Cc1c-2[nH]c2ccc(Cl)cc12] |
| XI′d | RA12 | 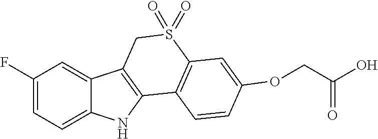 [SMILES: O=C(O)COc1ccc2c(c1)S(=O)(=O)Cc1c-2[nH]c2ccc(F)cc12] |
| XI′d | RA13 | 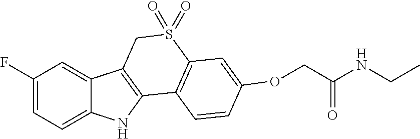 [SMILES: CCNC(=O)COc1ccc2c(c1)S(=O)(=O)Cc1c-2[nH]c2ccc(F)cc12] |
| XI′g | RA14 | 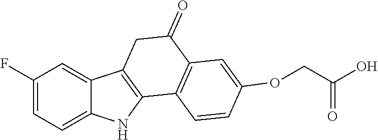 [SMILES: O=C(O)COc1ccc2c(c1)C(=O)Cc1c-2[nH]c2ccc(F)cc12] |
| X′c | RA15 | 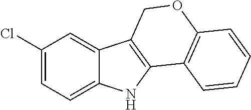 [SMILES: Clc1ccc2[nH]c3c(c2c1)COc1ccccc1-3] |
| X′c | RA16 | 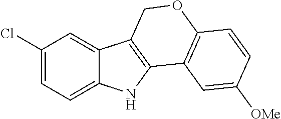 [SMILES: COc1ccc2c(c1)-c1[nH]c3ccc(Cl)cc3c1CO2] |
| XI′c | RA17 | 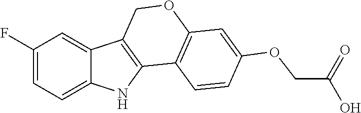 [SMILES: O=C(O)COc1ccc2c(c1)OCc1c-2[nH]c2ccc(F)cc12] |
| XI′c | RA18 | 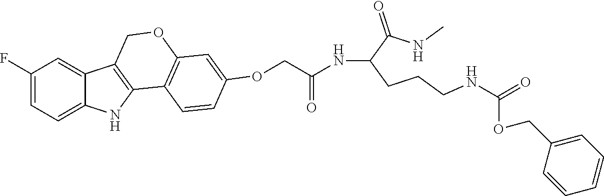 [SMILES: CNC(=O)C(CCCNC(=O)OCc1ccccc1)NC(=O)COc1ccc2c(c1)OCc1c-2[nH]c2ccc(F)cc12] |
| XI′c | RA19 | 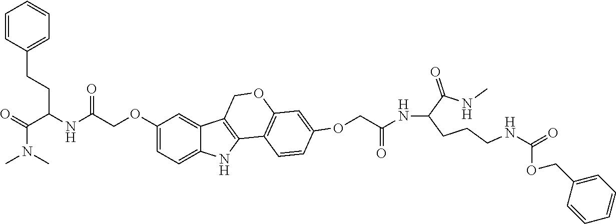 [SMILES: CNC(=O)C(CCCNC(=O)OCc1ccccc1)NC(=O)COc1ccc2c(c1)OCc1c-2[nH]c2ccc(OCC(=O)NC(CCc3ccccc3)C(=O)N(C)C)cc12] |
| XI′h | RA20 | 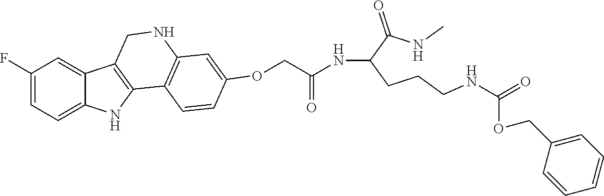 [SMILES: CNC(=O)C(CCCNC(=O)OCc1ccccc1)NC(=O)COc1ccc2c(c1)NCc1c-2[nH]c2ccc(F)cc12] |
| X′e | RA21 | 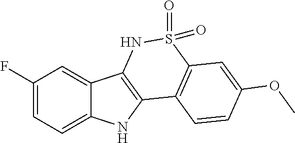 [SMILES: COc1ccc2c(c1)S(=O)(=O)Nc1c-2[nH]c2ccc(F)cc12] |
| XI′e | RA22 | 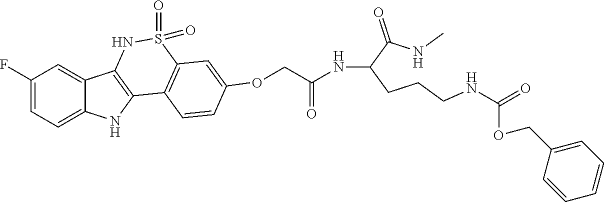 [SMILES: CNC(=O)C(CCCNC(=O)OCc1ccccc1)NC(=O)COc1ccc2c(c1)S(=O)(=O)Nc1c-2[nH]c2ccc(F)cc12] |
| XIV′a | GA09a | 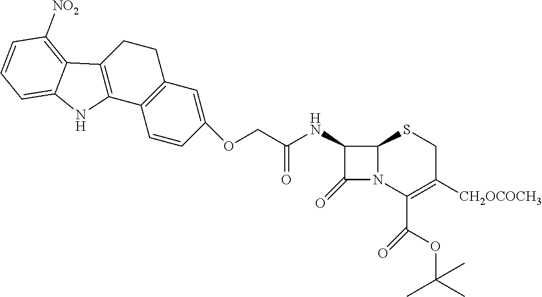 |
| XIV′a′ | GA09a′ | 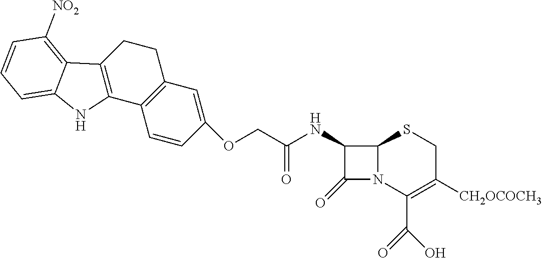 |
| XIV′a | GA09b | 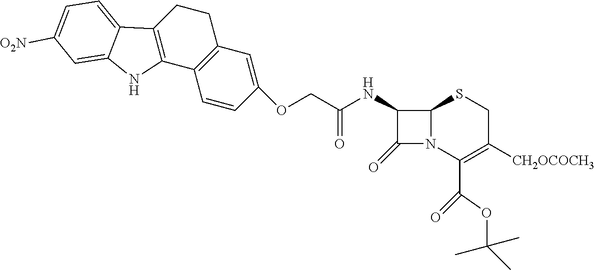 |
| XIV′a′ | GA09b′ | 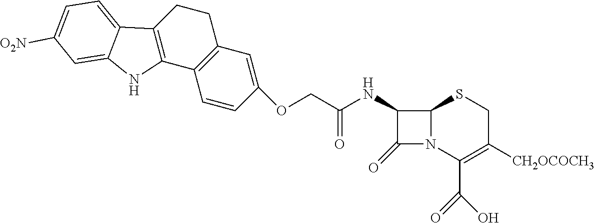 |
| XIV′a | RM36 | 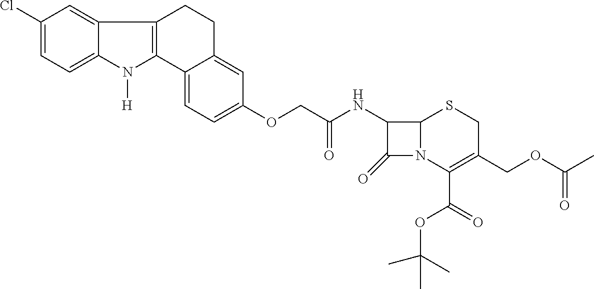 [SMILES: CC(=O)OCC1=C(C(=O)OC(C)(C)C)N2C(=O)C(NC(=O)COc3ccc4c(c3)CCc3c-4[nH]c4ccc(Cl)cc34)C2SC1] |
| XIV′a | RM37 | 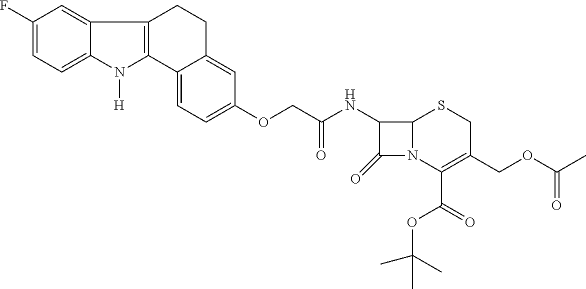 [SMILES: CC(=O)OCC1=C(C(=O)OC(C)(C)C)N2C(=O)C(NC(=O)COc3ccc4c(c3)CCc3c-4[nH]c4ccc(F)cc34)C2SC1] |
| XIV′a′ | RM53 | 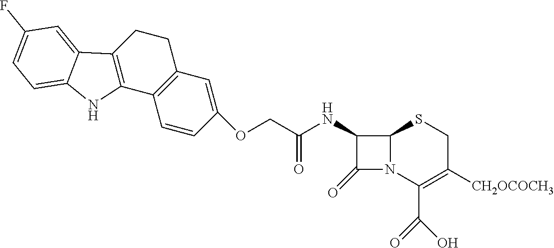 |
| XIV′a | RM47 | 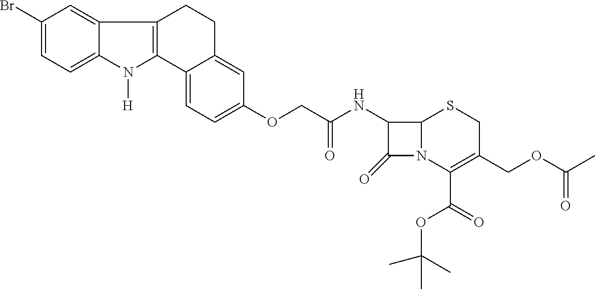 [SMILES: CC(=O)OCC1=C(C(=O)OC(C)(C)C)N2C(=O)C(NC(=O)COc3ccc4c(c3)CCc3c-4[nH]c4ccc(Br)cc34)C2SC1] |
| XIV′d | RA23 | 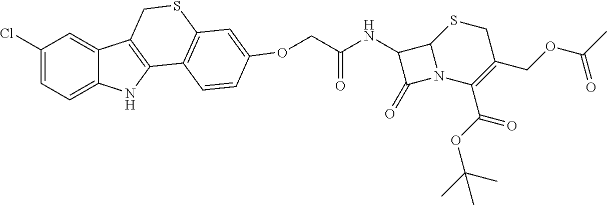 [SMILES: CC(=O)OCC1=C(C(=O)OC(C)(C)C)N2C(=O)C(NC(=O)COc3ccc4c(c3)SCc3c-4[nH]c4ccc(Cl)cc34)C2SC1] |
| XIV′d | RA24 | 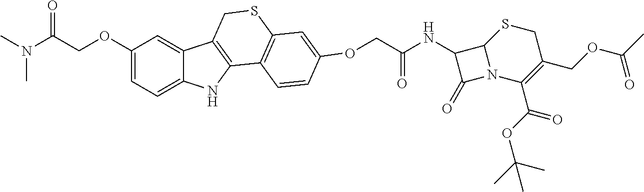 [SMILES: CC(=O)OCC1=C(C(=O)OC(C)(C)C)N2C(=O)C(NC(=O)COc3ccc4c(c3)SCc3c-4[nH]c4ccc(OCC(=O)N(C)C)cc34)C2SC1] |
| XIV′d | RA25 | 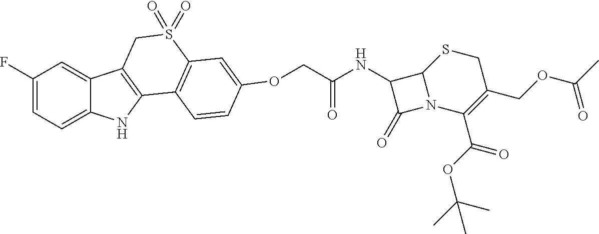 [SMILES: CC(=O)OCC1=C(C(=O)OC(C)(C)C)N2C(=O)C(NC(=O)COc3ccc4c(c3)S(=O)(=O)Cc3c-4[nH]c4ccc(F)cc34)C2SC1] |
| XV′e | RA26 | 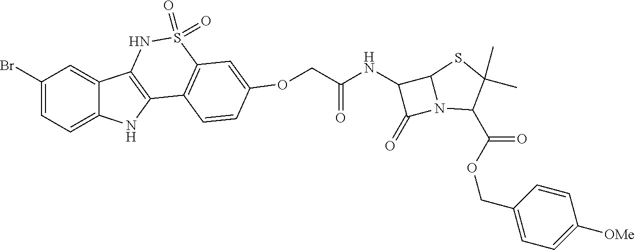 [SMILES: COc1ccc(COC(=O)C2N3C(=O)C(NC(=O)COc4ccc5c(c4)S(=O)(=O)Nc4c-5[nH]c5ccc(Br)cc45)C3SC2(C)C)cc1] |
| XV′d | RA27 | 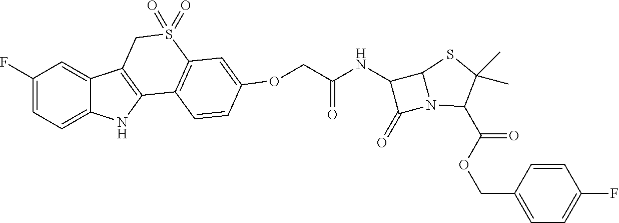 [SMILES: CC1(C)SC2C(NC(=O)COc3ccc4c(c3)S(=O)(=O)Cc3c-4[nH]c4ccc(F)cc34)C(=O)N2C1C(=O)OCc1ccc(F)cc1] |
| XV′d | RA28 | 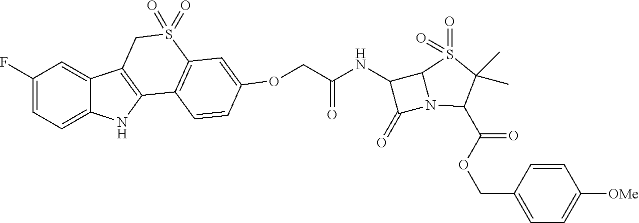 [SMILES: COc1ccc(COC(=O)C2N3C(=O)C(NC(=O)COc4ccc5c(c4)S(=O)(=O)Cc4c-5[nH]c5ccc(F)cc45)C3S(=O)(=O)C2(C)C)cc1] |
| XV′g′ | RA29 | 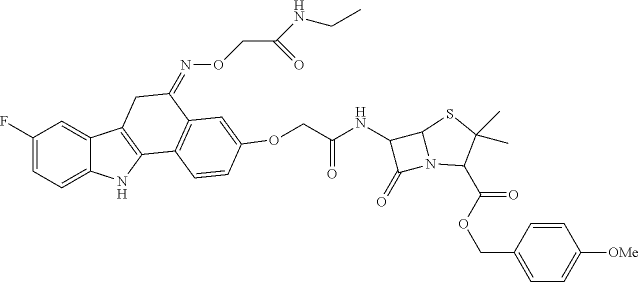 [SMILES: CCNC(=O)CO/N=C1/Cc2c([nH]c3ccc(F)cc23)-c2ccc(OCC(=O)NC3C(=O)N4C3SC(C)(C)C4C(=O)OCc3ccc(OC)cc3)cc21] |
| XIIId | RA30 | 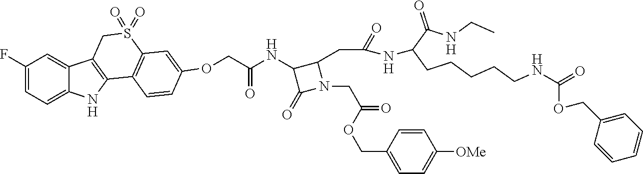 [SMILES: CCNC(=O)C(CCCCCNC(=O)OCc1ccccc1)NC(=O)CC1C(NC(=O)COc2ccc3c(c2)S(=O)(=O)Cc2c-3[nH]c3ccc(F)cc23)C(=O)N1CC(=O)OCc1ccc(OC)cc1] |
| XIV′c | RA31 | 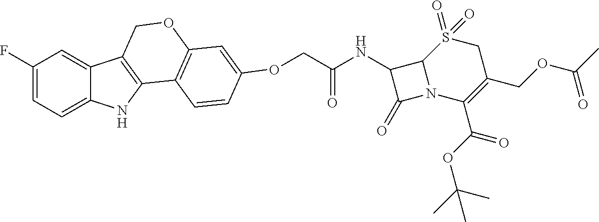 [SMILES: CC(=O)OCC1=C(C(=O)OC(C)(C)C)N2C(=O)C(NC(=O)COc3ccc4c(c3)OCc3c-4[nH]c4ccc(F)cc34)C2S(=O)(=O)C1] |
| XIV′c | RA32 | 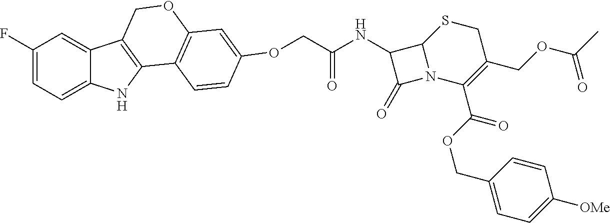 [SMILES: COc1ccc(COC(=O)C2=C(COC(C)=O)CSC3C(NC(=O)COc4ccc5c(c4)OCc4c-5[nH]c5ccc(F)cc45)C(=O)N23)cc1] |
| XIV′c | RA33 | 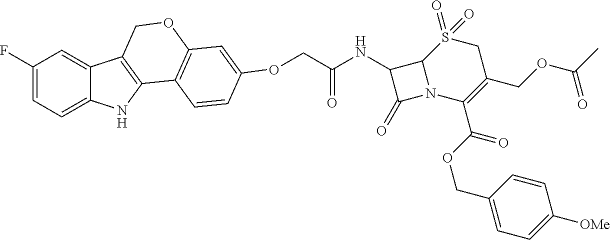 [SMILES: COc1ccc(COC(=O)C2=C(COC(C)=O)CS(=O)(=O)C3C(NC(=O)COc4ccc5c(c4)OCc4c-5[nH]c5ccc(F)cc45)C(=O)N23)cc1] |
| XIII′c | RA34 | 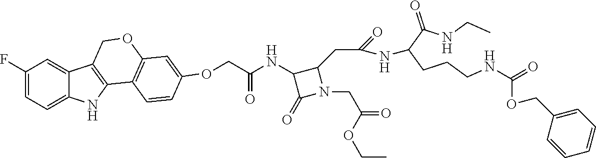 [SMILES: CCNC(=O)C(CCCNC(=O)OCc1ccccc1)NC(=O)CC1C(NC(=O)COc2ccc3c(c2)OCc2c-3[nH]c3ccc(F)cc23)C(=O)N1CC(=O)OCC] |
| XV′c | RA35 | 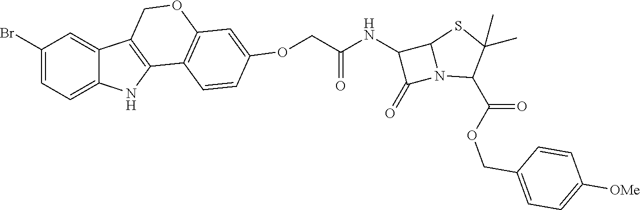 [SMILES: COc1ccc(COC(=O)C2N3C(=O)C(NC(=O)COc4ccc5c(c4)OCc4c-5[nH]c5ccc(Br)cc45)C3SC2(C)C)cc1] |
| XIV′e | RA36 | 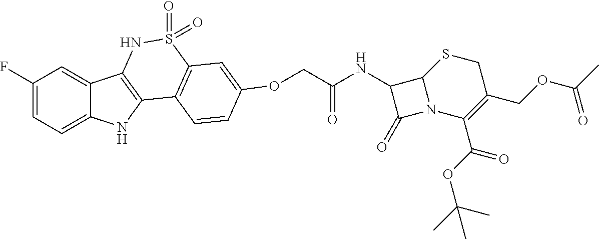 [SMILES: CC(=O)OCC1=C(C(=O)OC(C)(C)C)N2C(=O)C(NC(=O)COc3ccc4c(c3)S(=O)(=O)Nc3c-4[nH]c4ccc(F)cc34)C2SC1] |
| XIV′g′ | RA37 | 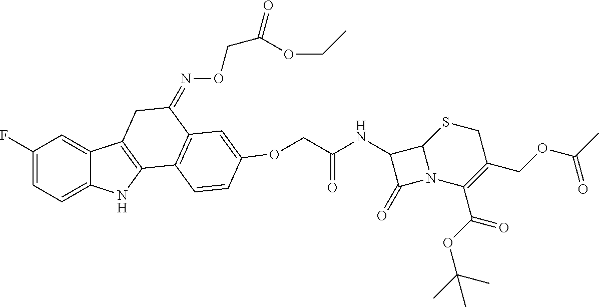 [SMILES: CCOC(=O)CO/N=C1/Cc2c([nH]c3ccc(F)cc23)-c2ccc(OCC(=O)NC3C(=O)N4C(C(=O)OC(C)(C)C)=C(COC(C)=O)CSC34)cc21] |
| XIV′g′ | RA38 | 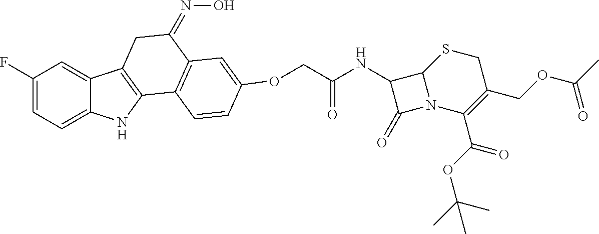 [SMILES: CC(=O)OCC1=C(C(=O)OC(C)(C)C)N2C(=O)C(NC(=O)COc3ccc4c(c3)/C(=N\O)Cc3c-4[nH]c4ccc(F)cc34)C2SC1] |
| XIV′g′ | RA39 | 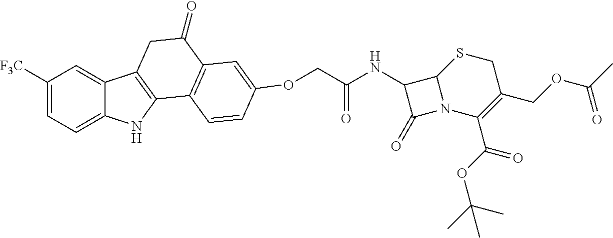 [SMILES: CC(=O)OCC1=C(C(=O)OC(C)(C)C)N2C(=O)C(NC(=O)COc3ccc4c(c3)C(=O)Cc3c-4[nH]c4ccc(C(F)(F)F)cc34)C2SC1] |
| XIVr | RA40 | 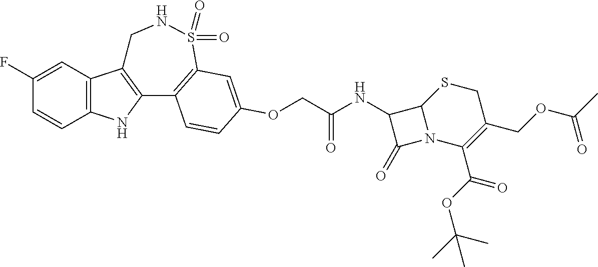 [SMILES: CC(=O)OCC1=C(C(=O)OC(C)(C)C)N2C(=O)C(NC(=O)COc3ccc4c(c3)S(=O)(=O)NCc3c-4[nH]c4ccc(F)cc34)C2SC1] |
| XIVc | RA41 | 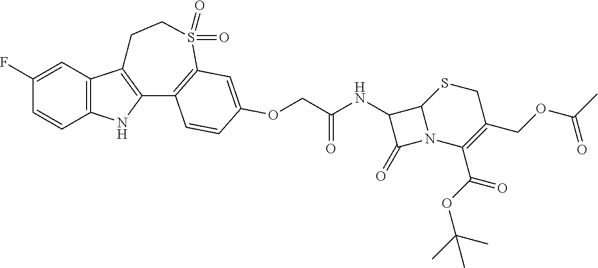 [SMILES: CC(=O)OCC1=C(C(=O)OC(C)(C)C)N2C(=O)C(NC(=O)COc3ccc4c(c3)S(=O)(=O)CCc3c-4[nH]c4ccc(F)cc34)C2SC1] |
| XVr | RA42 | 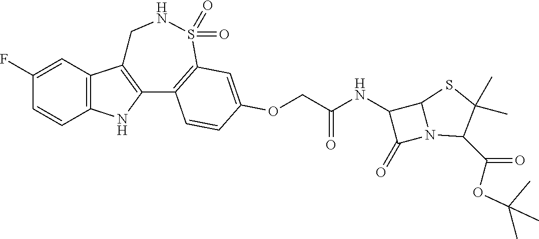 [SMILES: CC(C)(C)OC(=O)C1N2C(=O)C(NC(=O)COc3ccc4c(c3)S(=O)(=O)NCc3c-4[nH]c4ccc(F)cc34)C2SC1(C)C] |
| XIVc | RA43 | 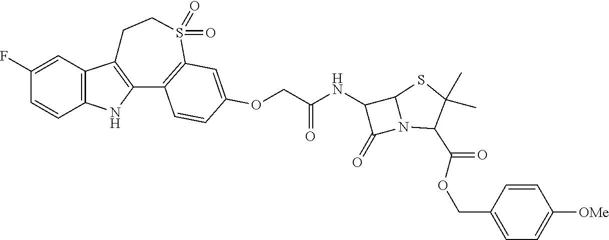 [SMILES: COc1ccc(COC(=O)C2N3C(=O)C(NC(=O)COc4ccc5c(c4)S(=O)(=O)CCc4c-5[nH]c5ccc(F)cc45)C3SC2(C)C)cc1] |
| XIIIr | RA44 | 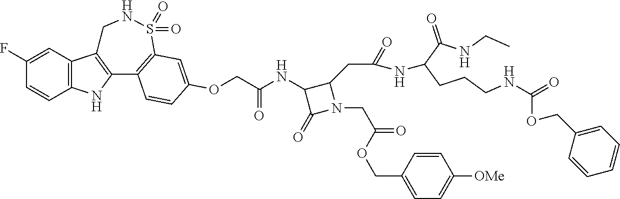 [SMILES: CCNC(=O)C(CCCNC(=O)OCc1ccccc1)NC(=O)CC1C(NC(=O)COc2ccc3c(c2)S(=O)(=O)NCc2c-3[nH]c3ccc(F)cc23)C(=O)N1CC(=O)OCc1ccc(OC)cc1] |

17. Compounds according to claim 7, wherein the compounds are selected from the group consisting of:

| # | # | |
|---|---|---|
| substructure | Abbreviation | Structure |
| XIc XII | RA1 | 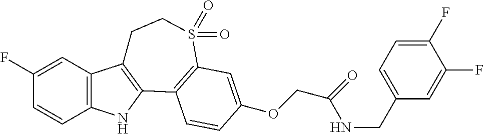 [SMILES: O=C(COc1ccc2c(c1)S(=O)(=O)CCc1c-2[nH]c2ccc(F)cc12)NCc1ccc(F)c(F)c1] |
| XIa | RA2 | 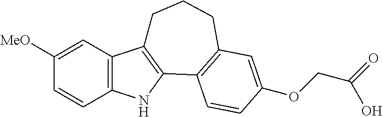 [SMILES: COc1ccc2[nH]c3c(c2c1)CCCc1cc(OCC(=O)O)ccc1-3] |
| XIr | RA3 | 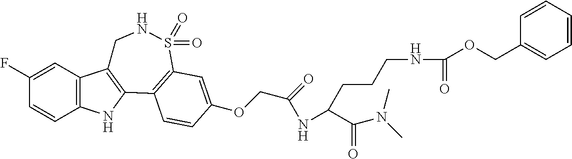 [SMILES: CN(C)C(=O)C(CCCNC(=O)OCc1ccccc1)NC(=O)COc1ccc2c(c1)S(=O)(=O)NCc1c-2[nH]c2ccc(F)cc12] |
| Xa | GA17M8 | 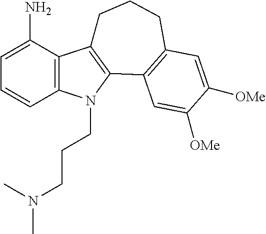 [SMILES: COc1cc2c(cc1OC)-c1c(c3c(N)cccc3n1CCCN(C)C)CCC2] |
| Xa | GA17M10 | 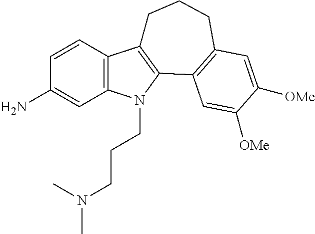 [SMILES: COc1cc2c(cc1OC)-c1c(c3ccc(N)cc3n1CCCN(C)C)CCC2] |
| Xlb | GA11S | 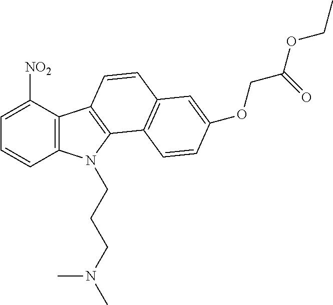 [SMILES: CCOC(=O)COc1ccc2c(ccc3c4c([N+](=O)[O-])cccc4n(CCCN(C)C)c23)c1] |
| Xlb | RM66 | 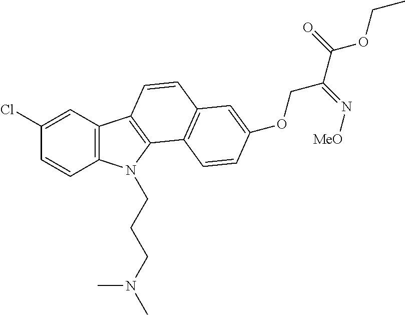 [SMILES: CCOC(=O)/C(COc1ccc2c(ccc3c4cc(Cl)ccc4n(CCCN(C)C)c23)c1)=N/OC] |
| Xlb | RM58 | 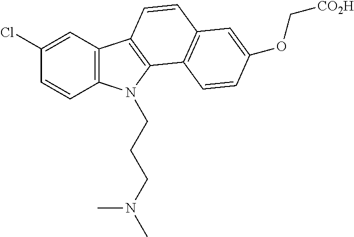 [SMILES: CN(C)CCCn1c2ccc(Cl)cc2c2ccc3cc(OCC(=O)O)ccc3c21] |
| Xlb | RM70 | 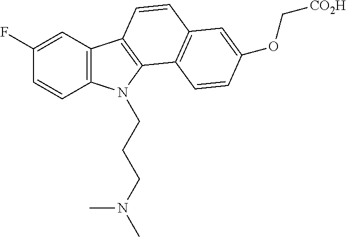 [SMILES: CN(C)CCCn1c2ccc(F)cc2c2ccc3cc(OCC(=O)O)ccc3c21] |
| Xlb | RM85 | 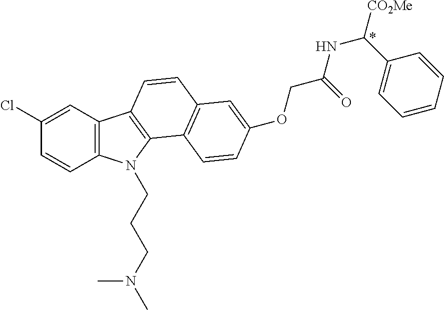 [SMILES: COC(=O)C(NC(=O)COc1ccc2c(ccc3c4cc(Cl)ccc4n(CCCN(C)C)c23)c1)c1ccccc1] |
| XI′d | RA6 | 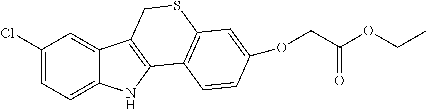 [SMILES: CCOC(=O)COc1ccc2c(c1)SCc1c-2[nH]c2ccc(Cl)cc12] |
| XI′d | RA7 | 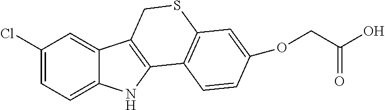 [SMILES: O=C(O)COc1ccc2c(c1)SCc1c-2[nH]c2ccc(Cl)cc12] |
| XI′d | RA9 | 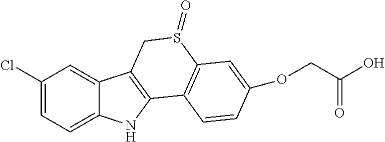 [SMILES: O=C(O)COc1ccc2c(c1)S(=O)Cc1c-2[nH]c2ccc(Cl)cc12] |
| XI′d | RA11 | 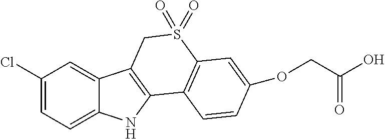 [SMILES: O=C(O)COc1ccc2c(c1)S(=O)(=O)Cc1c-2[nH]c2ccc(Cl)cc12] |
| XI′d | RA12 | 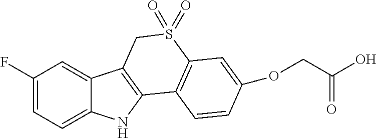 [SMILES: O=C(O)COc1ccc2c(c1)S(=O)(=O)Cc1c-2[nH]c2ccc(F)cc12] |
| XI′d | RA13 | 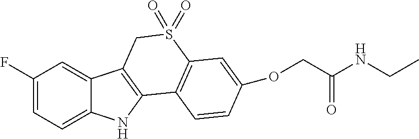 [SMILES: CCNC(=O)COc1ccc2c(c1)S(=O)(=O)Cc1c-2[nH]c2ccc(F)cc12] |
| XI′g | RA14 | 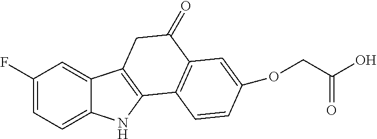 [SMILES: O=C(O)COc1ccc2c(c1)C(=O)Cc1c-2[nH]c2ccc(F)cc12] |
| X′c | RA15 | 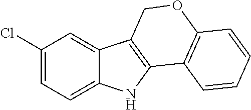 [SMILES: Clc1ccc2[nH]c3c(c2c1)COc1ccccc1-3] |
| X′c | RA16 | 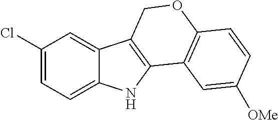 [SMILES: COc1ccc2c(c1)-c1[nH]c3ccc(Cl)cc3c1CO2] |
| XI′c | RA17 | 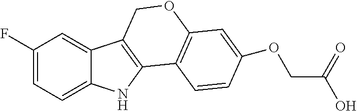 [SMILES: O=C(O)COc1ccc2c(c1)OCc1c-2[nH]c2ccc(F)cc12] |
| XI′C | RA18 | 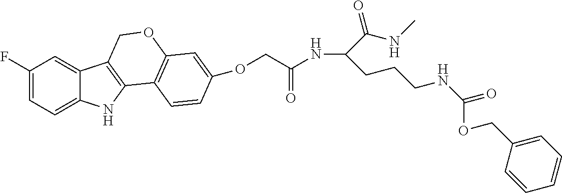 [SMILES: CNC(=O)C(CCCNC(=O)OCc1ccccc1)NC(=O)COc1ccc2c(c1)OCc1c-2[nH]c2ccc(F)cc12] |
| XI′C | RA19 | 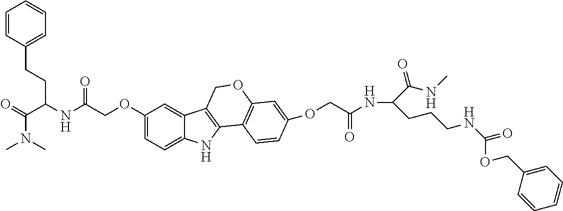 [SMILES: CNC(=O)C(CCCNC(=O)OCc1ccccc1)NC(=O)COc1ccc2c(c1)OCc1c-2[nH]c2ccc(OCC(=O)NC(CCc3ccccc3)C(=O)N(C)C)cc12] |
| XI′h | RA20 | 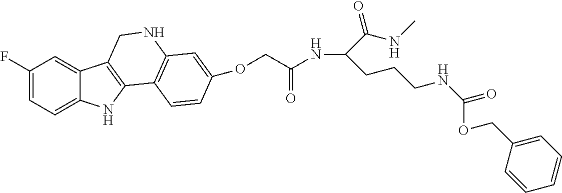 [SMILES: CNC(=O)C(CCCNC(=O)OCc1ccccc1)NC(=O)COc1ccc2c(c1)NCc1c-2[nH]c2ccc(F)cc12] |
| X′e | RA21 | 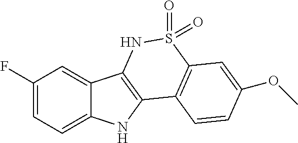 [SMILES: COc1ccc2c(c1)S(=O)(=O)Nc1c-2[nH]c2ccc(F)cc12] |
| X′e | RA22 | 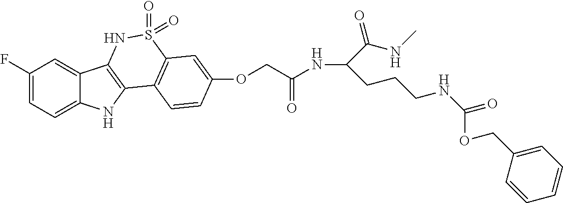 [SMILES: CNC(=O)C(CCCNC(=O)OCc1ccccc1)NC(=O)COc1ccc2c(c1)S(=O)(=O)Nc1c-2[nH]c2ccc(F)cc12] |
| XIV′a | GA09a | 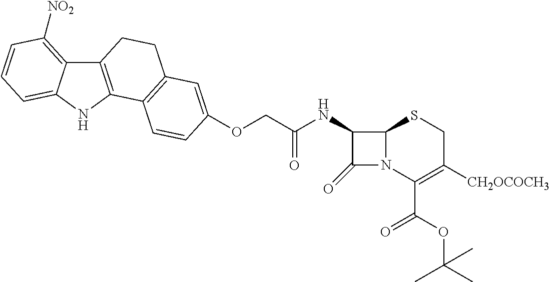 |
| XIV′a′ | GA09a′ | 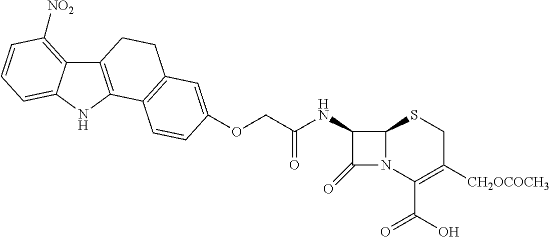 |
| XIV′a | GA09b | 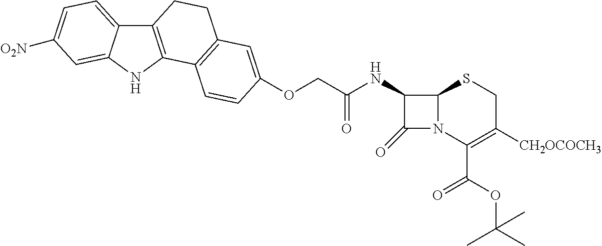 |
| XIV′a′ | GA09b′ | 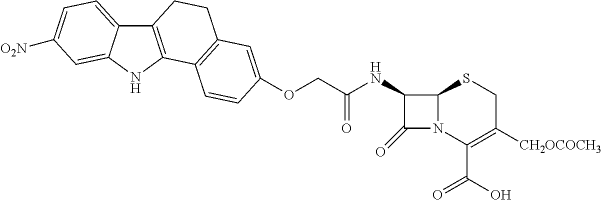 |
| XIV′a | RM36 | 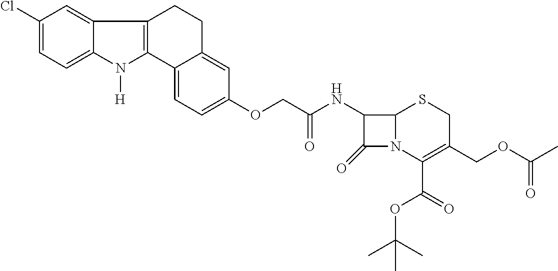 [SMILES: CC(=O)OCC1=C(C(=O)OC(C)(C)C)N2C(=O)C(NC(=O)COc3ccc4c(c3)CCc3c-4[nH]c4ccc(Cl)cc34)C2SC1] |
| XIV′a | RM37 | 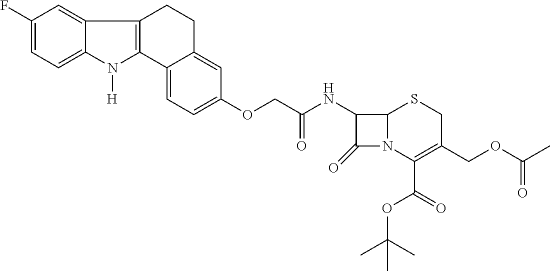 [SMILES: CC(=O)OCC1=C(C(=O)OC(C)(C)C)N2C(=O)C(NC(=O)COc3ccc4c(c3)CCc3c-4[nH]c4ccc(F)cc34)C2SC1] |
| XIV′a′ | RM53 | 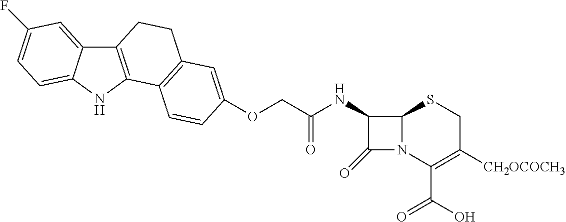 |
| XIV′a | RM47 | 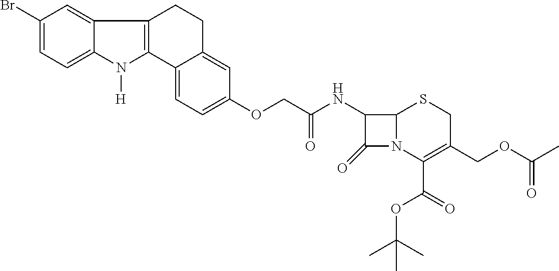 [SMILES: CC(=O)OCC1=C(C(=O)OC(C)(C)C)N2C(=O)C(NC(=O)COc3ccc4c(c3)CCc3c-4[nH]c4ccc(Br)cc34)C2SC1] |
| XIV′d | RA23 | 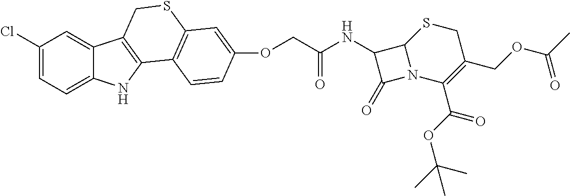 [SMILES: CC(=O)OCC1=C(C(=O)OC(C)(C)C)N2C(=O)C(NC(=O)COc3ccc4c(c3)SCc3c-4[nH]c4ccc(Cl)cc34)C2SC1] |
| XIV′d | RA24 | 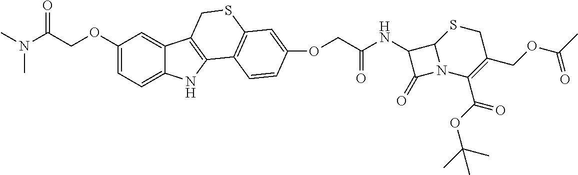 [SMILES: CC(=O)OCC1=C(C(=O)OC(C)(C)C)N2C(=O)C(NC(=O)COc3ccc4c(c3)SCc3c-4[nH]c4ccc(OCC(=O)N(C)C)cc34)C2SC1] |
| XIV′d | RA25 | 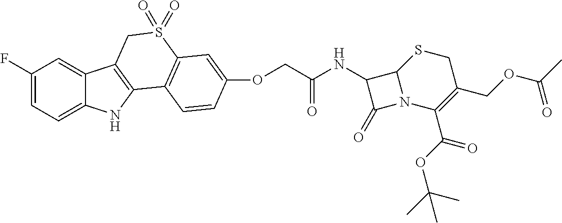 [SMILES: CC(=O)OCC1=C(C(=O)OC(C)(C)C)N2C(=O)C(NC(=O)COc3ccc4c(c3)S(=O)(=O)Cc3c-4[nH]c4ccc(F)cc34)C2SC1] |
| XV′e | RA26 | 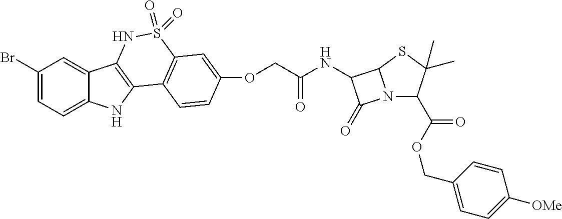 [SMILES: COc1ccc(COC(=O)C2N3C(=O)C(NC(=O)COc4ccc5c(c4)S(=O)(=O)Nc4c-5[nH]c5ccc(Br)cc45)C3SC2(C)C)cc1] |
| XV′d | RA27 | 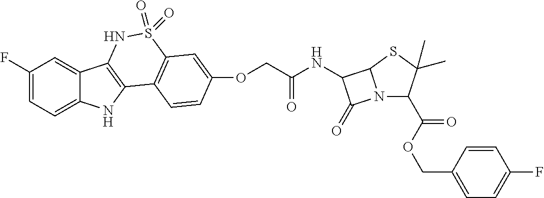 [SMILES: CC1(C)SC2C(NC(=O)COc3ccc4c(c3)S(=O)(=O)Nc3c-4[nH]c4ccc(F)cc34)C(=O)N2C1C(=O)OCc1ccc(F)cc1] |
| XV′d | RA28 | 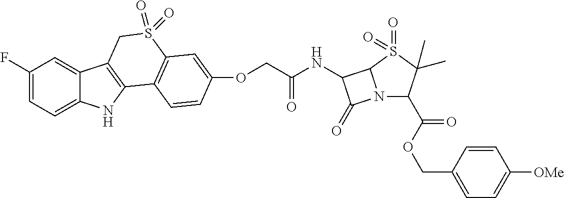 [SMILES: COc1ccc(COC(=O)C2N3C(=O)C(NC(=O)COc4ccc5c(c4)S(=O)(=O)Cc4c-5[nH]c5ccc(F)cc45)C3S(=O)(=O)C2(C)C)cc1] |
| XV′g′ | RA29 | 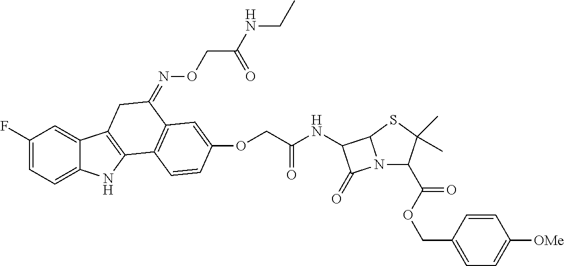 [SMILES: CCNC(=O)CO/N=C1/Cc2c([nH]c3ccc(F)cc23)-c2ccc(OCC(=O)NC3C(=O)N4C3SC(C)(C)C4C(=O)OCc3ccc(OC)cc3)cc21] |
| XIIId | RA30 | 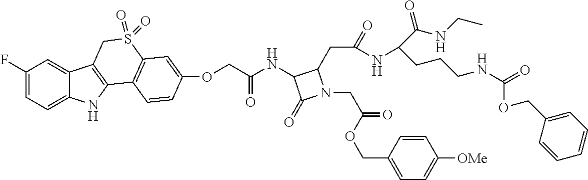 [SMILES: CCNC(=O)C(CCCNC(=O)OCc1ccccc1)NC(=O)CC1C(NC(=O)COc2ccc3c(c2)S(=O)(=O)Cc2c-3[nH]c3ccc(F)cc23)C(=O)N1CC(=O)OCc1ccc(OC)cc1] |
| XIV′c | RA31 | 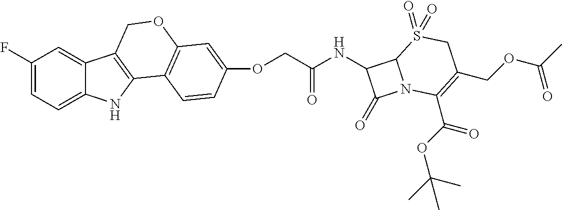 [SMILES: CC(=O)OCC1=C(C(=O)OC(C)(C)C)N2C(=O)C(NC(=O)COc3ccc4c(c3)OCc3c-4[nH]c4ccc(F)cc34)C2S(=O)(=O)C1] |
| XIV′c | RA32 | 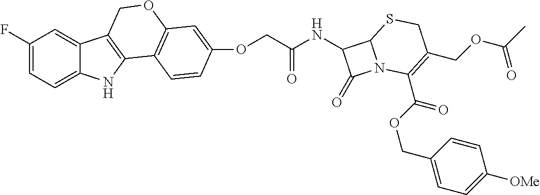 [SMILES: COc1ccc(COC(=O)C2=C(COC(C)=O)CSC3C(NC(=O)COc4ccc5c(c4)OCc4c-5[nH]c5ccc(F)cc45)C(=O)N23)cc1] |
| XIV′c | RA33 | 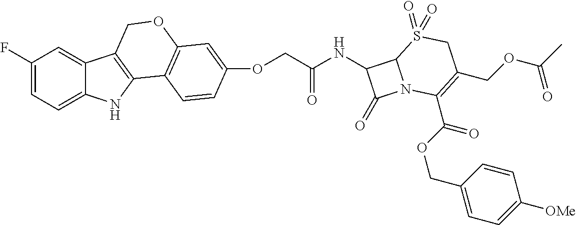 [SMILES: COc1ccc(COC(=O)C2=C(COC(C)=O)CS(=O)(=O)C3C(NC(=O)COc4ccc5c(c4)OCc4c-5[nH]c5ccc(F)cc45)C(=O)N23)cc1] |
| XIII′c | RA34 | 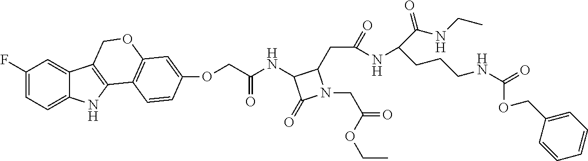 [SMILES: CCNC(=O)C(CCCNC(=O)OCc1ccccc1)NC(=O)CC1C(NC(=O)COc2ccc3c(c2)OCc2c-3[nH]c3ccc(F)cc23)C(=O)N1CC(=O)OCC] |
| XV′c | RA35 | 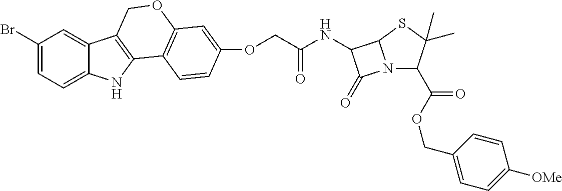 [SMILES: COc1ccc(COC(=O)C2N3C(=O)C(NC(=O)COc4ccc5c(c4)OCc4c-5[nH]c5ccc(Br)cc45)C3SC2(C)C)cc1] |
| XIV′e | RA36 | 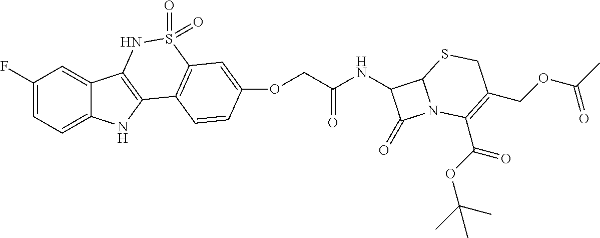 [SMILES: CC(=O)OCC1=C(C(=O)OC(C)(C)C)N2C(=O)C(NC(=O)COc3ccc4c(c3)S(=O)(=O)Nc3c-4[nH]c4ccc(F)cc34)C2SC1] |
| XIV′g′ | RA37 | 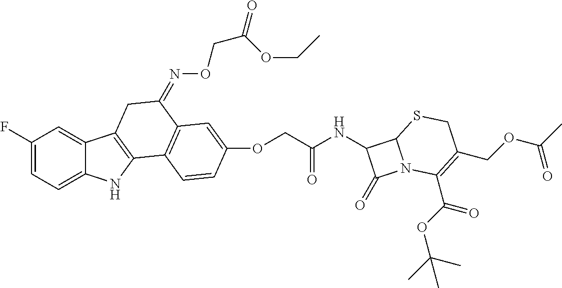 [SMILES: CCOC(=O)CO/N=C1/Cc2c([nH]c3ccc(F)cc23)-c2ccc(OCC(=O)NC3C(=O)N4C(C(=O)OC(C)(C)C)=C(COC(C)=O)CSC34)cc21] |
| XIV′g′ | RA38 | 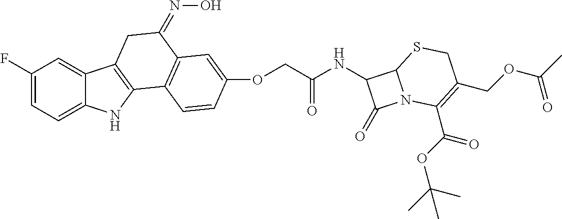 [SMILES: CC(=O)OCC1=C(C(=O)OC(C)(C)C)N2C(=O)C(NC(=O)COc3ccc4c(c3)/C(=N\O)Cc3c-4[nH]c4ccc(F)cc34)C2SC1] |
| XIV′g | RA39 | 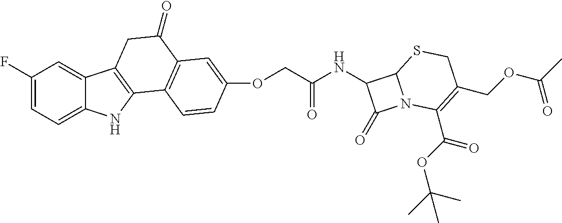 [SMILES: CC(=O)OCC1=C(C(=O)OC(C)(C)C)N2C(=O)C(NC(=O)COc3ccc4c(c3)C(=O)Cc3c-4[nH]c4ccc(F)cc34)C2SC1] |
| XIVr | RA40 | 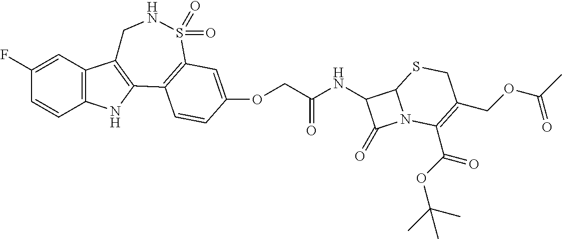 [SMILES: CC(=O)OCC1=C(C(=O)OC(C)(C)C)N2C(=O)C(NC(=O)COc3ccc4c(c3)S(=O)(=O)NCc3c-4[nH]c4ccc(F)cc34)C2SC1] |
| XIVc | RA41 | 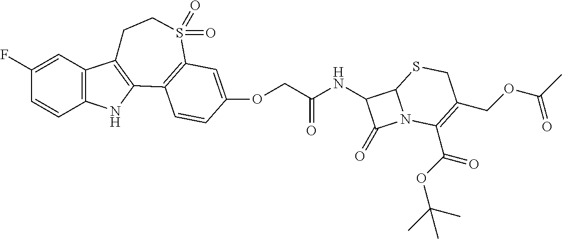 [SMILES: CC(=O)OCC1=C(C(=O)OC(C)(C)C)N2C(=O)C(NC(=O)COc3ccc4c(c3)S(=O)(=O)CCc3c-4[nH]c4ccc(F)cc34)C2SC1] |
| XVr | RA42 | 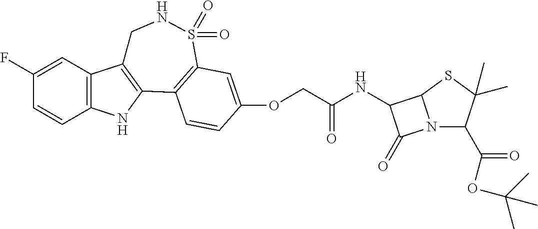 [SMILES: CC(C)(C)OC(=O)C1N2C(=O)C(NC(=O)COc3ccc4c(c3)S(=O)(=O)NCc3c-4[nH]c4ccc(F)cc34)C2SC1(C)C] |
| XIVc | RA43 | 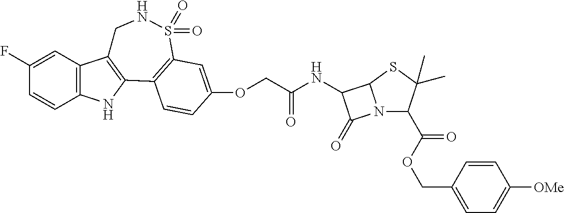 [SMILES: COc1ccc(COC(=O)C2N3C(=O)C(NC(=O)COc4ccc5c(c4)S(=O)(=O)NCc4c-5[nH]c5ccc(F)cc45)C3SC2(C)C)cc1] |
| XIIIr | RA44 | 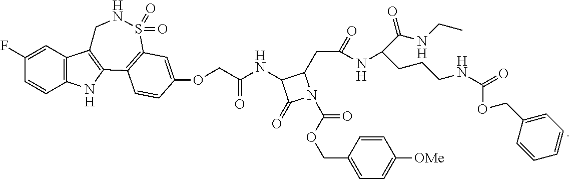 [SMILES: CCNC(=O)C(CCCNC(=O)OCc1ccccc1)NC(=O)CC1C(NC(=O)COc2ccc3c(c2)S(=O)(=O)NCc2c-3[nH]c3ccc(F)cc23)C(=O)N1C(=O)OCc1ccc(OC)cc1] |

18. Compounds according to claim 8, wherein the compounds are selected from the group consisting of:

| # | # | |
|---|---|---|
| substructure | Abbreviation | Structure |
| XIc XII | RA1 | 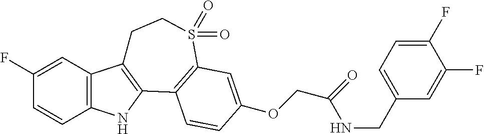 [SMILES: O=C(COc1ccc2c(c1)S(=O)(=O)CCc1c-2[nH]c2ccc(F)cc12)NCc1ccc(F)c(F)c1] |
| XIa | RA2 | 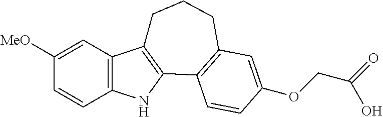 [SMILES: COc1ccc2[nH]c3c(c2c1)CCCc1cc(OCC(=O)O)ccc1-3] |
| XIr | RA3 | 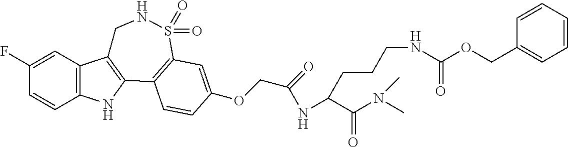 [SMILES: CN(C)C(=O)C(CCCNC(=O)OCc1ccccc1)NC(=O)COc1ccc2c(c1)S(=O)(=O)NCc1c-2[nH]c2ccc(F)cc12] |
| Xa | GA17M8 | 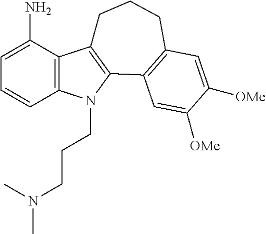 [SMILES: COc1cc2c(cc1OC)-c1c(c3c(N)cccc3n1CCCN(C)C)CCC2] |
| Xa | GA17M10 | 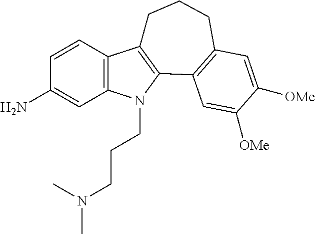 [SMILES: COc1cc2c(cc1OC)-c1c(c3ccc(N)cc3n1CCCN(C)C)CCC2] |
| XIb | GA11S | 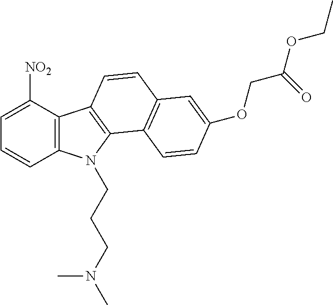 [SMILES: CCOC(=O)COc1ccc2c(ccc3c4c([N+](=O)[O-])cccc4n(CCCN(C)C)c23)c1] |
| XIb | RM66 | 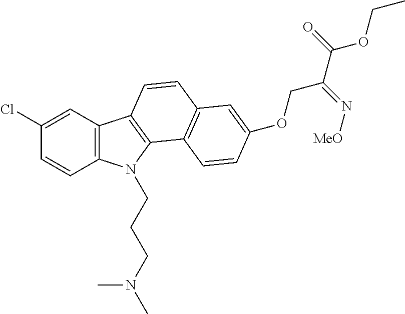 [SMILES: CCOC(=O)/C(COc1ccc2c(ccc3c4cc(Cl)ccc4n(CCCN(C)C)c23)c1)=N/OC] |
| XIb | RM58 | 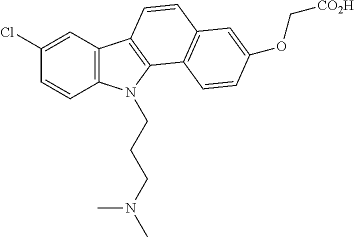 [SMILES: CN(C)CCCn1c2ccc(Cl)cc2c2ccc3cc(OCC(=O)O)ccc3c21] |
| XIb | RM70 | 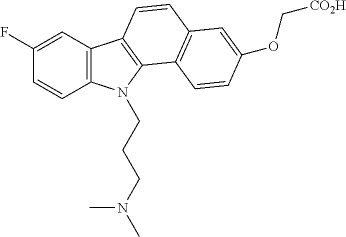 [SMILES: CN(C)CCCn1c2ccc(F)cc2c2ccc3cc(OCC(=O)O)ccc3c21] |
| XIb | RM85 | 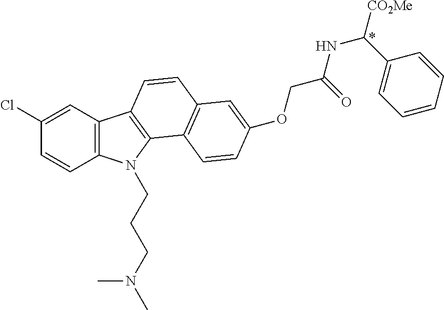 [SMILES: COC(=O)C(NC(=O)COc1ccc2c(ccc3c4cc(Cl)ccc4n(CCCN(C)C)c23)c1)c1ccccc1] |
| XI′d | RA6 | 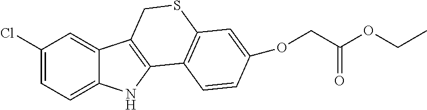 [SMILES: CCOC(=O)COc1ccc2c(c1)SCc1c-2[nH]c2ccc(Cl)cc12] |
| XI′d | RA7 | 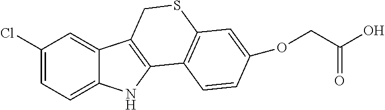 [SMILES: O=C(O)COc1ccc2c(c1)SCc1c-2[nH]c2ccc(Cl)cc12] |
| XI′d | RA9 | 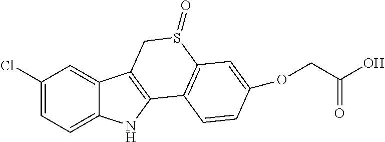 [SMILES: O=C(O)COc1ccc2c(c1)S(=O)Cc1c-2[nH]c2ccc(Cl)cc12] |
| XI′d | RA11 | 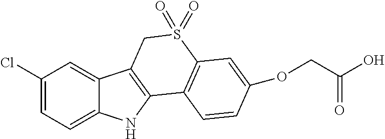 [SMILES: O=C(O)COc1ccc2c(c1)S(=O)(=O)Cc1c-2[nH]c2ccc(Cl)cc12] |
| XI′d | RA12 | 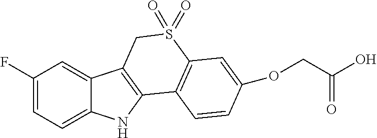 [SMILES: O=C(O)COc1ccc2c(c1)S(=O)(=O)Cc1c-2[nH]c2ccc(F)cc12] |
| XI′d | RA13 | 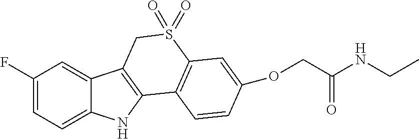 [SMILES: CCNC(=O)COc1ccc2c(c1)S(=O)(=O)Cc1c-2[nH]c2ccc(F)cc12] |
| XI′g | RA14 | 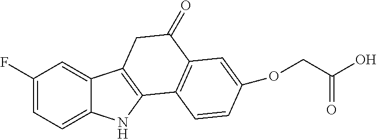 [SMILES: O=C(O)COc1ccc2c(c1)C(=O)Cc1c-2[nH]c2ccc(F)cc12] |
| X′c | RA15 | 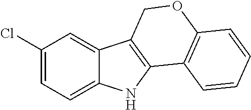 [SMILES: Clc1ccc2[nH]c3c(c2c1)COc1ccccc1-3] |
| X′c | RA16 | 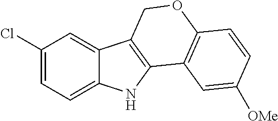 [SMILES: COc1ccc2c(c1)-c1[nH]c3ccc(Cl)cc3c1CO2] |
| XI′c | RA17 | 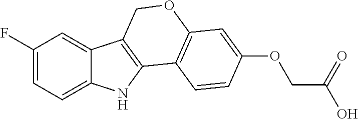 [SMILES: O=C(O)COc1ccc2c(c1)OCc1c-2[nH]c2ccc(F)cc12] |
| XI′c | RA18 | 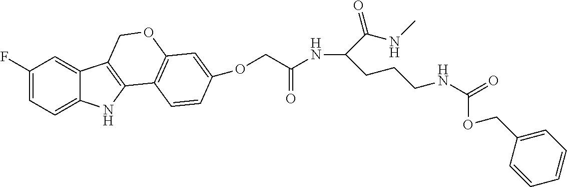 [SMILES: CNC(=O)C(CCCNC(=O)OCc1ccccc1)NC(=O)COc1ccc2c(c1)OCc1c-2[nH]c2ccc(F)cc12] |
| XI′c | RA19 | 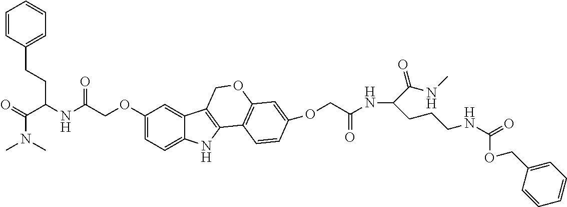 [SMILES: CNC(=O)C(CCCNC(=O)OCc1ccccc1)NC(=O)COc1ccc2c(c1)OCc1c-2[nH]c2ccc(OCC(=O)NC(CCc3ccccc3)C(=O)N(C)C)cc12] |
| XI′h | RA20 | 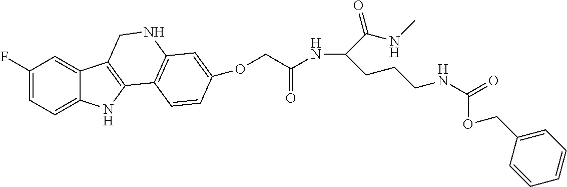 [SMILES: CNC(=O)C(CCCNC(=O)OCc1ccccc1)NC(=O)COc1ccc2c(c1)NCc1c-2[nH]c2ccc(F)cc12] |
| X′e | RA21 | 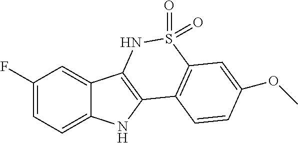 [SMILES: COc1ccc2c(c1)S(=O)(=O)Nc1c-2[nH]c2ccc(F)cc12] |
| XI′e | RA22 | 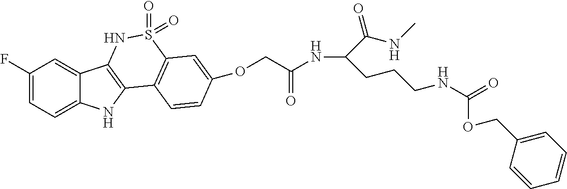 [SMILES: CNC(=O)C(CCCNC(=O)OCc1ccccc1)NC(=O)COc1ccc2c(c1)S(=O)(=O)Nc1c-2[nH]c2ccc(F)cc12] |
| XIV′a | GA09a | 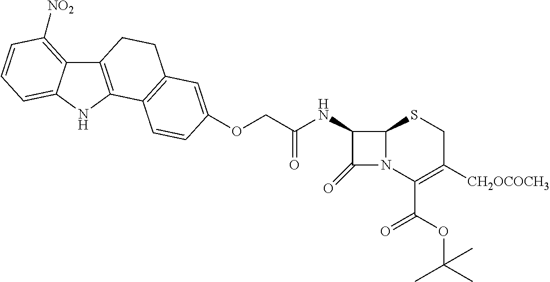 |
| XIV′a′ | GA09a′ | 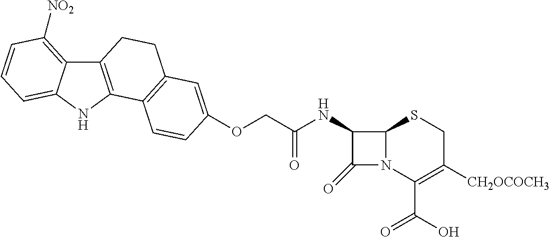 |
| XIV′a | GA09b | 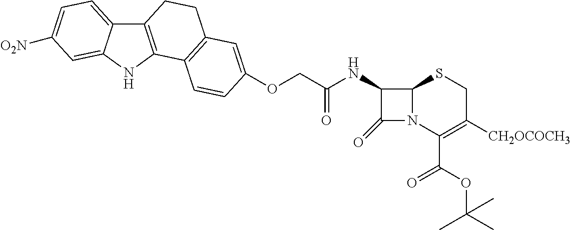 |
| XIV′a′ | GA09b′ | 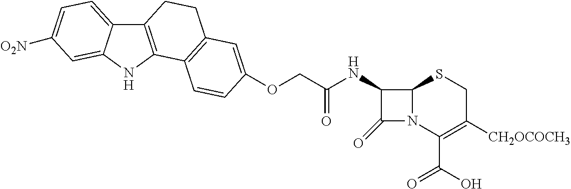 |
| XIV′a | RM36 | 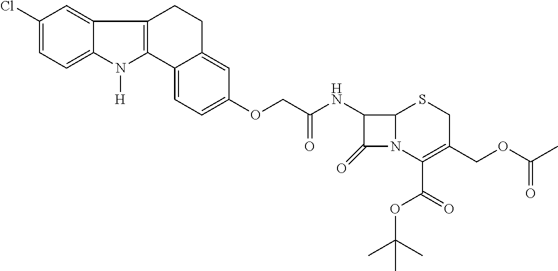 [SMILES: CC(=O)OCC1=C(C(=O)OC(C)(C)C)N2C(=O)C(NC(=O)COc3ccc4c(c3)CCc3c-4[nH]c4ccc(Cl)cc34)C2SC1] |
| XIV′a | RM37 | 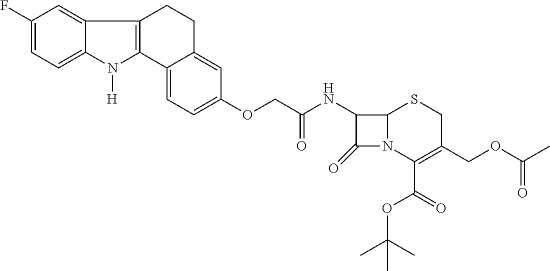 [SMILES: CC(=O)OCC1=C(C(=O)OC(C)(C)C)N2C(=O)C(NC(=O)COc3ccc4c(c3)CCc3c-4[nH]c4ccc(F)cc34)C2SC1] |
| XIV′a′ | RM53 | 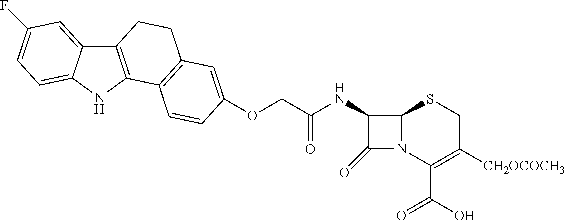 |
| XIV′a | RM47 | 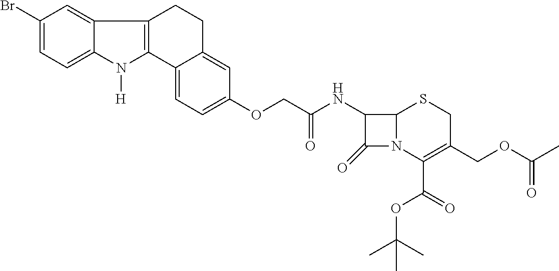 [SMILES: CC(=O)OCC1=C(C(=O)OC(C)(C)C)N2C(=O)C(NC(=O)COc3ccc4c(c3)CCc3c-4[nH]c4ccc(Br)cc34)C2SC1] |
| XIV′d | RA23 | 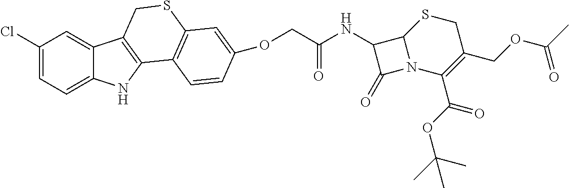 [SMILES: CC(=O)OCC1=C(C(=O)OC(C)(C)C)N2C(=O)C(NC(=O)COc3ccc4c(c3)SCc3c-4[nH]c4ccc(Cl)cc34)C2SC1] |
| XIV′d | RA24 | 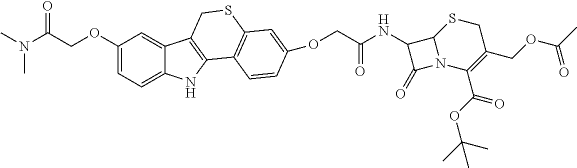 [SMILES: CC(=O)OCC1=C(C(=O)OC(C)(C)C)N2C(=O)C(NC(=O)COc3ccc4c(c3)SCc3c-4[nH]c4ccc(OCC(=O)N(C)C)cc34)C2SC1] |
| XIV′d | RA25 | 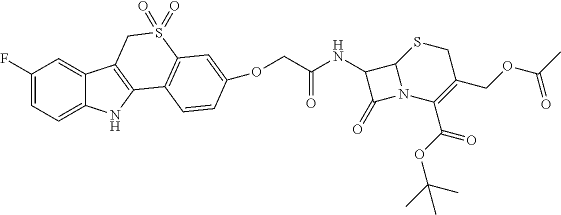 [SMILES: CC(=O)OCC1=C(C(=O)OC(C)(C)C)N2C(=O)C(NC(=O)COc3ccc4c(c3)S(=O)(=O)Cc3c-4[nH]c4ccc(F)cc34)C2SC1] |
| XV′e | RA26 | 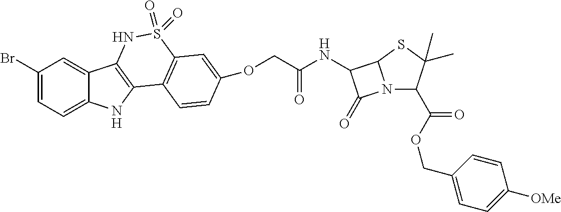 [SMILES: COc1ccc(COC(=O)C2N3C(=O)C(NC(=O)COc4ccc5c(c4)S(=O)(=O)Nc4c-5[nH]c5ccc(Br)cc45)C3SC2(C)C)cc1] |
| XV′d | RA27 | 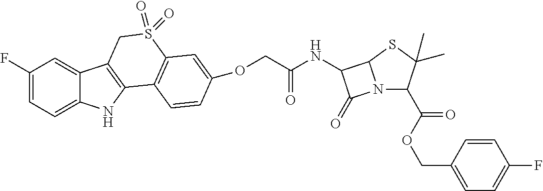 [SMILES: CC1(C)SC2C(NC(=O)COc3ccc4c(c3)S(=O)(=O)Cc3c-4[nH]c4ccc(F)cc34)C(=O)N2C1C(=O)OCc1ccc(F)cc1] |
| XV′d | RA28 | 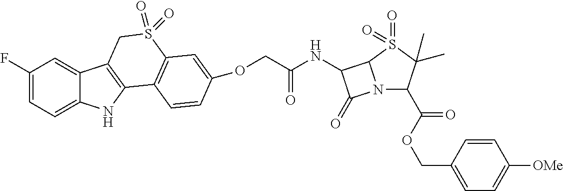 [SMILES: COc1ccc(COC(=O)C2N3C(=O)C(NC(=O)COc4ccc5c(c4)S(=O)(=O)Cc4c-5[nH]c5ccc(F)cc45)C3S(=O)(=O)C2(C)C)cc1] |
| XV′g′ | RA29 | 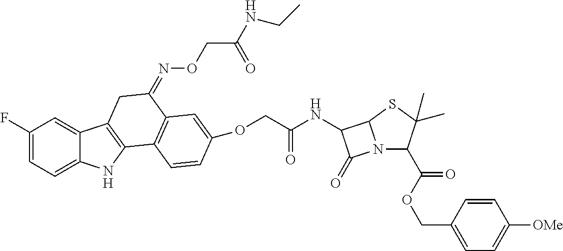 [SMILES: CCNC(=O)CO/N=C1/Cc2c([nH]c3ccc(F)cc23)-c2ccc(OCC(=O)NC3C(=O)N4C3SC(C)(C)C4C(=O)OCc3ccc(OC)cc3)cc21] |
| XIIId | RA30 | 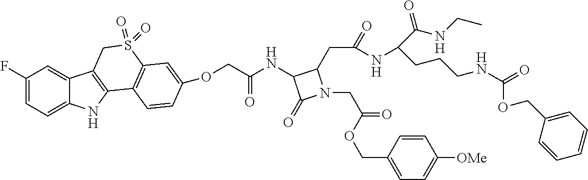 [SMILES: CCNC(=O)C(CCCNC(=O)OCc1ccccc1)NC(=O)CC1C(NC(=O)COc2ccc3c(c2)S(=O)(=O)Cc2c-3[nH]c3ccc(F)cc23)C(=O)N1CC(=O)OCc1ccc(OC)cc1] |
| XIV′c | RA31 | 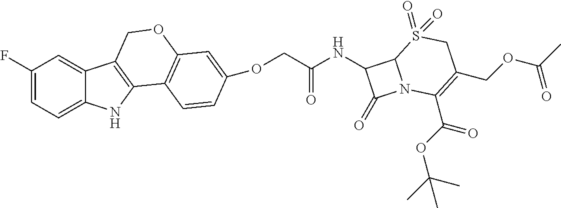 [SMILES: CC(=O)OCC1=C(C(=O)OC(C)(C)C)N2C(=O)C(NC(=O)COc3ccc4c(c3)OCc3c-4[nH]c4ccc(F)cc34)C2S(=O)(=O)C1] |
| XIV′c | RA32 | 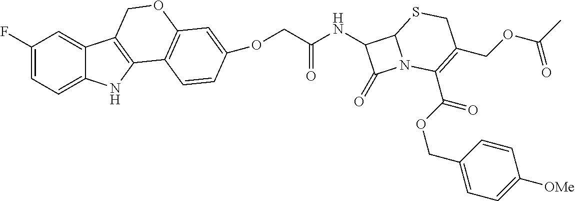 [SMILES: COc1ccc(COC(=O)C2=C(COC(C)=O)CSC3C(NC(=O)COc4ccc5c(c4)OCc4c-5[nH]c5ccc(F)cc45)C(=O)N23)cc1] |
| XIV′c | RA33 | 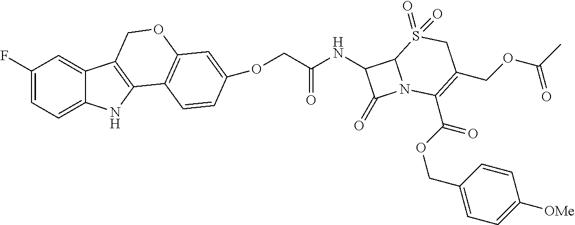 [SMILES: COc1ccc(COC(=O)C2=C(COC(C)=O)CS(=O)(=O)C3C(NC(=O)COc4ccc5c(c4)OCc4c-5[nH]c5ccc(F)cc45)C(=O)N23)cc1] |
| XIII′c | RA34 | 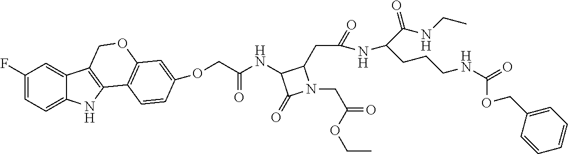 [SMILES: CCNC(=O)C(CCCNC(=O)OCc1ccccc1)NC(=O)CC1C(NC(=O)COc2ccc3c(c2)OCc2c-3[nH]c3ccc(F)cc23)C(=O)N1CC(=O)OCC] |
| XV′c | RA35 | 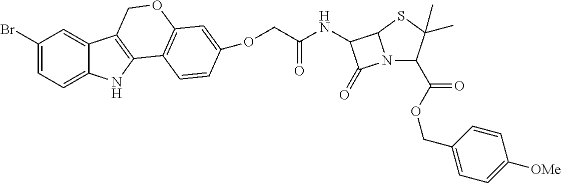 [SMILES: COc1ccc(COC(=O)C2N3C(=O)C(NC(=O)COc4ccc5c(c4)OCc4c-5[nH]c5ccc(Br)cc45)C3SC2(C)C)cc1] |
| XIV′e | RA36 | 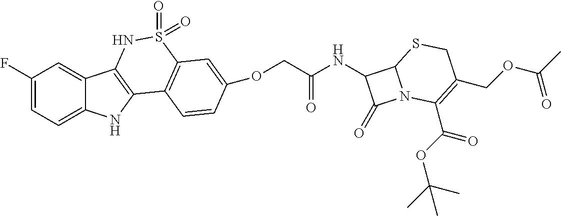 [SMILES: CC(=O)OCC1=C(C(=O)OC(C)(C)C)N2C(=O)C(NC(=O)COc3ccc4c(c3)S(=O)(=O)Nc3c-4[nH]c4ccc(F)cc34)C2SC1] |
| XIV′g′ | RA37 | 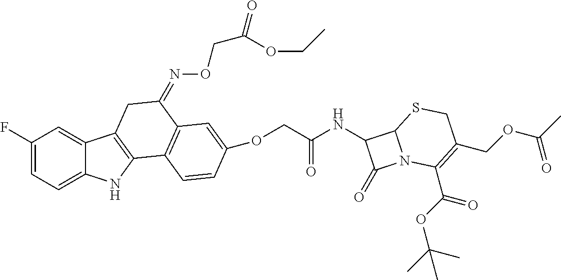 [SMILES: CCOC(=O)CO/N=C1/Cc2c([nH]c3ccc(F)cc23)-c2ccc(OCC(=O)NC3C(=O)N4C(C(=O)OC(C)(C)C)=C(COC(C)=O)CSC34)cc21] |
| XIV′g′ | RA38 | 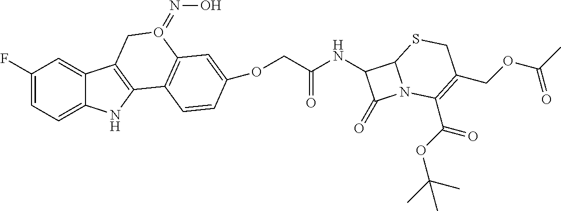 |
| XIV′g | RA39 | 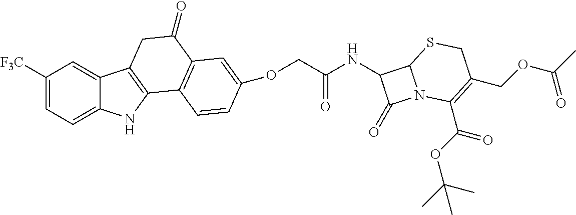 [SMILES: CC(=O)OCC1=C(C(=O)OC(C)(C)C)N2C(=O)C(NC(=O)COc3ccc4c(c3)C(=O)Cc3c-4[nH]c4ccc(C(F)(F)F)cc34)C2SC1] |
| XIVr | RA40 | 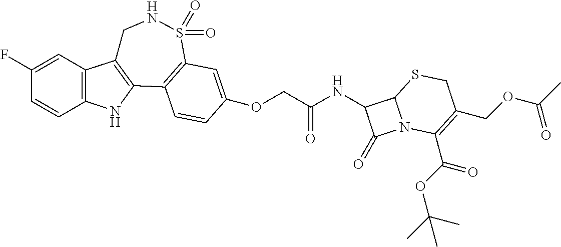 [SMILES: CC(=O)OCC1=C(C(=O)OC(C)(C)C)N2C(=O)C(NC(=O)COc3ccc4c(c3)S(=O)(=O)NCc3c-4[nH]c4ccc(F)cc34)C2SC1] |
| XIVc | RA41 | 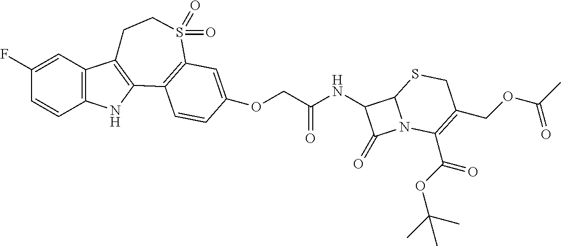 [SMILES: CC(=O)OCC1=C(C(=O)OC(C)(C)C)N2C(=O)C(NC(=O)COc3ccc4c(c3)S(=O)(=O)CCc3c-4[nH]c4ccc(F)cc34)C2SC1] |
| XVr | RA42 | 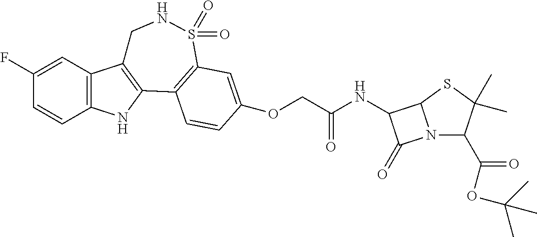 [SMILES: CC(C)(C)OC(=O)C1N2C(=O)C(NC(=O)COc3ccc4c(c3)S(=O)(=O)NCc3c-4[nH]c4ccc(F)cc34)C2SC1(C)C] |
| XIVc | RA43 | 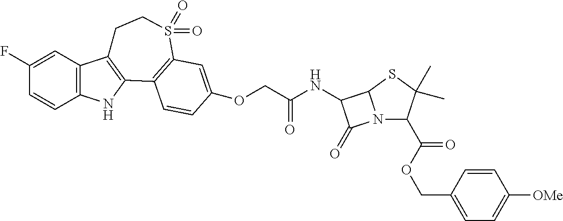 [SMILES: COc1ccc(COC(=O)C2N3C(=O)C(NC(=O)COc4ccc5c(c4)S(=O)(=O)CCc4c-5[nH]c5ccc(F)cc45)C3SC2(C)C)cc1] |
| XIIIr | RA44 | 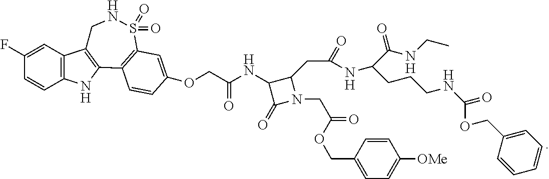 [SMILES: CCNC(=O)C(CCCNC(=O)OCc1ccccc1)NC(=O)CC1C(NC(=O)COc2ccc3c(c2)S(=O)(=O)NCc2c-3[nH]c3ccc(F)cc23)C(=O)N1CC(=O)OCc1ccc(OC)cc1] |

19. Compounds according to claim 9, wherein the compounds are selected from the group consisting of:

| # | # | |
|---|---|---|
| substructure | Abbreviation | Structure |
| XIC XII | RA1 | 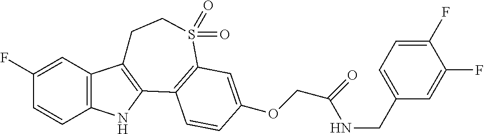 [SMILES: O=C(COc1ccc2c(c1)S(=O)(=O)CCc1c-2[nH]c2ccc(F)cc12)NCc1ccc(F)c(F)c1] |
| XIa | RA2 | 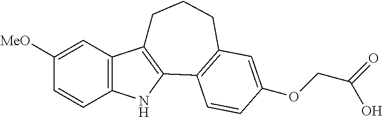 [SMILES: COc1ccc2[nH]c3c(c2c1)CCCc1cc(OCC(=O)O)ccc1-3] |
| XIr | RA3 | 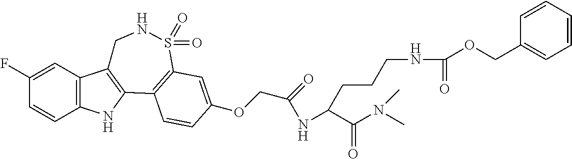 [SMILES: CN(C)C(=O)C(CCCNC(=O)OCc1ccccc1)NC(=O)COc1ccc2c(c1)S(=O)(=O)NCc1c-2[nH]c2ccc(F)cc12] |
| Xa | GA17M8 | 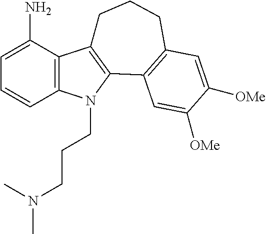 [SMILES: COc1cc2c(cc1OC)-c1c(c3c(N)cccc3n1CCCN(C)C)CCC2] |
| Xa | GA17M10 | 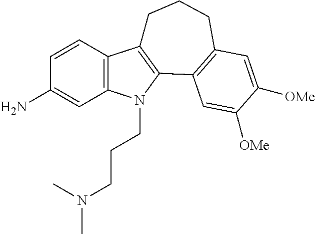 [SMILES: COc1cc2c(cc1OC)-c1c(c3ccc(N)cc3n1CCCN(C)C)CCC2] |
| XIb | GA11S | 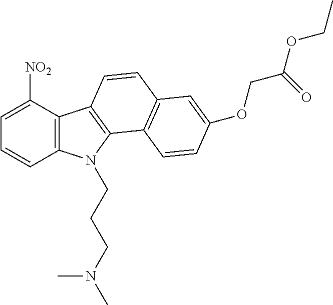 [SMILES: CCOC(=O)COc1ccc2c(ccc3c4c([N+](=O)[O-])cccc4n(CCCN(C)C)c23)c1] |
| XIb | RM66 | 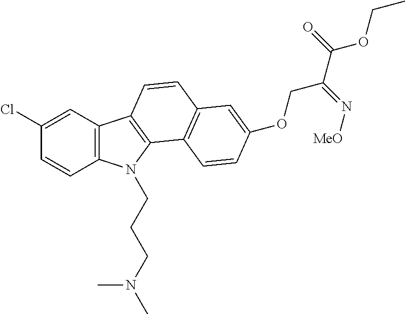 [SMILES: CCOC(=O)/C(COc1ccc2c(ccc3c4cc(Cl)ccc4n(CCCN(C)C)c23)c1)=N/OC] |
| XIb | RM58 | 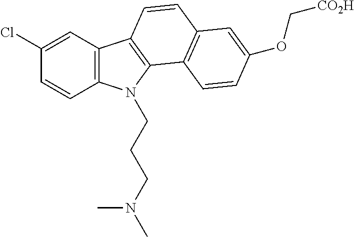 [SMILES: CN(C)CCCn1c2ccc(Cl)cc2c2ccc3cc(OCC(=O)O)ccc3c21] |
| XIb | RM70 | 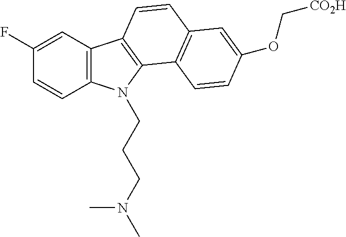 [SMILES: CN(C)CCCn1c2ccc(F)cc2c2ccc3cc(OCC(=O)O)ccc3c21] |
| XIb | RM85 | 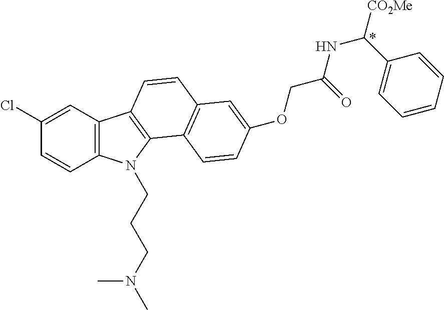 [SMILES: COC(=O)C(NC(=O)COc1ccc2c(ccc3c4cc(Cl)ccc4n(CCCN(C)C)c23)c1)c1ccccc1] |
| XI′d | RA6 | 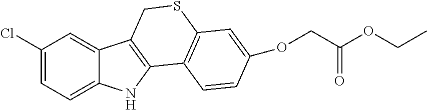 [SMILES: CCOC(=O)COc1ccc2c(c1)SCc1c-2[nH]c2ccc(Cl)cc12] |
| XI′d | RA7 | 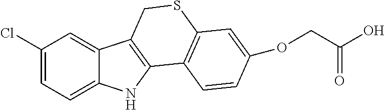 [SMILES: O=C(O)COc1ccc2c(c1)SCc1c-2[nH]c2ccc(Cl)cc12] |
| XI′d | RA9 | 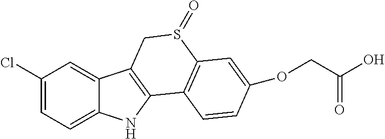 [SMILES: O=C(O)COc1ccc2c(c1)S(=O)Cc1c-2[nH]c2ccc(Cl)cc12] |
| XI′d | RA11 | 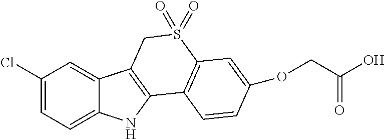 [SMILES: O=C(O)COc1ccc2c(c1)S(=O)(=O)Cc1c-2[nH]c2ccc(Cl)cc12] |
| XI′d | RA12 | 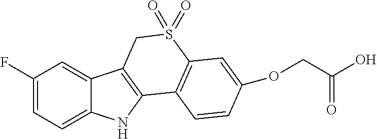 [SMILES: O=C(O)COc1ccc2c(c1)S(=O)(=O)Cc1c-2[nH]c2ccc(F)cc12] |
| XI′d | RA13 | 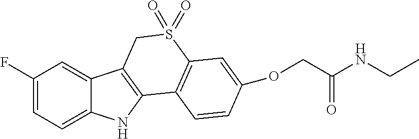 [SMILES: CCNC(=O)COc1ccc2c(c1)S(=O)(=O)Cc1c-2[nH]c2ccc(F)cc12] |
| XI′g | RA14 | 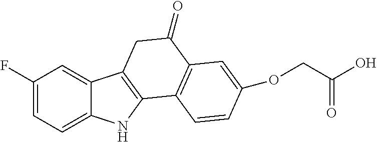 [SMILES: O=C(O)COc1ccc2c(c1)C(=O)Cc1c-2[nH]c2ccc(F)cc12] |
| X′c | RA15 | 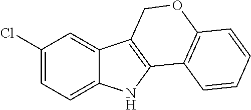 [SMILES: Clc1ccc2[nH]c3c(c2c1)COc1ccccc1-3] |
| X′c | RA16 | 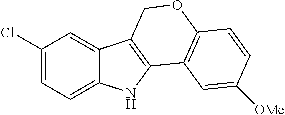 [SMILES: COc1ccc2c(c1)-c1[nH]c3ccc(Cl)cc3c1CO2] |
| XI′c | RA17 | 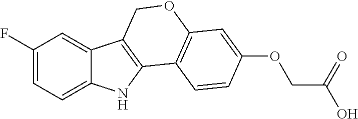 [SMILES: O=C(O)COc1ccc2c(c1)OCc1c-2[nH]c2ccc(F)cc12] |
| XI′c | RA18 | 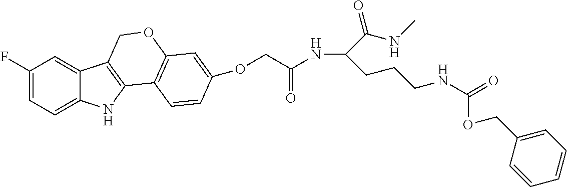 [SMILES: CNC(=O)C(CCCNC(=O)OCc1ccccc1)NC(=O)COc1ccc2c(c1)OCc1c-2[nH]c2ccc(F)cc12] |
| XI′c | RA19 | 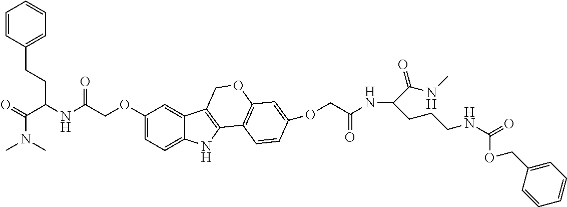 [SMILES: CNC(=O)C(CCCNC(=O)OCc1ccccc1)NC(=O)COc1ccc2c(c1)OCc1c-2[nH]c2ccc(OCC(=O)NC(CCc3ccccc3)C(=O)N(C)C)cc12] |
| XI′h | RA20 | 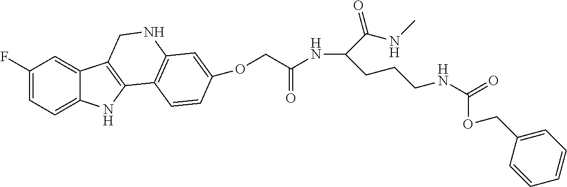 [SMILES: CNC(=O)C(CCCNC(=O)OCc1ccccc1)NC(=O)COc1ccc2c(c1)NCc1c-2[nH]c2ccc(F)cc12] |
| X′e | RA21 | 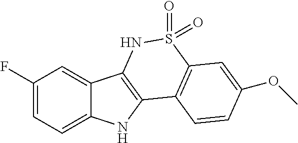 [SMILES: COc1ccc2c(c1)S(=O)(=O)Nc1c-2[nH]c2ccc(F)cc12] |
| XI′e | RA22 | 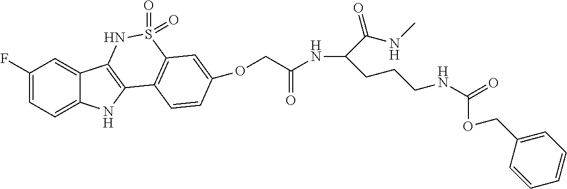 [SMILES: CNC(=O)C(CCCNC(=O)OCc1ccccc1)NC(=O)COc1ccc2c(c1)S(=O)(=O)Nc1c-2[nH]c2ccc(F)cc12] |
| XIV′a | GA09a | 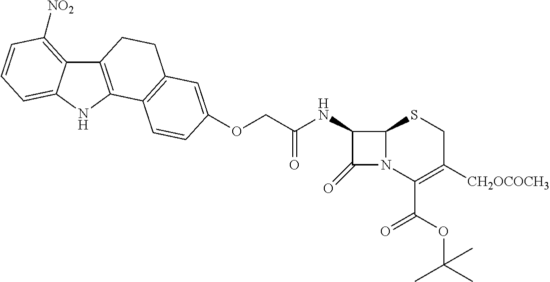 |
| XIV′a′ | GA09a′ | 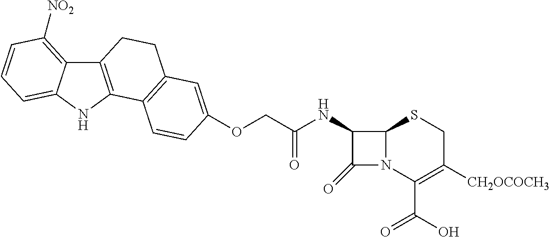 |
| XIV′a | GA09b | 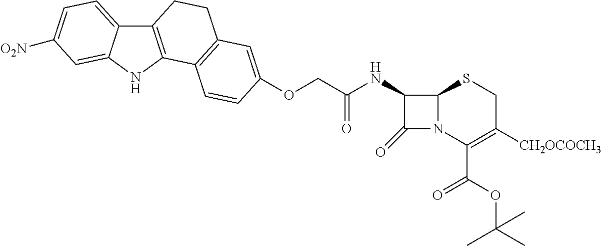 |
| XIV′a′ | GA09b′ | 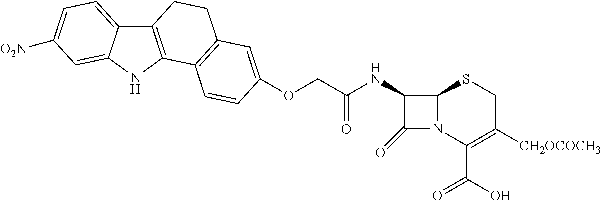 |
| XIV′a | RM36 | 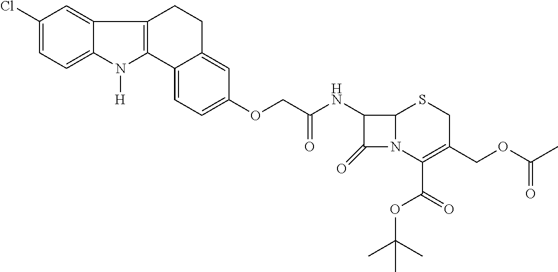 [SMILES: CC(=O)OCC1=C(C(=O)OC(C)(C)C)N2C(=O)C(NC(=O)COc3ccc4c(c3)CCc3c-4[nH]c4ccc(Cl)cc34)C2SC1] |
| XIV′a | RM37 | 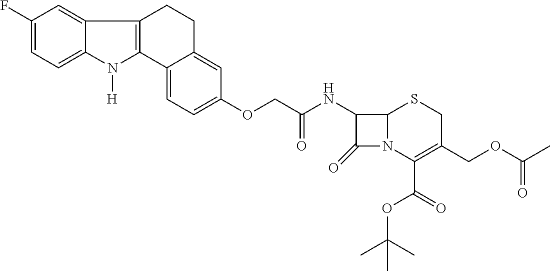 [SMILES: CC(=O)OCC1=C(C(=O)OC(C)(C)C)N2C(=O)C(NC(=O)COc3ccc4c(c3)CCc3c-4[nH]c4ccc(F)cc34)C2SC1] |
| XIV′a′ | RM53 | 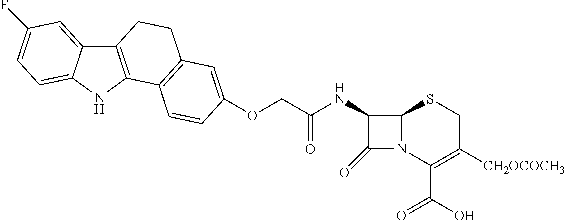 |
| XIV′a | RM47 | 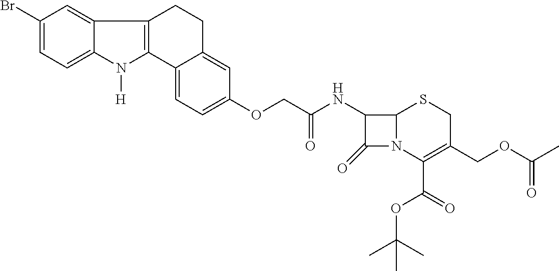 [SMILES: CC(=O)OCC1=C(C(=O)OC(C)(C)C)N2C(=O)C(NC(=O)COc3ccc4c(c3)CCc3c-4[nH]c4ccc(Br)cc34)C2SC1] |
| XIV′d | RA23 | 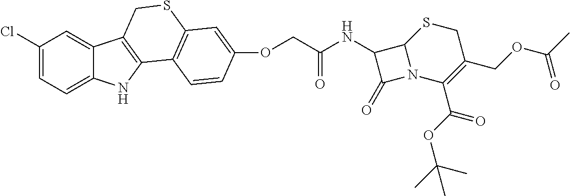 [SMILES: CC(=O)OCC1=C(C(=O)OC(C)(C)C)N2C(=O)C(NC(=O)COc3ccc4c(c3)SCc3c-4[nH]c4ccc(Cl)cc34)C2SC1] |
| XIV′d | RA24 | 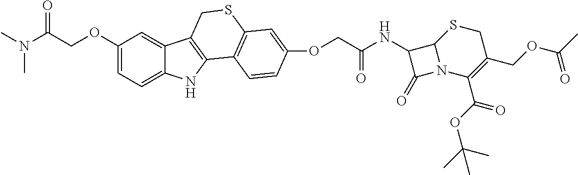 [SMILES: CC(=O)OCC1=C(C(=O)OC(C)(C)C)N2C(=O)C(NC(=O)COc3ccc4c(c3)SCc3c-4[nH]c4ccc(OCC(=O)N(C)C)cc34)C2SC1] |
| XIV′d | RA25 | 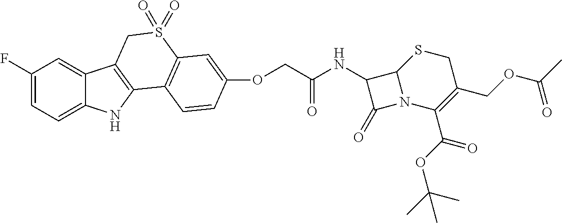 [SMILES: CC(=O)OCC1=C(C(=O)OC(C)(C)C)N2C(=O)C(NC(=O)COc3ccc4c(c3)S(=O)(=O)Cc3c-4[nH]c4ccc(F)cc34)C2SC1] |
| XV′e | RA26 | 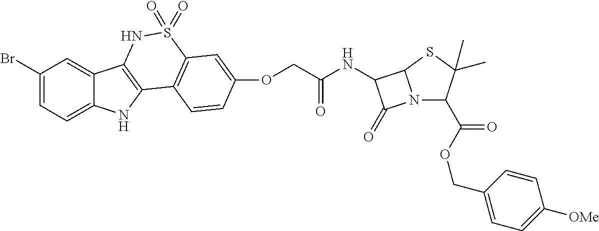 [SMILES: COc1ccc(COC(=O)C2N3C(=O)C(NC(=O)COc4ccc5c(c4)S(=O)(=O)Nc4c-5[nH]c5ccc(Br)cc45)C3SC2(C)C)cc1] |
| XV′d | RA27 | 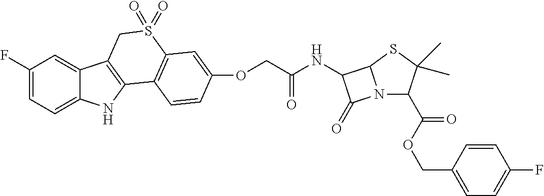 [SMILES: CC1(C)SC2C(NC(=O)COc3ccc4c(c3)S(=O)(=O)Cc3c-4[nH]c4ccc(F)cc34)C(=O)N2C1C(=O)OCc1ccc(F)cc1] |
| XV′d | RA28 | 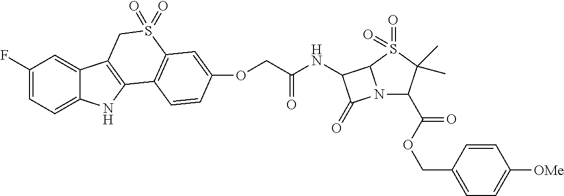 [SMILES: COc1ccc(COC(=O)C2N3C(=O)C(NC(=O)COc4ccc5c(c4)S(=O)(=O)Cc4c-5[nH]c5ccc(F)cc45)C3S(=O)(=O)C2(C)C)cc1] |
| XV′g′ | RA29 | 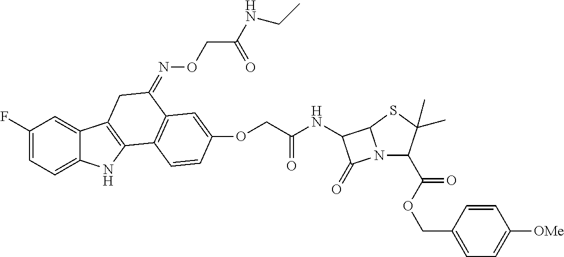 [SMILES: CCNC(=O)CO/N=C1/Cc2c([nH]c3ccc(F)cc23)-c2ccc(OCC(=O)NC3C(=O)N4C3SC(C)(C)C4C(=O)OCc3ccc(OC)cc3)cc21] |
| XIIId | RA30 | 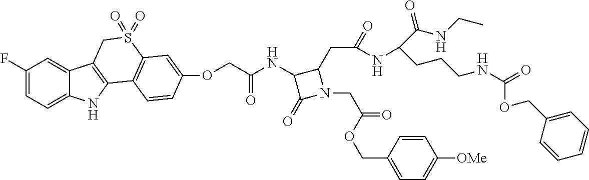 [SMILES: CCNC(=O)C(CCCNC(=O)OCc1ccccc1)NC(=O)CC1C(NC(=O)COc2ccc3c(c2)S(=O)(=O)Cc2c-3[nH]c3ccc(F)cc23)C(=O)N1CC(=O)OCc1ccc(OC)cc1] |
| XIV′c | RA31 | 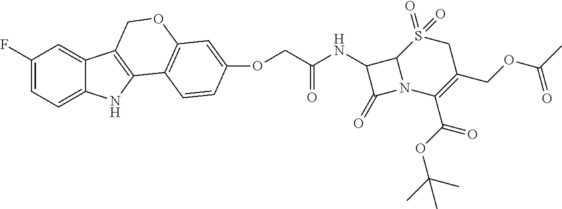 [SMILES: CC(=O)OCC1=C(C(=O)OC(C)(C)C)N2C(=O)C(NC(=O)COc3ccc4c(c3)OCc3c-4[nH]c4ccc(F)cc34)C2S(=O)(=O)C1] |
| XIV′c | RA32 | 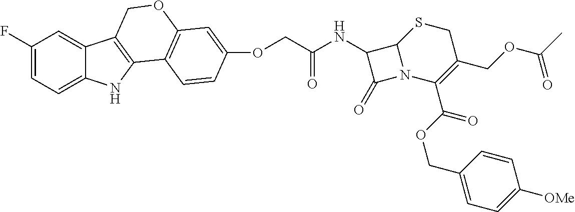 [SMILES: COc1ccc(COC(=O)C2=C(COC(C)=O)CSC3C(NC(=O)COc4ccc5c(c4)OCc4c-5[nH]c5ccc(F)cc45)C(=O)N23)cc1] |
| XIV′c | RA33 | 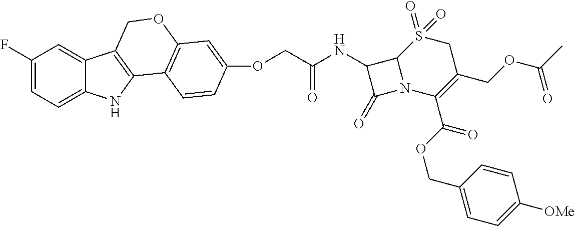 [SMILES: COc1ccc(COC(=O)C2=C(COC(C)=O)CS(=O)(=O)C3C(NC(=O)COc4ccc5c(c4)OCc4c-5[nH]c5ccc(F)cc45)C(=O)N23)cc1] |
| XIII′c | RA34 | 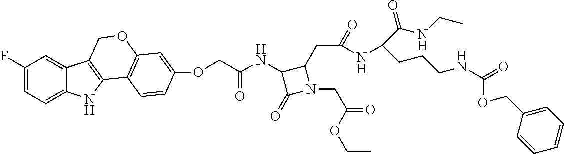 [SMILES: CCNC(=O)C(CCCNC(=O)OCc1ccccc1)NC(=O)CC1C(NC(=O)COc2ccc3c(c2)OCc2c-3[nH]c3ccc(F)cc23)C(=O)N1CC(=O)OCC] |
| XV′c | RA35 | 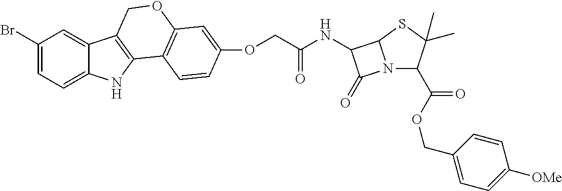 [SMILES: COc1ccc(COC(=O)C2N3C(=O)C(NC(=O)COc4ccc5c(c4)OCc4c-5[nH]c5ccc(Br)cc45)C3SC2(C)C)cc1] |
| XIV′e | RA36 | 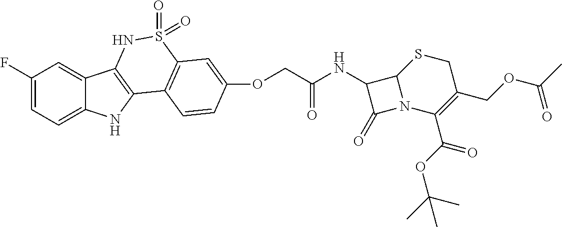 [SMILES: CC(=O)OCC1=C(C(=O)OC(C)(C)C)N2C(=O)C(NC(=O)COc3ccc4c(c3)S(=O)(=O)Nc3c-4[nH]c4ccc(F)cc34)C2SC1] |
| XIV′g′ | RA37 | 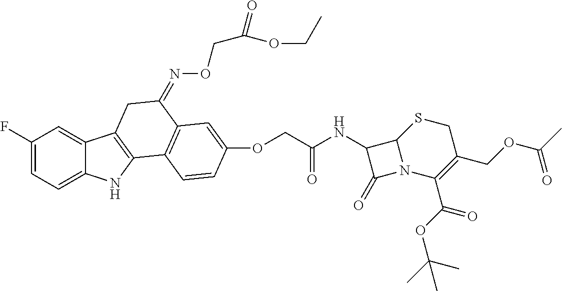 [SMILES: CCOC(=O)CO/N=C1/Cc2c([nH]c3ccc(F)cc23)-c2ccc(OCC(=O)NC3C(=O)N4C(C(=O)OC(C)(C)C)=C(COC(C)=O)CSC34)cc21] |
| XIV′g′ | RA38 | 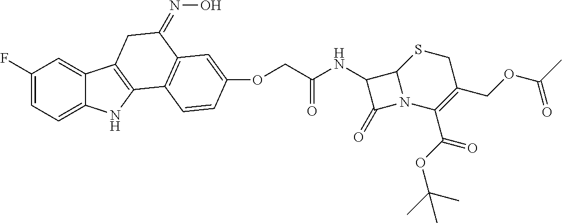 [SMILES: CC(=O)OCC1=C(C(=O)OC(C)(C)C)N2C(=O)C(NC(=O)COc3ccc4c(c3)/C(=N\O)Cc3c-4[nH]c4ccc(F)cc34)C2SC1] |
| XIV′g | RA39 | 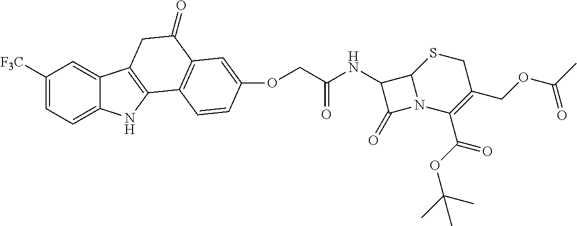 [SMILES: CC(=O)OCC1=C(C(=O)OC(C)(C)C)N2C(=O)C(NC(=O)COc3ccc4c(c3)C(=O)Cc3c-4[nH]c4ccc(C(F)(F)F)cc34)C2SC1] |
| XIVr | RA40 | 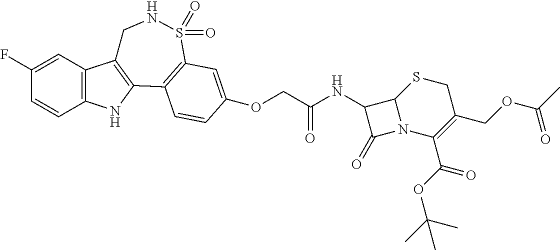 [SMILES: CC(=O)OCC1=C(C(=O)OC(C)(C)C)N2C(=O)C(NC(=O)COc3ccc4c(c3)S(=O)(=O)NCc3c-4[nH]c4ccc(F)cc34)C2SC1] |
| XIVc | RA41 | 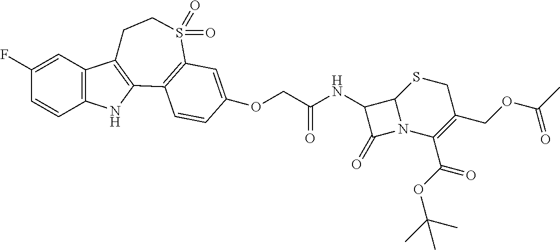 [SMILES: CC(=O)OCC1=C(C(=O)OC(C)(C)C)N2C(=O)C(NC(=O)COc3ccc4c(c3)S(=O)(=O)CCc3c-4[nH]c4ccc(F)cc34)C2SC1] |
| XVr | RA42 | 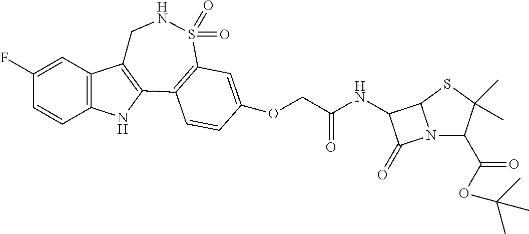 [SMILES: CC(C)(C)OC(=O)C1N2C(=O)C(NC(=O)COc3ccc4c(c3)S(=O)(=O)NCc3c-4[nH]c4ccc(F)cc34)C2SC1(C)C] |
| XIVc | RA43 | 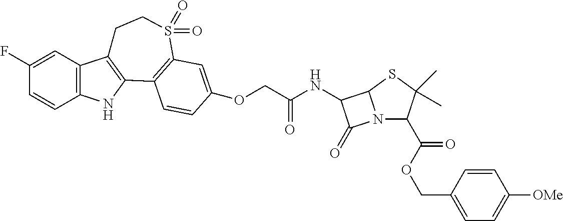 [SMILES: COc1ccc(COC(=O)C2N3C(=O)C(NC(=O)COc4ccc5c(c4)S(=O)(=O)CCc4c-5[nH]c5ccc(F)cc45)C3SC2(C)C)cc1] |
| XIIIr | RA44 | 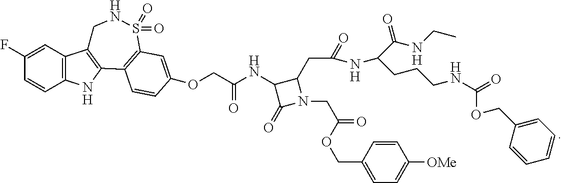 [SMILES: CCNC(=O)C(CCCNC(=O)OCc1ccccc1)NC(=O)CC1C(NC(=O)COc2ccc3c(c2)S(=O)(=O)NCc2c-3[nH]c3ccc(F)cc23)C(=O)N1CC(=O)OCc1ccc(OC)cc1] |

20. A method for treatment or diagnosis of degenerative pathologies characterized by high cell proliferation and/or tissue degeneration, comprising applying an effective amount of the compound of claim 2.
